(19) United States
(12) Patent Application Publication
(10) Pub. No.: US 2025/0026742 A1
(43) Pub. Date: Jan. 23, 2025

(54) COMPOUND AS ATR KINASE INHIBITOR

(71) Applicant: SUZHOU ARK BIOPHARMACEUTICAL CO., LTD., Sizhou (CN)

(72) Inventors: Cheng PENG, Suzhou (CN); Shaoyun ZHANG, Suzhou (CN); Nan ZHANG, Suzhou (CN); Mengfei QIAN, Suzhou (CN); Zhongyang LI, Suzhou (CN); Guowei SONG, Suzhou (CN); Gang ZOU, Suzhou (CN); Haiqing YUAN, Suzhou (CN)

(21) Appl. No.: 18/681,269

(22) PCT Filed: Aug. 11, 2022
(86) PCT No.: PCT/CN2022/111868

§ 371 Date: Feb. 5, 2024

(30) Foreign Application Priority Data
Aug. 13, 2021 (CN) 202110930540.X
Mar. 29, 2022 (CN) 202210319824.X

(51) Int. Cl. C07D 413/04; A61K 31/497; A61P 35/00; C07D 413/14; C07D 417/14

(52) U.S. Cl. CPC C07D 413/04; A61K 31/497; A61P 35/00; C07D 413/14; C07D 417/14

(57) ABSTRACT

An ATR kinase inhibitor as shown in formula I, and a preparation method therefor and the use thereof.

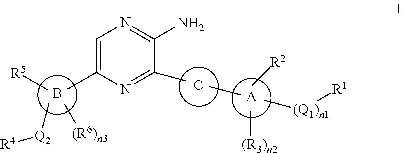

### TECHNICAL FIELD

The present disclosure belongs to the field of medicinal chemistry and relates, in particular, to an ATR kinase inhibitor and a preparation method therefor and the use thereof.

### BACKGROUND

ATR (Ataxia-telangiectasia and Rad3 related) kinase is an ATM Rad3-related protein kinase, and belongs to the PI3KKs (Phosphoinositide 3 kinase-related protein kinases) kinase family along with the ataxia-telangiectasia mutated (ATM) protein and DNA-PKcs (DNA-dependent protein kinase catalytic subunit).

Cells undergo DNA damage, in particular DNA single or double strand breaks (DSBs) and replication stress, as a result of the external stresses or the internal environment of the organism. In normal cells, DNA damages are rapidly recognised by the DNA-damage response (DDR) factor in order to maintain genomic stability, which activates cell cycle checkpoints and repairs DNA directly. Different DNA repair pathways are involved according to damage types, which together form a highly complex, interactive defense mechanism against genotoxic damage.

ATM and ATR are both activated by DNA damage and DNA replication stress. But, they differ in the DNA damage specificity and function. ATM is activated primarily by double-stranded DNA breaks, whereas ATR responds to a wide range of DNA damage, including DSBs and DNA damages caused by various interferences in replication. These two work in synergy to signal DNA damages and regulate downstream processes. However, many types of cancer cells are defective in some of their DNA repair processes (e.g., ATM signal transduction), making them more dependent on the remaining intact DNA repair proteins (e.g., ATR). In ATM-deficient cancer cells, inhibition of ATR is effective in suppressing cancer cell growth, thereby resulting in a synthetic lethality for cancer treatment.

Synthetic lethality refers to, in a functionally similar or compensatory pair of genes, the tolerance for cell survival under a single gene inactivation and occurrence of cell death under simultaneous disruption of multiple genes. Compared to cancer treatments such as conventional radiotherapy and chemotherapy, synthetic lethality-based therapeutic strategies have three advantages: firstly, as ATM is one of the most common abnormal genes in cancer, documented by COSMIC database that 167 kinds of ATM somatic mutations (excluding variants of unknown origin) have been observed in oncology and hematologic malignancies, synthetic lethality-based therapeutic strategies can be against most cancer mutations; secondly, corresponding patients can be simply identified based on the high selectivity of the mutations; and finally, the combined administration with chemotherapy drugs can lead to improved efficacy, reduced dose, and avoidance or mitigation of adverse effects.

ATR kinase inhibitors in preclinical and clinical research stages include Berzosertib (VX-970) disclosed in WO2010071837A1, Ceralaserti (AZD6738), Elimusertib (BAY-1895344), ART0380, RP-3500, VX-803 and M1774. Amongst, VX-970 is in Phase I/II or Phase II clinical trial for indications including prostate cancer, small cell lung cancer, ovarian cancer, uroepithelial cancer, and non-small cell lung cancer. AZD6738 is in Phase II clinical trial for the treatment of breast cancer, squamous cell carcinoma of the head and neck, gastric cancer, and non-small cell lung cancer. ART0380 is in Phase II clinical trial for the treatment of ovarian cancer. RP-3500 for the treatment of solid tumors, and VX-803 for breast cancer are in clinical Phase I/II. BAY-1895344 for lymphoma and solid tumors, and M1774 for solid tumors are in clinical Phase I.

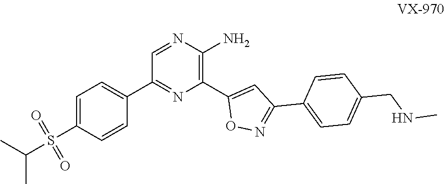 [SMILES: CNCc1ccc(-c2cc(-c3nc(-c4ccc(S(=O)(=O)C(C)C)cc4)cnc3N)on2)cc1]

The above clinical trials have shown that disruption of ATR function promotes cancer cell death in the presence or absence of DNA damaging agents. This suggests that ATR kinase inhibitors may be effective as a single medicament or as a sensitizer for radiotherapy or genotoxic chemotherapy.

### SUMMARY

### Technical Problem

The present compounds have an inhibitory activity against ATR kinase, but a high cytotoxicity and a low exposure in vivo PK. Therefore, there is still a need to develop more inhibitors with high ATR inhibition that own low cytotoxicity and high exposure.

### Solution to Problem

In order to solve the above-mentioned technical problem, the present disclosure provides:

A compound of formula I:

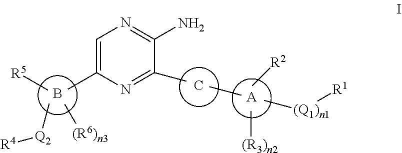

or a pharmaceutically acceptable salt, ester, isomer, solvate, prodrug or isotopically labeled compound thereof,
wherein:
Ring A, ring B and ring C are each independently selected from 6 to 10-membered aryl and 5 to 10-membered heteroaryl;
R¹ is selected from hydrogen, deuterium, cyano, halogen, and substituted or unsubstituted hydroxyl, amino, C₁₋₆ alkyl, C₁₋₆ alkoxy, 3- to 7-membered cycloalkyl, 3- to 7-membered heterocycloalkyl, guanidino, ureido, amido, aminosulfonamido, sulfonamido, and HN—C(═NH)—C₁₋₆ alkyl;
R² and R⁵ are each independently selected from hydrogen, deuterium, halogens, C₁₋₆ alkyl, amino and C₁₋₆ alkoxy;
or R¹ and R² together with the atoms to which they are attached form a 5 to 6-membered heteroaryl or heterocyclic group containing 1 to 3 atoms selected from oxygen, nitrogen and sulphur, said 5 to 6-membered heteroaryl or heterocyclic group having 0 or 1 methylene group on its ring optionally substituted with —C(═O)— or —C(═NR⁷)—;
if present, each R³ is independently selected from hydrogen, deuterium, halogen, C₁₋₆ alkyl and C₁₋₆ alkyl substituted with a substituent selected from cyano, amino, hydroxy, halogen, C₁₋₆ alkyl and C₁₋₆ alkoxy;
R⁴ is selected from hydrogen, deuterium, C₁₋₆ alkyl, 3- to 7-membered cycloalkyl, 3- to 7-membered heterocycloalkyl, C₁₋₆ alkylamino, and (C₁₋₃ alkyl)₂amino;
or R⁴ and R⁵ together with the atoms to which they are attached form a 5- to 7-membered heterocyclic group containing 1 to 3 atoms selected from oxygen, nitrogen and sulphur, said 5- to 7-membered heterocyclic group optionally substituted with a substituent selected from deuterium, hydroxyl, C₁₋₆ alkyl, halogen, C₁₋₆ alkoxy and C₁₋₆ alkylamino; said heterocyclic group having 1 to 3 methylene groups on its ring optionally substituted by —SO₂—, —SO(═NR⁷)—, —SO₂NH—, —CO—, —C(═O)NH—, C₁₋₆ alkylamino, or 3- to 6-membered cycloalkylamino; wherein said C₁₋₆ alkylamino or 3- to 6-membered cycloalkylamino has 0 to 2 methylene groups optionally substituted with oxygen, nitrogen, sulfur, or C₁₋₃ alkylamino;
if present, each R⁶ is independently selected from hydrogen, deuterium, halogen, amino, C₁₋₆ alkoxy, C₁₋₆ alkyl and C₁₋₆ alkyl substituted with cyano, amino, hydroxy, halogen, C₁₋₆ alkyl or C₁₋₆ alkoxy;
Q¹ is selected from C₁₋₆ alkylene, —CO—, amino, —SO—, —SO₂—, —C(═NR⁷)—, and 3- to 7-membered heterocyclic group, wherein said C₁₋₆ alkylene has 0 to 4 methylene groups optionally substituted with amino, —CO—, —SO—, —SO₂ or —C(═NR⁷)—;
Q² is selected from —SO₂—, SO, —SO(═NR⁷)—, —SO₂NR⁷— and —CO—;
each R⁷ is independently selected from hydrogen, deuterium, C₁₋₆ alkyl, cyano, 3- to 7-membered cycloalkyl, 3- to 7-membered heterocyclic group, C₁₋₆ alkoxy, C₁₋₆ alkylamino, and C₁₋₆ alkyl, 3- to 7-membered cycloalkyl and 3- to 7-membered heterocyclic group substituted by a substituent selected from cyano, amino, hydroxyl, halogen, C₁₋₆ alkyl, and C₁₋₆ alkoxy; and
each of n1, n2 and n3 is any integer from 0 to 2.

### Effects of the Disclosure

The present disclosure provides aminopyrazine compounds, which have high inhibitory activity against ATR, as well as excellent efficacy, in vitro/in vivo pharmacokinetic properties, and safety, thus offering a high prospect for clinical application.

### BRIEF DESCRIPTION OF THE DRAWINGS

FIG. 1: Changes of mice body weight after administration for different dosing groups

FIG. 2: Changes (absolute value) of mice tumor volume of after administration for different dosing groups

FIG. 3: Changes (percentage) of mice tumor volume of after administration for different dosing groups

### DETAILED DESCRIPTION

To make the technical solutions and beneficial effects of present disclosure more obvious and understandable, the detailed description is shown below by way of enumerating specific embodiments. Unless otherwise defined, technical and scientific terms used in the present disclosure have the same meanings as technical and scientific terms in the technical field to which the present application belongs.

The present disclosure provides a compound of formula I:

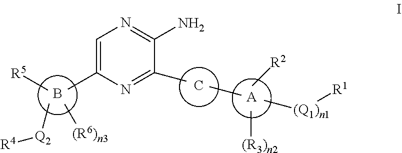

or a pharmaceutically acceptable salt, ester, isomer, solvate, prodrug or isotopically labeled compound thereof,
wherein:
Ring A, ring B and ring C are each independently selected from 6 to 10-membered aryl and 5 to 10-membered heteroaryl;
R¹ is selected from hydrogen, deuterium, cyano, halogen, and substituted or unsubstituted hydroxyl, amino, C₁₋₆ alkyl, C₁₋₆ alkoxy, 3- to 7-membered cycloalkyl, 3- to 7-membered heterocyclic group, guanidino, ureido, amido, aminosulfonamido, sulfonamido, and HN—C(═NH)—C₁₋₆ alkyl;
R² and R⁵ are each independently selected from hydrogen, deuterium, halogens, C₁₋₆ alkyl, amino and C₁₋₆ alkoxy;
or R¹ and R² together with the atoms to which they are attached form a 5 to 6-membered heteroaryl or heterocyclic group containing 1 to 3 atoms selected from oxygen, nitrogen and sulphur, said 5 to 6-membered heteroaryl or heterocyclic group having 0 or 1 methylene group on its ring optionally substituted with —C(═O)— or —C(═NR⁷)—;
if present, each R³ is independently selected from hydrogen, deuterium, halogen, C₁₋₆ alkyl and C₁₋₆ alkyl substituted with a substituent selected from cyano, amino, hydroxy, halogen, C₁₋₆ alkyl and C₁₋₆ alkoxy;
R⁴ is selected from hydrogen, deuterium, C₁₋₆ alkyl, 3- to 7-membered cycloalkyl, 3- to 7-membered heterocycloalkyl, C₁₋₆ alkylamino, and (C₁₋₃ alkyl)₂amino;
or R⁴ and R⁵ together with the atoms to which they are attached form a 5- to 7-membered heterocyclic group containing 1 to 3 atoms selected from oxygen, nitrogen and sulphur, said 5- to 7-membered heterocyclic group optionally substituted with a substituent selected from deuterium, hydroxyl, C₁₋₆ alkyl, halogen, C₁₋₆ alkoxy and C₁₋₆ alkylamino; said heterocyclic group having 1 to 3 methylene groups on its ring optionally substituted by —SO₂—, —SO(═NR⁷)—, —SO₂NH—, —CO—, —C(═O)NH—, C₁₋₆ alkylamino, or 3- to 6-membered cycloalkylamino; wherein said C₁₋₆ alkylamino or 3- to 6-membered cycloalkylamino has 0 to 2 methylene groups optionally substituted with oxygen, nitrogen, sulfur, or C₁₋₃ alkylamino;
if present, each R⁶ is independently selected from hydrogen, deuterium, halogen, amino, C₁₋₆ alkoxy, C₁₋₆ alkyl and C₁₋₆ alkyl substituted with cyano, amino, hydroxy, halogen, C₁₋₆ alkyl or C₁₋₆ alkoxy;
Q¹ is selected from C₁₋₆ alkylene, —CO—, amino, —SO—, —SO₂—, —C(═NR⁷)—, and 3- to 7-membered heterocyclic group, wherein said C₁₋₆ alkylene has 0 to 4 methylene groups optionally substituted with amino, —CO—, —SO—, —SO₂ or —C(═NR⁷)—;
Q² is selected from —SO₂—, SO, —SO(═NR⁷)—, —SO₂NR⁷— and —CO—;
each R⁷ is independently selected from hydrogen, deuterium, C₁₋₆ alkyl, cyano, 3- to 7-membered cycloalkyl, 3- to 7-membered heterocyclic group, C₁₋₆ alkoxy, C₁₋₆ alkylamino, and C₁₋₆ alkyl, 3- to 7-membered cycloalkyl and 3- to 7-membered heterocyclic group substituted by a substituent selected from cyano, amino, hydroxyl, halogen, C₁₋₆ alkyl, and C₁₋₆ alkoxy; and
each of n1, n2 and n3 is any integer from 0 to 2.

In some embodiments, for the compound of formula I or a pharmaceutically acceptable salt, ester, isomer, solvate, prodrug or isotopically labeled compound thereof, n1 is 0 or 1; n2 is 1 or 2; and n3 is preferably 0 or 1.

In some embodiments, for the compound of formula I or a pharmaceutically acceptable salt, ester, isomer, solvate, prodrug or isotopically labeled compound thereof, n1 is 0 or 1.

In some embodiments, for the compound of formula I or a pharmaceutically acceptable salt, ester, isomer, solvate, prodrug or isotopically labeled compound thereof, n2 is 0 or 1.

In some embodiments, for the compound of formula I or a pharmaceutically acceptable salt, ester, isomer, solvate, prodrug or isotopically labeled compound thereof, n3 is 0 or 1.

In some embodiments, for the compound of formula I or a pharmaceutically acceptable salt, ester, isomer, solvate, prodrug or isotopically labeled compound thereof, n1 is 0 or 1; n2 is 0 or 1; and n3 is 0 or 1.

In some embodiments, for the compound of formula I or a pharmaceutically acceptable salt, ester, isomer, solvate, prodrug or isotopically labeled compound thereof, said ring A and ring B are selected from phenyl and 5 to 6-membered heteroaryl; preferably, said ring A and ring B are selected from phenyl and 6-membered heteroaryl; more preferably, said ring A and ring B are selected from phenyl and pyridinyl, and most preferably, said ring A and ring B are phenyl.

In some embodiments, for the compound of formula I or a pharmaceutically acceptable salt, ester, isomer, solvate, prodrug or isotopically labeled compound thereof, said ring C is a 5 to 6-membered heteroaryl; preferably, said ring C is a 5-membered heteroaryl; more preferably, said ring C is selected from isoxazole, oxadiazole and thiadiazole; most preferably, said ring C is selected from isoxazole and oxadiazole. In some embodiments, the compound of formula I or a pharmaceutically acceptable salt, ester, isomer, solvate, prodrug or isotopically labeled compound thereof is a compound of formula I-a

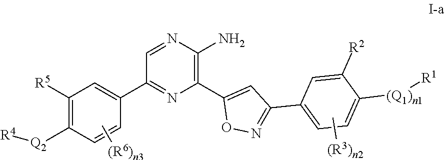

or a pharmaceutically acceptable salt, ester, isomer, solvate, prodrug or isotopically labeled compound thereof, wherein R¹, R², R³, R⁴, R⁵, R⁶, Q₁, Q₂, n₁, n₂ and n₃ are as defined in formula I.

In some embodiments, the compound of formula I or a pharmaceutically acceptable salt, ester, isomer, solvate, prodrug or isotopically labeled compound thereof is a compound of formula I-b:

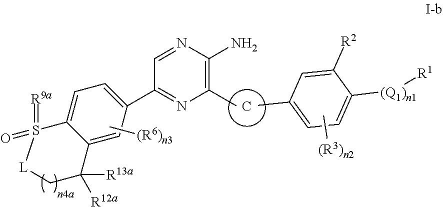

or a pharmaceutically acceptable salt, ester, isomer, solvate, prodrug or isotopically labeled compound thereof,
wherein:
Ring C is 5-membered heteroaryl;
L is CR^{10a}R^{11a} or NR^{14a};
n4a is 0, 1 or 2;
R^{9a} is selected from oxygen and NR⁷;
R^{10a} and R^{11a} are each independently selected from hydrogen, deuterium, C₁₋₆ alkyl, C₁₋₆ alkoxy, and C₁₋₆ alkylamino; or R¹⁰ and R¹¹ together with the atoms to which they are attached form a 3- to 7-membered cycloalkyl group or a 3- to 7-membered heterocyclic group containing 1 or 2 atoms selected from oxygen, nitrogen and sulfur;
R^{12a} and R^{13a} are each independently selected from hydrogen, deuterium, halogen, hydroxyl, C₁₋₆ alkyl, C₁₋₆ alkoxy and C₁₋₆ alkylamino; or R^{12a} and R^{13a} form ═O;
R^{14a} is selected from hydrogen, deuterium, substituted or unsubstituted C₁₋₆ alkyl, C₁₋₆ alkoxy, 3- to 7-membered cycloalkyl and 3- to 7-membered heterocyclic group, wherein said substitution is by a substituent selected from halogen, hydroxyl, amino, C₁₋₆ alkylamino, and (C₁₋₆ alkyl)₂ amino;
R¹, R², R³, R⁶, R⁷, Q₁, n1, n2 and n3 are as defined in Formula I.

In some embodiments, the compound of formula I or I-b, or a pharmaceutically acceptable salt, ester, isomer, solvate, prodrug or isotopically labeled compound thereof is a compound of formula I-b-1 or I-b-2 or a pharmaceutically acceptable salt, ester, isomer, solvate, prodrug or isotopically labeled compound thereof:

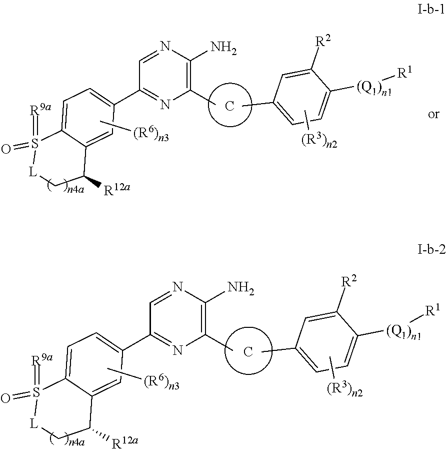

wherein:
ring C, R¹, R, R³, R⁶, R⁷, Q₁, n1, n2, n3, L, n4a, R^{9a} or R^{12a} are as defined in Formula I-b.

In some embodiments, for the compound of formula I, I-b, I-b-1 or I-b-2, or a pharmaceutically acceptable salt, ester, isomer, solvate, prodrug or isotopically labeled compound thereof, the ring C is a 5 to 6-membered heteroaryl; preferably, said ring C is a 5-membered heteroaryl; more preferably, said ring C is selected from isoxazole, oxadiazole and thiadiazole; most preferably, said ring C is selected from isoxazole and oxadiazole.

In some embodiments, for the compound of formula I, I-b, I-b-1 or I-b-2, or a pharmaceutically acceptable salt, ester, isomer, solvate, prodrug or isotopically labeled compound thereof, the ring C is selected from

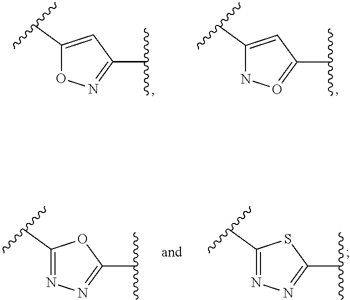

preferably from

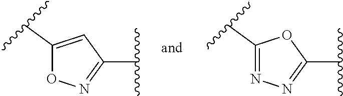 [SMILES: *c1cc(*)on1 ; *c1nnc(*)o1]

In some embodiments, for the compound of formula I-b, I-b-1 or I-b-2, or a pharmaceutically acceptable salt, ester, isomer, solvate, prodrug or isotopically labeled compound thereof, n4a is 0 or 1; preferably, n4a is 0.

In some embodiments, for the compound of formula I-b, I-b-1 or I-b-2, or a pharmaceutically acceptable salt, ester, isomer, solvate, prodrug or isotopically labeled compound thereof, R^{9a} is selected from oxygen and NH; preferably, R^{9a} is selected from oxygen.

In some embodiments, for the compound of formula I-b, I-b-1 or I-b-2, or a pharmaceutically acceptable salt, ester, isomer, solvate, prodrug or isotopically labeled compound thereof, L is CR^{10a}R^{11a}, R^{10a} and R^{11a} each independently selected from hydrogen, deuterium, C₁₋₆ alkyl and C₁₋₆ alkoxy; preferably, L is CR^{10a}R^{11a}, R^{10a} and R^{11a} each independently selected from hydrogen and C₁₋₆ alkyl; more preferably, L is CR^{10a}R^{11a}, R^{10a} and R^{11a} each being C₁₋₆ alkyl; most preferably, L is CR^{10a}R^{11a}, R^{10a} and R^{11a} each being methyl.

In some embodiments, for the compound of formula I-b, I-b-1 or I-b-2, or a pharmaceutically acceptable salt, ester, isomer, solvate, prodrug or isotopically labeled compound thereof, R¹⁰ and R¹¹ together with the atoms to which they are attached form a 3- to 7-membered cycloalkyl group or a 3- to 7-membered heterocyclic group containing 1 or 2 atoms selected from oxygen, nitrogen and sulfur, wherein the 3- to 7-membered cycloalkyl group is cyclopropyl.

In some embodiments, for the compound of formula I-b, I-b-1 or I-b-2, or a pharmaceutically acceptable salt, ester, isomer, solvate, prodrug or isotopically labeled compound thereof, R^{12a} and R^{13a} are each independently selected from hydrogen, deuterium, halogen, hydroxyl, C₁₋₆ alkyl and C₁₋₆ alkoxy; preferably, R^{12a} and R^{13a} are each independently selected from hydrogen, deuterium, halogen, hydroxyl and C₁₋₆ alkyl; more preferably, R^{12a} and R^{13a} are each independently selected from hydrogen, deuterium, methyl, fluorine and hydroxyl; further preferably, R^{12a} and R^{13a} are each independently selected from hydrogen and fluorine; most preferably, R^{12a} is hydrogen and R^{13a} is fluorine.

In some embodiments, for the compound of formula I-b, I-b-1 or I-b-2, or a pharmaceutically acceptable salt, ester, isomer, solvate, prodrug or isotopically labeled compound thereof, R^{11a} is selected from hydrogen, methyl, ethyl, isopropyl, cyclopropyl, hydroxyethyl and

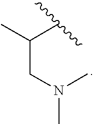 [SMILES: *C(C)CN(C)C]

In some embodiments, for the compound of formula I-b or a pharmaceutically acceptable salt, ester, isomer, solvate, prodrug or isotopically labeled compound thereof,

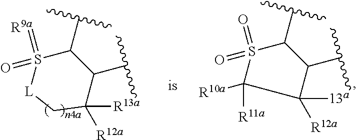

R^{10a}, R^{11a}, R^{12a} and R^{13a} as defined in formula I-b; preferably,

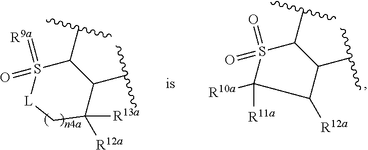

R^{10a}, R^{11a} and R^{12a} as defined in formula I-b; more preferably,

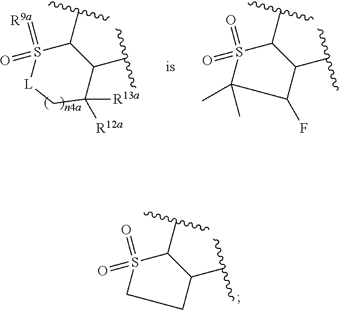

most preferably,

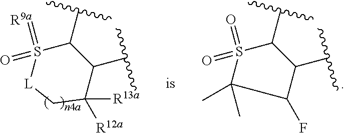

In some embodiments, for the compound of formula I-b-1 or a pharmaceutically acceptable salt, ester, isomer, solvate, prodrug or isotopically labeled compound thereof,

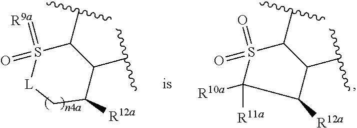

R^{10a}, R^{11a} and R^{12a} as defined in formula I-b-1; preferably,

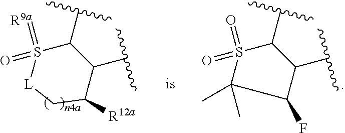

In some embodiments, for the compound of formula I-b-2 or a pharmaceutically acceptable salt, ester, isomer, solvate, prodrug or isotopically labeled compound thereof,

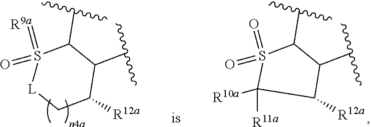

R^{10a}, R^{11a} and R^{12a} as defined in formula I-b-2; preferably,

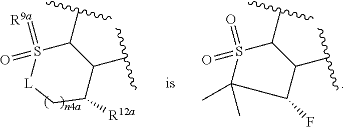

In some embodiments, for the compound of formula I, I-a, I-b, I-b-1 or I-b-2, or a pharmaceutically acceptable salt, ester, isomer, solvate, prodrug or isotopically labeled compound thereof, R¹ is selected from hydrogen, deuterium, cyano, halogen, and substituted or unsubstituted hydroxyl, amino, C₁₋₆ alkyl, C₁₋₆ alkoxy, 3- to 7-membered cycloalkyl, 3- to 7-membered heterocyclic group, guanidino, ureido, amido, aminosulfonamido, sulfonamido, and HN—C(═NH)—C₁₋₆ alkyl, said substitution being substituted by a substituent selected from C₁₋₆ alkyl or 3 to 7-membered cycloalkyl; preferably, R¹ is selected from hydrogen, substituted or unsubstituted amino, C₁₋₆ alkyl, guanidino, ureido, 3- to 7-membered heterocyclic, aminosulfonamido, and HN—C(═NH)—C₁₋₆ alkyl, said substitution being substituted by a substituent selected from C₁₋₆ alkyl or 3 to 7-membered cycloalkyl; more preferably, R¹ is selected from hydrogen, methyl, ethyl,

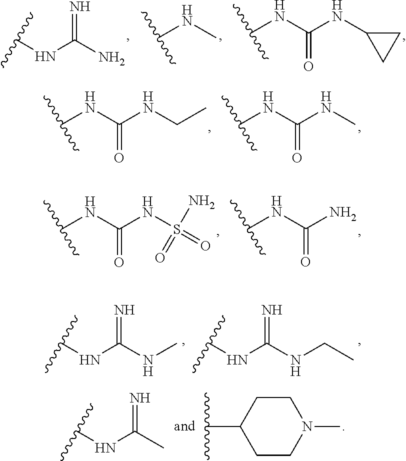

In some embodiments, for the compound of formula I, I-a, I-b, I-b-1 or I-b-2, or a pharmaceutically acceptable salt, ester, isomer, solvate, prodrug or isotopically labeled compound thereof, R² and R⁵ are each independently selected from hydrogen, deuterium, halogens and C₁₋₆ alkyl; preferably, R² and R⁵ are each independently selected from hydrogen or deuterium.

In some embodiments, for the compound of formula I, I-a, I-b, I-b-1 or I-b-2, or a pharmaceutically acceptable salt, ester, isomer, solvate, prodrug or isotopically labeled compound thereof, R¹ and R² together with the atoms to which they are attached form a 5 to 6-membered heteroaryl or heterocyclic group containing 1 to 3 atoms selected from oxygen, nitrogen and sulphur, said 5 to 6-membered heteroaryl or heterocyclic group having 0 or 1 methylene group on its ring optionally substituted with —C(═O)— or —C(═NR⁷)—. Said 5 to 6-membered heteroaryl or heterocyclic group may be any of the following structures:

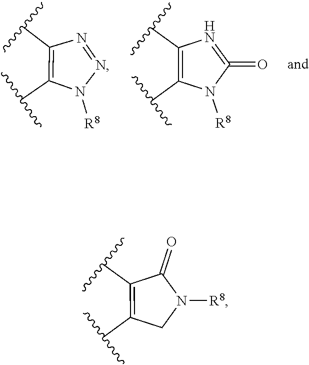

wherein R⁸ is selected from hydrogen, deuterium, C₁₋₆ alkyl and 3 to 7-membered cycloalkyl; preferably, R⁸ is hydrogen.

In some embodiments, for the compound of formula I, I-a, I-b, I-b-1 or I-b-2, or a pharmaceutically acceptable salt, ester, isomer, solvate, prodrug or isotopically labeled compound thereof, R³ is halogen or halogen-substituted C₁₋₆ alkyl, preferably F or CF₃.

In some embodiments, for the compound of formula I, I-a, I-b, I-b-1 or I-b-2, or a pharmaceutically acceptable salt, ester, isomer, solvate, prodrug or isotopically labeled compound thereof, R³ is selected from hydrogen, halogen, C₁₋₆ alkyl and halogen-substituted C₁₋₆ alkyl; preferably, R³ is selected from hydrogen, halogen and halogen-substituted C₁₋₆ alkyl; more preferably, R³ is selected from hydrogen, F and CF₃.

In some embodiments, for the compound of formula I or I-a, or a pharmaceutically acceptable salt, ester, isomer, solvate, prodrug or isotopically labeled compound thereof, R⁴ is selected from C₁₋₆ alkyl, 3 to 7-membered cycloalkyl, C₁₋₆ alkylamino and (C₁₋₃ alkyl)₂ amino; preferably, R⁴ is methyl or any of the following structures:

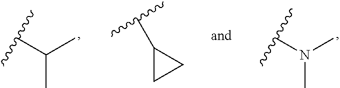

In some embodiments, for the compound of formula I or I-a, or a pharmaceutically acceptable salt, ester, isomer, solvate, prodrug or isotopically labeled compound thereof, R⁴ and R⁵ together with the atoms to which they are attached form a 5- to 7-membered heterocyclic group containing 1 to 3 atoms selected from oxygen, nitrogen and sulphur, said 5- to 7-membered heterocyclic group preferably being any of the following structures:

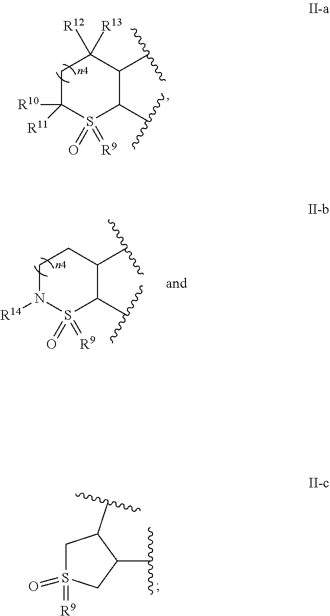

wherein, n4 is selected from an integer from 0 to 2;
R⁹ is selected from oxygen and NR;
R¹⁰ and R¹¹ are each independently selected from hydrogen, deuterium, C₁₋₆ alkyl, C₁₋₆ alkoxy and C₁₋₆ alkylamino; or R¹⁰ and R¹¹ together with the atoms to which they are attached form a 3 to 7-membered cycloalkyl group or a 3 to 7-membered heterocyclic group containing 1 or 2 atoms selected from oxygen, nitrogen and sulfur;
R¹² and R¹³ each independently selected from hydrogen, deuterium, halogen, hydroxyl, C₁₋₆ alkyl, C₁₋₆ alkoxy and C₁₋₆ alkylamino; or R¹² and R¹³ form ═O;
R¹⁴ is selected from hydrogen, deuterium, substituted or unsubstituted C₁₋₆ alkyl, C₁₋₆ alkoxy, 3- to 7-membered cycloalkyl and 3- to 7-membered heterocyclic group, wherein said substitution is substituted by a substituent selected from halogen, hydroxyl, amino, C₁₋₆ alkylamino, and (C₁₋₆ alkyl)₂ amino;
R⁷ is as defined in formula I.

In some embodiments, for the compound of formula I, I-a, I-b, I-b-1 or I-b-2, or a pharmaceutically acceptable salt, ester, isomer, solvate, prodrug or isotopically labeled compound thereof, each R⁶ is independently selected from hydrogen, deuterium and fluorine.

In some embodiments, for the compound of formula I, I-a, I-b, I-b-1 or I-b-2, or a pharmaceutically acceptable salt, ester, isomer, solvate, prodrug or isotopically labeled compound thereof, Q¹ is selected from C₁₋₆ alkylene, —CO—, amino, —SO—, —SO₂—, —C(═NR⁷)— or any of the following structures:

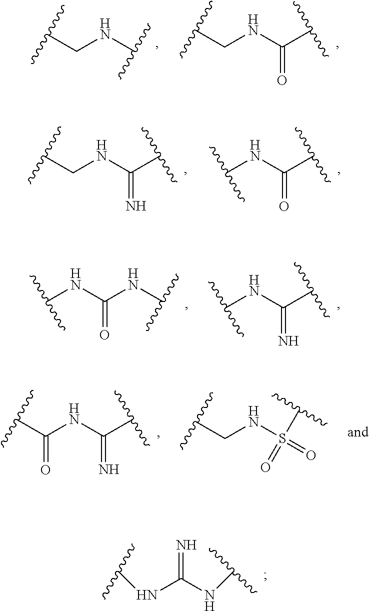

preferably, Q¹ is selected from C₁₋₆ alkylene, —CO—,

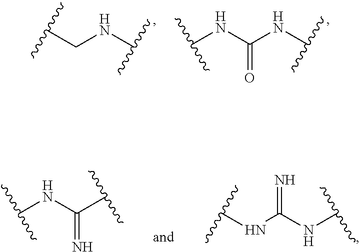

In some embodiments, for the compound of formula I or I-a, or a pharmaceutically acceptable salt, ester, isomer, solvate, prodrug or isotopically labeled compound thereof, Q² is selected from —SO₂—, —SO(═NH)—, —SO(═NMe)- and —CO—.

In some embodiments, for the compound of formula I, I-a, I-b, I-b-1 or I-b-2, or a pharmaceutically acceptable salt, ester, isomer, solvate, prodrug or isotopically labeled compound thereof, each R⁷ is independently selected from hydrogen, deuterium, methyl and cyano; preferably, each R⁷ is independently selected from hydrogen and methyl.

In some embodiments, in formula II-a or II-b, n4 is 0 or 1.

In some embodiments, in formula II-a, II-b or II-c, R⁹ is selected from oxygen and NH.

In some embodiments, in formula II-a, R¹⁰ and R¹¹ are each independently selected from hydrogen, deuterium, C₁₋₆ alkyl and C₁₋₆ alkoxy; preferably, R¹⁰ and R¹¹ are each independently selected from hydrogen and C₁₋₆ alkyl; more preferably, R¹⁰ and R¹¹ are each independently selected from hydrogen and methyl.

In some embodiments, in formula II-a, R¹⁰ and R¹¹ together with the atoms to which they are attached form a 3 to 7-membered cycloalkyl or a 3 to 7-membered heterocyclic group containing 1 or 2 atoms selected from oxygen, nitrogen and sulphur; said 3 to 7-membered cycloalkyl group being a cyclopropyl group.

In some embodiments, in formula II-a, R¹² and R¹³ are each independently selected from hydrogen, deuterium, halogen, hydroxyl, C₁₋₆ alkyl, C₁₋₆ alkoxy; preferably, R¹² and R¹³ are each independently selected from hydrogen, deuterium, halogen, hydroxyl, and C₁₋₆ alkyl; more preferably, R¹² and R¹³ are each independently selected from hydrogen, deuterium, methyl, fluorine, and hydroxyl; further preferably, R¹² and R¹³ are each independently selected from hydroxyl and fluoro; most preferably, R¹² is hydroxyl, and R¹³ is fluorine.

In some embodiments, in formula II-b, R¹⁴ is selected from hydrogen, methyl, ethyl, isopropyl, cyclopropyl, hydroxyethyl and

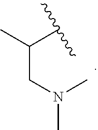 [SMILES: *C(C)CN(C)C]

TABLE A
Exemplary compounds of the present disclosure include, but are not limited to:

| Example | | Example | |
|---|---|---|---|
| No. | Structure | No. | Structure |
| | 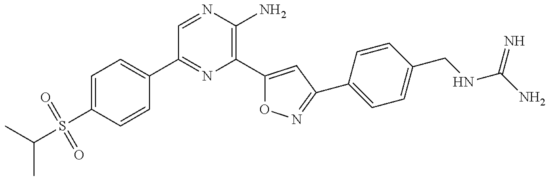 [SMILES: CC(C)S(=O)(=O)c1ccc(-c2cnc(N)c(-c3cc(-c4ccc(CNC(=N)N)cc4)no3)n2)cc1] | | 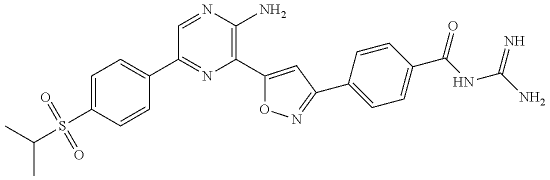 [SMILES: CC(C)S(=O)(=O)c1ccc(-c2cnc(N)c(-c3cc(-c4ccc(C(=O)NC(=N)N)cc4)no3)n2)cc1] |
| 2 | 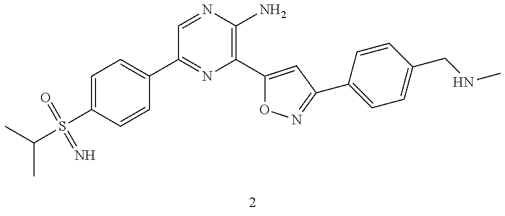 [SMILES: CNCc1ccc(-c2cc(-c3nc(-c4ccc(S(=N)(=O)C(C)C)cc4)cnc3N)on2)cc1] | | 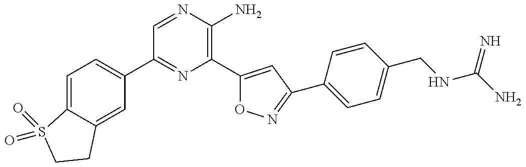 [SMILES: N=C(N)NCc1ccc(-c2cc(-c3nc(-c4ccc5c(c4)CCS5(=O)=O)cnc3N)on2)cc1] |
| 3 | 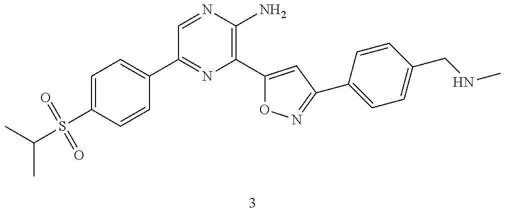 [SMILES: CNCc1ccc(-c2cc(-c3nc(-c4ccc(S(=O)(=O)C(C)C)cc4)cnc3N)on2)cc1] | | 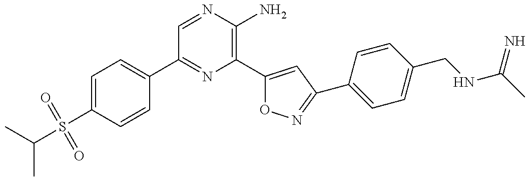 [SMILES: CC(=N)NCc1ccc(-c2cc(-c3nc(-c4ccc(S(=O)(=O)C(C)C)cc4)cnc3N)on2)cc1] |
| | 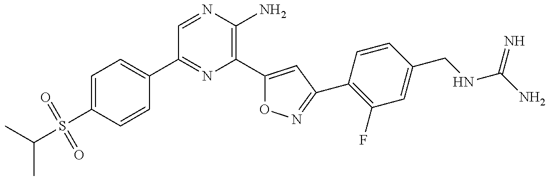 [SMILES: CC(C)S(=O)(=O)c1ccc(-c2cnc(N)c(-c3cc(-c4ccc(CNC(=N)N)cc4F)no3)n2)cc1] | | 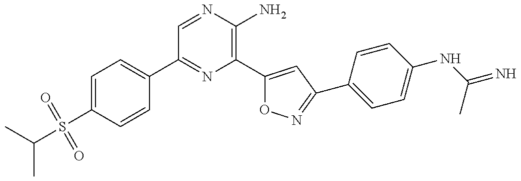 [SMILES: CC(=N)Nc1ccc(-c2cc(-c3nc(-c4ccc(S(=O)(=O)C(C)C)cc4)cnc3N)on2)cc1] |
| 5 | 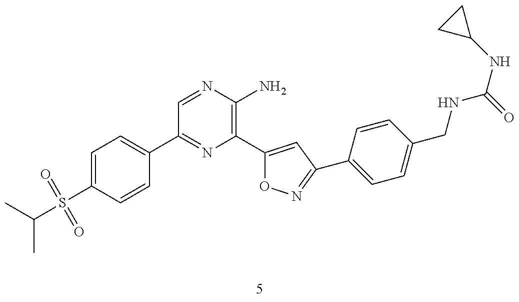 [SMILES: CC(C)S(=O)(=O)c1ccc(-c2cnc(N)c(-c3cc(-c4ccc(CNC(=O)NC5CC5)cc4)no3)n2)cc1] | | 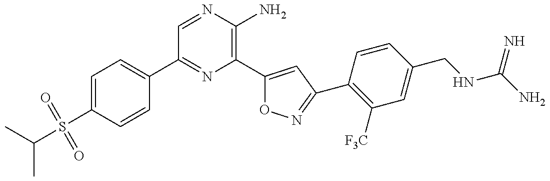 [SMILES: CC(C)S(=O)(=O)c1ccc(-c2cnc(N)c(-c3cc(-c4ccc(CNC(=N)N)cc4C(F)(F)F)no3)n2)cc1] |
| 6 | 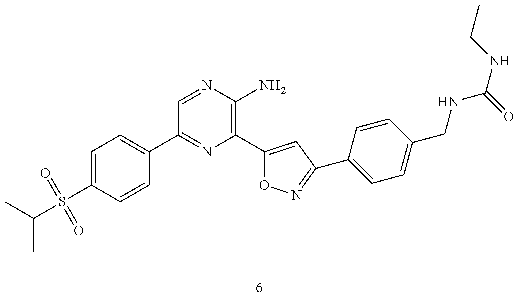 [SMILES: CCNC(=O)NCc1ccc(-c2cc(-c3nc(-c4ccc(S(=O)(=O)C(C)C)cc4)cnc3N)on2)cc1] | 31 | 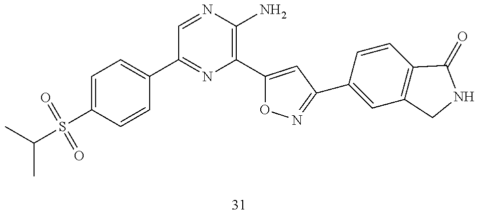 [SMILES: CC(C)S(=O)(=O)c1ccc(-c2cnc(N)c(-c3cc(-c4ccc5c(c4)CNC5=O)no3)n2)cc1] |
| 7 | 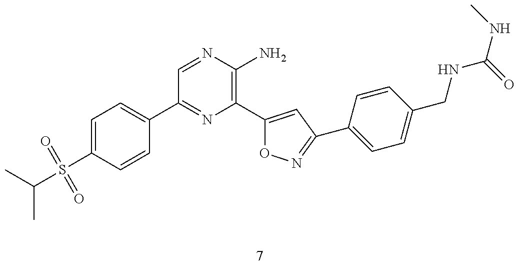 [SMILES: CNC(=O)NCc1ccc(-c2cc(-c3nc(-c4ccc(S(=O)(=O)C(C)C)cc4)cnc3N)on2)cc1] | 32 | 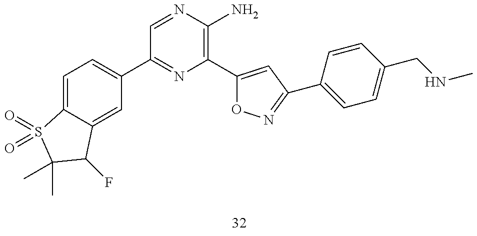 [SMILES: CNCc1ccc(-c2cc(-c3nc(-c4ccc5c(c4)C(F)C(C)(C)S5(=O)=O)cnc3N)on2)cc1] |
| 8 | 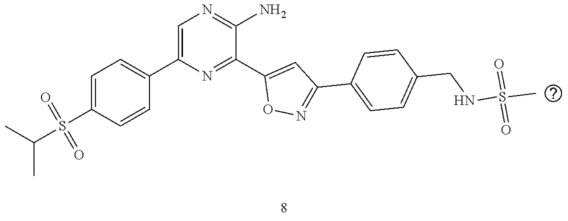 [SMILES: CC(C)S(=O)(=O)c1ccc(-c2cnc(N)c(-c3cc(-c4ccc(CNS(C)(=O)=O)cc4)no3)n2)cc1] | | 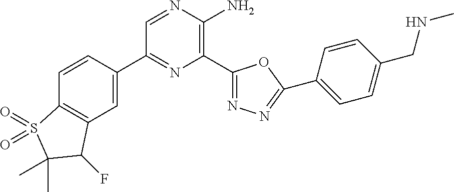 [SMILES: CNCc1ccc(-c2nnc(-c3nc(-c4ccc5c(c4)C(F)C(C)(C)S5(=O)=O)cnc3N)o2)cc1] |
| 9 | 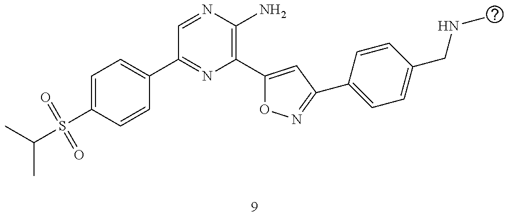 [SMILES: CNCc1ccc(-c2cc(-c3nc(-c4ccc(S(=O)(=O)C(C)C)cc4)cnc3N)on2)cc1] | 33-1 | 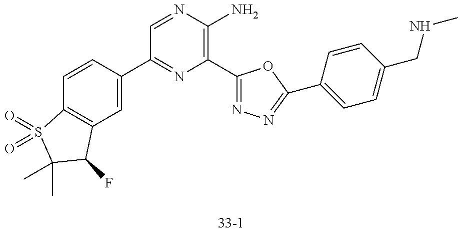 [SMILES: CNCc1ccc(-c2nnc(-c3nc(-c4ccc5c(c4)[C@@H](F)C(C)(C)S5(=O)=O)cnc3N)o2)cc1] |
| | 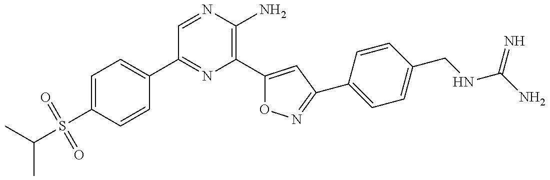 [SMILES: CC(C)S(=O)(=O)c1ccc(-c2cnc(N)c(-c3cc(-c4ccc(CNC(=N)N)cc4)no3)n2)cc1] | 33-2 | 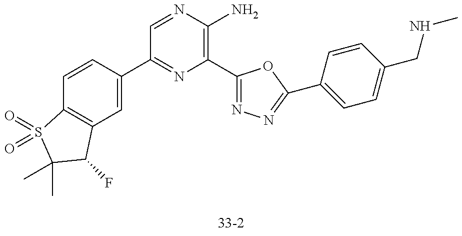 [SMILES: CNCc1ccc(-c2nnc(-c3nc(-c4ccc5c(c4)[C@H](F)C(C)(C)S5(=O)=O)cnc3N)o2)cc1] |
| | 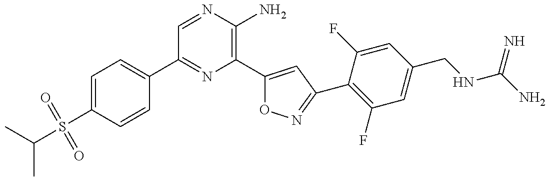 [SMILES: CC(C)S(=O)(=O)c1ccc(-c2cnc(N)c(-c3cc(-c4c(F)cc(CNC(=N)N)cc4F)no3)n2)cc1] | 34 | 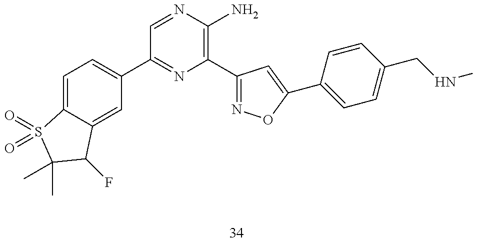 [SMILES: CNCc1ccc(-c2cc(-c3nc(-c4ccc5c(c4)C(F)C(C)(C)S5(=O)=O)cnc3N)no2)cc1] |
| | 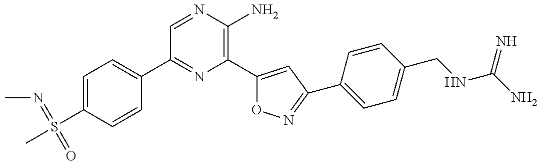 [SMILES: CN=S(C)(=O)c1ccc(-c2cnc(N)c(-c3cc(-c4ccc(CNC(=N)N)cc4)no3)n2)cc1] | | 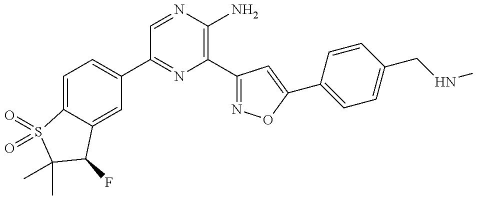 [SMILES: CNCc1ccc(-c2cc(-c3nc(-c4ccc5c(c4)[C@@H](F)C(C)(C)S5(=O)=O)cnc3N)no2)cc1] |
| | 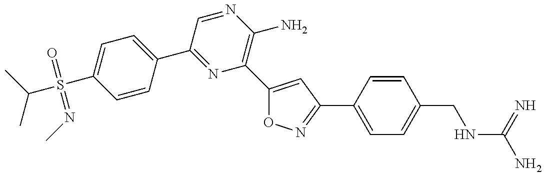 [SMILES: CN=S(=O)(c1ccc(-c2cnc(N)c(-c3cc(-c4ccc(CNC(=N)N)cc4)no3)n2)cc1)C(C)C] | | 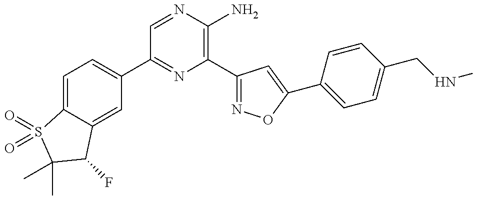 [SMILES: CNCc1ccc(-c2cc(-c3nc(-c4ccc5c(c4)[C@H](F)C(C)(C)S5(=O)=O)cnc3N)no2)cc1] |
| 14 | 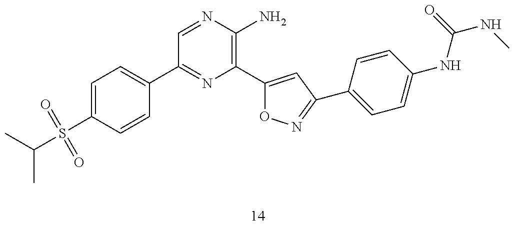 [SMILES: CNC(=O)Nc1ccc(-c2cc(-c3nc(-c4ccc(S(=O)(=O)C(C)C)cc4)cnc3N)on2)cc1] | 35-1 | 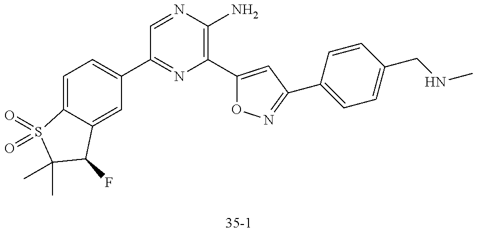 [SMILES: CNCc1ccc(-c2cc(-c3nc(-c4ccc5c(c4)[C@@H](F)C(C)(C)S5(=O)=O)cnc3N)on2)cc1] |
| 15 | 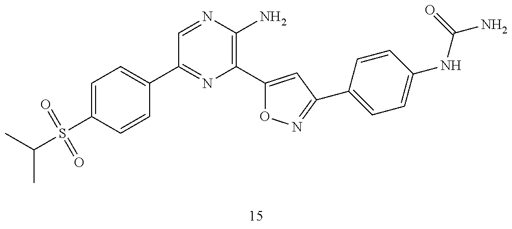 [SMILES: CC(C)S(=O)(=O)c1ccc(-c2cnc(N)c(-c3cc(-c4ccc(NC(N)=O)cc4)no3)n2)cc1] | 35-2 | 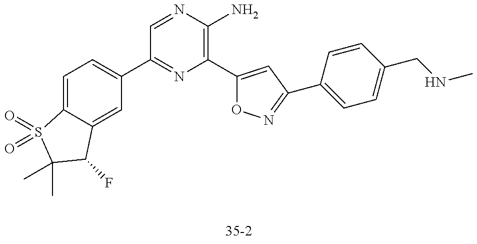 [SMILES: CNCc1ccc(-c2cc(-c3nc(-c4ccc5c(c4)[C@H](F)C(C)(C)S5(=O)=O)cnc3N)on2)cc1] |
| 16 | 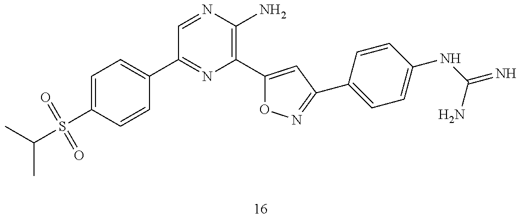 [SMILES: CC(C)S(=O)(=O)c1ccc(-c2cnc(N)c(-c3cc(-c4ccc(NC(=N)N)cc4)no3)n2)cc1] | 36 | 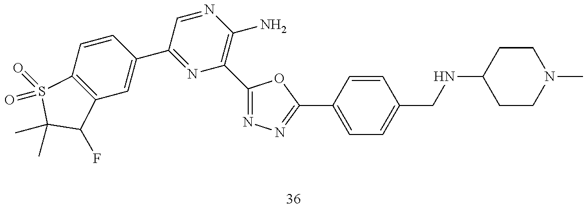 [SMILES: CN1CCC(NCc2ccc(-c3nnc(-c4nc(-c5ccc6c(c5)C(F)C(C)(C)S6(=O)=O)cnc4N)o3)cc2)CC1] |
| | 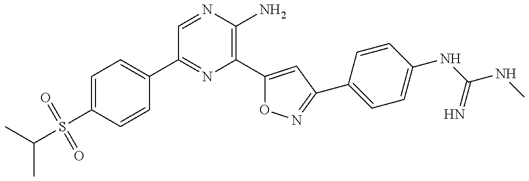 [SMILES: CNC(=N)Nc1ccc(-c2cc(-c3nc(-c4ccc(S(=O)(=O)C(C)C)cc4)cnc3N)on2)cc1] | | 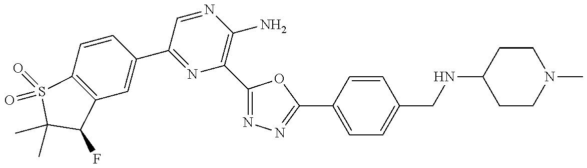 [SMILES: CN1CCC(NCc2ccc(-c3nnc(-c4nc(-c5ccc6c(c5)[C@@H](F)C(C)(C)S6(=O)=O)cnc4N)o3)cc2)CC1] |
| | 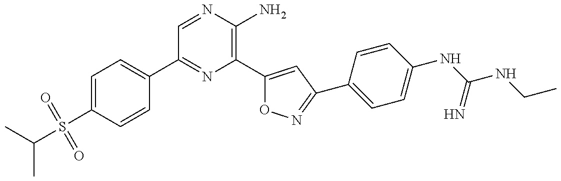 [SMILES: CCNC(=N)Nc1ccc(-c2cc(-c3nc(-c4ccc(S(=O)(=O)C(C)C)cc4)cnc3N)on2)cc1] | | 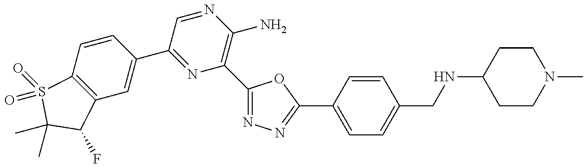 [SMILES: CN1CCC(NCc2ccc(-c3nnc(-c4nc(-c5ccc6c(c5)[C@H](F)C(C)(C)S6(=O)=O)cnc4N)o3)cc2)CC1] |
| | 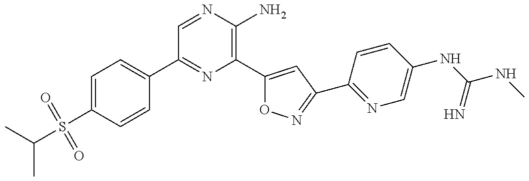 [SMILES: CNC(=N)Nc1ccc(-c2cc(-c3nc(-c4ccc(S(=O)(=O)C(C)C)cc4)cnc3N)on2)nc1] | 37 | 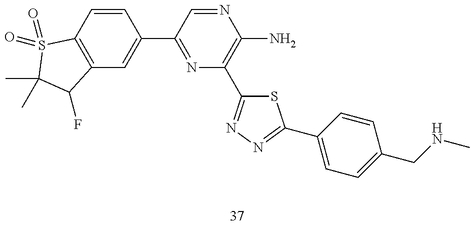 |
| | 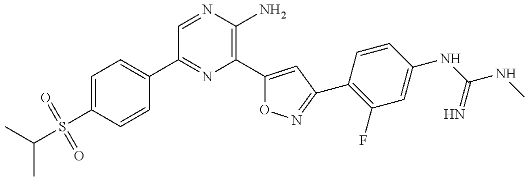 [SMILES: CNC(=N)Nc1ccc(-c2cc(-c3nc(-c4ccc(S(=O)(=O)C(C)C)cc4)cnc3N)on2)c(F)c1] | | 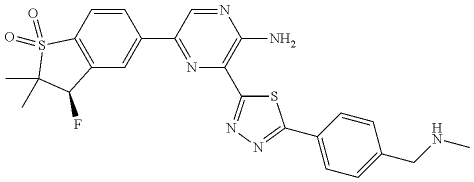 |
| 21 | 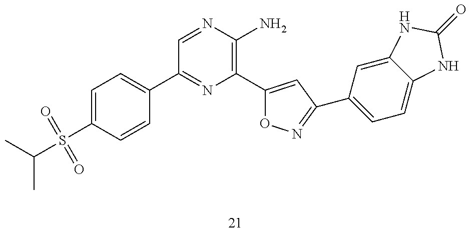 [SMILES: CC(C)S(=O)(=O)c1ccc(-c2cnc(N)c(-c3cc(-c4ccc5[nH]c(=O)[nH]c5c4)no3)n2)cc1] | | 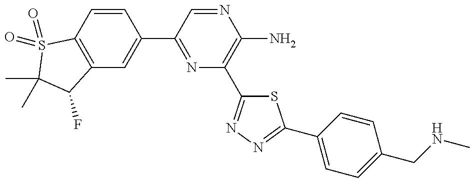 |
| | 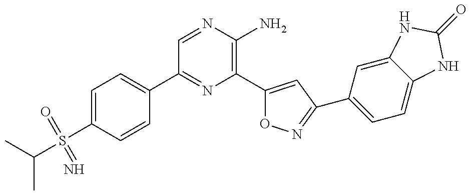 [SMILES: CC(C)S(=N)(=O)c1ccc(-c2cnc(N)c(-c3cc(-c4ccc5[nH]c(=O)[nH]c5c4)no3)n2)cc1] | | 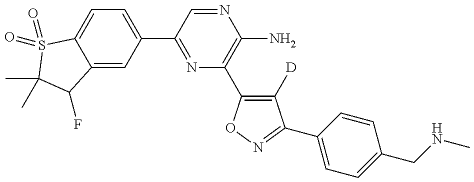 |
| | 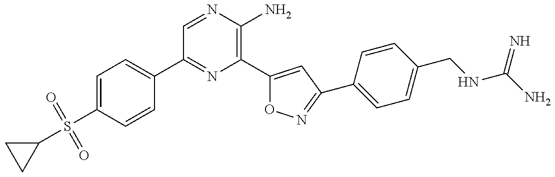 [SMILES: N=C(N)NCc1ccc(-c2cc(-c3nc(-c4ccc(S(=O)(=O)C5CC5)cc4)cnc3N)on2)cc1] | 38-1 | 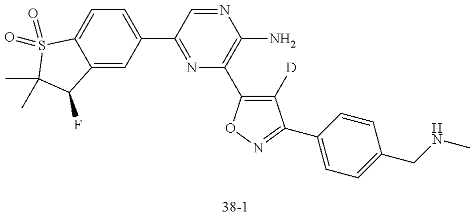 |
| | 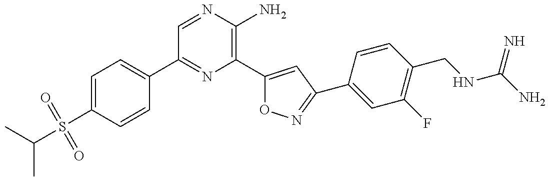 [SMILES: CC(C)S(=O)(=O)c1ccc(-c2cnc(N)c(-c3cc(-c4ccc(CNC(=N)N)c(F)c4)no3)n2)cc1] | 38-2 | 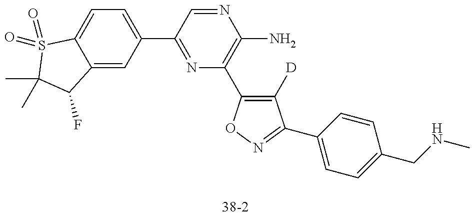 |
| | 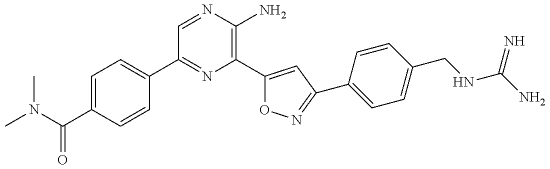 [SMILES: CN(C)C(=O)c1ccc(-c2cnc(N)c(-c3cc(-c4ccc(CNC(=N)N)cc4)no3)n2)cc1] | | |

TABLE B
The compounds disclosed in present disclosure include, but not limited to:

| Example | | Example | |
|---|---|---|---|
| No. | Structure | No. | Structure |
| 1 | 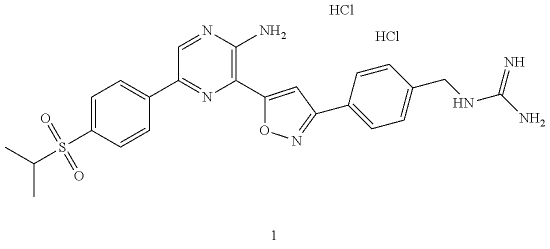 [SMILES: Cl ; CC(C)S(=O)(=O)c1ccc(-c2cnc(N)c(-c3cc(-c4ccc(CNC(=N)N)cc4)no3)n2)cc1 ; Cl] | 22 | 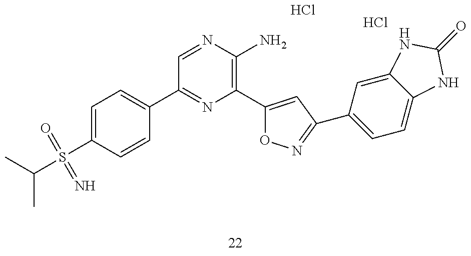 [SMILES: Cl ; Cl ; CC(C)S(=N)(=O)c1ccc(-c2cnc(N)c(-c3cc(-c4ccc5[nH]c(=O)[nH]c5c4)no3)n2)cc1] |
| 4 | 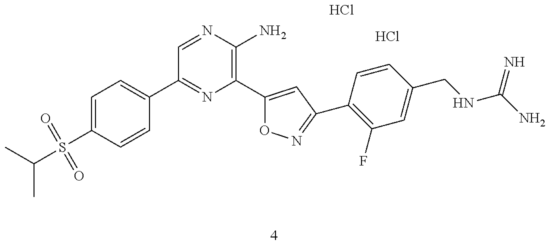 [SMILES: Cl ; CC(C)S(=O)(=O)c1ccc(-c2cnc(N)c(-c3cc(-c4ccc(CNC(=N)N)cc4F)no3)n2)cc1 ; Cl] | 23 | 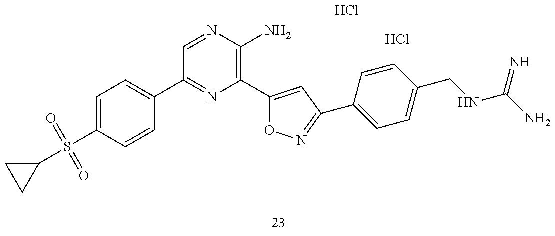 [SMILES: Cl ; N=C(N)NCc1ccc(-c2cc(-c3nc(-c4ccc(S(=O)(=O)C5CC5)cc4)cnc3N)on2)cc1 ; Cl] |
| 10 | 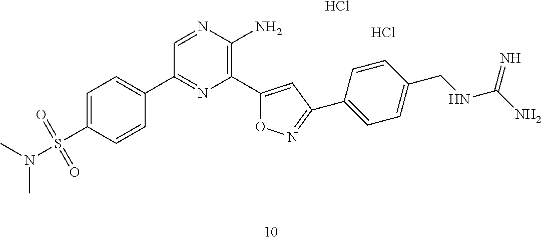 [SMILES: Cl ; CN(C)S(=O)(=O)c1ccc(-c2cnc(N)c(-c3cc(-c4ccc(CNC(=N)N)cc4)no3)n2)cc1 ; Cl] | 24 | 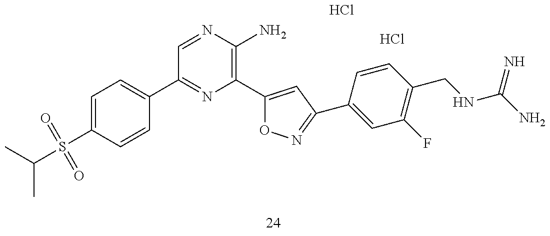 [SMILES: Cl ; CC(C)S(=O)(=O)c1ccc(-c2cnc(N)c(-c3cc(-c4ccc(CNC(=N)N)c(F)c4)no3)n2)cc1 ; Cl] |
| 11 | 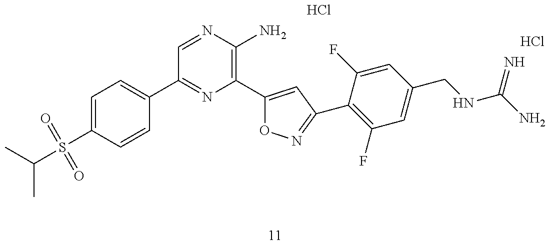 [SMILES: Cl ; CC(C)S(=O)(=O)c1ccc(-c2cnc(N)c(-c3cc(-c4c(F)cc(CNC(=N)N)cc4F)no3)n2)cc1 ; Cl] | 25 | 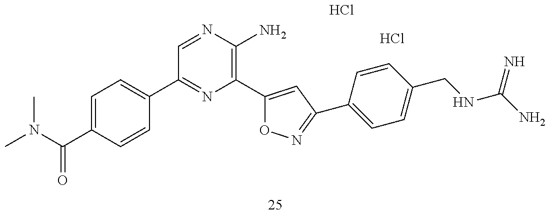 [SMILES: Cl ; CN(C)C(=O)c1ccc(-c2cnc(N)c(-c3cc(-c4ccc(CNC(=N)N)cc4)no3)n2)cc1 ; Cl] |
| 12 | 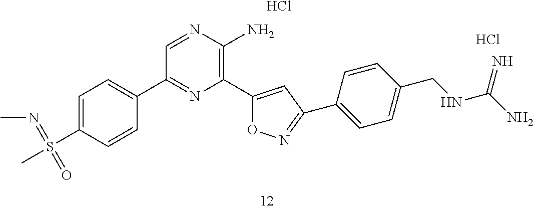 [SMILES: Cl ; CN=S(C)(=O)c1ccc(-c2cnc(N)c(-c3cc(-c4ccc(CNC(=N)N)cc4)no3)n2)cc1 ; Cl] | 26 | 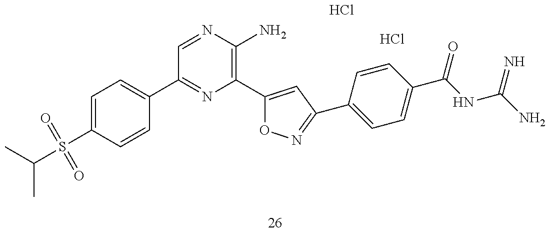 [SMILES: Cl ; CC(C)S(=O)(=O)c1ccc(-c2cnc(N)c(-c3cc(-c4ccc(C(=O)NC(=N)N)cc4)no3)n2)cc1 ; Cl] |
| 13 | 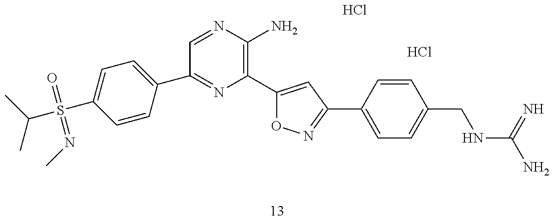 [SMILES: Cl ; CN=S(=O)(c1ccc(-c2cnc(N)c(-c3cc(-c4ccc(CNC(=N)N)cc4)no3)n2)cc1)C(C)C ; Cl] | 27 | 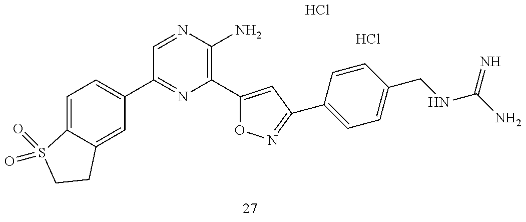 [SMILES: Cl ; N=C(N)NCc1ccc(-c2cc(-c3nc(-c4ccc5c(c4)CCS5(=O)=O)cnc3N)on2)cc1 ; Cl] |
| 17 | 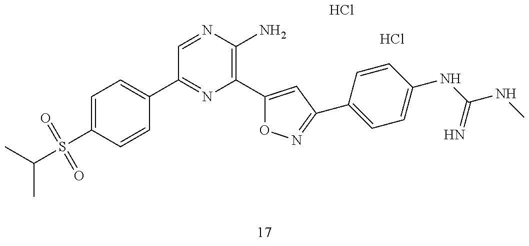 [SMILES: Cl ; CNC(=N)Nc1ccc(-c2cc(-c3nc(-c4ccc(S(=O)(=O)C(C)C)cc4)cnc3N)on2)cc1 ; Cl] | 28 | 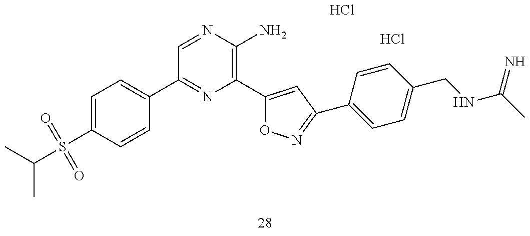 [SMILES: Cl ; CC(=N)NCc1ccc(-c2cc(-c3nc(-c4ccc(S(=O)(=O)C(C)C)cc4)cnc3N)on2)cc1 ; Cl] |
| 18 | 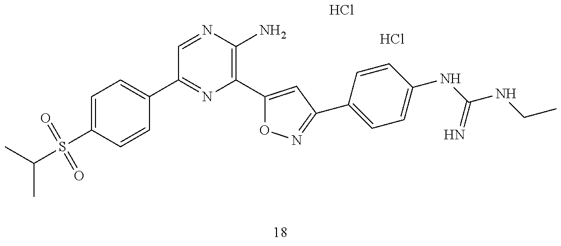 [SMILES: Cl ; CCNC(=N)Nc1ccc(-c2cc(-c3nc(-c4ccc(S(=O)(=O)C(C)C)cc4)cnc3N)on2)cc1 ; Cl] | 29 | 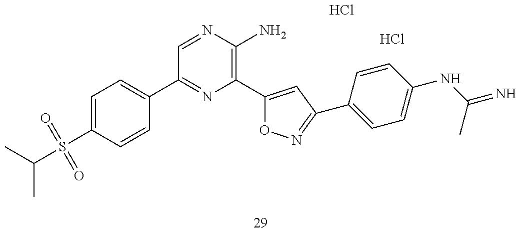 [SMILES: Cl ; CC(=N)Nc1ccc(-c2cc(-c3nc(-c4ccc(S(=O)(=O)C(C)C)cc4)cnc3N)on2)cc1 ; Cl] |
| 19 | 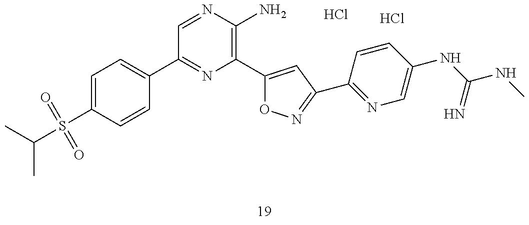 [SMILES: CNC(=N)Nc1ccc(-c2cc(-c3nc(-c4ccc(S(=O)(=O)C(C)C)cc4)cnc3N)on2)nc1 ; Cl ; Cl] | 30 | 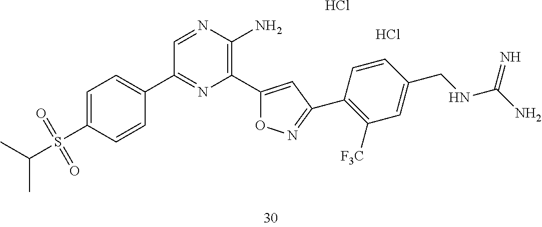 [SMILES: Cl ; CC(C)S(=O)(=O)c1ccc(-c2cnc(N)c(-c3cc(-c4ccc(CNC(=N)N)cc4C(F)(F)F)no3)n2)cc1 ; Cl] |
| 20 | 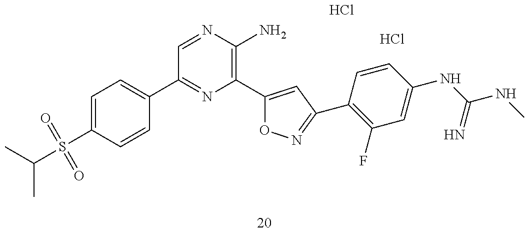 [SMILES: Cl ; CNC(=N)Nc1ccc(-c2cc(-c3nc(-c4ccc(S(=O)(=O)C(C)C)cc4)cnc3N)on2)c(F)c1 ; Cl] | | |

The present disclosure further provides schemes for preparing a compound of Formula I.

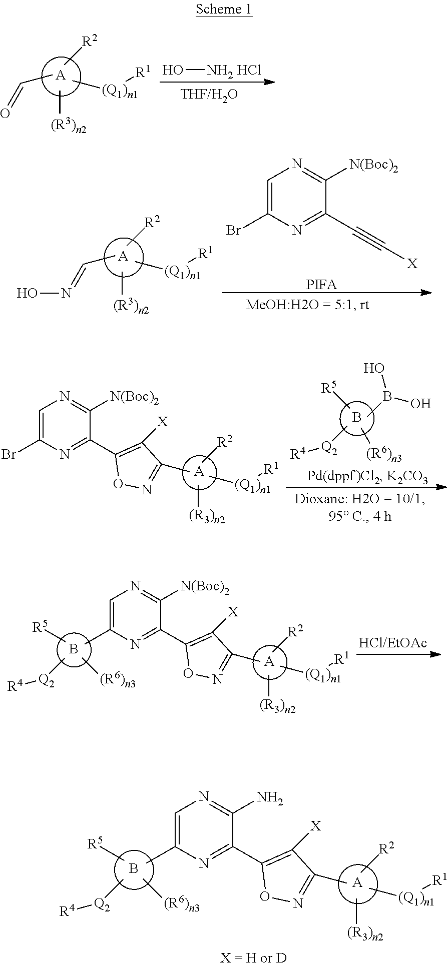

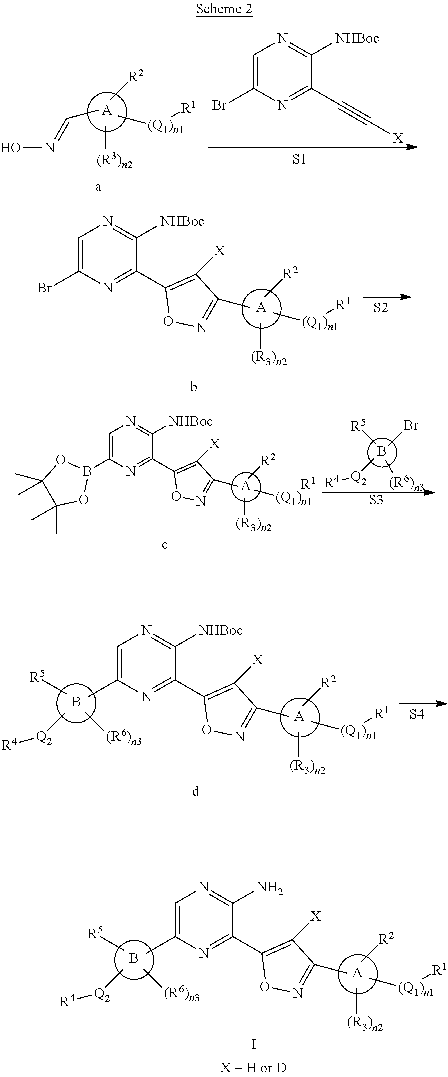

In some embodiments, Step S1 occurs in the presence of bis (trifluoroacetyl) iodobenzene.

In some embodiments, Step S2 occurs in the presence of bis(pinacolato)diboron.

In some embodiments, Step S2 occurs in the presence of a base.

In some embodiments, the base in step S2 is selected from inorganic base and organic base.

In some embodiments, the inorganic base in step S2 is one mor more selected from sodium hydride, potassium phosphate, potassium acetate, sodium carbonate, potassium carbonate, caesium carbonate, sodium hydroxide, lithium hydroxide and potassium hydroxide.

In some embodiments, the organic base in step S2 is one mor more selected from triethylamine, pyridine, N,N-diisopropylethylamine, n-butyllithium, diisopropylammonium lithium, sodium acetate, potassium acetate, sodium tert-butanol, potassium tert-butanol and 1,8-diazabicyclohexadec-7-ene.

In some embodiments, Step S2 occurs in the presence of [1,1′-Bis(diphenylphosphino)ferrocene]dichloropalladium(II).

In some embodiments, Step S3 occurs in the presence of a base.

In some embodiments, the base in step S3 is selected from inorganic base and organic base.

In some embodiments, the inorganic base in step S3 is one mor more selected from sodium hydride, potassium phosphate, potassium acetate, sodium carbonate, potassium carbonate, caesium carbonate, sodium hydroxide, lithium hydroxide and potassium hydroxide.

In some embodiments, the organic base in step S3 is one mor more selected from triethylamine, pyridine, N,N-diisopropylethylamine, n-butyllithium, diisopropylammonium lithium, sodium acetate, potassium acetate, sodium tert-butanol, potassium tert-butanol and 1,8-diazabicyclohexadec-7-ene.

In some embodiments, Step S3 occurs in the presence of [1,1′-Bis(diphenylphosphino)ferrocene]dichloropalladium(II).

In some embodiments, Step S4 occurs in the presence of an acid.

In some embodiments, the acid in step S4 is one or more selected from hydrochloric acid, sulphuric acid, formic acid, acetic acid and trifluoroacetic acid.

In some embodiments, when R¹ groups of compounds a, b, c, d and I includes an amino group, said scheme further includes the protection step and deprotection step for the amino group.

In some embodiments, the protecting group is selected from Boc and Cbz.

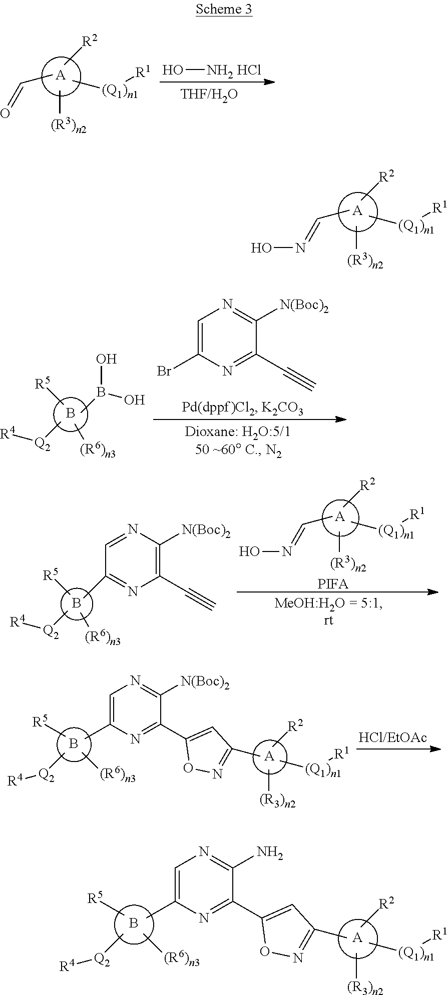

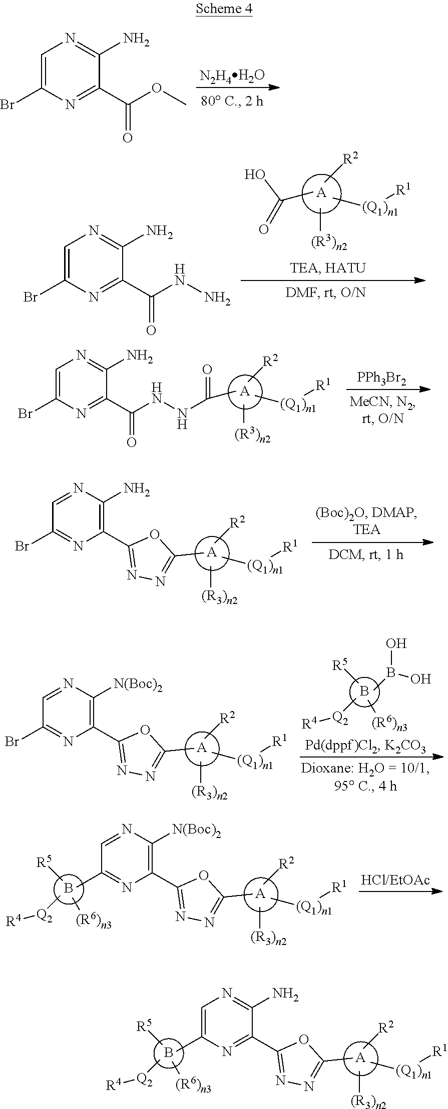

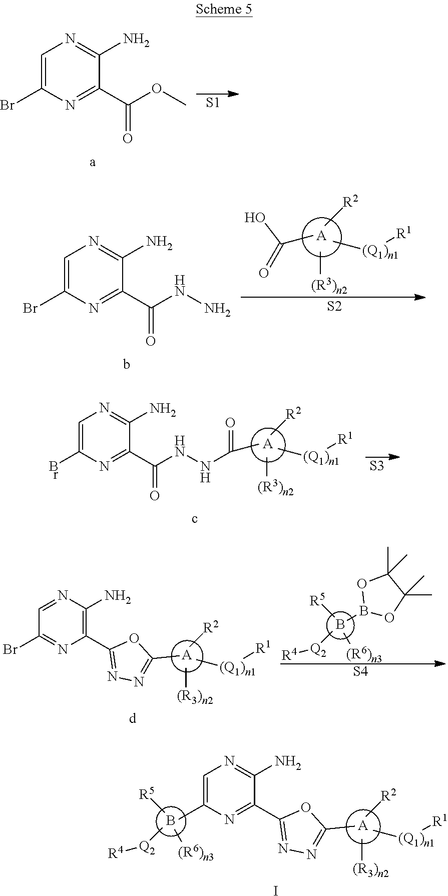

In some embodiments, Step S1 occurs in the presence of hydrazine hydrate.

In some embodiments, Step S2 occurs in the presence of a base.

In some embodiments, the base in step S2 is selected from inorganic base and organic base.

In some embodiments, the inorganic base in step S2 is one mor more selected from sodium hydride, potassium phosphate, potassium acetate, sodium carbonate, potassium carbonate, caesium carbonate, sodium hydroxide, lithium hydroxide and potassium hydroxide.

In some embodiments, the organic base in step S2 is one mor more selected from triethylamine, pyridine, N,N-diisopropylethylamine, n-butyllithium, diisopropylammonium lithium, sodium acetate, potassium acetate, sodium tert-butanol, potassium tert-butanol and 1,8-diazabicyclohexadec-7-ene.

In some embodiments, Step S2 occurs in the presence of O-(7-azabenzotriazol-1-yl)-N, N, N′, N′-tetramethylurea hexafluorophosphate ester.

In some embodiments, Step S3 occurs in the presence of a base.

In some embodiments, the base in step S3 is selected from inorganic base and organic base.

In some embodiments, the inorganic base in step S3 is one mor more selected from sodium hydride, potassium phosphate, potassium acetate, sodium carbonate, potassium carbonate, caesium carbonate, sodium hydroxide, lithium hydroxide and potassium hydroxide.

In some embodiments, the organic base in step S3 is one mor more selected from triethylamine, pyridine, N,N-diisopropylethylamine, n-butyllithium, diisopropylammonium lithium, sodium acetate, potassium acetate, sodium tert-butanol, potassium tert-butanol and 1,8-diazabicyclohexadec-7-ene.

In some embodiments, Step S3 occurs in the presence of triphenylphosphine dibromide.

In some embodiments, Step S4 occurs in the presence of a base.

In some embodiments, the base in step S4 is selected from inorganic base and organic base.

In some embodiments, the inorganic base in step S4 is one mor more selected from sodium hydride, potassium phosphate, potassium acetate, sodium carbonate, potassium carbonate, caesium carbonate, sodium hydroxide, lithium hydroxide and potassium hydroxide.

In some embodiments, the organic base in step S4 is one mor more selected from triethylamine, pyridine, N,N-diisopropylethylamine, n-butyllithium, diisopropylammonium lithium, sodium acetate, potassium acetate, sodium tert-butanol, potassium tert-butanol and 1,8-diazabicyclohexadec-7-ene.

In some embodiments, Step S4 occurs in the presence of [1,1′-Bis(diphenylphosphino)ferrocene]dichloropalladium(II).

In some embodiments, when R¹ groups of compounds c, d and I includes an amino group, said scheme further includes the protection step and deprotection step for the amino group.

In some embodiments, the protecting group is selected from Boc and Cbz.

The present disclosure further discloses a pharmaceutical composition, comprising a therapeutically effective amount of the compound of Formula I, Formula I-a, Formula I-b, Formula I-b-1, Formula I-b-2, Table A or Table B, or a pharmaceutically acceptable salt, ester, isomer, solvate, prodrug or isotopically labeled compound thereof, and one or more pharmaceutically acceptable carriers, diluents or excipients.

In some embodiments, the unit dose of the pharmaceutical composition described in the present disclosure is 0.001 mg-1000 mg.

In some embodiments, according to the total weight of the pharmaceutical composition, the pharmaceutical composition includes 0.01%-99.99% of a compound shown in Formula I, Formula I-a, Formula I-b, Formula I-b-1, Formula I-b-2, or Table A or Table B of the present disclosure, or pharmaceutically acceptable salts, esters, isomers, solvates, prodrugs or isotopically labeled compounds thereof. In some embodiments, the pharmaceutical composition includes 0.1%-99.9% of a compound shown in Formula I, Formula I-a, Formula I-b, Formula I-b-1, Formula I-b-2, or Table A or Table B of the present disclosure, or pharmaceutically acceptable salts, esters, isomers, solvates, prodrugs or isotopically labeled compounds thereof. In some embodiments, the pharmaceutical composition includes 0.5%-99.5% of a compound shown in Formula I, Formula I-a, Formula I-b, Formula I-b-1, Formula I-b-2, or Table A or Table B of the present disclosure, or pharmaceutically acceptable salts, esters, isomers, solvates, prodrugs or isotopically labeled compounds thereof. In some embodiments, the pharmaceutical composition includes 1%-99% of a compound shown in Formula I, Formula I-a, Formula I-b, Formula I-b-1, Formula I-b-2, or Table A or Table B of the present disclosure, or pharmaceutically acceptable salts, esters, isomers, solvates, prodrugs or isotopically labeled compounds thereof. In some embodiments, the pharmaceutical composition includes 2%-98% of a compound shown in Formula I, Formula I-a, Formula I-b, Formula I-b-1, Formula I-b-2, or Table A or Table B of the present disclosure, or pharmaceutically acceptable salts, esters, isomers, solvates, prodrugs or isotopically labeled compounds thereof.

In some embodiments, according to the total weight of the pharmaceutical composition, the pharmaceutical composition in present disclosure contains 0.01% to 99.99% of a pharmaceutically acceptable carrier, diluent or excipient. In some embodiments, the pharmaceutical composition in present disclosure contains 0.1% to 99.9% of a pharmaceutically acceptable carrier, diluent or excipient. In some embodiments, the pharmaceutical composition in present disclosure contains 0.5% to 99.5% of a pharmaceutically acceptable carrier, diluent or excipient. In some embodiments, the pharmaceutical composition in present disclosure contains 1% to 99% of a pharmaceutically acceptable carrier, diluent or excipient. In some embodiments, the pharmaceutical composition in present disclosure contains 2% to 98% of a pharmaceutically acceptable carrier, diluent or excipient.

The compounds of Formula I, Formula I-a, Formula I-b, Formula I-b-1, Formula I-b-2, or Table A or Table B involved in present disclosure, or the pharmaceutically acceptable salts, esters, isomers, solvates, prodrugs or isotopically labeled compounds thereof, or the mixtures or compositions comprising the same, can be administrated to a subject via any administration routes. Routes of administration may be oral administration, intravenous injection, intramuscular injection, subcutaneous injection, rectal administration, vaginal administration, sublingual administration, nasal inhalation, oral inhalation, eye drop, or local or systemic transdermal administration.

The compounds of Formula I, Formula I-a, Formula I-b, Formula I-b-1, Formula I-b-2, or Table A or Table B involved in present disclosure, or the pharmaceutically acceptable salts, esters, isomers, solvates, prodrugs or isotopically labeled compounds thereof, or the mixtures or compositions comprising the same, can be formulated into a single dose that contains the compound of present disclosure and carriers or excipients. The dosage form may be tablets, capsules, injections, granules, powders, suppositories, pills, creams, pastes, gels, dispersions, oral solutions, inhalants, suspensions, dry suspensions, patches and lotions, etc. These dosage forms may contain ingredients commonly used in pharmaceutical preparations, such as diluents, absorbents, wetting agents, binders, disintegrants, colorants, pH adjusters, antioxidants, bacteriostats, osmotic regulators, and anti-adhesives, etc.

Suitable formulations for each dosage forms are available from the public sources, such as Remington: The Science and Practice of Pharmacy, 21^{st} edition; Lippincott Williams & Wilkins Published in 2006 and Rowe, Raymond C. Handbook of Pharmaceutical Excipients, Chicago, Pharmaceutical Press, Published in 2005. Therefore, the skilled in the art can easily prepare them.

Different administration dosages can be selected depending on the nature and intensity of the disease, age, gender, weight and administration route of different individuals. The compounds of the present disclosure can be administered in a dose of 0.01 to 500 mg/kg per day, preferably in a dose of 1-100 mg/kg per day, which can be administered in a single or multiple doses.

Present disclosure further provides the compounds of Formula I, Formula I-a, Formula I-b, Formula I-b-1, Formula I-b-2, or Table A or Table B involved in present disclosure, or the pharmaceutically acceptable salts, esters, isomers, solvates, prodrugs or isotopically labeled compounds thereof, or the mixtures or compositions comprising the same, where said isotope is selected from ²H, ³H, ¹³C, ¹⁴C, ¹⁵N, ¹⁷O ¹⁸O, ³¹P, ³²P, ³⁵S, ¹⁸F and ³⁶Cl, preferably selected from ²H.

Present disclosure further provides the compounds of Formula I, Formula I-a, Formula I-b, Formula I-b-1, Formula I-b-2, or Table A or Table B involved in present disclosure, or the pharmaceutically acceptable salts, esters, isomers, solvates, prodrugs or isotopically labeled compounds thereof, or the mixtures or compositions comprising the same, where ring C is substituted by an isotope selected from ²H, ³H, ¹³C, ¹⁴C ¹⁵N, ¹⁷O, ¹⁸O, ³¹P, ³²P, ³⁵S, ¹⁸F and ³⁶Cl, preferably selected from ²H.

Present disclosure further provides the use of compounds of Formula I, Formula I-a, Formula I-b, Formula I-b-1, Formula I-b-2, or Table A or Table B involved in present disclosure, or the pharmaceutically acceptable salts, esters, isomers, solvates, prodrugs or isotopically labeled compounds thereof, or the mixtures or compositions comprising the same, for the preparation of a medicament useful for an inhibition of an ataxia telangiectasia and Rad3-related (ATR) kinase.

Present disclosure further provides the use of compounds of Formula I, Formula I-a, Formula I-b, Formula I-b-1, Formula I-b-2, or Table A or Table B involved in present disclosure, or the pharmaceutically acceptable salts, esters, isomers, solvates, prodrugs or isotopically labeled compounds thereof, or the mixtures or compositions comprising the same, for the preparation of a medicament useful for the treatment or prevention of diseases or conditions mediated by an ATR kinase.

Present disclosure further provides the use of compounds of Formula I, Formula I-a, Formula I-b, Formula I-b-1, Formula I-b-2, or Table A or Table B involved in present disclosure, or the pharmaceutically acceptable salts, esters, isomers, solvates, prodrugs or isotopically labeled compounds thereof, or the mixtures or compositions comprising the same, for the preparation of a medicament useful for the treatment or prevention of diseases or conditions mediated by an ATR kinase, wherein said diseases or conditions are diseases, disorders or conditions of excessive or abnormal cell proliferation, preferably, said diseases, disorders or conditions are selected from cancers and myeloproliferative disorders, more preferably, said diseases, disorders or conditions are selected from skin cancer, bladder cancer, ovarian cancer, breast cancer, gastric cancer, prostate cancer, colorectal cancer, lung cancer, bone cancer, brain cancer, oesophageal cancer, tongue cancer, stomach cancer, kidney cancer, cervical cancer, endometrial cancer, testicular cancer, urinary cancer, melanoma, squamous cell carcinoma, glioma, meningioma, Hodgkin's lymphoma, non-Hodgkin's lymphoma, acute lymphoblastic leukemia, chronic lymphatic leukemia, acute myelogenous leukemia, chronic myelogenous leukemia, adult T-cell leukemia lymphoma, liver cancer, bronchial cancer, multiple myeloma, basal cell tumour and sertoliomyeloma tumour; wherein said colorectal cancer is preferably colon cancer and rectal cancer; said kidney cancer is preferably parenchymal renal cancer; said glioma is preferably astrocytoma said liver cancer is preferably hepatocellular carcinoma; said lung cancer is preferably small cell lung cancer or non-small cell lung cancer; said sarcoma is preferably selected from rhabdomyosarcoma, chondrosarcoma, leiomyosarcoma and fibrosarcoma.

Present disclosure further provides compounds of Formula I, Formula I-a, Formula I-b, Formula I-b-1, Formula I-b-2, or Table A or Table B involved in present disclosure, or the pharmaceutically acceptable salts, esters, isomers, solvates, prodrugs or isotopically labeled compounds thereof, or compositions comprising the same, used as a medicament.

Present disclosure further provides compounds of Formula I, Formula I-a, Formula I-b, Formula I-b-1, Formula I-b-2, or Table A or Table B involved in present disclosure, or the pharmaceutically acceptable salts, esters, isomers, solvates, prodrugs or isotopically labeled compounds thereof, or compositions comprising the same, used as an ATR kinase inhibitor.

Present disclosure further provides compounds of Formula I, Formula I-a, Formula I-b, Formula I-b-1, Formula I-b-2, or Table A or Table B involved in present disclosure, or the pharmaceutically acceptable salts, esters, isomers, solvates, prodrugs or isotopically labeled compounds thereof, or compositions comprising the same, used for the prevention and/or treatment of ATR kinase-mediated diseases or conditions.

Present disclosure further provides compounds of Formula I, Formula I-a, Formula I-b, Formula I-b-1, Formula I-b-2, or Table A or Table B involved in present disclosure, or the pharmaceutically acceptable salts, esters, isomers, solvates, prodrugs or isotopically labeled compounds thereof, or compositions comprising the same, used for the prevention and/or treatment of ATR kinase-mediated diseases or conditions, wherein the diseases or conditions are diseases, disorders or conditions of excessive or abnormal cell proliferations, preferably, said diseases, disorders or conditions are selected from cancers and myeloproliferative disorders, more preferably, said cancer is selected from skin cancer, bladder cancer, ovarian cancer, breast cancer, gastric cancer, prostate cancer, colorectal cancer, lung cancer, bone cancer, brain cancer, oesophageal cancer, tongue cancer, stomach cancer, kidney cancer, cervical cancer, endometrial cancer, testicular cancer, urinary cancer, melanoma, squamous cell carcinoma, glioma, meningioma, Hodgkin's lymphoma, non-Hodgkin's lymphoma, acute lymphoblastic leukemia, chronic lymphatic leukemia, acute myelogenous leukemia, chronic myelogenous leukemia, adult T-cell leukemia lymphoma, liver cancer, bronchial cancer, multiple myeloma, basal cell tumour and sertoliomyeloma tumour; wherein said colorectal cancer is preferably colon cancer and rectal cancer; said kidney cancer is preferably parenchymal renal cancer; said glioma is preferably astrocytoma; said liver cancer is preferably hepatocellular carcinoma; said lung cancer is preferably small cell lung cancer or non-small cell lung cancer; said sarcoma is preferably selected from rhabdomyosarcoma, chondrosarcoma, leiomyosarcoma and fibrosarcoma.

Present disclosure further provides a method for preventing and/or treating an ATR kinase-mediated diseases or conditions, comprising administering a therapeutically effective amount of a compound of Formula I, Formula I-a, Formula I-b, Formula I-b-1, Formula I-b-2, or Table A or Table B involved in present disclosure, or the pharmaceutically acceptable salts, esters, isomers, solvates, prodrugs or isotopically labeled compounds thereof, or compositions comprising the same, to a subject needed thereof, wherein the diseases or conditions are diseases, disorders or conditions of excessive or abnormal cell proliferations, preferably, said diseases, disorders or conditions are selected from cancers and myeloproliferative disorders, more preferably, said cancer is selected from skin cancer, bladder cancer, ovarian cancer, breast cancer, gastric cancer, prostate cancer, colorectal cancer, lung cancer, bone cancer, brain cancer, oesophageal cancer, tongue cancer, stomach cancer, kidney cancer, cervical cancer, endometrial cancer, testicular cancer, urinary cancer, melanoma, squamous cell carcinoma, glioma, meningioma, Hodgkin's lymphoma, non-Hodgkin's lymphoma, acute lymphoblastic leukemia, chronic lymphatic leukemia, acute myelogenous leukemia, chronic myelogenous leukemia, adult T-cell leukemia lymphoma, liver cancer, bronchial cancer, multiple myeloma, basal cell tumour and sertoliomyeloma tumour; wherein said colorectal cancer is preferably colon cancer and rectal cancer; said kidney cancer is preferably parenchymal renal cancer; said glioma is preferably astrocytoma; said liver cancer is preferably hepatocellular carcinoma; said lung cancer is preferably small cell lung cancer or non-small cell lung cancer; said sarcoma is preferably selected from rhabdomyosarcoma, chondrosarcoma, leiomyosarcoma and fibrosarcoma.

Present disclosure further provides a method for preventing and/or treating an ATR kinase-mediated diseases of a mammal (which may be human) or a non-mammal, comprising administering a therapeutically effective amount of a compound of Formula I, Formula I-a, Formula I-b, Formula I-b-1, Formula I-b-2, or Table A or Table B involved in present disclosure, or the pharmaceutically acceptable salts, esters, isomers, solvates, prodrugs or isotopically labeled compounds thereof, or compositions comprising the same, to a mammal.

### Interpretation of Terms

Unless stated to the contrary, terms used in the specification and claims have the following meanings.

Term “C₁₋₆ alkyl” alone or in combination refers to a straight or branched alkyl chains consisting of 1 to 6, particularly 1 to 4 carbon atoms, including methyl, ethyl, propyl, isopropyl, butyl, sec-butyl, isobutyl, tert-butyl, n-pentyl, 2-pentyl, 3-pentyl, 2-methyl-2-butyl, 3-methyl-2-butyl, 3-methyl-1-butyl, 2-methyl-1-butyl, n-hexyl, 2-hexyl, 3-hexyl, 2-methyl-2-pentyl 3-methyl-2-pentyl, 4-methyl-2-pentyl, 3-methyl-3-pentyl, 2-methyl-3-pentyl, 2,3-dimethyl-2-butyl, 3,3, -dimethyl-2-butyl, etc. Preferably, “C₁₋₆ alkyl” is any of methyl, ethyl, isopropyl, and tert-butyl. Alkyl groups can be substituted or unsubstituted, and when substituted, the substituent can be attached to any available connection point. The substituent is preferably one or more of the following groups, which are independently selected from alkyl, alkynyl, alkynyl, alkoxy, alkylthio, alkylamino, halogen, thiol, hydroxyl, nitro, cyano, cycloalkyl, heterocyclic, aryl, heteroaryl, cycloalkoxy, heterocyclic alkoxy, cycloalkylthio, heterocyclic alkylthio, oxy, carboxyl or carboxylate ester group.

Term “C₁₋₆ alkylene” refers to a saturated straight or branched aliphatic hydrocarbon group having two residues derived from the removal of two hydrogen atoms from the same carbon atom or two different carbon atoms of the parent alkane, which is an alkylene group comprising 1 to 6 carbon atoms. Non-limiting examples of alkylene groups include, but are not limited to, methylene (—CH₂—), 1,1-ethylene (—CH(CH₃)—), 1,2-ethylene (—CH₂CH₂)—, 1,1-propylene (—CH(CH₂CH₃)—), 1,2-propylene (—CH₂CH(CH₃)—), 1,3-propylene (—CH₂CH₂CH₂—), 1,4-butylene (—CH₂CH₂CH₂CH₂—), and the like. The alkylene group may be substituted or unsubstituted, and when substituted, the substituent may be attached to any available connection point.

Term “C₃₋₇ cycloalkyl” refers to saturated or partially unsaturated monocyclic or polycyclic cyclic hydrocarbon groups, which represents saturated cycloalkyl groups having 3 to 7, in particular 3 to 6 carbon atoms, including cyclopropyl, cyclobutyl, cyclopentyl, cyclohexyl, cycloheptyl and the like. In particular, “C₃₋₇ cycloalkyl” is cyclopropyl, cyclopentyl, cyclohexyl and the like.

Term “C₁₋₆ alkylamino” alone or in combination refers to an amino group as defined above, wherein the hydrogen atom of the amino group is substituted by at least one C₁₋₆ alkyl group, and “C₁₋₆ alkyl” is as defined above. Accordingly, C₁₋₆ alkylamino includes methylamino, ethylamino, propylamino, isopropylamino, n-butylamino, isobutylamino, 2-butylamino, tert-butylamino, n-pentylamino, 2-pentylamino, 3-pentylamino, 2-methyl-2-butylamino, 3-methyl-2-butylamino, 3-methyl-1-butylamino, 2-methyl-1-butylamino, n-hexylamino, 2-hexylamino, 3 hexylamino, 2-methyl-2-pentylamino, 3-methyl-2-pentylamino, 4-methyl-2-pentylamino, 3-methyl-3-pentylamino, 2-methyl-3-pentylamino, 2,3-dimethyl-2-butylamino, 3,3-dimethyl-2-butylamino, and the like. In particular, “C₁₋₆ alkylamino” is methylamino acid, ethylamino acid, isopropylamino acid, tert-butylamino acid, and the like.

Term “C₁₋₆ alkylamino” alone or in combination refers to C₁₋₆ alkyl-O—, in which the C₁₋₆ alkyl-O— is as defined above.

Term “heterocyclic group” refers to a saturated or partially unsaturated (containing 1 or 2 double bonds) non-aromatic cyclic group consisting of carbon atoms and heteroatoms such as nitrogen, oxygen or sulphur atom. The heterocyclic group may be a monocyclic or bicyclic group. In present disclosure, the number of heteroatoms in the heterocyclic group is preferably 1, 2, 3 or 4, and the nitrogen, carbon or sulphur atoms in the heterocyclic group may optionally be oxidized. The hydrogen atom on the “heterocyclic group” is independently and optionally substituted by one or more of the substituents described in present disclosure. The “heterocyclic group” can be linked to the parent molecule by any ring atom on the ring.

Term “3- to 7-membered heterocyclic group” refers to a monocyclic heterocyclic group comprising 3 to 7 carbon atoms and heteroatoms; e.g., aziridinyl, azetidinyl, oxetidinyl, pyrrolidinyl, tetrahydrofuranyl, tetrahydrothiophene, piperidinyl, morpholino, morpholinyl, thiomorpholino, tetrahydropyranyl, 1,1-dioxothiomorpholino.

Term “3- to 7-membered cycloalkylamino” alone or in combination refers to an amino group as defined above wherein the hydrogen atom of the amino group is substituted by at least one C₃₋₇ cycloalkyl group as defined above.

Term “3-7-membered heterocyclic amino” alone or in combination refers to the amino group as defined above, where the hydrogen atom of the amino group is substituted by at least one 3-7-membered heterocyclic group as defined above.

Term “aryl” refers to any stable 6- to 10-membered single or bicyclic aromatic group, including phenyl, naphthyl, tetrahydronaphthyl, 2,3-dihydroindenyl, or biphenyl group.

Term “heteroaryl” refers to an aromatic ring group formed by the substitution of a carbon atom on the ring by at least one heteroatom selected from sulfur, oxygen or nitrogen. The aromatic ring group may be a 5- to 7-membered monocyclic or 7-12 bicyclic group. In present disclosure, the heterocyclic group preferably has 1, 2, 3 or 4 heteroatoms, such as thienyl, pyridyl, pyrimidyl, pyrazinyl, pyridazinyl, pyrrolyl, pyrazolyl, thiazolyl, 1,2,3-triazolyl, 1,2,4-triazolyl, imidazolyl, tetrazolyl, isothiazolyl, oxazolyl, isoxazolyl, thiadiazolyl, oxadiazolyl, naphthalenyl, benzo[b]thiopheneyl, indolyl, benzimidazolyl benzothiazolyl, benzofuranyl, quinolinyl, isoquinolinyl, quinazolinyl, etc.

Term “aryl C₁₋₆ alkyl” refers to a C₁₋₆ alkyl group substituted with one or more aryl groups, aryl and C₁₋₆ alkyl as defined above.

Term “heteroaryl C₁₋₆ alkyl” refers to a C₁₋₆ alkyl group substituted with one or more heteroaryl groups, heteroaryl and C₁₋₆ alkyl as defined above.

Term “amino”, alone or in combination, refers to primary amino (—NH₂), secondary amino (—NH—) or tertiary

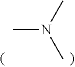

amino group.

The term “halogen” alone or in combination refers to fluorine, chlorine, bromine or iodine, particularly, to fluorine, chlorine or bromine.

Term “cyano”, alone or in combination, refers to —CN.

Term “carboxyl” alone or in combination, refers to —COOH.

Term “hydroxyl” alone or in combination, refers to —OH.

Term “guanidino”, alone or in combination, refers to —NH—C(═NH)—NH₂.

Term “ureido”, alone or in combination, refers to —NH—C(═O)—NH₂.

Term “amide group”, alone or in combination, refers to —C(═O)—NH₂.

Term “substituted” refers to one or more hydrogen atoms in the group, preferably up to 5, more preferably 1 to 3 hydrogen atoms, each independently substituted by a corresponding number of substituents. It goes without saying that substituents are only at their possible chemical positions, and the person skilled in the art is able to determine (by experiment or theory) possible or impossible substitutions without the excessive effort. For example, an amino or hydroxyl group having a free hydrogen may be unstable when combined with a carbon atom having an unsaturated (e.g., alkenyl) bond.

Term “replace” refers to the complete replacement of one group by another, e.g., the replacement of a methylene group by a heteroatom such as an oxygen atom or a nitrogen atom.

In the chemical structures of the compounds disclose in present disclosure, the bond “ [symbol] ” does not specify a configuration, i.e., the bond “ [symbol] ” may be “ [symbol] ” or “ [symbol] ”, or contain both “ [symbol] ” and “ [symbol] ” configurations. The bond “ [symbol] ” indicates a single conformation of “ [symbol] ” or “ [symbol] ”. The bond “ [symbol] ” in the chemical structure of the compounds disclosed in present disclosure does not specify a configuration, i.e., it can be a Z configuration or an E configuration, or contain both.

Term “isomer” encompasses all tautomeric forms including enantiomers, diastereomers and geometric isomers (including cis-trans isomers). Accordingly, the single stereochemical isomer, or mixtures of enantiomers, diastereomers, or geometric (or cis-trans) isomers of the compound designed in present disclosure fall within the scope of present invention.

The term “isotopically labeled” refers to the optional introduction of isotopes into any compound of present disclosure. The introduced isotope can be ²H, ³H, ¹³C, ¹⁴C, ¹⁵N, ¹⁷O, ¹⁸O, ³¹P, ³²P, ³⁵, ¹⁸F, ³⁶Cl, Preferably, the introduced isotope can be ²H, ³H, ¹³C, ¹⁴C. More preferably, the introduced isotope is ²H. Specific isotope derivatives can be prepared through conventional techniques.

Tenn “pharmaceutically acceptable salt” indicates that the compounds of present invention exist in the form of their pharmaceutical salts, including acid addition salts and base addition salts. Pharmaceutically acceptable salts are described in Pharmaceutically salts by S. M. Berge in J. Pharmaceutical Sciences (Vol. 66: pp. 1-19, 1977). In present disclosure, pharmaceutically acceptable non-toxic acid addition salts denote salts formed by the compounds of present disclosure with organic or inorganic acids which include, but not limited to, hydrochloric, sulfuric, hydrobromic, hydriodic, phosphoric, nitric, perchloric, acetic, oxalic, maleic, fumaric, tartaric, benzenesulfonic, mesylate, salicylic, succinic, citric, lactic, propionic, benzoic, p-toluenesulfonic, and malic. Pharmaceutically acceptable non-toxic base addition salts denote salts formed by the compounds of present disclosure with organic or inorganic bases which include, but not limited to, alkali metal salts such as lithium, sodium or potassium salts; alkaline earth metal salts such as calcium or magnesium salts; and organic base salts such as ammonium salts or N⁺(C₁₋₆ alkyl)₄ salts formed with organic bases containing N groups.

The terms involved in present invention has been defined above, and the skilled in the art can also understand the above terms in conjunction with existing technology. The following is a further description based on the content of present invention and the definition of the terms.

The preparations of compounds and medicinal salts of present disclosure are further described in conjunction with embodiments, but these embodiments are not intended to limit the scope of present disclosure.

The unspecified conditions in the experimental methods of embodiments of present disclosure, usually follow conventional conditions or conditions recommended by the raw material or commodity manufacturer. Reagents without specific source indicated are conventional reagents purchased from the market.

The abbreviations used in present disclosure are as follows:
CDCl₃: Deuterated chloroform
dioxane: 1,4-dioxane
CO₂: Carbon dioxide
cone. H₂SO₄: Concentrated sulfuric acid
DCM: Dichloromethane
DME: Diethylene glycol dimethyl ether
DMF: N,N-dimethylformamide
DMSO: Dimethyl sulfoxide
DMSO-d₆: Deuterated dimethyl sulfoxide
EtOH: Ethanol
EtONa: Sodium ethoxide
FeCl₃: Ferric chloride
g: Gram
HCOOH: Formic acid
Hz: Hertz
h: Hour
IC₅₀: Half-maximum inhibitory concentration
MeOH: Methanol
mg: Milligram
mL: Milliliter
mmol: Millimole
MHz: Megahertz
NaBH₃CN: Sodium cyanoborohydride
NaOH: Sodium hydroxide
NH₄OAc: Ammonium acetate
NMR: Nuclear Magnetic Resonance
M: Molar concentration
Oxalyl chloride: Oxalyl chloride
PBS: Phosphate buffer salt solution
PdCl₂(dppf): [1,1′-bis(diphenylphosphino)ferrocene]palladium(II) chloride
TCQ: Tetrachloroquinone
TLC: Thin Layer Chromatography
TsNHNH₂: p-Toluenesulfonyl hydrazide
uM: Micromole/Liter
μg: Microgram
μL: Microliter
δ: Chemical shift

The following describes the generalized test conditions in embodiments of the present disclosure:

First, reactions in the embodiments are generally carried out under nitrogen protection.

Further, the intermediates and final products are separated and purified by means of a chromatographic column, a preparative chromatographic plate and an ISCO rapid preparative chromatography system. The columns were packed with silica gel (300-400 mesh) produced by Shanghai Titan Technology Co. Ltd. The preparative plates were produced by Yantai Jiangyou Silica Gel Development Co. Ltd, and the columns used for ISCO (particle size 40-63 μm, 60 Å) were produced by Changzhou Suntai Technology Co.

Further, the LC-MS liquid-mass chromatography was performed using a Waters ACQUITY Arc equipped with a QDa Detector. A Waters XBridge C18 column (size 2.1×50 mm, 3.5 μm) was used. Mass spectrometry (MS) was performed using an ESI source, and indicates only the molecular weight M of the parent molecule, which is usually reported as [M+H]+. The injection volume was determined from the sample concentration. The flow rate was: 1.2 mL/min. HPLC peaks were read by UV-Vis wavelengths at 220 nm and 254 nm. The mobile phases were ultrapure water solution with 0.01% formic acid (mobile phase A) and acetonitrile solution with 0.01% formic acid (mobile phase B). The gradient elution conditions are shown in Tables C and D below:

TABLE C
Gradient elution conditions 1

| Time (min) | A(H₂O, 0.01% HCOOH) | B(CH₃CN, 0.01% HCOOH) |
|---|---|---|
| 0.0-0.3 | 95-85 | 5-15 |
| 0.3-3.2 | 85-20 | 15-80 |
| 3.2-3.8 | 20-5 | 80-95 |
| 3.8-3.81 | 5-95 | 95-5 |
| 3.81-4.0 | 95 | 5 |

TABLE D
Gradient elution conditions 1

| Time (min) | A(H₂O, 0.01% HCOOH) | B(CH₃CN, 0.01% HCOOH) |
|---|---|---|
| 0.00-5.90 | 95-5 | 5-95 |
| 5.90-5.91 | 5-95 | 95-5 |
| 5.91-6.00 | 95 | 5 |

Further, the NMR spectra uses the data obtained by a Varian 400 MHz NMR spectrometer with CDCl3, DMSO-d6 as solvent, and reported by the chemical shifts in ppm. The various peaks were described as follows: s (single peak), d (double peak), t (triple peak), q (quadruple peak), m (multiple peaks), and dd (double dichotomous peak). Coupling constants are represented by Hz.

### Example 1

### 1-(4-(5-(3-amino-6-(4-(isopropylsulfonyl)phenyl)pyrazin-2-yl)isoxazol-3-yl)benzyl)guanidine dihydrochloride 1

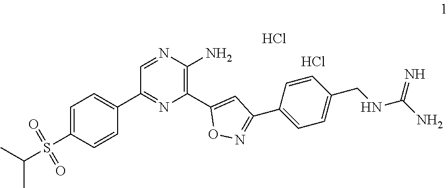 [SMILES: Cl ; CC(C)S(=O)(=O)c1ccc(-c2cnc(N)c(-c3cc(-c4ccc(CNC(=N)N)cc4)no3)n2)cc1 ; Cl]

### Step One

### (E)-4-((hydroxyimino)methyl)benzonitrile 1a

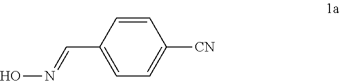 [SMILES: N#Cc1ccc(/C=N/O)cc1]

To a mixed solution of 4-cyanobenzaldehyde (5 g, 38.13 mmol) in tetrahydrofuran (30 mL) and water (3 mL) was added hydroxylamine hydrochloride (3.18 g, 45.75 mmol) at room temperature. The reaction mixture was stirred at room temperature (˜20° C.) for 16 hours. Water (40 mL) was added to the reaction system and the organic phase was separated. The aqueous phase was extracted three times with ethyl acetate (30 mL). The combined organic phase was dried with anhydrous sodium sulfate and filtered. The filtrate was concentrated under reduced pressure to give a yellow solid (E)-4-((hydroxyimino)methyl)benzonitrile 1a (4.5 g), which was a crude product and was used directly in the next step without a further purification.

### Step Two

### tert-butyl (5-bromo-3-(3-(4-cyanophenyl)isoxazol-5-yl)pyrazin-2-yl)(tert-butoxycarbonyl)carbamate 1b

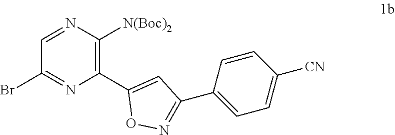

To a mixed solution of tert-butyl (5-bromo-3-ethynyl-2-pyrazinyl) (tert-butoxycarbonyl) carbamate (0.5 g, 1.26 mmol) in methanol (12 mL) and water (2.4 mL) were added sequentially (E)-4-((hydroxyimino) methyl) benzonitrile 1a (0.27 g, 1.88 mmol) and bis(trifluoroacetyl)iodobenzene (0.81 g, 1.88 mmol) and stirred overnight at room temperature. Ethyl acetate (40 mL) and water (40 mL) were added to the reaction solution. The organic phase was washed three times with water (20 mL). The organic phase was separated and dried with anhydrous sodium sulfate, filtered, concentrated and isolated by column chromatography to obtain tert-butyl (5-bromo-3-(3-(4-cyanophenyl) isoxazol-5-yl) pyrazin-2-yl) (tert-butoxycarbonyl) carbamate 1b (0.45 g).

### Step Three

### tert-butyl (tert-butoxycarbonyl) (3-(3-(4-cyanophenyl)isoxazol-5-yl)-5-(4-(isopropylsulfonyl)phenyl)pyrazin-2-yl) carbamate 1c

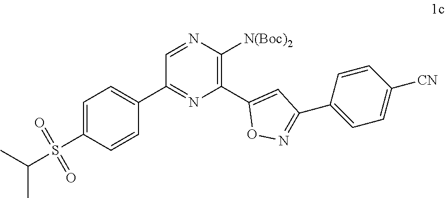

Under nitrogen protection, to a mixed solution of tert-butyl (5-bromo-3-(3-(4-cyanophenyl)isoxazol-5-yl)pyrazin-2-yl)(tert-butoxycarbonyl) carbamate 1b (1.5 g, 3.39 mmol) in 1,4-dioxane (15 mL) and water (1.5 mL), 4-isopropylsulfonyl benzeneboronic acid (0.77 g 3.39 mmol) were added potassium carbonate (0.94 g, 6.78 mmol) and [1,1′-bis(diphenylphosphino) ferrocene]palladium dichloride (0.5 g, 0.678 mmol) sequentially at room temperature. After displacing nitrogen three times, the reaction solution was stirred at 95° C. for three hours and then cooled to room temperature. Ethyl acetate (40 mL) and water (40 mL) were added to the reaction solution. The mixed solution was filtered by diatomaceous earth and the resulting solution was separated. The aqueous phase was extracted three times with ethyl acetate (20 mL), and the combined organic phase was concentrated and isolated by column chromatography to obtain tert-butyl (tert-butyloxycarbonyl) (3-(3-(4-cyanophenyl)isoxazol-5-yl)-5-(4-(isopropylsulfonyl)phenyl)pyrazin-2-yl)carbamate 1c (0.6 g).

### Step Four

### tert-butyl (3-(3-(4-(aminomethyl)phenyl)isoxazol-5-yl)-5-(4-(isopropylsulfonyl)phenyl)pyrazin-2-yl) (tert-butoxycarbonyl) carbamate 1d

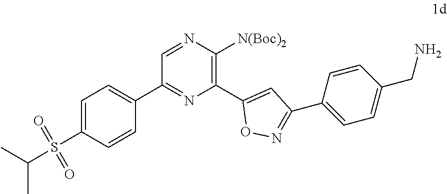

To a solution of tert-butyl (tert-butoxycarbonyl) (3-(3-(4-cyanophenyl)isoxazol-5-yl)-5-(4-(isopropylsulfonyl) phenyl)pyrazin-2-yl) carbamate 1c (540 mg, 0.99 mmol) in methanol (15 mL) were added nickel chloride hexahydrate (705 mg, 2.97 mmol) and sodium borohydride (112 mg, 2.97 mmol) sequentially at room temperature. The reaction solution was stirred for 1 hour at room temperature and then quenched with ammonium chloride (2 mL). Ethyl acetate (40 mL) and saturated aqueous sodium bicarbonate (40 mL) were then added. The mixed solution was filtered through diatomaceous earth and the filtrate was poured into a separatory funnel. The aqueous phase was extracted three times with ethyl acetate (40 mL) and the combined organic phase was concentrated and isolated by column chromatography to obtain tert-butyl (3-(3-(4-(aminomethyl) phenyl)isoxazol-5-yl)-5-(4-(isopropylsulfonyl)phenyl)pyrazin-2-yl)(tert-butoxycarbonyl) carbamate 1d (300 mg).

### Step Five

### tert-butyl (tert-butoxycarbonyl) (3-(3-(4-(guanidinylmethylene)phenyl)isoxazol-5-yl)-5-(4-(isopropylsulfonyl)phenyl)pyrazin-2-yl) carbamate 1e

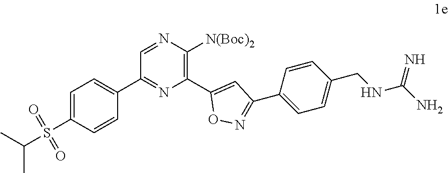

To a solution of tert-butyl (3-(3-(4-(aminomethyl)phenyl)isoxazol-5-yl)-5-(4-(isopropylsulfonyl)phenyl) pyrazin-2-yl)(tert-butoxycarbonyl) carbamate 1d (36 mg, 0.053 mmol) in acetonitrile (3 mL) were added 1H-pyrazole-1-carboximidamide hydrochloride (7.5 mg, 0.051 mmol) and N,N-diisopropylethylamine (10 μL) sequentially. The reaction solution was stirred for 16 hours at room temperature. To the reaction solution were added ethyl acetate (20 mL) and water (20 mL), and the resulting solution was separated. The aqueous phase was extracted three times with ethyl acetate (20 mL), and the combined organic phase was concentrated and isolated by column chromatography to obtain tert-butyl (tert-butoxycarbonyl) (3-(3-(4-(guanidinylmethyl)phenyl)isoxazol-5-yl)-5-(4-(isopropylsulfonyl)phenyl)pyrazin-2-yl) carbamate 1e (27 mg).

### Step Six

### 1-(4-(5-(3-amino-6-(4-(isopropylsulfonyl)phenyl)pyrazin-2-yl)isoxazol-3-yl)benzyl)guanidine dihydrochloride 1

To a solution of tert-butyl (tert-butoxycarbonyl) (3-(3-(4-(guanidinylmethyl)phenyl)isoxazol-5-yl)-5-(4-(isopropylsulfonyl)phenyl)pyrazin-2-yl)carbamate 1e (27 mg, 0.04 mmol) in ethyl acetate (2 mL) was added a mixture (4 M, 2 mL) of hydrochloric acid and ethyl acetate. After the reaction solution was stirred for 1 hour at room temperature, the reaction solution was concentrated to give a yellow solid 1-(4-(4-(5-(3-amino-6-(4-(isopropylsulfonyl)phenyl)pyrazin-2-yl)isoxazol-3-yl)benzyl)guanidine dihydrochloride 1 (22 mg).

¹H NMR (400 MHz, DMSO-d₆): δ 8.94 (s, 1H), 8.38-8.36 (m, 2H), 8.05-8.03 (m, 2H), 7.94-7.92 (m, 2H), 7.80 (s, 1H), 7.53-7.51 (m, 2H), 7.48 (brs, 3H), 7.35 (brs, 2H), 7.22 (brs, 3H), 4.52-4.50 (m, 2H), 3.49-3.42 (m, 1H), 1.20-1.18 (m, 6H).

MS measured value (ESI⁺) [(M+H)⁺]: 492.

### Example 2

### (4-(5-amino-6-(3-(4-((methylamino)methyl)phenyl)isoxazol-5-yl)pyrazin-2-yl)phenyl)(imino)(isopropyl)-λ⁶-sulfoxide 2

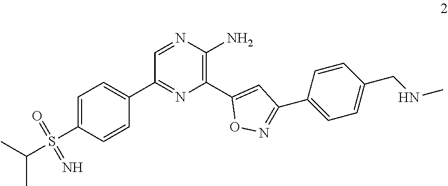 [SMILES: CNCc1ccc(-c2cc(-c3nc(-c4ccc(S(=N)(=O)C(C)C)cc4)cnc3N)on2)cc1]

### Step One

### (4-(propyl-2-sulfinylimino)phenyl)boronic acid 2a

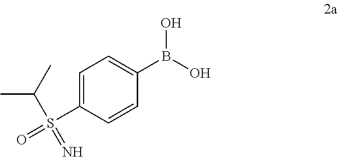

To a solution of 4-(isopropylthio)phenylboronic acid (2.20 g, 11.22 mmol) in ethanol (50 mL) were added iodophenyl diacetate (10.84 g, 33.66 mmol) and amine acetate (3.46 g, 44.88 mmol). The reaction solution was heated to 75° C. and stirred for 4 hours, and then ethyl acetate (50 mL) and water (50 mL) were added to the reaction solution. The aqueous phase was concentrated to a solid. Ethyl acetate (50 mL) and methanol (30 mL) were added to the solid, which was stirred for 0.5 h and filtrated. The filtrate was concentrated to give a black oil (4-(propyl-2-sulfinylimino)phenyl)boronic acid 2a (1.3 g), which was a crude product and was used directly in the next step without a further purification.

### Step Two

### 1-(4-(diethoxymethyl)phenyl)-N-methylmethyleneamine 2b

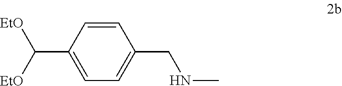 [SMILES: CCOC(OCC)c1ccc(CNC)cc1]

To a solution of terephthalaldehyde diethyl acetal (2 g, 9.60 mmol) in methanol (10 mL), triethylamine (1.6 mL) and methylamine hydrochloride (0.78 g, 11.52 mmol) were added sequentially. The reaction solution was stirred at room temperature for 0.5 h, then sodium borohydride (0.58 g, 15.37 mmol) was added. The reaction solution continued to be stirred at room temperature for another 1 hour. Ice water (0.1 mL) was added to quench the reaction followed by the addition of ethyl acetate (50 mL) and water (50 mL). The separated aqueous phase was then extracted three times with ethyl acetate (20 mL), and the combined organic phase was concentrated and isolated by column chromatography to give 1-(4-(diethoxymethyl)phenyl)-N-methyl methyleneamine 2b (2.78 g).

### Step Three

### tert-butyl (4-(diethoxymethyl)benzyl)(methylene)carbamate 2c

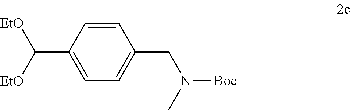 [SMILES: CCOC(OCC)c1ccc(CN(C)C(=O)OC(C)(C)C)cc1]

To a solution of 1-(4-(diethoxymethyl)phenyl)-N-methylmethyleneamine 2b (2.78 g, 9.58 mmol) in dichloromethane (20 mL) were added triethylamine (2.91 g, 28.75 mmol) and di-tert-butyl dicarbonate (6.27 g, 28.75 mmol) sequentially. The reaction solution was stirred at room temperature for 4 hours. Water (20 mL) was added to the reaction solution. After separating, the aqueous phase was extracted three times with dichloromethane (20 mL). The combined organic phase was concentrated and isolated by column chromatography to give tert-butyl (4-(diethoxymethyl)benzyl) (methylene) carbamate 2c (2.9 g).

### Step Four

### Tert-butyl (E)-(4-(((hydroxyimine) methyl) benzyl) (methyl) carbamate 2d

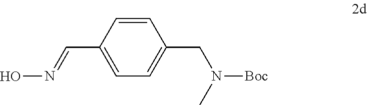 [SMILES: CN(Cc1ccc(/C=N/O)cc1)C(=O)OC(C)(C)C]

The yellow oil tert-butyl (E)-(4-((hydroxyimine) methyl) benzyl) (methyl) carbamate 2d (1.8 g) was prepared by tert-butyl (E)-(4-((hydroxyimine) methyl) benzyl) (methyl) carbamate 2c (2.9 g, 8.97 mmol) and hydroxylamine hydrochloride (0.75 g, 10.76 mmol) using a method similar to the step one of Example 1.

### Step Five

### Tert-butyl (5-bromo-3-(3-(4-(((tert-butoxycarbonyl) (methyl) amino) methyl) phenyl) isoxazole-5-yl) pyrazine-2-yl) (tert-butoxycarbonyl) carbamate 2e

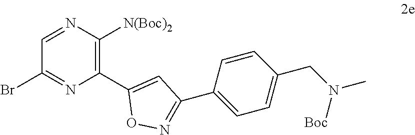

Tert-butyl (5-bromo-3-(3-(4-(((tert-butoxycarbonyl)(methyl)amino)methyl)phenyl)isoxazol-5-yl)pyrazin-2-yl)(tert-butoxycarbonyl)carbamate 2e (680 mg) was prepared by Tert-butyl (E)-(4-((hydroxyimino)methyl) benzyl)(methyl) carbamate 2d (500 mg, 1.88 mmol), (E)-4-((isonitroso)methyl)benzonitrile (500 mg, 1.26 mmol) and bis(trifluoroacetyl) iodobenzene (810 mg, 1.88 mmol) using a method similar to second step 2 of Example 1.

### Step Six

### tert-Butyl (tert-butoxycarbonyl) (3-(3-(4-(4-(((tert-butoxycarbonyl)(methyl)amino)methyl)phenyl)isoxazol-5-yl)-5-(4-(propyl-2-(propan-2-ylsulfinyl)amino)phenyl)pyrazin-2-yl) carbamate 2f

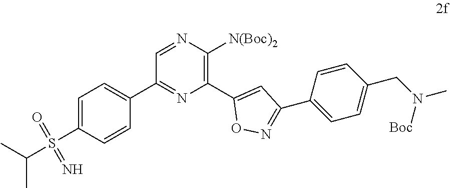

The yellow solid tert-butyl (tert-butoxycarbonyl)(3-(3-(3-(4-(((tert-butoxycarbonyl)(methyl) amino)methyl)phenyl) isoxazole-5-yl)-5-(4-(4-(propyl-2-sulfinylimino)phenyl)pyrazin-2-yl) carbamate 2f (65 mg) was prepared by tert-butyl (5-bromo-3-(3-(4-(4-(((tert-butoxycarbonyl) (methyl) amino) methyl)phenyl)isoxazol-5-yl)pyrazin-2-yl)(tert-butoxycarbonyl) carbamate 2e (100 mg, 0.151 mmol) and (4-(propyl-2-yl-sulfinylimino)phenyl)boronic acid (68 mg, 0.302 mmol), potassium carbonate (42 mg, 0.302 mmol) and [1,1′-bis(diphenylphosphino)ferrocene]palladium dichloride (22 mg, 0.03 mmol) using a method similar to step three of Example 1.

### Step Seven

### (4-(5-amino-6-(3-(4-((methylamino)methyl)phenyl)isoxazol-5-yl)pyrazin-2-yl)phenyl)(imino)(isopropyl)-λ⁶-sulfoxide 2

To a solution of tert-butyl (tert-butoxycarbonyl) (3-(3-(3-(4-(((tert-butoxycarbonyl)(methyl)amino)methyl)phenyl) isoxazol-5-yl)-5-(4-(propyl-2-ylsulfinylamino)phenyl)pyrazin-2-yl)carbamate 2f (35 mg, 0.046 mmol) in ethyl acetate (2 mL) was added a mixture (4 M, 2 mL) of hydrochloric acid and ethyl acetate. The reaction solution was stirred at room temperature for 1 h. After the reaction solution was concentrated, ethyl acetate (20 mL) and saturated aqueous sodium bicarbonate solution (20 mL) were added. Then, the solution was separated, and the organic phase was washed with water (20 mL), concentrated and isolated by column chromatography to give (4-(5-amino-6-(3-(4-((methylamino)methyl) phenyl)isoxazol-5-yl)pyrazin-2-yl)phenyl)(imino)(isopropyl)-λ⁶-sulfoxide 2 (14.5 mg).

¹H NMR (400 MHz, DMSO-d₆): δ9.29 (s, 2H), 8.93 (s, 1H), 8.33-8.31 (m, 2H), 8.11-8.09 (m, 2H), 7.95-7.92 (m, 2H), 7.84 (s, 1H), 7.74-7.72 (m, 2H), 7.23-7.17 (m, 2H), 4.20 (s, 2H), 3.30-3.24 (m, 1H), 2.58 (s, 3H), 1.17-1.15 (m, 6H).

MS measured value (ESI⁺) [(M+H)⁺]: 463.

### Example 3

### 3-(3-(1H-benzo[d][1,2,3]triazol-5-yl)isoxazol-5-yl)-5-(4-(isopropylsulfonyl)phenyl)pyrazin-2-amine 3

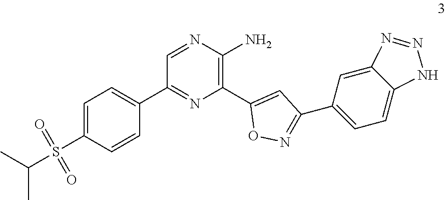 [SMILES: CC(C)S(=O)(=O)c1ccc(-c2cnc(N)c(-c3cc(-c4ccc5[nH]nnc5c4)no3)n2)cc1]

### Step One

### 1H-benzo[d][1,2,3]triazole-5-carbonitrile 3a

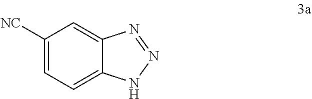 [SMILES: N#Cc1ccc2[nH]nnc2c1]

To a solution of 3,4-diaminobenzonitrile (1.0 g, 7.51 mmol) in acetic acid (15 mL) was added an aqueous solution of sodium nitrite (0.62 g sodium nitrite dissolved in 15 mL of water). The reaction solution was stirred for 2 hours at room temperature. Ethyl acetate (40 mL) and aqueous solution (20 mL) were added. After separating, the organic phase was concentrated to give a white solid 1H-benzo[d][1,2,3]triazole-5-carbonitrile 3a (0.8 g) as a crude product which was used directly in the next step without a further purification.

### Step Two

### Tert-butyl-5-cyano-1H-benzo[d][1,2,3]triazole-1-aminoformate 3b

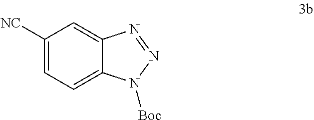 [SMILES: CC(C)(C)OC(=O)n1nnc2cc(C#N)ccc21]

Tert-butyl-5-cyano-1H-benzo[d] [1,2,3]triazole-1-amino formate 3b (1.23 g) was prepared by tert-butyl-5-cyano-1H-benzo[d] [1,2,3]triazole-5-acetonitrile 3a (0.8 g, 5.55 mmol), triethylamine (2.27 mL), and di-tert-butyl dicarbonate (3.63 g, 16.65 mmol) using a method similar to step three of Example 2.

### Step Three

### 1H-benzo[d] [1,2,3]triazole-5-formaldehyde 3c

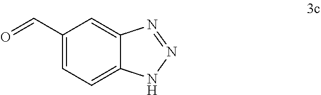 [SMILES: O=Cc1ccc2[nH]nnc2c1]

To a toluene (10 mL) solution of tert-butyl-5-cyano-1H-benzo[d] [1,2,3]triazole-1-amino formate 3b (1.23 g, 5.04 mmol), DABAL-H (12 mL, 1 M in PhMe) was added slowly at −40° C. The reaction solution was stirred at −40° C. for 3 hours, and was quenched by the addition of L-tartaric acid aqueous solution (1 mL, 1M). Then, ethyl acetate (20 mL) was added, and the system was filtered. The filtrate was washed with water (20 mL) and saturated saline water (20 mL). The organic phase was concentrated to obtain 1H-benzo[d] [1,2,3]triazole-5-formaldehyde 3c (160 mg), which is a crude product and directly used in the next step without a further purification.

### Step Four

### Tert-butyl-5-formyl-1H-benzo[d] [1,2,3]triazole-1-carbamate 3d

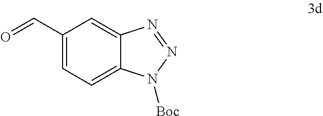 [SMILES: CC(C)(C)OC(=O)n1nnc2cc(C=O)ccc21]

Tert-butyl-5-formyl-1H-benzo[d][1,2,3]triazole-1-carbamate 3d (140 mg) was prepared by 1H-benzo[d] [1,2,3]triazole-5-carbaldehyde 3c (0.16 g, 1.09 mmol), triethylamine (0.44 mL), and di-tert-butyl dicarbonate (0.72 g, 3.26 mmol) using a method similar to step three of Example 2.

### Step Five

### Tert-butyl (E)-5-(hydroxyimine) methylene)-1H-benzo[d] [1,2,3]triazol-1-carboxylate 3e

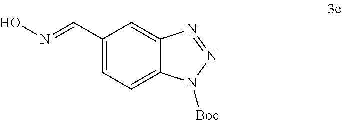 [SMILES: CC(C)(C)OC(=O)n1nnc2cc(/C=N/O)ccc21]

The yellow oil Tert-butyl (E)-5-(hydroxyimine) methylene)-1H-benzo[d] [1,2,3]triazol-1-carboxylate 3e (75 mg) was prepared by Tert-butyl-5-formyl-1H-benzo[d][1,2,3]triazole-1-carbamate 3d (0.14 g, 0.566 mmol) and hydroxylamine hydrochloride (0.048 g, 0.68 mmol) using a method similar to step one of Example 1.

### Step Six

### Tert-butyl 5-(5-(6-bromo-3-(N,N-bis(tert-butoxycarbonyl)amino)pyrazin-2-yl)isoxazol-3-yl)-1H-benzo[d][1,2,3]triazol-1-carboxylate 3f

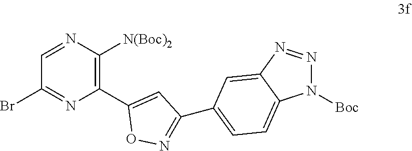

The yellow solid tert-butyl 5-(5-(6-bromo-3-(N,N-bis(tert-butoxycarbonyl)amino)pyrazin-2-yl)isoxazol-3-yl)-1H-benzo[d][1,2,3]triazole-1-carboxylate 3f (30 mg) was obtained from Tert-butyl (E)-5-(hydroxyimine) methylene)-1H-benzo[d] [1,2,3]triazole-1-carboxylate 3e, (E)-4-((isonitroso)methyl)benzonitrile (75 mg, 0.188 mmol) and bis(trifluoroacetyl) iodobenzene (121 mg, 0.282 mmol) using the method similar to step two of Example 1.

### Step Seven

### tert-butyl (3-(3-(1H-benzo[d][1,2,3]triazol-5-yl)isoxazol-5-yl)-5-(4-(isopropylsulfonyl)phenyl)pyrazin-2-yl)carbamate 3g

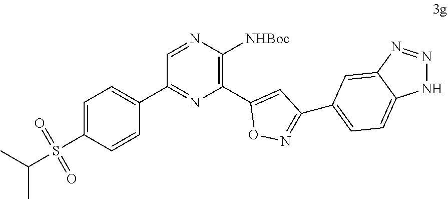 [SMILES: CC(C)S(=O)(=O)c1ccc(-c2cnc(NC(=O)OC(C)(C)C)c(-c3cc(-c4ccc5[nH]nnc5c4)no3)n2)cc1]

The yellow solid tert-butyl (3-(3-(1H-benzo[d][1,2,3]triazol-5-yl)isoxazol-5-yl)-5-(4-(isopropylsulfonyl) phenyl)pyrazine-2-yl) carbamate 3g (15 mg) was prepared by tert-butyl 5-(5-(6-bromo-3-(N,N-bis(tert-butoxycarbonyl)amino)pyrazin-2-yl)isoxazol-3-yl)-1H-benzo[d] [1,2,3]triazole-1-carboxylate 3f (30 mg, 0.045 mmol) and 4-isopropylsulfonylphenyl boronic acid (10 mg, 0.045 mmol), potassium carbonate (13 mg. 0.09 mmol) and [1,1′-bis(diphenylphosphino) ferrocene] palladium dichloride (7 mg, 0.009 mmol) using a method similar to step three of Example 1.

### Step Eight

### 3-(3-(1H-benzo[d][1,2,3]triazol-5-yl)isoxazol-5-yl)-5-(4-(isopropylsulfonyl)phenyl)pyrazin-2-amine 3

The yellow solid 3-(3-(1H-benzo[d][1,2,3]triazol-5-yl)isoxazol-5-yl)-5-(4-(isopropylsulfonyl) phenyl)pyrazin-2-amine 3 (6.9 mg) was prepared by tert-butyl (3-(3-(1H-benzo[d][1,2,3]triazol-5-yl)isoxazol-5-yl)-5-(4-(isopropylsulfonyl) phenyl)pyrazine-2-yl) carbamate 3g (15 mg, 0.027 mmol) and a mixture (2 mL, 4 M) of hydrochloric acid and ethyl acetate using a method similar to Step seven of Example 2.

¹H NMR (DMSO-d₆, 400 MHz): δ 8.96 (s, 1H), 8.65 (brs, 1H), 8.41-8.39 (m, 2H), 8.13-8.08 (m, 2H), 8.00 (s, 1H), 7.95-7.93 (m, 2H), 7.25-7.24 (m, 3H), 3.50-3.43 (m, 1H), 1.20-1.18 (m, 6H).

MS measured value (ESI⁺) [(M+H)⁺]: 462.

### Example 4

### 1-(4-(5-(3-amino-6-(4-isopropylsulfonyl)phenyl)pyrazin-2-yl)isoxazol-3-yl)-3-fluorobenzyl)guanidine dihydrochloride 4

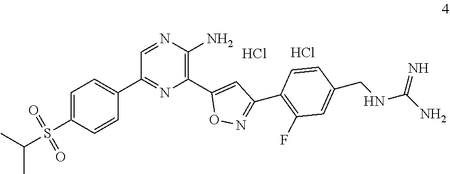 [SMILES: CC(C)S(=O)(=O)c1ccc(-c2cnc(N)c(-c3cc(-c4ccc(CNC(=N)N)cc4F)no3)n2)cc1 ; Cl ; Cl]

### Step One

### (E)-3-fluoro-4-((hydroxyimino)methylene)benzonitrile 4a

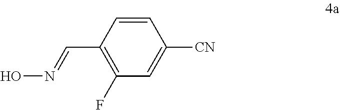 [SMILES: N#Cc1ccc(/C=N/O)c(F)c1]

To a mixed solution of 4-cyano-2-fluoroformaldehyde (2.0 g, 13.41 mmol) in methanol (30 mL) and water (3 mL) were added hydroxylamine hydrochloride (1.86 g, 26.82 mmol) and potassium carbonate (1.85 g, 13.41 mmol). The reaction mixture was stirred at 75° C. for 3 hours. Ethyl acetate (40 mL) and water (40 mL) were added to the reaction system. The organic phase was separated out, and the aqueous phase was extracted with ethyl acetate (30 mL) three times. The combined organic phase was dried with anhydrous sodium sulfate and filtered, and the filtrate was concentrated under reduced pressure to give a white solid (E)-3-fluoro-4-((hydroxyimino)methylene)benzonitrile 4a (2.1 g), which was a crude product and was used directly in the next step without a further purification.

### Step Two

### tert-butyl (tert-butoxycarbonyl)(5-bromo-3-(3-(4-cyano-2-fluorophenyl)isoxazol-5-yl)pyrazin-2-yl)carbamate 4b

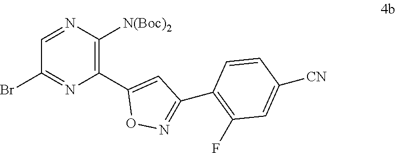

The yellow solid tert-butyl (tert-butoxycarbonyl)(5-bromo-3-(3-(4-cyano-2-fluorophenyl)isoxazol-5-yl)pyrazin-2-yl)carbamate 4b was prepared by (E)-3-fluoro-4-((hydroxyimino)methylene)benzonitrile 4a (300 mg, 1.88 mmol), tert-butyl (5-bromo-3-ethynyl-2-pyrazinyl)(tert-butyloxycarbonyl)carbamate (500 mg, 1.26 mmol), and bis(trifluoroacetyl) iodobenzene (0.81 g, 1.88 mmol) using the method similar to step two of Example 1.

### Step Three

### tert-butyl (tert-butoxycarbonyl) (3-(3-(4-cyano-2-fluorophenyl)isoxazol-5-yl)-5-(4-(isopropylsulfonyl)phenyl)pyrazin-2-y 1 carbamate 4c

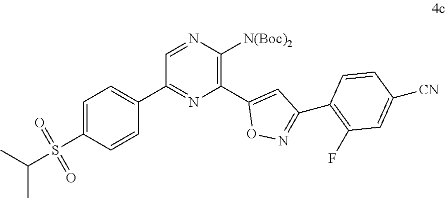

The light yellow solid tert-butyl (tert-butoxycarbonyl) (3-(3-(4-cyano-2-fluorophenyl)isoxazol-5-yl)-5-(4-(isopropylsulfonyl)phenyl)pyrazin-2-yl) carbamate 4c (230 mg) was prepared by tert-butyl (tert-butoxycarbonyl)(5-bromo-3-(3-(4-cyano-2-fluorophenyl)isoxazol-5-yl)pyrazin-2-yl)carbamate 4b (450 mg, 0.803 mmol), 4-isopropylsulfonylbenzeneboronic acid (183 mg, 0.803 mmol), potassium carbonate (222 mg, 1.61 mmol), and [1,1′-bis(diphenylphosphino)ferrocene]palladium dichloride (117 mg, 0.16 mmol) using a method similar to step three of Example 1.

### Step Four

### tert-butyl (3-(3-(4-(aminomethyl)-2-fluorophenyl)isoxazol-5-yl)-5-(4-(isopropylsulfonyl)phenyl)pyrazin-2-yl)(tert-butoxycarbonyl) carbamate 4d

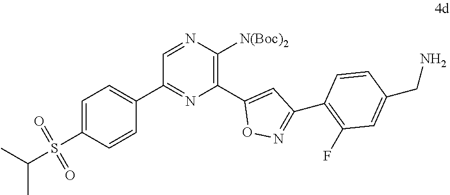

The light yellow solid tert-butyl (3-(3-(4-(aminomethyl)-2-fluorophenyl)isoxazol-5-yl)-5-(4-(isopropylsulfonyl) phenyl)pyrazin-2-yl)(tert-butoxycarbonyl) carbamate 4d (28 mg) was prepared by tert-butyl (tert-butoxycarbonyl) (3-(3-(4-cyano-2-fluorophenyl)isoxazol-5-yl)-5-(4-(isopropylsulfonyl)phenyl)-pyrazin-2-yl) carbamate 4c (100 mg, 0.177 mmol), nickel chloride hexahydrate (127 mg, 0.532 mmol) and sodium borohydride (20 mg, 0.532 mmol) using a method similar to step four of Example 1.

### Step Five

### tert-butyl (tert-butoxycarbonyl) (3-(3-(3-(2-fluoro-4-(guanidinylmethylene)phenyl)isoxazol-5-yl)-5-(4-(isopropylsulfonyl)phenyl)pyrazin-2-yl) carbamate 4e

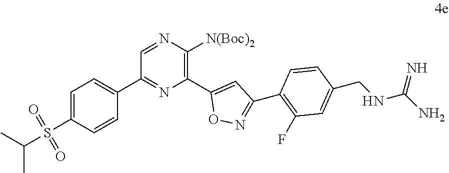

The light yellow solid tert-butyl (tert-butoxycarbonyl) (3-(3-(3-(2-fluoro-4-(guanidinylmethylene)phenyl)isoxazol-5-yl)-5-(4-(isopropylsulfonyl)phenyl)pyrazin-2-yl) carbamate 4e (15 mg) was prepared by tert-butyl (3-(3-(4-(aminomethyl)-2-fluorophenyl)isoxazol-5-yl)-5-(4-(isopropylsulfonyl) phenyl)pyrazin-2-yl)(tert-butoxycarbonyl) carbamate 4d (28 mg, 0.05 mmol), 1H-pyrazole-1-carboximidamide hydrochloride acid (7 mg, 0.05 mmol) and N,N-diisopropylethylamine (10 μL) using a method similar to step five of Example 1.

### Step Six

### 1-(4-(5-(3-amino-6-(4-isopropylsulfonyl)phenyl)pyrazin-2-yl)isoxazol-3-yl)-3-fluorobenzyl)guanidine dihydrochloride 4

The yellow solid 1-(4-(5-(3-amino-6-(4-isopropylsulfonyl)phenyl)pyrazin-2-yl)isoxazol-3-yl)-3-fluorobenzyl) guanidine dihydrochloride 4 (9.3 mg) was prepared by tert-butyl (tert-butoxycarbonyl) (3-(3-(3-(2-fluoro-4-(guanidinylmethylene)phenyl)isoxazol-5-yl)-5-(4-(isopropylsulfonyl)phenyl)pyrazin-2-yl) carbamate 4e (15 mg, 0.025 mmol) and a mixture (4 M, 2 mL) of hydrochloric acid and ethyl acetate using a method similar to step six of Example 1.

¹H NMR (400 MHz, DMSO-d₆): δ 8.95 (s, 1H), 8.50-8.47 (m, 1H), 8.37-8.35 (m, 2H), 8.06-8.02 (m, 1H), 7.94-7.92 (m, 2H), 7.61-7.60 (m, 1H), 7.47 (brs, 3H), 7.34 (brs, 3H), 7.21 (s, 3H), 4.55-4.53 (m, 2H), 3.49-3.42 (m, 1H), 1.19-1.17 (m, 6H).

MS measured value (ESI⁺) [(M+H)⁺]: 510.

### Example 5

### 1-(4-(5-(3-amino-6-(4-(isopropylsulfonyl)phenyl)pyrazin-2-yl)isoxazol-3-yl)benzyl)-3-cyclopropylurea

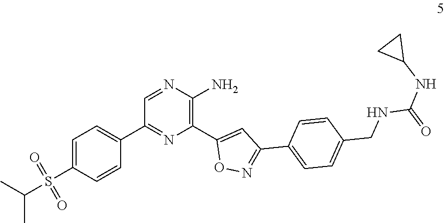 [SMILES: CC(C)S(=O)(=O)c1ccc(-c2cnc(N)c(-c3cc(-c4ccc(CNC(=O)NC5CC5)cc4)no3)n2)cc1]

### Step One

### tert-butyl (tert-butoxycarbonyl) (5-(4-(isopropylsulfonyl)phenyl)-3-(3-(4-(((phenoxycarbonyl)amino)methylene)phenyl)isoxazol-5-yl)pyrazin-2-yl) carbamate 5a

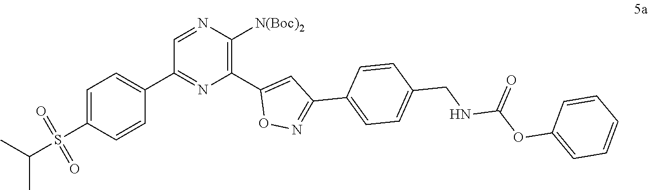

To a solution of tert-butyl (3-(3-(4-(aminomethyl)phenyl)isoxazol-5-yl)-5-(4-(isopropylsulfonyl)phenyl)pyrazin-2-yl) (tert-butoxycarbonyl) carbamate 1d (200 mg, 0.364 mmol) in dichloromethane (20 mL) were added triethylamine (0.15 mL) and phenyl chloroformate (114 mg, 0.728 mmol). The mixture was stirred at 25° C. for 2 hours. The mixture was concentrated and purified by column chromatography to give a yellow oil tert-butyl (tert-butoxycarbonyl) (5-(4-(isopropylsulfonyl)phenyl)-3-(3-(4-(((phenoxycarbonyl)amino)methylene)phenyl)isoxazol-5-yl)pyrazin-2-yl) carbamate 5a (152 mg).

### Step Two

### tert-butyl (tert-butoxycarbonyl) (3-(3-(4-((3-cyclopropylureido)methyl)phenyl)isoxazol-5-yl)-5-(4-(isopropylsulfonyl)phenyl)pyrazin-2-yl) carbamate 5b

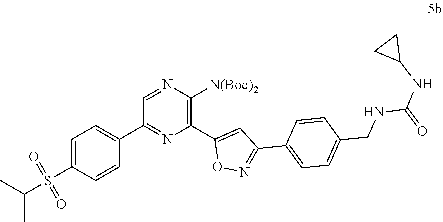

To a solution of tert-butyl (tert-butoxycarbonyl) (5-(4-(isopropylsulfonyl)phenyl)-3-(3-(4-(((phenoxycarbonyl) amino)methylene)phenyl)isoxazol-5-yl)pyrazin-2-yl) carbamate 5a (13 mg, 0.019 mmol) in acetonitrile (3 mL) was added cyclopropylamine (22 mg), and the mixture was stirred for 2 h at 70° C. Ethyl acetate (20 mL) and water (20 mL) were added to the reaction solution, and the separated aqueous phase was then extracted three times with ethyl acetate (20 mL). The combined organic phase was concentrated and isolated by column chromatography to give a white solid tert-butyl (tert-butoxycarbonyl) (3-(3-(4-((3-cyclopropylureido)methyl)phenyl)isoxazol-5-yl)-5-(4-(isopropylsulfonyl)phenyl)pyrazin-2-yl) carbamate 5b (10 mg).

### Step Three

### 1-(4-(5-(3-amino-6-(4-(isopropylsulfonyl)phenyl)pyrazin-2-yl)isoxazol-3-yl)benzyl)-3-cyclopropylurea

The yellow solid 1-(4-(5-(3-amino-6-(4-(isopropylsulfonyl)phenyl)pyrazin-2-yl)isoxazol-3-yl)benzyl)-3-cyclopropylurea 5 was prepared by tert-butyl (tert-butoxycarbonyl) (3-(3-(4-((3-cyclopropylureido)methyl)phenyl) isoxazol-5-yl)-5-(4-(isopropylsulfonyl)phenyl)pyrazin-2-yl) carbamate 5b (10 mg, 0.015 mmol) and a mixture (4 M, 2 mL) of hydrochloric acid and ethyl acetate using a method similar to step seven of Example 2.

¹H NMR (DMSO-d₆, 400 MHz): δ 8.93 (s, 1H), 8.38-8.36 (m, 2H), 7.97-7.92 (m, 4H), 7.75 (s, 1H), 7.45-7.43 (m, 2H), 7.17 (s, 2H), 6.49-6.48 (m, 1H), 6.26 (s, 1H), 4.31-4.30 (m, 2H), 3.49-3.42 (m, 1H), 2.47-2.44 (m, 1H), 1.20-1.18 (m, 6H), 0.62-0.58 (m, 2H), 0.38-0.37 (m, 2H)

MS measured value (ESI⁺) [(M+H)⁺]: 533

### Example 6

### 1-(4-(5-(3-amino-6-(4-(isopropylsulfonyl)phenyl)pyrazin-2-yl)isoxazol-3-yl)benzyl)-3-ethylurea 6

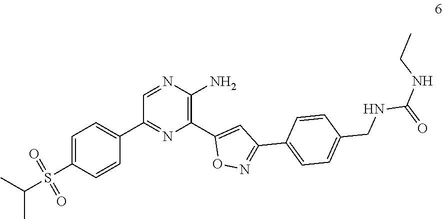 [SMILES: CCNC(=O)NCc1ccc(-c2cc(-c3nc(-c4ccc(S(=O)(=O)C(C)C)cc4)cnc3N)on2)cc1]

### Step One

### tert-butyl (tert-butoxycarbonyl) (3-(3-(3-(4-((3-ethylureido)methylene)phenyl)isoxazol-5-yl)-5-(4-(isopropylsulfonyl)phenyl)pyrazin-2-yl)carbamate 6a

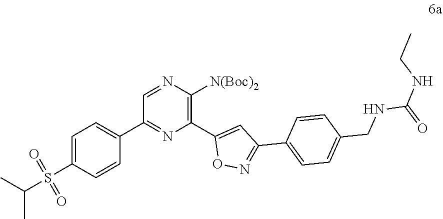

The white solid tert-butyl (tert-butoxycarbonyl) (3-(3-(3-(4-((3-ethylureido)methylene)phenyl)isoxazol-5-yl)-5-(4-(isopropylsulfonyl)phenyl)pyrazin-2-yl)carbamate 6a (7 mg) was prepared by tert-butyl (tert-butoxycarbonyl) (5-(4-(isopropylsulfonyl)phenyl)-3-(3-(4-(((phenoxycarbonyl)amino)methylene)phenyl)isoxazol-5-yl)pyrazin-2-yl) carbamate 5a (22 mg, 0.03 mmol), triethylamine (4.5 μL) and ethylchloride (27 mg, 0.3 mmol) using a method similar to step two of Example 5.

### Step Two

### 1-(4-(5-(3-amino-6-(4-(isopropylsulfonyl)phenyl)pyrazin-2-yl)isoxazol-3-yl)benzyl)-3-ethylurea 6

The yellow solid 1-(4-(5-(3-amino-6-(4-(isopropylsulfonyl)phenyl)pyrazin-2-yl)isoxazol-3-yl)benzyl)-3-ethylurea 6 (1.6 mg) was prepared by tert-butyl (tert-butoxycarbonyl) (3-(3-(3-(4-((3-ethylureido)methylene)phenyl)isoxazol-5-yl)-5-(4-(isopropylsulfonyl)phenyl)pyrazin-2-yl)carbamate 6a (7 mg, 0.01 mmol) and a mixture (4 M, 2 mL) of hydrochloric acid and ethyl acetate using a method similar to step seven of Example 2.

¹H NMR (DMSO-d₆, 400 MHz): δ 8.93 (s, 1H), 8.38-8.36 (m, 2H), 7.97-7.92 (m, 4H), 7.75 (s, 1H), 7.44-7.42 (m, 2H), 7.17 (s, 2H), 6.40-6.37 (m, 1H), 5.95-5.92 (m, 1H), 4.29-4.28 (m, 2H), 3.49-3.42 (m, 1H), 3.08-3.02 (m, 2H), 1.20-1.18 (m, 6H), 1.03-1.00 (m, 3H)

MS measured value (ESI⁺) [(M+H)⁺]: 521

### Example 7

### 1-(4-(5-(3-amino-6-(4-(isopropylsulfonyl)phenyl)pyrazin-2-yl)isoxazol-3-yl)benzyl)-3-methylurea 7

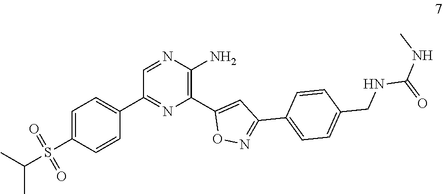 [SMILES: CNC(=O)NCc1ccc(-c2cc(-c3nc(-c4ccc(S(=O)(=O)C(C)C)cc4)cnc3N)on2)cc1]

### Step One

### tert-butyl (tert-butoxycarbonyl) (3-(3-(3-(4-((3-methylureido)methylene)phenyl)isoxazol-5-yl)-5-(4-(isopropylsulfonyl)phenyl)pyrazin-2-yl)carbamate 7a

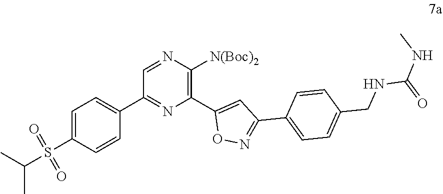

The white solid tert-butyl (tert-butoxycarbonyl) (3-(3-(3-(4-((3-methylureido)methylene)phenyl)isoxazol-5-yl)-5-(4-(isopropylsulfonyl)phenyl)pyrazin-2-yl)carbamate 7a (11 mg) was prepared by tert-butyl (tert-butoxycarbonyl) (5-(4-(isopropylsulfonyl)phenyl)-3-(3-(4-(((phenoxycarbonyl)amino)methylene)phenyl)isoxazol-5-yl)pyrazin-2-yl) carbamate 5a (15 mg, 0.02 mmol), triethylamine (3 μL), and methylchloride (15 mg, 0.2 mmol) using a method similar to step two of Example 5.

### Step Two

### 1-(4-(5-(3-amino-6-(4-(isopropylsulfonyl)phenyl)pyrazin-2-yl)isoxazol-3-yl)benzyl)-3-methylurea 7

The yellow solid 1-(4-(4-(5-(3-amino-6-(4-(isopropylsulfonyl)phenyl)pyrazin-2-yl)isoxazol-3-yl)benzyl)-3-methylurea 7 (6.3 mg) was prepared by tert-butyl (tert-butoxycarbonyl) (3-(3-(3-(4-((3-methylureido)methylene)phenyl)isoxazol-5-yl)-5-(4-(isopropylsulfonyl)phenyl)pyrazin-2-yl)carbamate 7a (11 mg, 0.018 mmol) and a mixture (4 M, 2 mL) of hydrochloric acid and ethyl acetate using a method similar to step seven of Example 2.

¹H NMR (DMSO-d₆, 400 MHz): δ 8.93 (s, 1H), 8.38-8.36 (m, 2H), 7.97-7.92 (m, 4H), 7.75 (s, 1H), 7.44-7.42 (m, 2H), 7.16 (s, 2H), 6.49-6.46 (m, 1H), 5.87-5.86 (m, 1H), 4.29-4.27 (m, 2H), 3.47-3.44 (m, 1H), 2.59-2.58 (m, 3H), 1.20-1.18 (m, 6H)

MS measured value (ESI⁺) [(M+H)⁺]: 507

### Example 8

### 3-(3-(4-((aminosulfonyl)aminomethylene)phenyl)isoxazol-5-yl)-5-(4-(isopropylsulfonyl)phenyl)pyrazin-2-amine 8

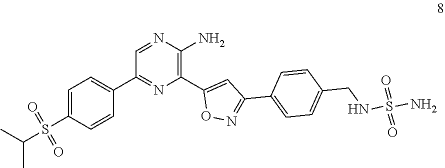 [SMILES: CC(C)S(=O)(=O)c1ccc(-c2cnc(N)c(-c3cc(-c4ccc(CNS(N)(=O)=O)cc4)no3)n2)cc1]

### Step One

### tert-butyl (tert-butoxycarbonyl) (3-(3-(3-(4-(((N-(tert-butoxycarbonyl)sulfamoyl)amino)methylene)phenyl)isoxazol-5-yl)-5-(4-(isopropylsulfonyl)phenyl)pyrazin-2-yl)carbamate 8a

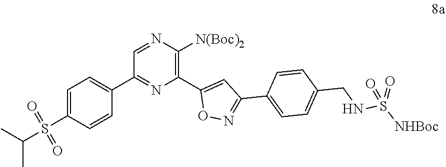

The light yellow solid tert-butyl (tert-butoxycarbonyl) (3-(3-(3-(4-(((N-(tert-butoxycarbonyl)sulfamoyl)amino) methylene)phenyl)isoxazol-5-yl)-5-(4-(isopropylsulfonyl)phenyl)pyrazin-2-yl)carbamate 8a (13 mg) was prepared by tert-butyl (3-(3-(4-(aminomethyl)phenyl)isoxazol-5-yl)-5-(4-(isopropylsulfonyl)phenyl)pyrazin-2-yl) (tert-butoxycarbonyl) carbamate 1d (30 mg, 0.05 mmol), triethylamine (20 μL), and tert-butyl chlorosulfonyl carbamate (24 mg, 0.1 mmol) using a method similar to step one of Example 1.

### Step Two

### 3-(3-(4-((aminosulfonyl)aminomethylene)phenyl)isoxazol-5-yl)-5-(4-(isopropylsulfonyl)phenyl)pyrazin-2-amine 8

The yellow solid 3-(3-(4-((aminosulfonyl)aminomethylene)phenyl)isoxazol-5-yl)-5-(4-(isopropylsulfonyl)phenyl) pyrazin-2-amine 8 (6.2 mg) was prepared by tert-butyl (tert-butoxycarbonyl) (3-(3-(3-(4-(((N-(tert-butoxycarbonyl) sulfamoyl)amino)methylene)phenyl)isoxazol-5-yl)-5-(4-(isopropylsulfonyl)phenyl)pyrazin-2-yl)carbamate 8a (13 mg, 0.02 mmol) and a mixture (4 M, 2 mL) of hydrochloric acid and ethyl acetate using a method similar to step seven of Example 2.

¹H NMR: δ (DMSO-d₆, 400 MHz) 8.93 (s, 1H), 8.39-8.36 (m, 2H), 7.99-7.92 (m, 4H), 7.78 (s, 1H), 7.56-7.54 (m, 2H), 7.17-7.13 (m, 3H), 6.66 (s, 2H), 4.18-4.16 (m, 2H), 3.47-3.44 (m, 1H), 1.20-1.18 (m, 6H)

MS measured value (ESI⁺) [(M+H)⁺]: 529

### Example 9

### 1-(4-(5-(3-amino-6-(4-(isopropylsulfonyl)phenyl)pyrazin-2-yl)isoxazol-3-yl)benzyl)-3-urea 9

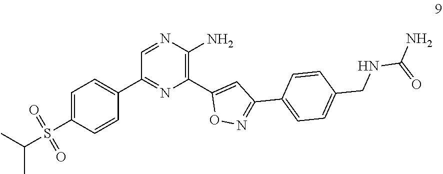 [SMILES: CC(C)S(=O)(=O)c1ccc(-c2cnc(N)c(-c3cc(-c4ccc(CNC(N)=O)cc4)no3)n2)cc1]

### Step One

### tert-butyl (tert-butoxycarbonyl) (5-(4-(isopropylsulfonyl)phenyl)-3-(3-(4-(ureidomethyl)phenyl)isoxazol-5-yl)pyrazin-2-yl) carbamate 9a

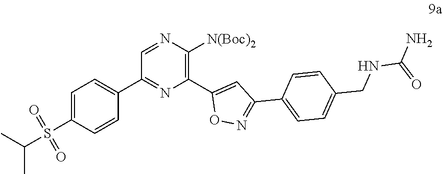

The white solid tert-butyl (tert-butoxycarbonyl) (5-(4-(isopropylsulfonyl)phenyl)-3-(3-(4-(ureidomethyl)phenyl) isoxazol-5-yl)pyrazin-2-yl) carbamate 9a (16 mg) was prepared by tert-butyl (tert-butoxycarbonyl) (5-(4-(isopropylsulfonyl) phenyl)-3-(3-(4-(((phenoxycarbonyl)amino)methylene)phenyl)isoxazol-5-yl)pyrazin-2-yl) carbamate 5a and ammonia methanol (2 mL, 7 M) using a method similar to step two of Example 5.

### Step Two

### 1-(4-(5-(3-amino-6-(4-(isopropylsulfonyl)phenyl)pyrazin-2-yl)isoxazol-3-yl)benzyl)-3-urea 9

The yellow solid 1-(4-(5-(3-amino-6-(4-(isopropylsulfonyl)phenyl)pyrazin-2-yl)isoxazol-3-yl)benzyl)-3-urea 9 (5.7 mg) was prepared by tert-butyl (tert-butoxycarbonyl) (5-(4-(isopropylsulfonyl)phenyl)-3-(3-(4-(ureidomethyl)phenyl) isoxazol-5-yl)pyrazin-2-yl) carbamate 9a (16 mg, 0.03 mmol) and a solution (4 M, 2 mL) of HCl in ethyl acetate using a method similar to step seven of Example 2.

¹H NMR (DMSO-d₆, 400 MHz): δ 8.94 (s, 1H), 8.39-8.37 (m, 2H), 7.98-7.92 (m, 4H), 7.77 (s, 1H), 7.44-7.42 (m, 2H), 7.20 (s, 2H), 6.54-6.51 (m, 1H), 5.59 (s, 2H), 4.27-4.25 (m, 2H), 3.49-3.46 (m, 1H), 1.19-1.18 (m, 6H)

MS measured value (ESI⁺) [(M+H)⁺]: 493

### Example 10

### 4-(5-amino-6-(3-(4-(guanidinomethyl)phenyl)isoxazol-5-yl)pyrazin-2-yl)-N,N-dimethylbenzenesulfonamide dihydrochloride 10

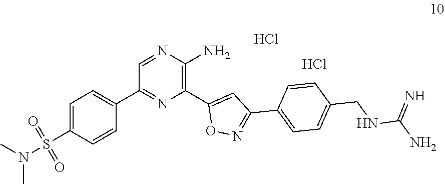 [SMILES: Cl ; CN(C)S(=O)(=O)c1ccc(-c2cnc(N)c(-c3cc(-c4ccc(CNC(=N)N)cc4)no3)n2)cc1 ; Cl]

### Step One

### tert-butyl (tert-butoxycarbonyl) (3-(3-(4-cyanophenyl)isoxazol-5-yl)-5-(4-(N,N-dimethylaminosulfonyl)phenyl)pyrazin-2-yl) carbamate 10a

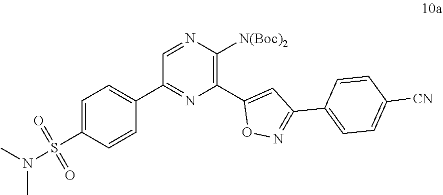

To a mixed solution of tert-butyl (5-bromo-3-(3-(4-cyanophenyl) 5-isoxazolyl)2-pyrazinyl(tert-butoxycarbonyl) carbamate (200 mg, 0.37 mmol) in 1,4-dioxane (3 mL) and water (0.6 mL) was added sequentially, under nitrogen protection, 4-isopropylsulfonylphenylboronic acid (127 mg, 0.41 mmol), potassium carbonate (102 mg, 0.74 mmol), and [1,1′-bis(diphenylphosphino)ferrocene] palladium(II) chloride (27 mg, 0.037 mmol) at room temperature (˜20° C.). After three times of nitrogen displacement, the reaction solution was stirred at 95° C. for three hours. After cooling to room temperature, ethyl acetate (10 mL) and water (5 mL) were added to the reaction solution. The mixed solution was filtered through diatomaceous earth and the resulting solution was separated. The aqueous phase was extracted three times with ethyl acetate (10 mL). Then, the combined organic phase was concentrated and isolated by column chromatography to give tert-butyl (tert-butoxycarbonyl) (3-(3-(4-cyanophenyl)isoxazol-5-yl)-5-(4-(N,N-dimethylaminosulfonyl)phenyl) pyrazin-2-yl) carbamate 10a (205 mg).

### Step Two

### tert-butyl (3-(3-(4-(aminomethyl)phenyl)isoxazol-5-yl)-5-(4-(N,N-dimethylaminosulfonyl)phenyl)pyrazin-2-yl)(tert-butoxycarbonyl) carbamate 10b

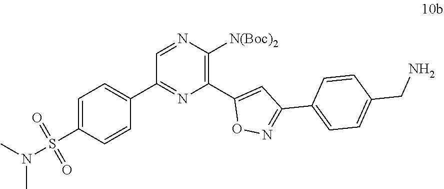

To a solution of tert-butyl (tert-butoxycarbonyl) (3-(3-(4-cyanophenyl)isoxazol-5-yl)-5-(4-(N,N-dimethylaminosulfonyl)phenyl)pyrazin-2-yl) carbamate 10a (205 mg, 0.32 mmol) in methanol (3 mL) were added nickel chloride hexahydrate (228 mg, 0.96 mmol) and sodium borohydride (36 mg, 0.96 mmol) sequentially at room temperature. The reaction solution was stirred at room temperature for 1 hour and then quenched with saturated aqueous ammonium chloride (2 mL). Ethyl acetate (10 mL) and saturated aqueous sodium bicarbonate solution (10 mL) were then added. The mixed solution was filtered through diatomaceous earth and the filtrate was poured into a separatory funnel. The aqueous phase was extracted three times with ethyl acetate (10 mL) and the combined organic phase was concentrated and isolated by column chromatography to give tert-butyl (3-(3-(4-(aminomethyl)phenyl)isoxazol-5-yl)-5-(4-(N,N-dimethylaminosulfonyl)phenyl)pyrazin-2-yl)(tert-butoxycarbonyl) carbamate 10b (33 mg).

### Step Three

### tert-butyl (tert-butoxycarbonyl)(5-(4-(N,N-dimethylaminosulfonyl)phenyl)-3-(3-(4-(guanidinomethyl)phenyl)isoxazol-5-yl)pyrazin-2-yl) carbamate 10c

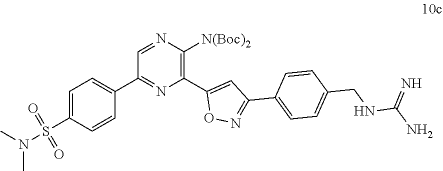

To a solution of tert-butyl (3-(3-(4-(aminomethyl)phenyl)isoxazol-5-yl)-5-(4-(N,N-dimethylaminosulfonyl) phenyl)pyrazin-2-yl)(tert-butoxycarbonyl) carbamate 10b (33 mg, 0.050 mmol) in acetonitrile (2 mL) were added 1H-pyrazole-1-carboximidamide hydrochloride (7.3 mg. 0.050 mmol) and N,N-diisopropylethylamine (10 uL) sequentially. The reaction solution was stirred at room temperature (20° C.) for 16 hours. Ethyl acetate (10 mL) and water (10 mL) were added to the reaction solution, and the resulting solution was separated. The aqueous phase was extracted three times with ethyl acetate (10 mL), and the combined organic phase was concentrated and isolated by column chromatography to give tert-butyl (tert-butoxycarbonyl) (5-(4-(N,N-dimethylaminosulfonyl)phenyl)-3-(3-(4-(guanidinomethyl)phenyl) isoxazole-5-yl)pyrazin-2-yl) carbamate 10c (29 mg).

### Step Four

### 4-(5-amino-6-(3-(4-(guanidinomethyl)phenyl)isoxazol-5-yl)pyrazin-2-yl)-N,N-dimethylbenzenesulfonamide dihydrochloride 10

To a solution of tert-butyl (tert-butoxycarbonyl)(5-(4-(N,N-dimethylaminosulfonyl)phenyl)-3-(3-(4-(guanidinomethyl)phenyl)isoxazol-5-yl)pyrazin-2-yl) carbamate 10c (29 mg, 0.04 mmol) in ethyl acetate (2 mL) was added a mixture (4 M, 2 mL) of hydrochloric acid and ethyl acetate. After the reaction solution was stirred for 1 hour at room temperature, the reaction solution was concentrated to give a yellow solid 4-(5-amino-6-(3-(4-(guanidinomethyl)phenyl) isoxazol-5-yl)pyrazin-2-yl)-N,N-dimethylbenzenesulfonamide dihydrochloride 10 (12.4 mg).

¹H NMR (400 MHz, DMSO-d₆): δ 8.96 (s, 1H), 8.40-8.38 (m, 2H), 8.36-8.33 (m, 1H), 8.08-8.06 (m, 2H), 7.87-7.85 (m, 2H), 7.82 (s, 1H), 7.55-7.53 (m, 2H), 7.44 (brs, 2H), 7.31 (brs, 2H), 7.18 (brs, 3H), 4.53-4.52 (m, 2H), 2.68 (s, 6H)

MS measured value (ESI⁺) [(M+H)⁺]: 493

### Example 11

### 1-(4-(5-(3-amino-6-(4-(isopropylsulfonyl)phenyl)pyrazin-2-yl)isoxazol-3-yl)-3,5-difluorobenzyl)guanidine dihydrochloride 11

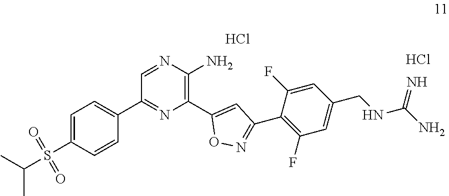 [SMILES: Cl ; Cl ; CC(C)S(=O)(=O)c1ccc(-c2cnc(N)c(-c3cc(-c4c(F)cc(CNC(=N)N)cc4F)no3)n2)cc1]

### Step One

### tert-butyl (tert-butoxycarbonyl)(3-ethynyl-5-(4-(isopropylsulfonyl)phenyl)pyrazin-2-yl)carbamate 11a

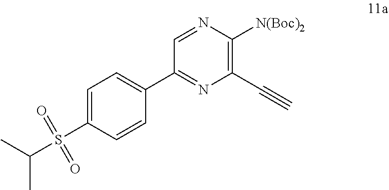

To a mixed solution of 4-isopropylsulfonylphenylboronic acid (500 mg, 2.19 mmol) in 1,4-dioxane (5 mL) and water (1 mL) was sequentially added tert-butyl N-(5-bromo-3-ethynylpyrazin-2-yl)-N-[(tert-butoxy)carbonyl]carbamate (582 mg, 1.46 mmol), potassium carbonate (606 mg, 4.38 mmol) and [1,1′-bis(diphenylphosphino)ferrocene] palladium(II) chloride (107 mg, 0.15 mmol) at room temperature (˜20° C.). After three times of nitrogen displacements, the reaction solution was stirred at 55° C. for two hours and cooled to room temperature. Ethyl acetate (40 mL) and water (40 mL) were added to the reaction solution. The mixed solution was filtered through diatomaceous earth, and the resulting solution was separated. The aqueous phase was extracted three times with ethyl acetate (20 mL), and the combined organic phase was concentrated and isolated by column chromatography to give tert-butyl (tert-butoxycarbonyl)(3-ethynyl-5-(4-(isopropylsulfonyl) phenyl)pyrazin-2-yl)carbamate 11a (433 mg).

### Step Two

### (E)-3,5-difluoro-4-(hydroxyimino) methyl) benzonitrile 11b

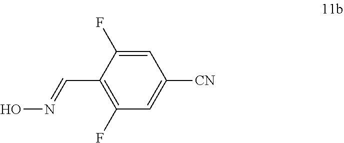 [SMILES: N#Cc1cc(F)c(/C=N/O)c(F)c1]

To a mixed solution of 3,5-difluoro-4-formylbenzonitrile (1 g, 5.98 mmol) in tetrahydrofuran (10 mL) and water (1 mL) was added hydroxylamine hydrochloride (499 mg, 7.18 mmol) at room temperature. The reaction mixture was stirred at room temperature (˜20° C.) for 16 hours. Water (60 mL) was added to the reaction system and the organic phase was separated out. The aqueous phase was extracted three times with ethyl acetate (20 mL). The combined organic phase was dried with anhydrous sodium sulfate, and filtered, and the filtrate was concentrated under reduced pressure to give a white solid (E)-3,5-difluoro-4-(hydroxyimino) methyl) benzonitrile 11b (433 mg), which was a crude product and was directly used in the next step without a further purification.

### Step Three

### tert-butyl (3-(3-(4-cyano-2,6-difluorophenyl)isoxazol-5-yl)-5-(4-(isopropylsulfonyl)phenyl)pyrazin-2-yl)carbamate 11c

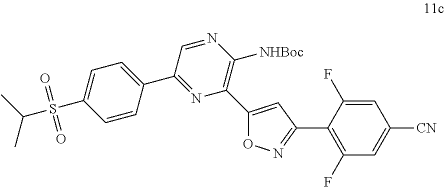 [SMILES: CC(C)S(=O)(=O)c1ccc(-c2cnc(NC(=O)OC(C)(C)C)c(-c3cc(-c4c(F)cc(C#N)cc4F)no3)n2)cc1]

To a mixed solution of (E)-3,5-difluoro-4-(hydroxyimino) methyl) benzonitrile 11b (80 mg, 0.44 mmol) in methanol (5 mL) and water (1 mL) were sequentially added tert-butyl (tert-butoxycarbonyl)(3-ethynyl-5-(4-(isopropylsulfonyl) phenyl)pyrazin-2-yl)carbamate 11a (147 mg. 0.29 mL) and [bis(trifluoroacetyl)iodo]benzene (189 mg, 0.44 mmol). The solution was stirred overnight at room temperature (˜20° C.). Ethyl acetate (40 mL) and water (40 mL) were added to the reaction solution. The organic phase was washed three times with water (20 mL). The organic phase was separated and dried with anhydrous sodium sulfate, filtered, concentrated and isolated by column chromatography to give tert-butyl (3-(3-(4-cyano-2,6-difluorophenyl)isoxazol-5-yl)-5-(4-(isopropylsulfonyl)phenyl)pyrazin-2-yl)carbamate 11c (177 mg).

### Step Four

### tert-butyl (3-(3-(4-(aminomethyl)-2,6-difluorophenyl)isoxazol-5-yl)-5-(4-(isopropylsulfonyl)phenyl)pyrazin-2-yl)carbamate 11d

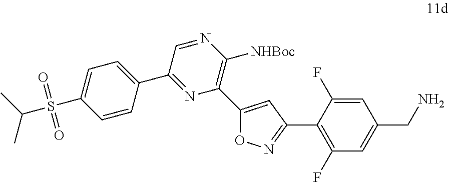 [SMILES: CC(C)S(=O)(=O)c1ccc(-c2cnc(NC(=O)OC(C)(C)C)c(-c3cc(-c4c(F)cc(CN)cc4F)no3)n2)cc1]

To a solution of tert-butyl (3-(3-(4-cyano-2,6-difluorophenyl)isoxazol-5-yl)-5-(4-(isopropylsulfonyl)phenyl)pyrazin-2-yl)carbamate 11c (177 mg, 0.26 mmol) in methanol (2 mL) was sequentially added nickel chloride hexahydrate (185 mg, 0.78 mmol) and sodium borohydride (29 mg, 0.78 mmol) at room temperature. The reaction solution was stirred for 1 hour at room temperature and then quenched with ammonium chloride (2 mL). Ethyl acetate (40 mL) and saturated aqueous sodium bicarbonate (40 mL) were then added. The mixed solution was filtered through diatomaceous earth and the filtrate was poured into a separatory funnel. The aqueous phase was extracted three times with ethyl acetate (40 mL) and the combined organic phase was concentrated and isolated by column chromatography to give tert-butyl (3-(3-(4-(aminomethyl)-2,6-difluorophenyl)isoxazol-5-yl)-5-(4-(isopropylsulfonyl)phenyl)pyrazin-2-yl)carbamate 11d (25 mg).

### Step Five

### tert-butyl (3-(3-(2,6-difluoro-4-(guanidinomethyl)phenyl)isoxazol-5-yl)-5-(4-(isopropylsulfonyl)phenyl)pyrazin-2-yl) carbamate 11e

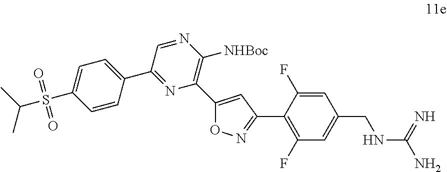 [SMILES: CC(C)S(=O)(=O)c1ccc(-c2cnc(NC(=O)OC(C)(C)C)c(-c3cc(-c4c(F)cc(CNC(=N)N)cc4F)no3)n2)cc1]

To a solution of tert-butyl (3-(3-(4-(aminomethyl)-2,6-difluorophenyl)isoxazol-5-yl)-5-(4-(isopropylsulfonyl) phenyl)pyrazin-2-yl)carbamate 11d (25 mg, 0.042 mmol) in acetonitrile (2 mL) was sequentially added 1H-pyrazole-1-carboxamidine hydrochloride (6.3 mg, 0.043 mmol) and N,N-diisopropylethylamine (6 mg, 0.045 mmol). The reaction was stirred at room temperature (˜20° C.) for 16 hours. Ethyl acetate (20 mL) and water (20 mL) were added to the reaction solution, and the resulting solution was separated. The aqueous phase was extracted three times with ethyl acetate (20 mL), and the combined organic phase was concentrated and isolated by column chromatography to give tert-butyl (3-(3-(2,6-difluoro-4-(guanidinomethyl)phenyl)isoxazol-5-yl)-5-(4-(isopropylsulfonyl)phenyl)pyrazin-2-yl) carbamate 11e (20 mg).

### Step Six

### 1-(4-(5-(3-amino-6-(4-(isopropylsulfonyl)phenyl)pyrazin-2-yl)isoxazol-3-yl)-3,5-difluorobenzyl)guanidine dihydrochloride 11

To a solution of tert-butyl (3-(3-(2,6-difluoro-4-(guanidinomethyl)phenyl)isoxazol-5-yl)-5-(4-(isopropylsulfonyl) phenyl)pyrazin-2-yl) carbamate 11e (20 mg, 0.032 mmol) in ethyl acetate (2 mL) was added a mixture (4 M, 2 mL) of hydrochloric acid and ethyl acetate. After the reaction solution was stirred for 1 hour at room temperature, the reaction solution was concentrated to give a yellow solid 1-(4-(4-(5-(3-amino-6-(4-(isopropylsulfonyl)phenyl)pyrazin-2-yl)isoxazol-3-yl)-3,5-difluorobenzyl)guanidine dihydrochloride 11 (13 mg).

¹H NMR (400 MHz, DMSO-d₆): δ 8.96 (s, 1H), 8.43-8.40 (m, 1H), 8.36-8.34 (m, 2H), 7.93-7.91 (m, 2H), 7.52 (s, 1H), 7.38 (s, 2H), 7.32-7.30 (m, 2H), 7.25 (s, 2H), 7.22 (s, 1H), 7.13 (s, 2H), 4.55-4.54 (m, 2H), 3.48-3.45 (m, 1H), 1.19-1.17 (m, 6H)

MS measured value (ESI⁺) [(M+H)⁺]: 528

### Example 12

### 1-(4-(5-(3-amino-6-(4-(N,S-dimethylsulfonylimino)phenyl)pyrazin-2-yl)isoxazol-3-yl)benzyl)guanidine dihydrochloride 12

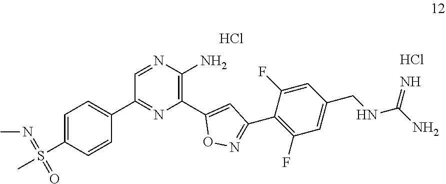 [SMILES: Cl ; Cl ; CN=S(C)(=O)c1ccc(-c2cnc(N)c(-c3cc(-c4c(F)cc(CNC(=N)N)cc4F)no3)n2)cc1]

### Step One

### (4-bromophenyl)(methyl)(methylimino)-λ⁶-sulfanone 12a

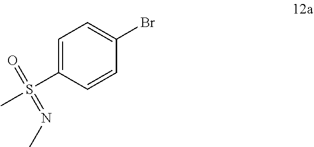

To (4-bromophenyl)(imino)(methyl)-λ⁶-sulfanone (300 mg, 1.28 mmol) in N,N-dimethylformamide (3 mL) was added sodium hydride (60% in mineral oil, 154 mg, 3.84 mmol). The resulting mixture was stirred at room temperature for 5 min and iodomethane (0.4 mL, 6.41 mmol) was added. The reaction mixture was stirred overnight. The mixture was concentrated in vacuo, diluted with water and extracted with ethyl acetate. The extracts were combined and washed with brine, dried with MgSO₄ and concentrated in vacuo. The residue was purified by silica gel chromatography to give (4-bromophenyl)(methyl) (methylimino)-λ⁶-sulfanone 12a (330 mg).

### Step Two

### (4-(N,S-dimethylsulfinyl)phenyl)boronic acid 12b

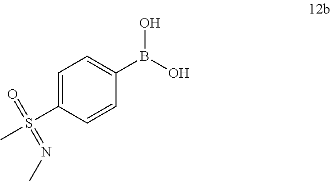

Under nitrogen protection, (4-bromophenyl)(methyl) (methylimino)-λ⁶-sulfanone 12a (230 mg, 0.93 mmol), 4,4,4,4,5,5,5,5-octamethyl-2,2-bis(1,3,2-dioxaborolane) (353 mg, 1.39 mmol), potassium acetate (273 mg, 2.78 mmol) and [1,1′-bis(diphenylphosphino)ferrocene] palladium(II) chloride (68 mg, 0.09 mmol) were dissolved in a solution of anhydrous 1,4-dioxane (4 mL). After three times of nitrogen replacement, the reaction solution was stirred at 90° C. for two hours. After cooling to room temperature, ethyl acetate (40 mL) and water (40 mL) were added to the reaction solution. The mixed solution was filtered through diatomaceous earth and the resulting solution was separated. The aqueous phase was extracted three times with ethyl acetate (20 mL), and the combined organic phase was concentrated and isolated by column chromatography to give (4-(N,S-dimethylsulfinyl)phenyl)boronic acid 12b (197 mg).

### Step Three

### tert-butyl (tert-butoxy carbonyl) (3-(3-(4-cyanophenyl)isoxazol-5-yl)-5-(4-(N,S-dimethylsulfonylimino)phenyl)pyrazin-2-yl) carbamate 12c

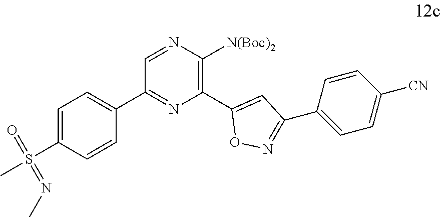

Under nitrogen protection, (4-(N,S-dimethylsulfinyl)phenyl)boronic acid 12b (197 mg, 0.93 mmol), tert-butyl N-{5-bromo-3-[3-(4-cyanophenyl)-1,2-oxazol-5-yl]pyrazin-2-yl}-N-[(tert-butoxy)carbonyl]carbamate (300 mg, 0.55 mmol), potassium carbonate (299 mg, 1.66 mmol) and [1,1′-bis(diphenylphosphino)ferrocene]palladium(II) chloride (41 mg, 0.06 mmol) were dissolved in a mixture of 1,4-dioxane (4 mL) and water (1 mL). After three times of nitrogen replacement, the reaction solution was stirred at 90° C. for two hours. After cooling to room temperature, ethyl acetate (40 mL) and water (40 mL) were added to the reaction solution. The mixed solution was filtered through diatomaceous earth and the resulting solution was separated. The aqueous phase was extracted three times with ethyl acetate (20 mL). The combined organic phase was concentrated and isolated by column chromatography to give tert-butyl (tert-butoxy carbonyl)(3-(3-(4-cyanophenyl)isoxazol-5-yl)-5-(4-(N,S-dimethylsulfonylimino)phenyl)pyrazin-2-yl) carbamate 12c (145 mg).

### Step Four

### tert-butyl (tert-butoxy carbonyl) (3-(3-(4-(aminomethyl)phenyl)isoxazol-5-yl)-5-(4-(N,S-dimethylsulfonylimino)phenyl)pyrazin-2-yl) carbamate 12d

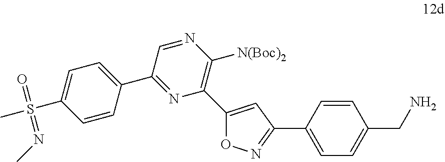

To a solution of tert-butyl (tert-butoxy carbonyl)(3-(3-(4-cyanophenyl)isoxazol-5-yl)-5-(4-(N,S-dimethylsulfonylimino)phenyl)pyrazin-2-yl) carbamate 12c (145 mg, 0.23 mmol) in methanol (2 mL) was added nickel chloride hexahydrate (164 mg, 0.69 mmol) and sodium borohydride (26 mg, 0.69 mmol) sequentially at room temperature. The reaction solution was stirred at room temperature for 1 hour and then quenched with saturated aqueous ammonium chloride (2 mL). Ethyl acetate (40 mL) and saturated aqueous sodium bicarbonate solution (40 mL) were then added. The mixed solution was filtered through diatomaceous earth and the filtrate was poured into a separatory funnel. The aqueous phase was extracted three times with ethyl acetate (40 mL) and the combined organic phase was concentrated and isolated by column chromatography to give tert-butyl (tert-butoxy carbonyl) (3-(3-(4-(aminomethyl)phenyl)isoxazol-5-yl)-5-(4-(N,S-dimethylsulfonylimino)phenyl)pyrazin-2-yl) carbamate 12d (15 mg).

### Step Five

### tert-butyl (tert-butoxycarbonyl)(5-(4-(N,S-dimethylsulfonylimino)phenyl)-3-(3-(4-(guanidinylmethyl)phenyl)isoxazol-5-yl)pyrazin-2-yl) carbamate 12e

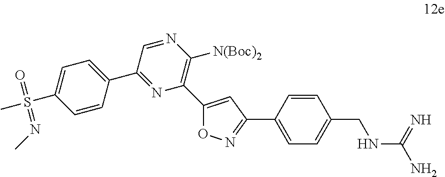

To a solution of tert-butyl (tert-butoxy carbonyl) (3-(3-(4-(aminomethyl)phenyl)isoxazol-5-yl)-5-(4-(N,S-dimethylsulfonylimino)phenyl)pyrazin-2-yl) carbamate 12d (15 mg, 23.63 gmol) in acetonitrile (1 mL) were added 1H-pyrazole-1-carboximidamide hydrochloride (7 mg, 47.26 gmol) and N,N-diisopropylethylamine (9 mg, 70.89 gmol) sequentially. The reaction solution was stirred at room temperature (˜20° C.) for 16 hours. Ethyl acetate (20 mL) and water (20 mL) were added to the reaction solution and the resulting solution was separated. The aqueous phase was extracted three times with ethyl acetate (20 mL), and the combined organic phase was concentrated and isolated by column chromatography to give tert-butyl (tert-butoxycarbonyl) (5-(4-(N,S-dimethylsulfonylimino)phenyl)-3-(3-(4-(guanidinylmethyl)phenyl)isoxazol-5-yl)pyrazin-2-yl) carbamate 12e (15 mg).

### Step Six

### 1-(4-(5-(3-amino-6-(4-(N,S-dimethylsulfonylimino)phenyl)pyrazin-2-yl)isoxazol-3-yl)benzyl)guanidine dihydrochloride 12

To a solution of tert-butyl (tert-butoxycarbonyl) (5-(4-(N,S-dimethylsulfonylimino)phenyl)-3-(3-(4-(guanidinylmethyl)phenyl)isoxazol-5-yl)pyrazin-2-yl) carbamate 12e (15 mg, 22.16 gmol) in ethyl acetate (2 mL) was added an ethyl acetate solution of HCl (4 M, 2 mL). After the reaction solution was stirred for 1 hour at room temperature, the reaction solution was concentrated to give a yellow solid 1-(4-(5-(3-amino-6-(4-(N,S-dimethylsulfonylimino) phenyl)pyrazin-2-yl)isoxazol-3-yl)benzyl)guanidine dihydrochloride 12 (11 mg).

¹H NMR (400 MHz, DMSO-d₆): δ 9.01 (s, 1H), 8.50-8.48 (m, 2H), 8.30-8.27 (m, 1H), 8.13-8.11 (m, 2H), 8.05-8.03 (m, 1H), 7.81 (s, 1H), 7.52-7.51 (m, 2H), 7.37 (s, 2H), 7.24 (s, 2H), 7.12 (s, 2H), 4.50-4.48 (m, 2H), 3.93-3.92 (m, 2H), 2.66 (s, 3H), 1.23 (s, 3H)

MS measured value (ESI⁺) [(M+H)⁺]: 477

### Example 13

### 1-(4-(5-(3-amino-6-(4-(N-methylpropan-2-yl-sulfonimido)phenyl)pyrazin-2-yl)isoxazol-3-yl)benzyl)guanidine dihydrochloride 13

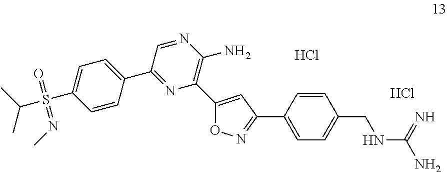 [SMILES: CN=S(=O)(c1ccc(-c2cnc(N)c(-c3cc(-c4ccc(CNC(=N)N)cc4)no3)n2)cc1)C(C)C ; Cl ; Cl]

### Step One

### (4-bromophenyl)(imino)(isopropyl)-λ⁶-sulfanone 13a

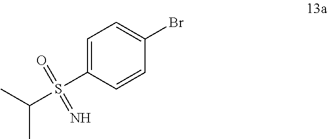

To a solution of (4-bromophenyl)(isopropyl)sulfonamide (1 g, 4.33 mmol) in ethanol (10 mL) were added iodobenzene diacetic acid (4.18 g, 12.98 mmol) and ammonium acetate (1.34 g, 17.30 mmol) at room temperature. The reaction mixture was stirred at room temperature (˜20° C.) for 3 hours. Water (60 mL) was added to the reaction system and the organic phase was separated. The aqueous phase was extracted three times with ethyl acetate (20 mL), and the combined organic phase was dried with anhydrous sodium sulfate, filtered, concentrated and isolated by column chromatography to give (4-bromophenyl)(imino)(isopropyl)-λ⁶-sulfanone 13a (1.07 g).

### Step Two

### (4-bromophenyl) (isopropyl) (methylimino)-λ⁶-sulfanone 13b

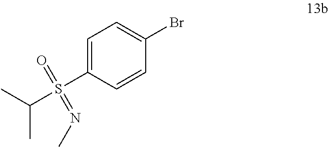

To (4-bromophenyl) (isopropyl) (methylimino)-λ⁶-sulfanone 13b (500 mg, 1.91 mmol) in N,N-dimethylformamide (5 mL) was added sodium hydride (60% in mineral oil, 229 mg, 5.72 mmol), and the resulting mixture was stirred at room temperature for 5 min. Iodomethane (0.6 mL, 9.54 mmol) was added and the reaction mixture was stirred overnight. The mixture was concentrated in vacuo, diluted with water and extracted with ethyl acetate. The extracts were washed with brine and combined, dried with MgSO₄ and concentrated in vacuo. The crude product was purified by silica gel chromatography to give (4-bromophenyl) (isopropyl) (methylimino)-λ⁶-sulfanone 13b (490 mg).

### Step Three

### (4-(N-methylpropan-2-ylsulfonimidoyl)phenyl)boronic acid 13c

Under nitrogen protection, (4-bromophenyl) (isopropyl) (methylimino)-λ6-sulfanone 13b (250 mg, 0.91 mmol), 4,4,4,4,5,5,5,5-octamethyl-2,2-bis(1,3,2-dioxaborolane) (345 mg, 1.36 mmol), potassium acetate (267 mg, 2.72 mmol) and [1,1′-bis(diphenylphosphino)ferrocene]palladium(II) chloride (66 mg, 0.09 mmol) were dissolved in a solution of anhydrous 1,4-dioxane (4 mL). After three times of nitrogen replacements, the reaction solution was stirred at 90° C. for two hours. After cooling to room temperature, ethyl acetate (40 mL) and water (40 mL) were added to the reaction solution. The mixed solution was filtered through diatomaceous earth and the resulting solution was separated. The aqueous phase was extracted three times with ethyl acetate (20 mL), and the combined organic phase was concentrated and isolated by column chromatography to give (4-(N-methylpropan-2-ylsulfonimidoyl)phenyl)boronic acid 13c (218 mg).

### Step Four

### tert-butyl (tert-butoxycarbonyl)(3-(3-(4-cyanophenyl)isoxazol-5-yl)-5-(4-(N-methylpropyl-2-sulfonylimino)phenyl)pyrazin-2-yl) carbamate 13d

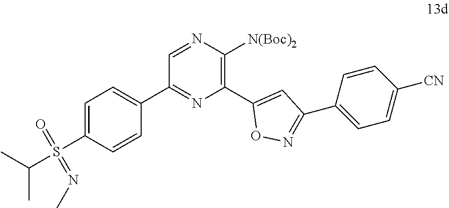

Under nitrogen protection, (4-(N-methylpropan-2-ylsulfonimidoyl)phenyl)boronic acid 13c (218 mg, 0.91 mmol), tert-butyl (5-bromo-3-(3-(4-cyanophenyl) isoxazol-5-yl) pyrazin-2-yl) (tert-butoxycarbonyl) carbamate 1b (300 mg, 0.55 mmol), potassium carbonate (229 mg. 1.66 mmol) and [1,1′-bis(diphenylphosphino)ferrocene]palladium(II) chloride (40 mg, 0.06 mmol) were dissolved in a mixture of 1,4-dioxane (4 mL) and water (1 mL). After three times of nitrogen replacements, the reaction solution was stirred at 90° C. for two hours. After cooling to room temperature, ethyl acetate (40 mL) and water (40 mL) were added to the reaction solution. The mixed solution was filtered through diatomaceous earth and the resulting solution was separated. The aqueous phase was extracted three times with ethyl acetate (20 mL), and the combined organic phase was concentrated and isolated by column chromatography to give tert-butyl (tert-butoxycarbonyl)(3-(3-(4-cyanophenyl)isoxazol-5-yl)-5-(4-(N-methylpropyl-2-sulfonylimino)phenyl)pyrazin-2-yl) carbamate 13d (180 mg).

### Step Five

### tert-butyl (tert-butoxycarbonyl)(3-(3-(4-(aminomethyl)phenyl)isoxazol-5-yl)-5-(4-(N-methylpropyl-2-sulfonylimino)phenyl)pyrazin-2-yl)carbamate 13e

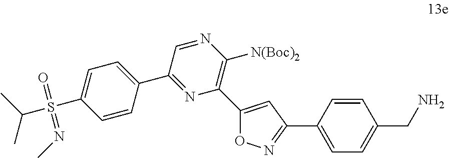

To a solution of tert-butyl (tert-butoxycarbonyl)(3-(3-(4-cyanophenyl)isoxazol-5-yl)-5-(4-(N-methylpropyl-2-sulfonylimino)phenyl)pyrazin-2-yl) carbamate 13d (180 mg, 0.27 mmol) in methanol (2 mL) was added sequentially, at room temperature, nickel chloride hexahydrate (195 mg, 0.82 mmol) and sodium borohydride (31 mg, 0.82 mmol). The reaction solution was stirred for 1 hour at room temperature and then quenched with saturated aqueous ammonium chloride (2 mL). Ethyl acetate (40 mL) and saturated aqueous sodium bicarbonate solution (40 mL) were then added. The mixed solution was filtered through diatomaceous earth and the filtrate was poured into a separatory funnel. The aqueous phase was extracted three times with ethyl acetate (40 mL) and the combined organic phase was concentrated and isolated by column chromatography to give tert-butyl (tert-butoxycarbonyl)(3-(3-(4-(aminomethyl)phenyl)isoxazol-5-yl)-5-(4-(N-methylpropyl-2-sulfonylimino)phenyl)pyrazin-2-yl)carbamate 13e (16 mg).

### Step Six

### tert-butyl (tert-butoxycarbonyl)(3-(3-(4-(guanidinylmethyl)phenyl)isoxazol-5-yl)-5-(4-(N-methylpropyl-2-sulfonimido)phenyl)-pyrazin-2-yl)carbamate 13f

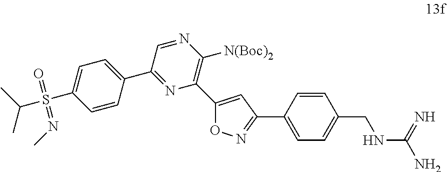

To a solution of tert-butyl (tert-butoxycarbonyl)(3-(3-(4-(aminomethyl)phenyl)isoxazol-5-yl)-5-(4-(N-methylpropyl-2-sulfonylimino)phenyl)pyrazin-2-yl)carbamate 13e (16 mg, 24.14 gmol) in acetonitrile (1 mL) were added 1H-pyrazole-1-carboxamidine hydrochloride (7 mg. 48.28 gmol) and N,N-diisopropylethylamine (9 mg, 72.42 gmol) sequentially. The reaction was stirred at room temperature (˜20° C.) for 16 hours. Ethyl acetate (20 mL) and water (20 mL) were added to the reaction solution and the resulting solution was separated. The aqueous phase was extracted three times with ethyl acetate (20 mL), and the combined organic phase was concentrated and isolated by column chromatography to give tert-butyl (tert-butoxycarbonyl)(3-(3-(4-(guanidinylmethyl)phenyl)isoxazol-5-yl)-5-(4-(N-methylpropyl-2-sulfonimido)phenyl)-pyrazin-2-yl)carbamate 13f (13 mg).

### Step Seven

### 1-(4-(5-(3-amino-6-(4-(N-methylpropan-2-yl-sulfonimido)phenyl)pyrazin-2-yl)isoxazol-3-yl)benzyl)guanidine dihydrochloride 13

To a solution of tert-butyl (tert-butoxycarbonyl)(3-(3-(4-(guanidinylmethyl)phenyl)isoxazol-5-yl)-5-(4-(N-methylpropyl-2-sulfonimido)phenyl)-pyrazin-2-yl)carbamate 13f (13 mg, 18.44 gmol) in ethyl acetate (2 mL) was added a mixture (4 M, 2 mL) of hydrochloric acid and ethyl acetate. The reaction solution was stirred at room temperature for 1 hour and then concentrated to give a yellow solid 1-(4-(5-(3-amino-6-(4-(N-methylpropan-2-yl-sulfonimido)phenyl) pyrazin-2-yl)isoxazol-3-yl)benzyl)guanidine dihydrochloride 13 (8 mg).

¹H NMR (400 MHz, DMSO-d₆): δ 9.00 (s, 1H), 8.52-8.50 (m, 2H), 8.34-8.31 (m, 1H), 8.05-8.03 (m, 4H), 7.82 (s, 1H), 7.52-7.50 (m, 2H), 7.40 (s, 2H), 7.27 (s, 2H), 7.14 (s, 2H), 4.50-4.49 (m, 2H), 4.28 (s, 1H), 3.93 (s, 1H), 2.67 (s, 3H), 1.45-1.44 (m, 3H), 1.26-1.25 (m, 3H)

MS measured value (ESI⁺) [(M+H)⁺]: 505

### Example 14

### 1-(4-(5-(3-amino-6-(4-(isopropylsulfonyl)phenyl)pyrazin-2-yl)isoxazol-3-yl)phenyl)-3-methylurea 14

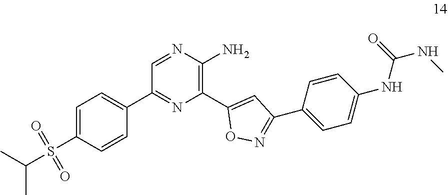 [SMILES: CNC(=O)Nc1ccc(-c2cc(-c3nc(-c4ccc(S(=O)(=O)C(C)C)cc4)cnc3N)on2)cc1]

### Step One

### (E)-4-nitrobenzaldehyde oxime 14a

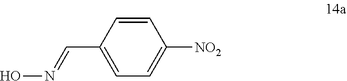 [SMILES: O=[N+]([O-])c1ccc(/C=N/O)cc1]

To a solution of 4-nitrobenzaldehyde (2 g, 13.24 mmol) in tetrahydrofuran (30 mL) was added hydroxylamine hydrochloride (1.02 g, 15.89 mmol) at room temperature. The reaction mixture was stirred at room temperature (˜20° C.) for 16 hours. Water (40 mL) was added to the reaction system and the organic phase was separated. The aqueous phase was extracted three times with ethyl acetate (30 mL), and the combined organic phase was dried with anhydrous sodium sulfate and filtered. The filtrate was concentrated under reduced pressure to give the yellow solid (E)-4-nitrobenzaldehyde oxime 14a (2.2 g) as a crude product, which was used directly in the next step without a further purification.

### Step Two

### tert-butyl (5-bromo-3-(3-(4-nitrophenyl)isoxazol-5-yl)pyrazin-2-yl)(tert-butoxycarbonyl)carbamate 14b

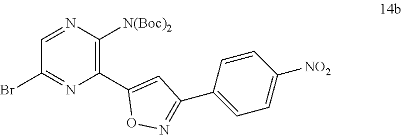

To a mixed solution of tert-butyl (5-bromo-3-(3-(4-nitrophenyl)isoxazol-5-yl)pyrazin-2-yl)(tert-butoxycarbonyl) carbamate (0.48 g, 1.20 mmol) in methanol (18 mL) and water (3.6 mL) were added sequentially (E)-4-nitrobenzaldehyde oxime 14a (0.3 g, 1.81 mmol) and bis(trifluoroacetyl)iodobenzene (0.78 g, 1.81 mmol) and stirred overnight at room temperature. Ethyl acetate (40 mL) and water (40 mL) were added to the reaction solution. The organic phase was washed three times with water (20 mL). The organic phase was separated and dried with anhydrous sodium sulfate, filtered, concentrated and isolated by column chromatography to give tert-butyl (5-bromo-3-(3-(4-nitrophenyl)isoxazol-5-yl)pyrazin-2-yl)(tert-butoxycarbonyl)carbamate 14b (0.41 g).

### Step Three

### tert-butyl (tert-butoxycarbonyl)(5-(4-(isopropylsulfonyl)phenyl)-3-(3-(4-nitrophenyl)isoxazol-5-yl)-pyrazin-2-yl) carbamate 14c

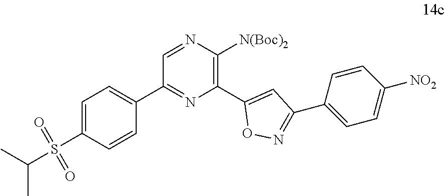

To a mixed solution of tert-butyl (5-bromo-3-(3-(4-nitrophenyl)isoxazol-5-yl)pyrazin-2-yl)(tert-butoxycarbonyl) carbamate 14b (0.37 g, 0.66 mmol) in 1,4-dioxane (15 mL) and water (1.5 mL) at room temperature were sequentially added 4-isopropylsulfonylbenzeneboronic acid (0.17 g 0.73 mmol), potassium carbonate (0.18 g, 1.32 mmol) and [1,1′-bis(diphenylphosphino)ferrocene]palladium(II) chloride (0.10 g, 0.13 mmol) at room temperature and under nitrogen protection. After three times of nitrogen replacements, the reaction solution was stirred at 95° C. for three hours. After cooling to room temperature, ethyl acetate (40 mL) and water (40 mL) were added to the reaction solution. The mixed solution was filtered through diatomaceous earth and the resulting solution was separated. The aqueous phase was extracted three times with ethyl acetate (20 mL), and the combined organic phase was concentrated and isolated by column chromatography to give tert-butyl (tert-butoxycarbonyl)(5-(4-(isopropylsulfonyl)phenyl)-3-(3-(4-nitrophenyl)isoxazol-5-yl)-pyrazin-2-yl) carbamate 14c (0.17 g).

### Step Four

### tert-butyl (3-(3-(4-aminophenyl)isoxazol-5-yl)-5-(4-(isopropylsulfonyl)phenyl)pyrazin-2-yl)(tert-butoxycarbonyl)carbamate 14d

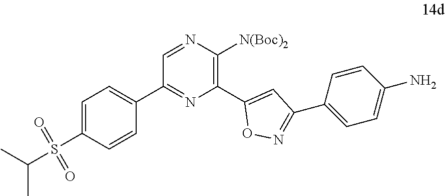

To a solution of tert-butyl (tert-butoxycarbonyl)(5-(4-(isopropylsulfonyl)phenyl)-3-(3-(4-nitrophenyl)isoxazol-5-yl)-pyrazin-2-yl) carbamate 14c (130 mg, 0.20 mmol) in acetic acid (8 mL) was added reduced iron powder (109 mg, 2.0 mmol) at room temperature. The reaction solution was stirred for 16 hours at room temperature, then diluted with ethyl acetate (40 mL). The mixture was filtered through diatomaceous earth, washed to be neutral with aqueous sodium bicarbonate (40 mL), and then poured into a separatory funnel. The aqueous phase was extracted three times with ethyl acetate (40 mL) and the combined organic phase was concentrated and isolated by column chromatography to give tert-butyl (3-(3-(4-aminophenyl)isoxazol-5-yl)-5-(4-(isopropylsulfonyl)phenyl)pyrazin-2-yl)(tert-butoxycarbonyl)carbamate 14d (100 mg).

### Step Five

### tert-butyl (tert-butoxycarbonyl)(5-(4-(isopropylsulfonyl)phenyl)-3-(3-(4-((phenoxycarbonyl)amino)phenyl)isoxazol-5-yl)pyrazin-2-yl)carbamate 14e

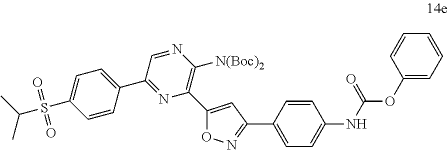

To a solution of tert-butyl (3-(3-(4-aminophenyl)isoxazol-5-yl)-5-(4-(isopropylsulfonyl)phenyl)pyrazin-2-yl)(tert-butoxycarbonyl)carbamate 14d (27 mg, 0.042 mmol) in dichloromethane (2 mL) were added triethylamine (0.017 mL) and phenyl chloroformate (7.3 mg, 0.047 mmol). The mixture was stirred at 25° C. for 2 hours. The mixture was concentrated and purified by thin layer chromatography to give a yellow oil tert-butyl (tert-butoxycarbonyl)(5-(4-(isopropylsulfonyl) phenyl)-3-(3-(4-((phenoxycarbonyl)amino)phenyl)isoxazol-5-yl)pyrazin-2-yl)carbamate 14e (20 mg).

### Step Six

### tert-butyl (tert-butoxycarbonyl)(5-(4-(isopropylsulfonyl)phenyl)-3-(3-(4-(3-methylureido)phenyl)isoxazol-5-yl)pyrazin-2-yl)carbamate 14f

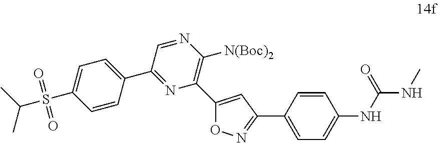

To a solution of tert-butyl (tert-butoxycarbonyl)(5-(4-(isopropylsulfonyl) phenyl)-3-(3-(4-((phenoxycarbonyl)amino) phenyl)isoxazol-5-yl)pyrazin-2-yl)carbamate 14e (20 mg, 0.026 mmol) in acetonitrile (3 mL) was added methylamine hydrochloride (4 mg, 0.052 mmol) and the mixture was stirred for 2 h at 50° C. Ethyl acetate (10 mL) and water (10 mL) were added to the reaction solution. The separated aqueous phase was extracted three times with ethyl acetate (20 mL), and the combined organic phase was concentrated and isolated by column chromatography to give a white solid tert-butyl (tert-butoxycarbonyl)(5-(4-(isopropylsulfonyl)phenyl)-3-(3-(4-(3-methylureido)phenyl)isoxazol-5-yl)pyrazin-2-yl) carbamate 14f (12 mg).

### Step Seven

### 1-(4-(5-(3-amino-6-(4-(isopropylsulfonyl)phenyl)pyrazin-2-yl)isoxazol-3-yl)phenyl)-3-methylurea 14

A reaction solution of tert-butyl (tert-butoxycarbonyl)(5-(4-(isopropylsulfonyl)phenyl)-3-(3-(4-(3-methylureido)phenyl) isoxazol-5-yl)pyrazin-2-yl) carbamate 14f (12 mg, 0.015 mmol) and ethyl acetate solution of HCl (4 M, 2 mL) was stirred for 1 hour at room temperature, and then concentrated. The ethyl acetate (20 mL) and saturated aqueous sodium bicarbonate solution (20 mL) were added. The separated organic phase was washed with water (20 mL). Then, the organic phase was concentrated and isolated by column chromatography to obtain a yellow solid 1-(4-(5-(3-amino-6-(4-(isopropylsulfonyl)phenyl)pyrazin-2-yl)isoxazol-3-yl)phenyl)-3-methylurea 14 (6.7 mg).

¹H NMR (DMSO-d₆, 400 MHz): δ 8.93 (s, 1H), 8.82 (s, 1H), 8.38-8.36 (m, 2H), 7.94-7.92 (m, 2H), 7.88-7.86 (m, 2H), 7.69 (s, 1H), 7.59-7.57 (m, 2H), 7.17 (s, 2H), 6.13-6.11 (m, 1H), 3.48-3.44 (m, 1H), 2.67-2.66 (m, 3H), 1.20-1.18 (m, 6H)

MS measured value (ESI⁺) [(M+H)J]: 493

### Example 15

### 1-(4-(5-(3-amino-6-(4-(isopropylsulfonyl)phenyl)pyrazin-2-yl)isoxazol-3-yl)phenyl)urea 15

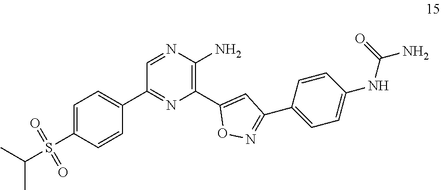 [SMILES: CC(C)S(=O)(=O)c1ccc(-c2cnc(N)c(-c3cc(-c4ccc(NC(N)=O)cc4)no3)n2)cc1]

### Step One

### tert-butyl (tert-butoxycarbonyl)(5-(4-(isopropylsulfonyl)phenyl)-3-(3-(4-ureidophenyl)isoxazol-5-yl)pyrazin-2-yl) carbamate 15a

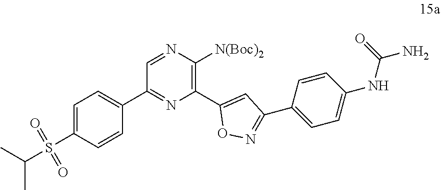

The tert-butyl (tert-butoxycarbonyl)(5-(4-(isopropylsulfonyl) phenyl)-3-(3-(4-((phenoxycarbonyl)amino)phenyl) isoxazole-5-yl)pyrazin-2-yl)carbamate 14e (20 mg, 0.026 mmol) was dissolved in a solution of ammonia-methanol solution (7 M, 3 mL), and the mixture was stirred for 2 hours at 50° C. Ethyl acetate (10 mL) and water (10 mL) were added to the reaction solution. The aqueous phase from separatory was then extracted three times with ethyl acetate (20 mL), and the combined organic phase was concentrated and isolated by column chromatography to give a white solid tert-butyl (tert-butoxycarbonyl)(5-(4-(isopropylsulfonyl)phenyl)-3-(3-(4-ureidophenyl)isoxazol-5-yl)pyrazin-2-yl) carbamate 15a (12 mg).

### Step Two

### 1-(4-(5-(3-amino-6-(4-(isopropylsulfonyl)phenyl)pyrazin-2-yl)isoxazol-3-yl)phenyl)urea 15

The reaction solution of tert-butyl (tert-butoxycarbonyl)(5-(4-(isopropylsulfonyl)phenyl)-3-(3-(4-ureidophenyl) isoxazol-5-yl)pyrazin-2-yl) carbamate 15a (12 mg, 0.015 mmol) and ethyl acetate solution of HCl (4 M, 2 mL) was stirred for 1 hour at room temperature, and then concentrated. Ethyl acetate (20 mL) and saturated aqueous sodium bicarbonate solution (20 mL) were added. After separating, the organic phase was washed with water (20 mL), concentrated and isolated by column chromatography to obtain a yellow solid 1-(4-(5-(3-amino-6-(4-(isopropylsulfonyl)phenyl)pyrazin-2-yl)isoxazol-3-yl)phenyl)urea 15.

¹H NMR (DMSO-d₆, 400 MHz): δ 8.92 (s, 1H), 8.86 (s, 1H), 8.38-8.36 (m, 2H), 7.94-7.92 (m, 2H), 7.88-7.86 (m, 2H), 7.68 (s, 1H), 7.59-7.57 (m, 2H), 7.15 (s, 2H), 6.97 (m, 2H), 3.48-3.44 (m, 1H), 1.20-1.18 (m, 6H)

MS measured value (ESI⁺) [(M+H)⁺]: 479

### Example 16

### 1-(4-(5-(3-amino-6-(4-(isopropylsulfonyl)phenyl)pyrazin-2-yl)isoxazol-3-yl)phenyl)guanidine 16

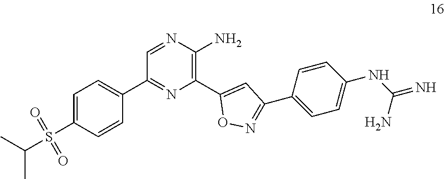 [SMILES: CC(C)S(=O)(=O)c1ccc(-c2cnc(N)c(-c3cc(-c4ccc(NC(=N)N)cc4)no3)n2)cc1]

To a solution of tert-butyl (3-(3-(4-aminophenyl)isoxazol-5-yl)-5-(4-(isopropylsulfonyl)phenyl)pyrazin-2-yl)(tert-butoxycarbonyl)carbamate 14d (50 mg, 0.079 mmol) in dioxane and hydrochloride (4M, 3 mL) was added cyanamide (10 mg, 0.14 mmol) and the mixture was stirred at 80° C. for 2 hours. After the reaction was cooled to room temperature, ethyl acetate (20 mL) and aqueous sodium bicarbonate (10 mL) were added to the reaction solution. After separating, the aqueous phase was extracted with ethyl acetate (20 mL) three times, and the combined organic phase was concentrated and isolated by column chromatography to give a white solid 1-(4-(5-(3-amino-6-(4-(isopropylsulfonyl)phenyl)pyrazin-2-yl)isoxazol-3-yl)phenyl)guanidine 16 (18.3 mg).

¹H NMR (DMSO-d₆, 400 MHz): δ 8.95 (s, 1H), 8.39-8.37 (m, 2H), 8.10-8.07 (m, 2H), 7.95-7.93 (m, 2H), 7.82 (s, 1H), 7.71 (m, 3H), 7.44-7.42 (m, 2H), 7.19 (s, 2H), 5.40 (s, 2H), 3.50-3.43 (m, 1H), 1.20-1.18 (m, 6H)

MS measured value (ESI⁺) [(M+H)⁺]: 478

### Example 17

### 1-(4-(5-(3-amino-6-(4-(isopropylsulfonyl)phenyl)pyrazin-2-yl)isoxazol-3-yl)phenyl)-3-methylguanidine dihydrochloride 17

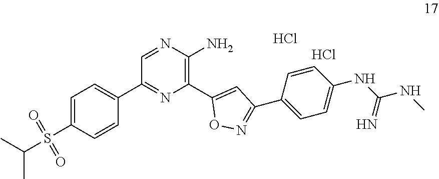 [SMILES: Cl ; CNC(=N)Nc1ccc(-c2cc(-c3nc(-c4ccc(S(=O)(=O)C(C)C)cc4)cnc3N)on2)cc1 ; Cl]

### Step One

### tert-butyl (tert-butoxycarbonyl) (5-(4-(isopropylsulfonyl)phenyl)-3-(3-(4-isothiocyanatophenyl)isoxazol-5-yl)pyrazin-2-yl) carbamate 17a

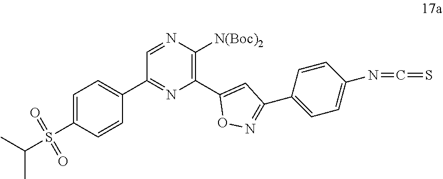

To a solution of tert-butyl (3-(3-(4-aminophenyl)isoxazol-5-yl)-5-(4-(isopropylsulfonyl)phenyl)pyrazin-2-yl)(tert-butoxycarbonyl)carbamate 14d (110 mg, 0.21 mmol) in dichloromethane (4 mL) was added an aqueous solution of sodium hydroxide (1M, 0.6 mL) and phenyl chlorothionocarbonate (43 mg. 0.25 mmol). The mixture was stirred at 25° C. for 2 hours. Dichloromethane (20 mL) and aqueous solution (10 mL) were added to the reaction solution. After separating, the aqueous phase was extracted three times with dichloromethane (10 mL), and the combined organic phase was concentrated and isolated by column chromatography to give the yellow oil tert-butyl (tert-butoxycarbonyl) (5-(4-(isopropylsulfonyl)phenyl)-3-(3-(4-isothiocyanatophenyl)isoxazol-5-yl)pyrazin-2-yl) carbamate 17a (70 mg).

### Step Two

### tert-butyl (tert-butoxycarbonyl)(5-(4-(isopropylsulfonyl)phenyl)-3-(3-(4-(3-methylthioureido)phenyl)isoxazol-5-yl)pyrazin-2-yl) carbamate 17b

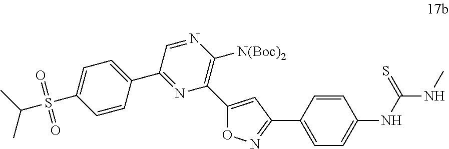

To a solution of tert-butyl (tert-butoxycarbonyl) (5-(4-(isopropylsulfonyl)phenyl)-3-(3-(4-isothiocyanatophenyl) isoxazol-5-yl)pyrazin-2-yl) carbamate 17a (200 mg, 0.42 mmol) in acetonitrile (5 mL) was added methylamine hydrochloride (28 mg, 0.42 mmol), and the reaction solution was stirred at 50° C. for 1 hour. Then, ethyl acetate (20 mL) and water (20 mL) were added. After separating, the organic phase was washed with water (20 mL), concentrated and isolated by column chromatography to obtain yellow solid tert-butyl (tert-butoxycarbonyl)(5-(4-(isopropylsulfonyl)phenyl)-3-(3-(4-(3-methylthioureido)phenyl)isoxazol-5-yl)pyrazin-2-yl) carbamate 17b (150 mg).

### Step Three

### tert-butyl (tert-butoxycarbonyl) (5-(4-(isopropylsulfonyl)phenyl)-3-(3-(4-(3-methylguanidino)phenyl)isoxazol-5-yl)pyrazin-2-yl)carbamate 17c

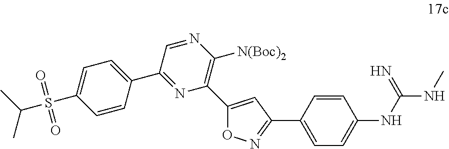

To the reaction solution of tert-butyl (tert-butoxycarbonyl)(5-(4-(isopropylsulfonyl)phenyl)-3-(3-(4-(3-methylthioureido)phenyl)isoxazol-5-yl)pyrazin-2-yl) carbamate 17b (26 mg, 0.04 mmol) in acetonitrile (1.5 mL) and ammonia (1.5 mL) was added 2-iodoacyl benzoic acid (13 mg, 0.05 mmol). After the reaction solution was stirred at 50° C. for 1 hour, ethyl acetate (20 mL) and water (20 mL) were added, and the organic phase was washed with water (20 mL). after separating, the organic phase was concentrated and isolated by column chromatography to obtain a yellow solid tert-butyl (tert-butoxycarbonyl) (5-(4-(isopropylsulfonyl)phenyl)-3-(3-(4-(3-methylguanidino)phenyl)isoxazol-5-yl)pyrazin-2-yl)carbamate 17c (5 mg).

### Step Four

### 1-(4-(5-(3-amino-6-(4-(isopropylsulfonyl)phenyl)pyrazin-2-yl)isoxazol-3-yl)phenyl)-3-methylguanidine dihydrochloride 17

The reaction solution of tert-butyl (tert-butoxycarbonyl) (5-(4-(isopropylsulfonyl)phenyl)-3-(3-(4-(3-methylguanidino)phenyl)isoxazol-5-yl)pyrazin-2-yl)carbamate 17c (5 mg, 0.007 mmol) and a mixed solution (4 M, 2 mL) of hydrochloric acid and ethyl acetate was stirred for 1 hour at room temperature. Then, the reaction solution was concentrated to give 1-(4-(5-(3-amino-6-(4-(isopropylsulfonyl)phenyl)pyrazin-2-yl)isoxazol-3-yl)phenyl)-3-methylguanidine dihydrochloride 17 (1.7 mg).

¹H NMR (400 MHz, DMSO-d₆): δ 8.98 (s, 1H), 8.42-8.40 (m, 2H), 8.12-8.10 (m, 2H), 8.02-8.00 (m, 2H), 7.86 (s, 1H), 7.46-7.44 (m, 2H), 3.51-3.48 (m, 1H), 2.89-2.88 (m, 3H), 1.22-1.21 (m, 6H)

MS measured value (ESI⁺) [(M+H)⁺]: 492

### Example 18

### 1-(4-(5-(3-amino-6-(4-(isopropylsulfonyl)phenyl)pyrazin-2-yl)isoxazol-3-yl)phenyl)-3-ethylguanidine dihydrochloride 18

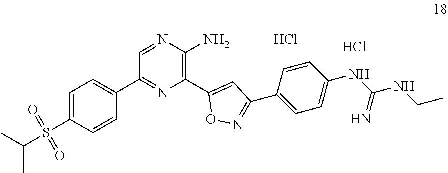 [SMILES: Cl ; CCNC(=N)Nc1ccc(-c2cc(-c3nc(-c4ccc(S(=O)(=O)C(C)C)cc4)cnc3N)on2)cc1 ; Cl]

### Step One

### tert-butyl (tert-butoxycarbonyl) (3-(3-(3-(4-(3-ethylthioureido)phenyl)isoxazol-5-yl)-5-(4-(isopropylsulfonyl)phenyl)-pyrazin-2-yl) carbamate 18a

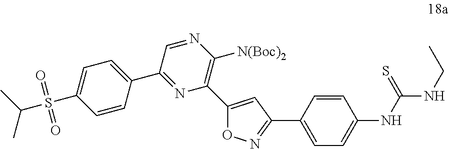

To a solution of tert-butyl (tert-butoxycarbonyl) (5-(4-(isopropylsulfonyl)phenyl)-3-(3-(4-isothiocyanatophenyl) isoxazol-5-yl)pyrazin-2-yl) carbamate 17a (120 mg, 0.42 mmol) in acetonitrile (5 mL) were added ethylamine hydrochloride (25 mg, 0.13 mmol) and triethylamine (63 mg, 0.62 mmol). After the reaction solution was stirred at 50° C. for 1 hour, ethyl acetate (20 mL) and water (20 mL) were added. After separating, the organic phase was washed with water (20 mL), and concentrated and isolated by column chromatography to obtain a yellow solid tert-butyl (tert-butoxycarbonyl) (3-(3-(3-(4-(3-ethylthioureido)phenyl)isoxazol-5-yl)-5-(4-(isopropylsulfonyl)phenyl)-pyrazin-2-yl) carbamate 18a (100 mg).

### Step Two

### tert-butyl (tert-butoxycarbonyl)(3-(3-(4-(3-ethylguanidino)phenyl)isoxazol-5-yl)(5-(4-(isopropylsulfonyl)phenyl)-pyrazin-2-yl) carbamate 18b

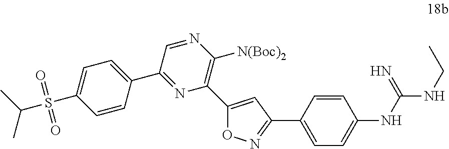

To the solution of tert-butyl (tert-butoxycarbonyl) (3-(3-(3-(4-(3-ethylthioureido)phenyl)isoxazol-5-yl)-5-(4-(isopropylsulfonyl)phenyl)-pyrazin-2-yl) carbamate 18a (100 mg, 0.16 mmol) in acetonitrile (2 mL) and ammonia (2 mL) was added 2-iodobenzoic acid (50 mg, 0.18 mmol). The reaction solution was stirred at 50° C. for 1 hour, and then ethyl acetate (20 mL) and water (20 mL) were added. After separating, the organic phase was washed with water (20 mL), concentrated and isolated by column chromatography to obtain a yellow solid tert-butyl (tert-butoxycarbonyl)(3-(3-(4-(3-ethylguanidino)phenyl)isoxazol-5-yl)(5-(4-(isopropylsulfonyl)phenyl)-pyrazin-2-yl) carbamate 18b (5 mg).

### Step Three

### 1-(4-(5-(3-amino-6-(4-(isopropylsulfonyl)phenyl)pyrazin-2-yl)isoxazol-3-yl)phenyl)-3-ethylguanidine dihydrochloride 18

The reaction solution of tert-butyl (tert-butoxycarbonyl)(3-(3-(4-(3-ethylguanidino)phenyl)isoxazol-5-yl)(5-(4-(isopropylsulfonyl)phenyl)-pyrazin-2-yl) carbamate 18b (45 mg, 0.063 mmol) and mixture (4 M, 2 mL) of hydrochloric acid and ethyl acetate was stirred for 1 hour at room temperature. Then, the reaction solution was concentrated to give a yellow solid 1-(4-(5-(3-amino-6-(4-(isopropylsulfonyl)phenyl)pyrazin-2-yl)isoxazol-3-yl)phenyl)-3-ethylguanidine dihydrochloride 18 (34.5 mg).

¹H NMR (400 MHz, DMSO-d₆): δ 9.94 (s, 1H), 8.96 (s, 1H), 8.39-8.37 (m, 2H), 8.17-8.15 (m, 1H), 8.09-8.07 (m, 2H), 7.95-7.93 (m, 2H) 7.83 (s, 1H), 7.42-7.40 (m, 2H), 3.50-3.45 (m, 1H), 3.33-2.29 (m, 2H), 1.20-1.15 (m, 9H)

MS measured value (ESI⁺) [(M+H)⁺]: 506

### Example 19

### 1-(6-(5-(3-amino-6-(4-(isopropylsulfonyl)phenyl)pyrazin-2-yl)isoxazol-3-yl)pyridin-3-yl)-3-methylguanidine dihydrochloride 19

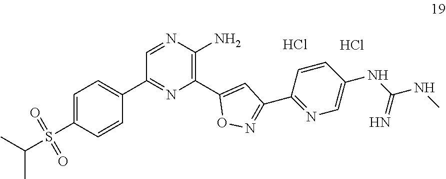 [SMILES: CNC(=N)Nc1ccc(-c2cc(-c3nc(-c4ccc(S(=O)(=O)C(C)C)cc4)cnc3N)on2)nc1 ; Cl ; Cl]

### Step One

### (E)-5-nitropyridinecarboxaldehyde oxime 19a

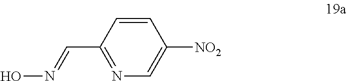 [SMILES: O=[N+]([O-])c1ccc(/C=N/O)nc1]

To a solution of 5-nitropyridinecarboxaldehyde (1 g, 6.57 mmol) in tetrahydrofuran (15 mL) was added hydroxylamine hydrochloride (0.55 g, 7.89 mmol) at room temperature. The reaction mixture was stirred at room temperature for 16 hours. Water (40 mL) was added to the reaction system and the organic phase was separated out. The aqueous phase was extracted three times with ethyl acetate (30 mL). The combined organic phase was dried with anhydrous sodium sulfate, and filtered. The filtrate was concentrated under reduced pressure to give (E)-5-nitropyridinecarboxaldehyde oxime 19a (1.1 g) which was a crude product and used directly in the next step without a further purification.

### Step Two

### tert-butyl (5-bromo-3-(3-(5-nitropyridin-2-yl)isoxazol-5-yl)pyrazin-2-yl)(tert-butoxycarbonyl) carbamate 19b

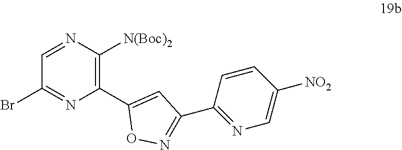

To a solution of tert-butyl (5-bromo-3-ethynyl-2-pyrazinyl)(tert-butoxycarbonyl)carbamate (0.80 g, 1.99 mmol) in a mixture of methanol (18 mL) and water (3.6 mL) were sequentially added (E)-5-nitropyridinecarboxaldehyde oxime 19a (0.50 g, 2.99 mmol) and bis(trifluoroacetyl)iodobenzene (1.29 g, 2.99 mmol). The reaction solution was stirred at room temperature overnight. Ethyl acetate (40 mL) and water (40 mL) were added to the reaction solution. Then, the organic phase was washed three times with water (20 mL). The organic phase was separated, dried with anhydrous sodium sulfate, filtered, concentrated and isolated by column chromatography to give tert-butyl (5-bromo-3-(3-(5-nitropyridin-2-yl) isoxazol-5-yl)pyrazin-2-yl)(tert-butoxycarbonyl) carbamate 19b (0.20 g).

### Step Three

### tert-butyl (tert-butoxycarbonyl)(5-(4-(isopropylsulfonyl)phenyl)-3-(3-(5-nitropyridin-2-yl)isoxazol-5-yl)pyrazin-2-yl) carbamate 19c

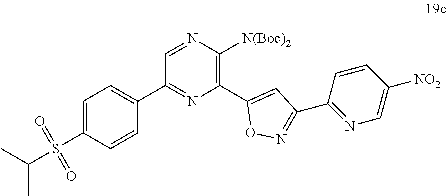

Under nitrogen protection, to a solution of tert-butyl (5-bromo-3-(3-(5-nitropyridin-2-yl) isoxazol-5-yl)pyrazin-2-yl)(tert-butoxycarbonyl) carbamate 19b (0.20 g, 0.36 mmol) in a mixture of 1,4-dioxane (15 mL) and water (1.5 mL) were sequentially added 4-isopropylsulfonylphenylboronic acid (0.082 g, 0.36 mmol), potassium carbonate (0.15 g, 1.07 mmol) and [1,1′-bis(diphenylphosphino)ferrocene]palladium(II) chloride (0.05 g, 0.071 mmol) at room temperature (˜20° C.).

After three times of nitrogen replacements, the reaction solution was stirred at 95° C. for three hours. After cooling to room temperature, ethyl acetate (40 mL) and water (40 mL) were added to the reaction solution. The mixed solution was filtered through diatomaceous earth and the resulting solution was separated. The aqueous phase was extracted three times with ethyl acetate (20 mL), and the combined organic phase was concentrated and isolated by column chromatography to give tert-butyl (tert-butoxycarbonyl)(5-(4-(isopropylsulfonyl)phenyl)-3-(3-(5-nitropyridin-2-yl)isoxazol-5-yl)pyrazin-2-yl) carbamate 19c (0.12 g).

### Step Four

### tert-butyl (3-(3-(5-aminopyridin-2-yl)isoxazol-5-yl)-5-(4-(isopropylsulfonyl)phenyl)pyrazin-2-yl) (tert-butoxycarbonyl) carbamate 19d

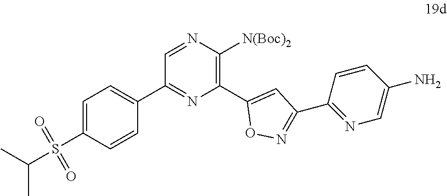

To a solution of tert-butyl (tert-butoxycarbonyl)(5-(4-(isopropylsulfonyl)phenyl)-3-(3-(5-nitropyridin-2-yl)isoxazol-5-yl)pyrazin-2-yl) carbamate 19c (120 mg, 0.20 mmol) in acetic acid (8 mL) was added reduced iron powder (109 mg, 2.0 mmol) at room temperature. The reaction solution was stirred for 16 hours at room temperature, then diluted with ethyl acetate (40 mL). The mixture was filtered through diatomaceous earth, washed to be neutral with aqueous sodium bicarbonate (40 mL) and then poured into a separatory funnel. The aqueous phase was extracted three times with ethyl acetate (40 mL) and the combined organic phase was concentrated and isolated by column chromatography to give tert-butyl (3-(3-(5-aminopyridin-2-yl)isoxazol-5-yl)-5-(4-(isopropylsulfonyl)phenyl)pyrazin-2-yl) (tert-butoxycarbonyl) carbamate 19d (75 mg).

### Step Five

### tert-butyl (tert-butoxycarbonyl)(5-(4-(isopropylsulfonyl)phenyl)-3-(3-(5-isothiocyanatopyridin-2-yl)isoxazol-5-yl)pyrazin-2-yl)carbamate 19e

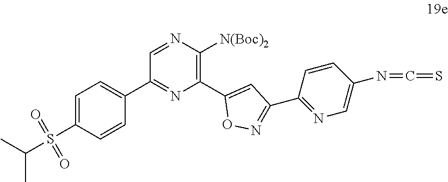

To a solution of tert-butyl (3-(3-(5-aminopyridin-2-yl)isoxazol-5-yl)-5-(4-(isopropylsulfonyl)phenyl)pyrazin-2-yl) (tert-butoxycarbonyl) carbamate 19d (75 mg, 0.11 mmol) in dichloromethane (4 mL) were added an aqueous sodium hydroxide (1 M, 0.35 mL) solution and phenyl thiocarbonate (23 mg, 0.13 mmol). The mixture was stirred at 25° C. for 2 hours. Dichloromethane (20 mL) and aqueous solution (10 mL) were added to the reaction solution. The aqueous phase from separating was then extracted three times with dichloromethane (10 mL), and the organic phases were combined, concentrated and isolated by column chromatography to give the yellow oil tert-butyl (tert-butoxycarbonyl)(5-(4-(isopropylsulfonyl)phenyl)-3-(3-(5-isothiocyanatopyridin-2-yl)isoxazol-5-yl)pyrazin-2-yl)carbamate 19e (53 mg).

### Step Six

### tert-butyl (tert-butoxycarbonyl)(5-(4-(isopropylsulfonyl)phenyl)-3-(3-(5-(3-methylthioureido)pyridin-2-yl)isoxazol-5-yl)pyrazin-2-yl) carbamate 19f

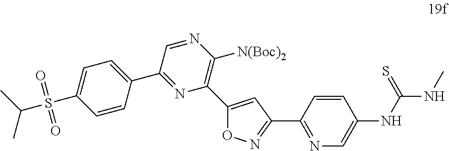

To a solution of tert-butyl (tert-butoxycarbonyl)(5-(4-(isopropylsulfonyl)phenyl)-3-(3-(5-isothiocyanatopyridin-2-yl)isoxazol-5-yl)pyrazin-2-yl)carbamate 19e (45 mg, 0.078 mmol) in acetonitrile (5 mL) were added methylamine hydrochloride (8 mg, 0.12 mmol) and triethylamine (24 mg, 0.23 mmol). The reaction solution was stirred at 50° C. for 1 hour, and then ethyl acetate (20 mL) and water (20 mL) were added. The organic phase from separating was washed with water (20 mL), and was concentrated and isolated by column chromatography to obtain a yellow solid tert-butyl (tert-butoxycarbonyl) (5-(4-(isopropylsulfonyl)phenyl)-3-(3-(5-(3-methylthioureido)pyridin-2-yl)isoxazol-5-yl)pyrazin-2-yl) carbamate 19f (38 mg).

### Step Seven

### tert-butyl (tert-butoxycarbonyl)(5-(4-(isopropylsulfonyl)phenyl)-3-(3-(5-(3-methylguanidino)pyridin-2-yl)isoxazol-5-yl)pyrazin-2-yl)carbamate 19g

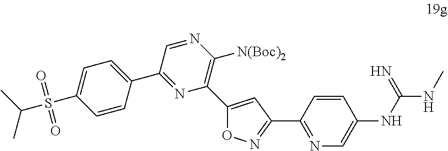

To a solution of tert-butyl (tert-butoxycarbonyl) (5-(4-(isopropylsulfonyl)phenyl)-3-(3-(5-(3-methylthioureido)pyridin-2-yl)isoxazol-5-yl)pyrazin-2-yl) carbamate 19f (38 mg, 0.075 mmol) in acetonitrile (1.5 mL) and ammonia (1.5 mL) was added 2-iodoxybenzoic acid (25 mg, 0.089 mmol). After the reaction solution was stirred at 50° C. for 1 hour, ethyl acetate (20 mL) and water (20 mL) were added. After the organic phase from separating was washed with water (20 mL), the organic phase was concentrated and isolated by column chromatography to obtain a yellow solid tert-butyl (tert-butoxycarbonyl) (5-(4-(isopropylsulfonyl)phenyl)-3-(3-(5-(3-methylguanidino)pyridin-2-yl)isoxazol-5-yl)pyrazin-2-yl) carbamate 19g (15 mg).

### Step Eight

### 1-(6-(5-(3-amino-6-(4-(isopropylsulfonyl)phenyl)pyrazin-2-yl)isoxazol-3-yl)pyridin-3-yl)-3-methylguanidine dihydrochloride 19

The reaction solution of tert-butyl (tert-butoxycarbonyl) (5-(4-(isopropylsulfonyl)phenyl)-3-(3-(5-(3-methylguanidino) pyridin-2-yl)isoxazol-5-yl)pyrazin-2-yl) carbamate 19g (15 mg, 0.021 mmol) and mixture (4 M, 2 mL) of hydrochloric acid and ethyl acetate was stirred for 1 hour at room temperature. The reaction solution was concentrated to give a yellow solid 1-(6-(5-(3-amino-6-(4-(isopropylsulfonyl)phenyl)pyrazin-2-yl)isoxazol-3-yl)pyridin-3-yl)-3-methylguanidine dihydrochloride 19 (11.7 mg).

¹H NMR (400 MHz, DMSO-d₆): δ 10.13 (s, 1H), 8.96 (s, 1H), 8.68 (m, 1H), 8.39-8.37 (m, 2H), 8.21-8.17 (m, 2H), 7.95-7.93 (m, 2H), 7.90-7.87 (m, 2H), 7.71 (s, 1H), 3.49-3.45 (m, 1H), 2.87 (m, 3H), 1.20-1.18 (m, 6H)

MS measured value (ESI⁺) [(M+H)⁺]: 493

### Example 20

### 1-(4-(5-(3-amino-6-(4-(isopropylsulfonyl)phenyl)pyrazin-2-yl)isoxazol-3-yl)-3-fluorophenyl)-3-methylguanidine dihydrochloride 20

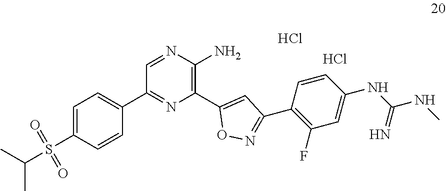 [SMILES: Cl ; CNC(=N)Nc1ccc(-c2cc(-c3nc(-c4ccc(S(=O)(=O)C(C)C)cc4)cnc3N)on2)c(F)c1 ; Cl]

### Step One

### (E)-2-fluoro-4-nitrobenzaldehyde oxime 20a

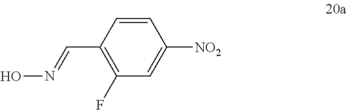 [SMILES: O=[N+]([O-])c1ccc(/C=N/O)c(F)c1]

To a solution of 2-fluoro-4-nitrobenzaldehyde (0.5 g, 2.96 mmol) in tetrahydrofuran (8 mL) was added hydroxylamine hydrochloride (0.25 g, 3.55 mmol) at room temperature. The reaction mixture was stirred at room temperature (˜20° C.) for 16 hours. Then, water (40 mL) was added to the reaction system and the organic phase was separated out. The aqueous phase was extracted three times with ethyl acetate (30 mL). The organic phases were combined, dried with anhydrous sodium sulfate, and filtered. The filtrate was concentrated under reduced pressure to give a yellow solid, (E)-2-fluoro-4-nitrobenzaldehyde oxime 20a (0.7 g), which was a crude product and was used directly in the next step without a further purification.

### Step Two

### tert-butyl (5-bromo-3-(3-(2-fluoro-4-nitrophenyl)isoxazol-5-yl)pyrazin-2-yl)(tert-butoxycarbonyl)carbamate 20b

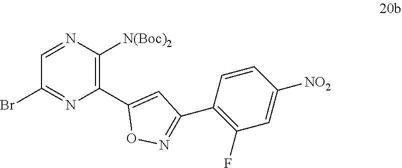

To a mixed solution of tert-butyl (5-bromo-3-ethynyl-2-pyrazinyl)(tert-butoxycarbonyl)carbamate (0.85 g, 2.14 mmol) in methanol (18 mL) and water (3.6 mL) were added sequentially (E)-2-fluoro-4-nitrobenzaldehyde oxime 20a (0.59 g, 3.21 mmol) and bis(trifluoroacetyl)iodobenzene (1.38 g, 3.21 mmol). 3.21 mmol). The reaction solution was stirred at room temperature overnight and then added with ethyl acetate (40 mL) and water (40 mL). The organic phase was washed three times with water (20 mL). The organic phase was separated and dried by anhydrous sodium sulfate, filtered, concentrated and isolated by column chromatography to give tert-butyl (5-bromo-3-(3-(2-fluoro-4-nitrophenyl)isoxazol-5-yl)pyrazin-2-yl)(tert-butoxycarbonyl)carbamate 20b (0.72 g).

### Step Three

### tert-butyl (tert-butoxycarbonyl) (3-(3-(2-fluoro-4-nitrophenyl)isoxazol-5-yl)-5-(4-(isopropylsulfonyl)phenyl)pyrazin-2-yl) carbamate 20c

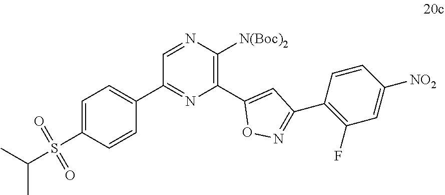

Under nitrogen protection, to a mixed solution of tert-butyl (5-bromo-3-(3-(2-fluoro-4-nitrophenyl)isoxazol-5-yl)pyrazin-2-yl)(tert-butoxycarbonyl)carbamate 20b (0.30 g, 0.52 mmol) in 1,4-dioxane (15 mL) and water (1.5 mL) were sequentially added 4-isopropylsulfonylphenylboronic acid (0.12 g, 0.52 mmol), potassium carbonate (0.22 g, 1.55 mmol) and [1,1′-bis(diphenylphosphino)ferrocene]palladium(II) chloride (0.076 g, 0.01 mmol) at room temperature (˜20° C.). After three times of nitrogen replacements, the reaction solution was stirred at 95° C. for three hours. After cooling to room temperature, ethyl acetate (40 mL) and water (40 mL) were added to the reaction solution. The mixed solution was filtered through diatomaceous earth, and the resulting solution was separated. The aqueous phase was extracted three times with ethyl acetate (20 mL), and the combined organic phase was concentrated and isolated by column chromatography to give tert-butyl (tert-butoxycarbonyl) (3-(3-(2-fluoro-4-nitrophenyl)isoxazol-5-yl)-5-(4-(isopropylsulfonyl)phenyl)pyrazin-2-yl) carbamate 20c (0.13 g).

### Step Four

### tert-butyl (tert-butoxycarbonyl) (3-(3-(4-amino-2-fluorophenyl)isoxazol-5-yl)-5-(4-(isopropylsulfonyl)phenyl)pyrazin-2-yl)carbamate 20d

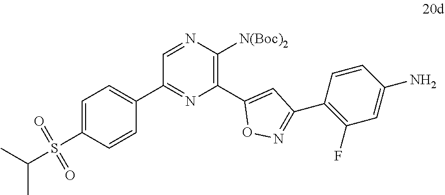

To a solution of tert-butyl (tert-butoxycarbonyl) (3-(3-(2-fluoro-4-nitrophenyl)isoxazol-5-yl)-5-(4-(isopropylsulfonyl) phenyl)pyrazin-2-yl) carbamate 20c (130 mg, 0.19 mmol) in acetic acid (8 mL) was added reduced iron powder (106 mg, 1.9 mmol) at room temperature. The reaction solution was stirred at room temperature for 16 hours and then diluted with ethyl acetate (40 mL). The mixture was filtered through diatomaceous earth, washed to be neutral with aqueous sodium bicarbonate (40 mL) and poured into a separatory funnel. The aqueous phase was extracted three times with ethyl acetate (40 mL) and the combined organic phase was concentrated and isolated by column chromatography to give tert-butyl (tert-butoxycarbonyl) (3-(3-(4-amino-2-fluorophenyl)isoxazol-5-yl)-5-(4-(isopropylsulfonyl)phenyl)pyrazin-2-yl)carbamate 20d (85 mg).

### Step Five

### tert-butyl (tert-butoxycarbonyl)(3-(3-(2-fluoro-4-isothiocyanatophenyl)isoxazol-5-yl)-(5-(4-(isopropylsulfonyl)phenyl)-pyrazin-2-yl) carbamate 20e

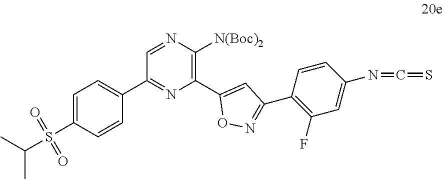

To a solution of tert-butyl (tert-butoxycarbonyl) (3-(3-(4-amino-2-fluorophenyl)isoxazol-5-yl)-5-(4-(isopropylsulfonyl) phenyl)pyrazin-2-yl) carbamate 20d (75 mg, 0.11 mmol) in dichloromethane (4 mL) were added an aqueous sodium hydroxide solution (1M, 0.35 mL) and phenyl thiocarbamate (23 mg, 0.13 mmol). The mixture was stirred at 25° C. for 2 hours. Dichloromethane (20 mL) and aqueous solution (10 mL) were added to the reaction solution. After separating, the aqueous phase was extracted three times with dichloromethane (10 mL), and the combined organic phase was concentrated and isolated by column chromatography to give a yellow oil tert-butyl (tert-butoxycarbonyl) (3-(3-(2-fluoro-4-isothiocyanatophenyl)isoxazol-5-yl)-(5-(4-(isopropylsulfonyl)phenyl)-pyrazin-2-yl)carbamate 20e (56 mg).

### Step Six

### tert-butyl (tert-butoxycarbonyl) (3-(3-(3-(2-fluoro-4-(3-methylthioureido)phenyl)isoxazol-5-yl)-5-(4-(isopropylsulfonyl)phenyl)pyrazin-2-yl) carbamate 20f

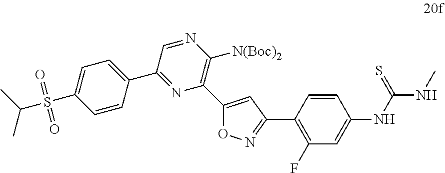

To a solution of tert-butyl (tert-butoxycarbonyl) (3-(3-(2-fluoro-4-isothiocyanatophenyl)isoxazol-5-yl)-(5-(4-(isopropylsulfonyl)phenyl)-pyrazin-2-yl)carbamate 20e (56 mg, 0.078 mmol) in acetonitrile (5 mL) were added methylamine hydrochloride (8 mg, 0.12 mmol) and triethylamine (24 mg, 0.23 mmol). The reaction solution was stirred at 50° C. for 1 hour, and then ethyl acetate (20 mL) and water (20 mL) were added. After separating, the organic phase was washed with water (20 mL), then concentrated and isolated by column chromatography to obtain the yellow solid tert-butyl (tert-butoxycarbonyl) (3-(3-(3-(2-fluoro-4-(3-methylthioureido)phenyl)isoxazol-5-yl)-5-(4-(isopropylsulfonyl)phenyl)pyrazin-2-yl) carbamate 20f (38 mg).

### Step Seven

### tert-butyl (tert-butoxycarbonyl)(3-(3-(3-(2-fluoro-4-(3-methylguanidino)phenyl)isoxazol-5-yl)-5-(4-(isopropylsulfonyl)phenyl)pyrazin-2-yl)carbamate 20g

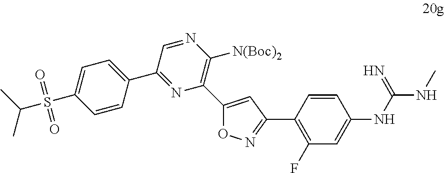

To a reaction solution of tert-butyl (tert-butoxycarbonyl) (3-(3-(3-(2-fluoro-4-(3-methylthioureido)phenyl)isoxazol-5-yl)-5-(4-(isopropylsulfonyl)phenyl)pyrazin-2-yl) carbamate 20f (38 mg, 0.075 mmol) in acetonitrile (1.5 mL) and ammonia (1.5 mL) was added 2-iodoylbenzoic acid (25 mg, 0.089 mmol). The reaction solution was stirred at 50° C. for 1 hour, and then ethyl acetate (20 mL) and water (20 mL) were added. After separating, the organic phase was washed with water (20 mL), and then was concentrated and isolated by column chromatography to obtain the yellow solid tert-butyl (tert-butoxycarbonyl) (3-(3-(3-(2-fluoro-4-(3-methylguanidino)phenyl)isoxazol-5-yl)-5-(4-(isopropylsulfonyl)phenyl)pyrazin-2-yl)carbamate 20g (28 mg).

### Step Eight

### 1-(4-(5-(3-amino-6-(4-(isopropylsulfonyl)phenyl)pyrazin-2-yl)isoxazol-3-yl)-3-fluorophenyl)-3-methylguanidine dihydrochloride 20

The reaction solution of tert-butyl (tert-butoxycarbonyl) (3-(3-(3-(2-fluoro-4-(3-methylguanidino)phenyl)isoxazol-5-yl)-5-(4-(isopropylsulfonyl)phenyl)pyrazin-2-yl)carbamate 20g (28 mg, 0.039 mmol) and a mixture (4 M, 2 mL) of hydrochloric acid and ethyl acetate was stirred for 1 hour at room temperature. Then, the reaction solution was concentrated to give a yellow solid 1-(4-(5-(3-amino-6-(4-(isopropylsulfonyl)phenyl)pyrazin-2-yl)isoxazol-3-yl)-3-fluorophenyl)-3-methylguanidine dihydrochloride 20 (19.1 mg).

¹H NMR (400 MHz, DMSO-d₆): δ 10.16 (s, 1H), 8.99 (s, 1H), 8.40-8.38 (m, 2H), 8.23-8.22 (m, 1H), 8.11-8.07 (m, 2H), 7.98-7.96 (m, 2H), 7.63-7.62 (m, 1H), 7.42-7.39 (m, 1H), 7.29-7.26 (m, 2H), 3.52-3.46 (m, 1H), 2.91-2.89 (m, 3H), 1.23-1.21 (m, 6H)

MS measured value (ESI⁺) [(M+H)⁺]: 510

### Example 21

### 5-(5-(3-amino-6-(4-(isopropylsulfonyl)phenyl)pyrazin-2-yl)isoxazol-3-yl)-1,3-dihydro-2H-benzo[d]imidazol-2-one 21

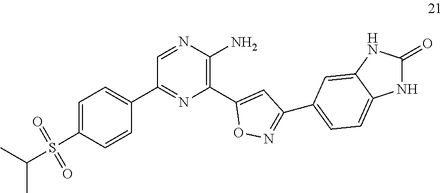 [SMILES: CC(C)S(=O)(=O)c1ccc(-c2cnc(N)c(-c3cc(-c4ccc5[nH]c(=O)[nH]c5c4)no3)n2)cc1]

### Step One

### (E)-2-oxo-2,3-dihydro-1H-benzo[d]imidazole-5-carbaldehyde oxime 21a

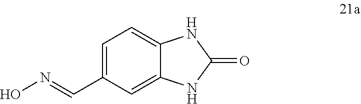 [SMILES: O=c1[nH]c2ccc(/C=N/O)cc2[nH]1]

To a solution of 2-oxo-2,3-dihydro-benzimidazole-5-carbaldehyde (0.7 g, 4.31 mmol) in tetrahydrofuran (16 mL) was added hydroxylamine hydrochloride (0.36 g, 5.18 mmol) at room temperature. The reaction mixture was stirred at room temperature (˜20° C.) for 16 hours. Water (40 mL) was added to the reaction system. The organic phase was separated out, and the aqueous phase was extracted three times with ethyl acetate (30 mL). The combined organic phase was dried with anhydrous sodium sulfate and filtered. The filtrate was concentrated under reduced pressure to give the yellow solid (E)-2-oxo-2,3-dihydro-1H-benzo[d]imidazole-5-carbaldehyde oxime 21a which was a crude product and used directly in the next step without a further purification.

### Step Two

### tert-butyl (5-bromo-3-(3-(2-oxo-2,3-dihydro-1H-benzo[d]imidazol-5-yl)isoxazol-5-yl)pyrazin-2-yl)(tert-butoxycarbonyl) carbamate 21b

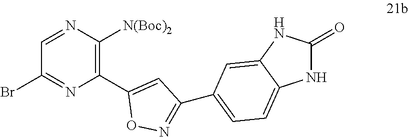

To a mixed solution of tert-butyl (5-bromo-3-ethynyl-2-pyrazinyl)(tert-butoxycarbonyl)carbamate (1.05 g, 2.14 mmol) in methanol (25 mL) and water (5 mL) were added (E)-2-oxo-2,3-dihydro-1H-benzo[d]imidazole-5-carbaldehyde oxime 21a (0.7 g, 3.95 mmol) and bistrifluoroacetyliodobenzene (1.7 g, 3.95 mmol). The reaction solution was stirred overnight at room temperature (about 20° C.), and then was added with ethyl acetate (40 mL) and water (40 mL). The organic phase was washed three times with water (20 mL), then separated out and dried with anhydrous sodium sulfate, filtered, concentrated and isolated by column chromatography to give tert-butyl (5-bromo-3-(3-(2-oxo-2,3-dihydro-1H-benzo[d]imidazol-5-yl)isoxazol-5-yl)pyrazin-2-yl)(tert-butoxycarbonyl) carbamate 21b (0.33 g).

### Step Three

### tert-butyl (tert-butoxycarbonyl)(5-(4-(isopropylsulfonyl)phenyl)-3-(3-(2-oxo-2,3-dihydro-1H-benzo[d]imidazol-5-yl)isoxazol-5-yl)pyrazin-2-yl)carbamate 21c

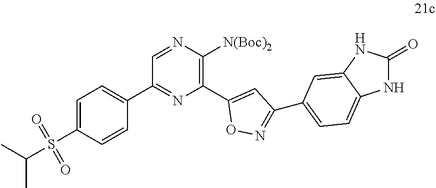

Under nitrogen protection, to a mixed solution of tert-butyl (5-bromo-3-(3-(2-oxo-2,3-dihydro-1H-benzo[d]imidazol-5-yl)isoxazol-5-yl)pyrazin-2-yl)(tert-butoxycarbonyl) carbamate 21b (0.150, 0.26 mmol) in a mixture of 1,4-dioxane (7 mL) and water (1.4 mL) were sequentially added 4-isopropylsulfonylphenylboronic acid (0.066 g, 0.29 mmol), potassium carbonate (0.073 g, 0.52 mmol) and [1,1′-bis(diphenylphosphino)ferrocene]palladium(II) chloride (0.038 g, 0.05 mmol) at room temperature. After three times of nitrogen replacements, the reaction solution was stirred at 95° C. for three hours. After cooling to room temperature, ethyl acetate (40 mL) and water (40 mL) were added to the reaction solution. The mixed solution was filtered through diatomaceous earth and the resulting solution was separated. The aqueous phase was extracted three times with ethyl acetate (20 mL), and the combined organic phase was concentrated and isolated by column chromatography to give tert-butyl (tert-butoxycarbonyl)(5-(4-(isopropylsulfonyl)phenyl)-3-(3-(2-oxo-2,3-dihydro-1H-benzo[d]imidazol-5-yl)isoxazol-5-yl)pyrazin-2-yl)carbamate 21c (0.06 g).

### Step Four

### 5-(5-(3-amino-6-(4-(isopropylsulfonyl)phenyl)pyrazin-2-yl)isoxazol-3-yl)-1,3-dihydro-2H-benzo[d]imidazol-2-one 21

The reaction solution of tert-butyl (tert-butoxycarbonyl)(5-(4-(isopropylsulfonyl)phenyl)-3-(3-(2-oxo-2,3-dihydro-1H-benzo[d]imidazol-5-yl)isoxazol-5-yl)pyrazin-2-yl)carbamate 21c (60 mg, 0.13 mmol) and HCl in ethyl acetate solution (4 M, 2 mL) was stirred at room temperature for 1 hour. After that, the reaction solution was diluted with ethyl acetate (400 mL) and washed to be neutral with a saturated solution of sodium bicarbonate. The organic phases were combined and dried, and concentrated to give a yellow solid 5-(5-(3-amino-6-(4-(isopropylsulfonyl)phenyl)pyrazin-2-yl)isoxazol-3-yl)-1,3-dihydro-2H-benzo[d]imidazol-2-one 21 (39.4 mg).

¹H NMR (400 MHz, DMSO-d₆): δ 8.92 (s, 1H), 8.38-8.36 (m, 2H), 7.94-7.92 (m, 2H), 7.70 (s, 1H), 7.62-7.60 (m, 1H), 7.53 (s, 1H), 7.15 (s, 2H), 7.07-7.05 (m, 1H), 3.52-3.46 (m, 1H), 1.20-1.18 (m, 6H)

MS measured value (ESI⁺) [(M+H)⁺]: 477

### Example 22

### 5-(5-(3-amino-6-(4-(propan-2-ylsulfonimido)phenyl)pyrazin-2-yl)isoxazol-3-yl)-1,3-dihydro-benzimidazol-2-one dihydrochloride 22

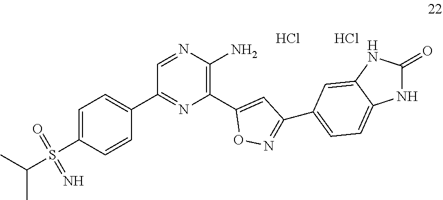 [SMILES: Cl ; Cl ; CC(C)S(=N)(=O)c1ccc(-c2cnc(N)c(-c3cc(-c4ccc5[nH]c(=O)[nH]c5c4)no3)n2)cc1]

### Step One

### tert-butyl (tert-butoxycarbonyl) (3-(3-(3-(2-oxo-2,3-dihydro-1H-benzo[d]imidazol-5-yl)isoxazol-5-yl)-5-(4-(propan-2-ylsulfinyl)phenyl)pyrazin-2-yl)carbamate 22a

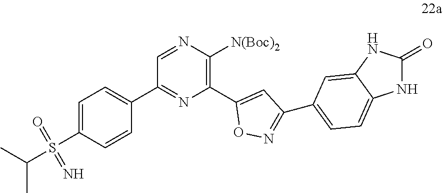

Under nitrogen protection, to a mixed solution of tert-butyl (5-bromo-3-(3-(2-oxo-2,3-dihydro-1H-benzo[d]imidazol-5-yl)isoxazol-5-yl)pyrazin-2-yl)(tert-butoxycarbonyl) carbamate 21b (0.150, 0.26 mmol) in 1,4-dioxane (7 mL) and water (1.4 mL), were sequentially added (4-(propan-2-ylsulfonimidoyl)phenyl)boronic acid (0.120 g, 0.52 mmol), potassium carbonate (0.11 g, 0.79 mmol), and [1,1′-bis(diphenylphosphino)ferrocene]palladium(II) chloride (0.038 g, 0.05 mmol) at room temperature (˜20° C.). After three times of nitrogen replacements, the reaction solution was stirred at 95° C. for three hours. After cooling to room temperature, ethyl acetate (40 mL) and water (40 mL) were added to the reaction solution. The mixed solution was filtered through diatomaceous earth and the resulting solution was separated. The aqueous phase was extracted three times with ethyl acetate (20 mL), and the combined organic phase was concentrated and isolated by column chromatography to give tert-butyl (tert-butoxycarbonyl) (3-(3-(3-(2-oxo-2,3-dihydro-1H-benzo[d]imidazol-5-yl)isoxazol-5-yl)-5-(4-(propan-2-ylsulfinyl)phenyl)pyrazin-2-yl)carbamate 22a (0.07 g).

### Step Two

### 5-(5-(3-amino-6-(4-(propan-2-ylsulfonimido)phenyl)pyrazin-2-yl)isoxazol-3-yl)-1,3-dihydro-benzimidazol-2-one dihydrochloride 22

The reaction solution of tert-butyl (tert-butoxycarbonyl) (3-(3-(3-(2-oxo-2,3-dihydro-1H-benzo[d]imidazol-5-yl)isoxazol-5-yl)-5-(4-(propan-2-ylsulfinyl)phenyl)pyrazin-2-yl)carbamate 22a (70 mg, 0.18 mmol) and the mixture (4 M, 2 mL) of HCl and ethyl acetate solution was stirred for 1 hour at room temperature. The reaction solution was concentrated to give a yellow solid 5-(5-(3-amino-6-(4-(propan-2-ylsulfonimido)phenyl)pyrazin-2-yl)isoxazol-3-yl)-1,3-dihydro-benzimidazol-2-one dihydrochloride 22 (48.1 mg).

¹H NMR (400 MHz, DMSO-d₆): δ 10.97-10.95 (m, 2H), 9.04 (s, 1H), 8.55-8.53 (m, 2H), 8.15-8.13 (m, 2H), 7.78 (s, 1H), 7.67-7.65 (m, 1H), 7.57 (s, 1H), 7.13-7.11 (m, 2H), 4.25-4.18 (m, 1H), 1.44-1.42 (m, 3H), 1.33-1.31 (in, 3H)

MS measured value (ESI⁺) [(M+H)⁺]: 476

### Example 23

### 1-(4-(5-(3-amino-6-(4-(cyclopropylsulfonyl)phenyl)pyrazin-2-yl)isoxazol-3-yl)benzyl)guanidine dihydrochloride 23

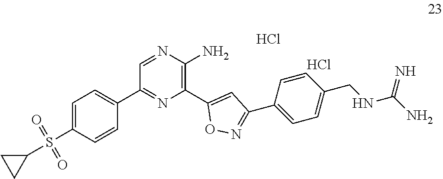 [SMILES: Cl ; N=C(N)NCc1ccc(-c2cc(-c3nc(-c4ccc(S(=O)(=O)C5CC5)cc4)cnc3N)on2)cc1 ; Cl]

### Step One

### (4-(cyclopropylsulfonyl)phenyl)boronic acid 23a

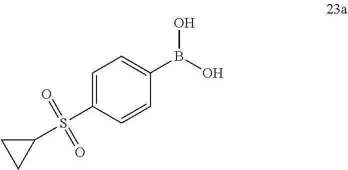 [SMILES: O=S(=O)(c1ccc(B(O)O)cc1)C1CC1]

To a mixed solution of 1-bromo-4-(cyclopropylsulfonyl)benzene (500 mg, 2.03 mmol) in N,N-dimethylformamide (12 mL) was sequentially added bis-pinacol borate (775 mg, 3.05 mmol), potassium acetate (497 mg, 0.51 mmol), and [1,1′-bis(diphenylphosphino)ferrocene]palladium(II) chloride (150 mg, 0.20 mmol) at room temperature. After three times of nitrogen replacements, the reaction solution was stirred at 95° C. for three hours. After cooling to room temperature, ethyl acetate (40 mL) and water (40 mL) were added to the reaction solution. The mixed solution was filtered through diatomaceous earth and the resulting solution was separated. The aqueous phase was extracted three times with ethyl acetate (20 mL), and the combined organic phase was concentrated to give (4-(cyclopropylsulfonyl)phenyl)boronic acid 23a (800 mg) which was a crude product and used directly in the next step without a further purification.

### Step Two

### tert-butyl (tert-butoxycarbonyl)(3-(3-(4-cyanophenyl)isoxazol-5-yl)-5-(4-(cyclopropylsulfonyl)phenyl)pyrazin-2-yl) carbamate 23b

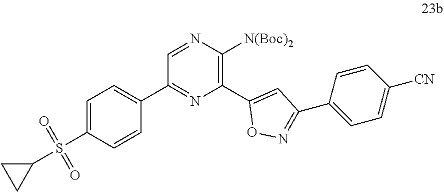

Under nitrogen protection, to a mixed solution of tert-butyl (5-bromo-3-(3-(4-cyanophenyl)isoxazol-5-yl)pyrazin-2-yl)(tert-butoxycarbonyl) carbamate 1b (0.6 g, 1.11 mmol) in 1,4-dioxane (15 mL) and water (1.5 mL) were added sequentially (4-(cyclopropylsulfonyl)phenyl)boronic acid 23a (0.31 g, 1.33 mmol), potassium carbonate (0.44 g, 3.33 mmol) and [1,1′-bis(diphenylphosphino)ferrocene]palladium(II) chloride (0.16 g, 0.22 mmol) at room temperature. After three times of nitrogen replacements, the reaction solution was stirred at 95° C. for three hours. After cooling to room temperature, ethyl acetate (40 mL) and water (40 mL) were added to the reaction solution. The mixed solution was filtered through diatomaceous earth and the resulting solution was separated. The aqueous phase was extracted three times with ethyl acetate (20 mL), and the combined organic phase was concentrated and isolated by column chromatography to give tert-butyl (tert-butoxycarbonyl)(3-(3-(4-cyanophenyl)isoxazol-5-yl)-5-(4-(cyclopropylsulfonyl)phenyl)pyrazin-2-yl) carbamate 23b (0.13 g).

### Step Three

### tert-butyl (3-(3-(4-aminophenyl)isoxazol-5-yl)-5-(4-(cyclopropylsulfonyl)phenyl)pyrazin-2-yl)(tert-butoxycarbonyl)carbamate 23c

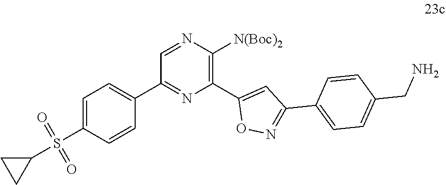

To a solution of tert-butyl (tert-butoxycarbonyl)(3-(3-(4-cyanophenyl)isoxazol-5-yl)-5-(4-(cyclopropylsulfonyl) phenyl)pyrazin-2-yl) carbamate 23b (125 mg, 0.20 mmol) in methanol (5 mL) was added sequentially nickel chloride hexahydrate (142 mg, 0.60 mmol) and sodium borohydride (23 mg, 0.60 mmol) at room temperature. The reaction solution was stirred for 1 hour at room temperature and then quenched with ammonium chloride (2 mL). Ethyl acetate (40 mL) and saturated aqueous sodium bicarbonate (40 mL) were then added. The mixed solution was filtered through diatomaceous earth and the filtrate was poured into a separatory funnel. The aqueous phase was extracted three times with ethyl acetate (40 mL) and the combined organic phase was concentrated and isolated by column chromatography to give the tert-butyl (3-(3-(4-aminophenyl)isoxazol-5-yl)-5-(4-(cyclopropylsulfonyl)phenyl)pyrazin-2-yl)(tert-butoxycarbonyl)carbamate 23c (21 mg).

### Step Four

### tert-butyl (tert-butoxycarbonyl) (5-(4-(cyclopropylsulfonyl)phenyl)-3-(3-(4-(guanidinylmethyl)phenyl)isoxazol-5-yl)pyrazin-2-yl)carbamate 23d

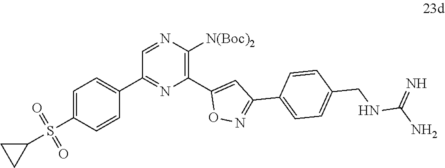

To a solution of tert-butyl (3-(3-(4-aminophenyl)isoxazol-5-yl)-5-(4-(cyclopropylsulfonyl)phenyl)pyrazin-2-yl)(tert-butoxycarbonyl)carbamate 23c (21 mg, 0.034 mmol) in acetonitrile (3 mL) were sequentially added 1H-pyrazole-1-carboxamidinium hydrochloride (6 mg, 0.038 mmol) and N,N-diisopropylethylamine (8 μL). The reaction was stirred at room temperature for 16 hours. Ethyl acetate (20 mL) and water (20 mL) were added to the reaction solution, and the resulting solution was separated. The aqueous phase was extracted three times with ethyl acetate (20 mL), and the combined organic phase was concentrated and separated by column chromatography to give tert-butyl (tert-butoxycarbonyl) (5-(4-(cyclopropylsulfonyl)phenyl)-3-(3-(4-(guanidinylmethyl)phenyl)isoxazol-5-yl)pyrazin-2-yl)carbamate 23d (6 mg).

### Step Five

### 1-(4-(5-(3-amino-6-(4-(cyclopropylsulfonyl)phenyl)pyrazin-2-yl)isoxazol-3-yl)benzyl)guanidine dihydrochloride 23

To a solution of tert-butyl (tert-butoxycarbonyl) (5-(4-(cyclopropylsulfonyl)phenyl)-3-(3-(4-(guanidinylmethyl)phenyl) isoxazol-5-yl)pyrazin-2-yl) carbamate 23d (6 mg, 0.01 mmol) in ethyl acetate (2 mL) was added a mixture (4 M, 2 mL) of hydrochloric acid and ethyl acetate. The reaction solution was stirred at room temperature for 1 hour and then concentrated to give a yellow solid 1-(4-(5-(3-amino-6-(4-(cyclopropylsulfonyl)phenyl)pyrazin-2-yl)isoxazol-3-yl)benzyl)guanidine dihydrochloride 23 (3 mg).

¹H NMR (400 MHz, DMSO-d₆): δ 8.96 (s, 1H), 8.39-8.37 (m, 2H), 8.22-8.20 (m, 1H), 8.09-8.07 (m, 2H), 8.02-7.99 (m, 2H), 7.82 (s, 1H), 7.54-7.52 (m, 2H), 7.35 (s, 2H), 7.22 (s, 3H), 7.10 (s, 2H), 4.52-4.51 (m, 2H), 2.97-2.91 (m, 1H), 1.40-1.37 (m, 2H), 1.32-1.29 (m, 2H)

MS measured value (ESI⁺) [(M+H)⁺]: 490

### Example 24

### 1-(4-(5-(3-amino-6-(4-(isopropylsulfonyl)phenyl)pyrazin-2-yl)isoxazol-3-yl)-2-fluorobenzyl)guanidine dihydrochloride 24

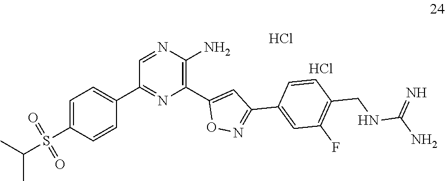 [SMILES: Cl ; CC(C)S(=O)(=O)c1ccc(-c2cnc(N)c(-c3cc(-c4ccc(CNC(=N)N)c(F)c4)no3)n2)cc1 ; Cl]

### Step One

### (E)-2-fluoro-4-((hydroxyimino)methyl)benzonitrile 24a

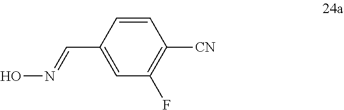 [SMILES: N#Cc1ccc(/C=N/O)cc1F]

To a solution of 2-fluoro-4-formylbenzonitrile (500 mg, 3.36 mmol) in tetrahydrofuran (30 mL) was added hydroxylamine hydrochloride (466 mg, 6.71 mmol) at room temperature. The reaction mixture was stirred at room temperature for 16 hours. Water (40 mL) was added to the reaction system and the organic phase was separated out. The aqueous phase was extracted three times with ethyl acetate (30 mL). The combined organic phase was dried with anhydrous sodium sulfate and filtered. The filtrate was concentrated under reduced pressure to give a yellow solid (E)-2-fluoro-4-((hydroxyimino)methyl)benzonitrile 24a (430 mg), which was a crude product and was used in the next step without a further purification.

### Step Two

### tert-butyl (5-bromo-3-(3-(4-cyano-3-fluorophenyl)isoxazol-5-yl)pyrazin-2-yl)(tert-butoxycarbonyl)carbamate 24b

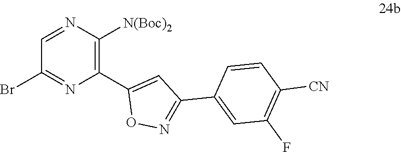

To a mixed solution of tert-butyl (5-bromo-3-ethynyl-2-pyrazinyl)(tert-butoxycarbonyl)carbamate (0.4 g, 4.88 mmol) in methanol (12 mL) and water (2.4 mL) were sequentially added (E)-2-fluoro-4-((hydroxyimino)methyl)benzonitrile 24a (0.65 g, 3.25 mmol) and bis(trifluoroacetyl)iodobenzene (1.05 g, 4.88 mmol). The reaction solution was stirred overnight at room temperature, and then was added with ethyl acetate (40 mL) and water (40 mL). The organic phase was washed three times with water (20 mL), separated out and dried with anhydrous sodium sulfate, filtered, concentrated and isolated by column chromatography to give tert-butyl (5-bromo-3-(3-(4-cyano-3-fluorophenyl)isoxazol-5-yl)pyrazin-2-yl)(tert-butoxycarbonyl)carbamate 24b (0.24 g).

### Step Three

### tert-butyl (tert-butoxycarbonyl) (3-(3-(4-cyano-3-fluorophenyl)isoxazol-5-yl)-5-(4-(isopropylsulfonyl)phenyl)pyrazin-2-yl) carbamate 24c

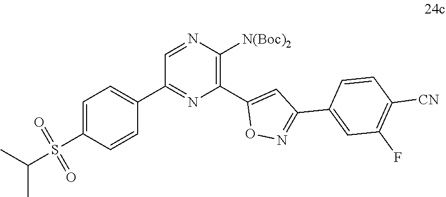

Under nitrogen protection, to a mixed solution of tert-butyl (5-bromo-3-(3-(4-cyano-3-fluorophenyl)isoxazol-5-yl)pyrazin-2-yl)(tert-butoxycarbonyl)carbamate 24b (0.2 g, 0.36 mmol) in 1,4-dioxane (15 mL) and water (1.5 mL) were added sequentially 4-isopropylsulfonylphenylboronic acid (0.80 g, 0.36 mmol), potassium carbonate (0.15 g, 1.08 mmol) and [1,1′-bis(diphenylphosphino)ferrocene]palladium(II) dichloride (0.052 g, 0.072 mmol) at room temperature. After three times of nitrogen replacements, the reaction solution was stirred at 95° C. for three hours. After cooling to room temperature, ethyl acetate (40 mL) and water (40 mL) were added to the reaction solution. The mixed solution was filtered through diatomaceous earth and the resulting solution was separated. The aqueous phase was extracted three times with ethyl acetate (20 mL), and the combined organic phase was concentrated and isolated by column chromatography to give tert-butyl (tert-butoxycarbonyl) (3-(3-(4-cyano-3-fluorophenyl)isoxazol-5-yl)-5-(4-(isopropylsulfonyl)phenyl)pyrazin-2-yl) carbamate 24c (0.11 g).

### Step Four

### tert-butyl (3-(3-(4-(aminomethyl)-3-fluorophenyl)isoxazol-5-yl)-5-(4-(isopropylsulfonyl)phenyl)pyrazin-2-yl)(tert-butoxycarbonyl)carbamate 24d

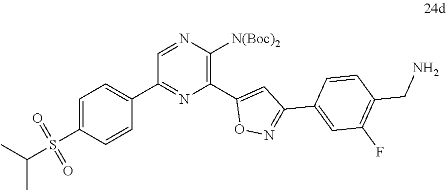

To a solution of tert-butyl (tert-butoxycarbonyl) (3-(3-(4-cyano-3-fluorophenyl)isoxazol-5-yl)-5-(4-(isopropylsulfonyl) phenyl) pyrazin-2-yl) carbamate 24c (107 mg, 0.16 mmol) in methanol (5 mL) were sequentially added nickel chloride hexahydrate (115 mg, 0.48 mmol) and sodium borohydride (19 mg, 0.48 mmol) at room temperature. The reaction solution was stirred for 1 hour at room temperature and quenched with ammonium chloride (2 mL). Ethyl acetate (40 mL) and saturated aqueous sodium bicarbonate (40 mL) were then added. The mixed solution was filtered through diatomaceous earth and the filtrate was poured into a separatory funnel. The aqueous phase was extracted three times with ethyl acetate (40 mL) and the combined organic phase was concentrated and isolated by column chromatography to give tert-butyl (3-(3-(4-(aminomethyl)-3-fluorophenyl)isoxazol-5-yl)-5-(4-(isopropylsulfonyl)phenyl)pyrazin-2-yl)(tert-butoxycarbonyl) carbamate 24d (26 mg).

### Step Five

### tert-butyl (tert-butoxycarbonyl) (3-(3-(3-(3-fluoro-4-(guanidinomethyl)phenyl)isoxazol-5-yl)-5-(4-(isopropylsulfonyl)phenyl)pyrazin-2-yl)carbamate 24e

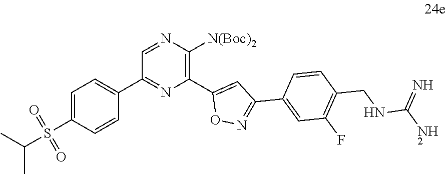

To a solution of tert-butyl (3-(3-(4-(aminomethyl)-3-fluorophenyl)isoxazol-5-yl)-5-(4-(isopropylsulfonyl)phenyl) pyrazin-2-yl)(tert-butoxycarbonyl) carbamate 24d (26 mg, 0.046 mmol) in acetonitrile (3 mL) were sequentially added 1H-pyrazole-1-carboximidamide hydrochloride (7 mg, 0.046 mmol) and N,N-diisopropylethylamine (9 μL). The reaction solution was stirred at room temperature (˜20° C.) for 16 hours, and then added with ethyl acetate (20 mL) and water (20 mL). The resulting solution was separated, and the aqueous phase was extracted three times with ethyl acetate (20 mL). The combined organic phase was concentrated and isolated by column chromatography to give tert-butyl (tert-butoxycarbonyl) (3-(3-(3-(3-fluoro-4-(guanidinomethyl)phenyl)isoxazol-5-yl)-5-(4-(isopropylsulfonyl)phenyl)pyrazin-2-yl)carbamate 24e (12 mg).

### Step Six

### 1-(4-(5-(3-amino-6-(4-(isopropylsulfonyl)phenyl)pyrazin-2-yl)isoxazol-3-yl)-2-fluorobenzyl)guanidine dihydrochloride 24

To a solution of tert-butyl (tert-butoxycarbonyl) (3-(3-(3-(3-fluoro-4-(guanidinomethyl)phenyl)isoxazol-5-yl)-5-(4-(isopropylsulfonyl)phenyl)pyrazin-2-yl)carbamate 24e (12 mg, 0.02 mmol) in ethyl acetate (2 mL) was added a solution of ethyl acetate in HCl (4 M, 2 mL). After the reaction solution was stirred for 1 hour at room temperature, and then was concentrated to give a yellow solid 1-(4-(5-(3-amino-6-(4-(isopropylsulfonyl)phenyl)pyrazin-2-yl)isoxazol-3-yl)-2-fluorobenzyl)guanidine dihydrochloride 24 (8 mg).

¹H NMR (400 MHz, DMSO-d₆): δ 8.99 (s, 1H), 8.42-8.40 (m, 2H), 8.06 (s, 1H), 7.98-7.94 (m, 4H), 7.93-7.90 (m, 1H), 7.60-7.56 (m, 1H), 7.25 (s, 2H), 4.57-4.56 (m, 2H), 3.54-3.48 (m, 1H), 1.23-1.21 (m, 6H)

MS measured value (ESI⁺) [(M+H)⁺]: 510

### Example 25

### 4-(5-amino-6-(3-(4-(guanidinomethyl)phenyl)isoxazol-5-yl)pyrazin-2-yl)-N,N-dimethylbenzamide dihydrochloride 25

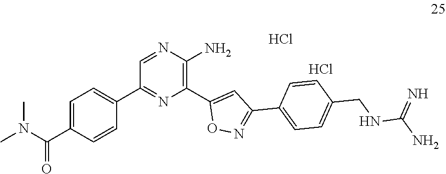 [SMILES: Cl ; CN(C)C(=O)c1ccc(-c2cnc(N)c(-c3cc(-c4ccc(CNC(=N)N)cc4)no3)n2)cc1 ; Cl]

### Step One

### tert-butyl (tert-butoxycarbonyl) (3-(3-(4-cyanophenyl)isoxazol-5-yl)-5-(4-(dimethylcarbamoyl)phenyl)pyrazin-2-yl) carbamate 25a

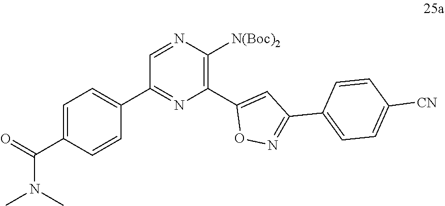

Under nitrogen protection, to a mixed solution of tert-butyl (5-bromo-3-(3-(4-cyanophenyl)isoxazol-5-yl)pyrazin-2-yl)(tert-butoxycarbonyl) carbamate 1b (0.4 g, 0.74 mmol) in 1,4-dioxane (10 mL) and water (2 mL) were added sequentially N,N-dimethyl-4-(4,4,5,5-tetramethyl-1,3,2-dioxobenzaldehyde-2-yl)benzamide (0.24 g, 0.89 mmol), potassium carbonate (0.30 g, 2.22 mmol) and [1,1′-bis(diphenylphosphino)ferrocene]palladium(II) dichloride (0.10 g, 0.15 mmol). After three times of nitrogen replacements, the reaction solution was stirred at 95° C. for three hours. After cooling to room temperature, ethyl acetate (40 mL) and water (40 mL) were added to the reaction solution. The mixed solution was filtered through diatomaceous earth, and the resulting solution was separated. The aqueous phase was extracted three times with ethyl acetate (20 mL), and the combined organic phase was concentrated and isolated by column chromatography to give tert-butyl (tert-butoxycarbonyl) (3-(3-(4-cyanophenyl)isoxazol-5-yl)-5-(4-(dimethylcarbamoyl)phenyl)pyrazin-2-yl) carbamate 25a (0.16 g).

### Step Two

### tert-butyl (3-(3-(4-(aminomethyl)phenyl)isoxazol-5-yl)-5-(4-(dimethylcarbamoyl)phenyl)pyrazin-2-yl)(tert-butoxycarbonyl) carbamate 25b

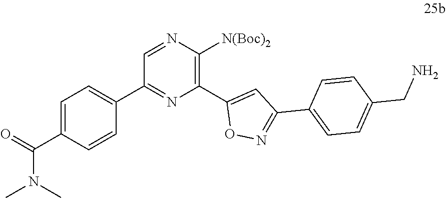

To a solution of tert-butyl (tert-butoxycarbonyl) (3-(3-(4-cyanophenyl)isoxazol-5-yl)-5-(4-(dimethylcarbamoyl)phenyl) pyrazin-2-yl) carbamate 25a (160 mg, 0.26 mmol) in methanol (5 mL) was added nickel chloride hexahydrate (187 mg, 0.79 mmol) and sodium borohydride (30 mg, 0.79 mmol) sequentially at room temperature. The reaction solution was stirred for 1 hour at room temperature and then quenched with ammonium chloride (2 mL). Ethyl acetate (40 mL) and saturated aqueous sodium bicarbonate (40 mL) were then added. The mixed solution was filtered through diatomaceous earth and the filtrate was poured into a separatory funnel. The aqueous phase was extracted three times with ethyl acetate (40 mL) and the combined organic phase was concentrated and isolated by column chromatography to give tert-butyl (3-(3-(4-(aminomethyl)phenyl)isoxazol-5-yl)-5-(4-(dimethylcarbamoyl)phenyl)pyrazin-2-yl)(tert-butoxycarbonyl) carbamate 25b (21 mg).

### Step Three

### tert-butyl (tert-butoxycarbonyl)(5-(4-(dimethylcarbamoyl)phenyl)-3-(3-(4-(guanidinomethyl)phenyl)isoxazol-5-yl)pyrazin-2-yl)carbamate 25c

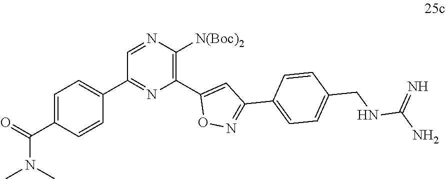

To a solution of tert-butyl (3-(3-(4-(aminomethyl)phenyl)isoxazol-5-yl)-5-(4-(dimethylcarbamoyl)phenyl)pyrazin-2-yl)(tert-butoxycarbonyl) carbamate 25b (21 mg, 0.034 mmol) in acetonitrile (3 mL) were added 1H-pyrazole-1-carboxamidinium hydrochloride (6 mg, 0.038 mmol) and N,N-diisopropylethylamine (8 μL) in sequence. The reaction solution was stirred at room temperature (˜20° C.) for 16 hours, and then added with ethyl acetate (20 mL) and water (20 mL). The resulting solution was separated, and the aqueous phase was extracted three times with ethyl acetate (20 mL). The combined organic phase was concentrated and isolated by column chromatography to give tert-butyl (tert-butoxycarbonyl) (5-(4-(dimethylcarbamoyl)phenyl)-3-(3-(4-(guanidinomethyl)phenyl)isoxazol-5-yl)pyrazin-2-yl)carbamate 25c (15 mg).

### Step Four

### 4-(5-amino-6-(3-(4-(guanidinomethyl)phenyl)isoxazol-5-yl)pyrazin-2-yl)-N,N-dimethylbenzamide dihydrochloride 25

To a solution of tert-butyl (tert-butoxycarbonyl) (5-(4-(dimethylcarbamoyl)phenyl)-3-(3-(4-(guanidinomethyl)phenyl) isoxazol-5-yl)pyrazin-2-yl)carbamate 25c (15 mg, 0.03 mmol) in ethyl acetate (2 mL) was added a solution of ethyl acetate in HCl (4 M, 2 mL). The reaction solution was stirred at room temperature for 1 h. The reaction solution was concentrated to give a yellow solid 4-(5-amino-6-(3-(4-(guanidinomethyl)phenyl)isoxazol-5-yl)pyrazin-2-yl)-N,N-dimethylbenzamide dihydrochloride 25 (5 mg).

¹H NMR (400 MHz, DMSO-d₆): δ 8.87 (s, 1H), 8.25-8.23 (m, 1H), 8.21-8.15 (m, 2H), 8.06-8.04 (m, 2H), 7.77 (s, 1H), 7.53-7.49 (m, 4H), 7.36 (s, 3H), 7.23 (s, 2H), 7.11 (s, 2H), 4.50-4.48 (m, 2H), 3.00-2.97 (m, 6H)

MS measured value (ESI⁺) [(M+H)⁺]: 457

### Example 26

### 4-(5-(3-amino-6-(4-(isopropylsulfonyl)phenyl)pyrazin-2-yl)isoxazol-3-yl)-N-carbamoylbenzamide dihydrochloride 26

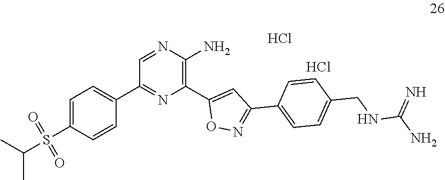 [SMILES: Cl ; CC(C)S(=O)(=O)c1ccc(-c2cnc(N)c(-c3cc(-c4ccc(CNC(=N)N)cc4)no3)n2)cc1 ; Cl]

### Step One

### Methyl (E)-4-((hydroxyimino)methyl)benzoate 26a

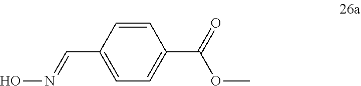

To a solution of methyl 4-formylbenzoate (1 g, 6.10 mmol) in tetrahydrofuran (30 mL) was added hydroxylamine hydrochloride (0.51 g, 7.31 mmol) at room temperature. The reaction mixture was stirred at room temperature for 16 hours. Water (40 mL) was added to the reaction system and the organic phase was separated out. The aqueous phase was extracted three times with ethyl acetate (30 mL), and the combined organic phase was dried with anhydrous sodium sulfate and filtered. The filtrate was concentrated under reduced pressure to give a yellow solid, methyl (E)-4-((hydroxyimino) methyl)benzoate 26a (1.2 g) which was a crude product and used directly in the next step without a further purification.

### Step Two

### Methyl 4-(5-(3-(bis(tert-butoxycarbonyl)amino)-6-bromo-pyrazin-2-yl)isoxazol-3-yl)benzoate 26b

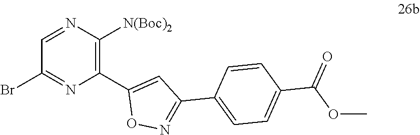

To a mixed solution of tert-butyl (5-bromo-3-ethynyl-2-pyrazinyl)(tert-butoxycarbonyl)carbamate (0.89 g, 2.23 mmol) in methanol (12 mL) and water (2.4 mL) were sequentially added methyl (E)-4-((hydroxyimino) methyl)benzoate 26a (0.60 g, 3.35 mmol) and bis(trifluoroacetimidobenzene) (1.44 g, 3.35 mmol). The reaction solution was stirred at room temperature overnight, and then added with ethyl acetate (40 mL) and water (40 mL). The organic phase was washed three times with water (20 mL). The organic phase was separated out and dried by anhydrous sodium sulfate, filtered, concentrated and isolated by column chromatography to give methyl 4-(5-(3-(bis(tert-butoxycarbonyl)amino)-6-bromo-pyrazin-2-yl)isoxazol-3-yl)benzoate 26b (0.96 g).

### Step Three

### Methyl 4-(5-(3-((tert-butoxycarbonyl)amino)-6-(4-(isopropylsulfonyl)phenyl)pyrazin-2-yl)isoxazol-3-yl)benzoate 26c

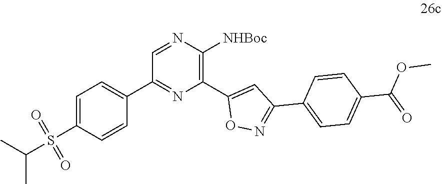 [SMILES: COC(=O)c1ccc(-c2cc(-c3nc(-c4ccc(S(=O)(=O)C(C)C)cc4)cnc3NC(=O)OC(C)(C)C)on2)cc1]

Under nitrogen protection, to a mixed solution of methyl 4-(5-(3-(bis(tert-butoxycarbonyl)amino)-6-bromo-pyrazin-2-yl)isoxazol-3-yl)benzoate 26b (0.4 g, 0.70 mmol) in 1,4-dioxane (15 mL) and water (1.5 mL) was sequentially added 4-isopropylsulfonylphenylboronic acid (0.16 g, 0.70 mmol), potassium carbonate (0.29 g, 2.10 mmol) and [1,1′-bis(diphenylphosphino)ferrocene]palladium(II) chloride (0.10 g, 0.14 mmol) at room temperature (˜20° C.). After three times of nitrogen replacements, the reaction solution was stirred at 95° C. for three hours. After cooling to room temperature, ethyl acetate (40 mL) and water (40 mL) were added to the reaction solution. The mixed solution was filtered through diatomaceous earth and the resulting solution was separated. The aqueous phase was extracted three times with ethyl acetate (20 mL), and the combined organic phase was concentrated and isolated by column chromatography to give methyl 4-(5-(3-((tert-butoxycarbonyl)amino)-6-(4-(isopropylsulfonyl)phenyl)pyrazin-2-yl)isoxazol-3-yl)benzoate 26c (0.33g).

### Step Four

### 4-(5-(3-((tert-butoxycarbonyl)amino)-6-(4-(isopropylsulfonyl)phenyl)pyrazin-2-yl)isoxazol-3-yl)benzoic acid 26d

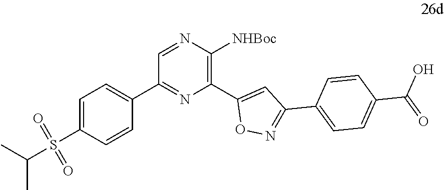 [SMILES: CC(C)S(=O)(=O)c1ccc(-c2cnc(NC(=O)OC(C)(C)C)c(-c3cc(-c4ccc(C(=O)O)cc4)no3)n2)cc1]

To a solution of methyl 4-(5-(3-((tert-butoxycarbonyl)amino)-6-(4-(isopropylsulfonyl)phenyl)pyrazin-2-yl)isoxazol-3-yl)benzoate 26c (150 mg, 0.26 mmol) in methanol (5 mL) was added lithium hydroxide (33 mg, 0.78 mmol) at room temperature. The reaction solution was stirred for 1 hour at room temperature, and then was adjusted to acidity with aqueous hydrochloric acid (1 M) and extracted three times through the addition of ethyl acetate (20 mL). The combined organic phase was concentrated to give 4-(5-(3-((tert-butoxycarbonyl)amino)-6-(4-(isopropylsulfonyl)phenyl)pyrazin-2-yl)isoxazol-3-yl)benzoic acid 26d (150 mg), which was a crude product and used directly in the next step without a further purification.

### Step Five

### tert-butyl (3-(3-(4-(amidinocarbamoyl)phenyl)isoxazol-5-yl)-5-(4-(isopropylsulfonyl)phenyl)pyrazin-2-yl) carbamate 26e

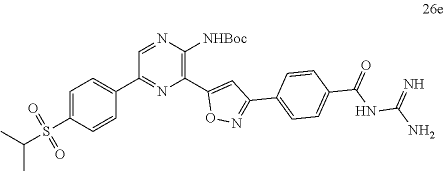 [SMILES: CC(C)S(=O)(=O)c1ccc(-c2cnc(NC(=O)OC(C)(C)C)c(-c3cc(-c4ccc(C(=O)NC(=N)N)cc4)no3)n2)cc1]

To a solution of 4-(5-(3-((tert-butoxycarbonyl)amino)-6-(4-(isopropylsulfonyl)phenyl)pyrazin-2-yl)isoxazol-3-yl) benzoic acid 26d (75 mg, 0.013 mmol) in N-methylpyrrolidinone (3 mL) was added 2-chloro-1-methylpyridinium iodide (45 mg, 0.17 mmol). The reaction solution was stirred at 50° C. for 1 hour, and then 1-(tert-butoxycarbonyl)guanidine (28 mg, 0.17 mmol) and N,N-diisopropylethylamine (66 uL) were added sequentially. The reaction solution was stirred at room temperature for 16 hours. Ethyl acetate (20 mL) and water (20 mL) were added to the reaction solution, and the resulting solution was separated. The aqueous phase was extracted three times with ethyl acetate (20 mL), and the combined organic phase was concentrated and isolated by column chromatography to give tert-butyl (3-(3-(4-(amidinocarbamoyl)phenyl) isoxazol-5-yl)-5-(4-(isopropylsulfonyl)phenyl)pyrazin-2-yl) carbamate 26e (60 mg).

### Step Six

### 4-(5-(3-amino-6-(4-(isopropylsulfonyl)phenyl)pyrazin-2-yl)isoxazol-3-yl)-N-carbamoylbenzamide dihydrochloride 26

To a solution of tert-butyl (3-(3-(4-(amidinocarbamoyl)phenyl) isoxazol-5-yl)-5-(4-(isopropylsulfonyl)phenyl)pyrazin-2-yl) carbamate 26e (60 mg, 0.10 mmol) in ethyl acetate (2 mL) was added a mixed solution (4 M, 2 mL) of hydrochloric acid and ethyl acetate. After the reaction solution was stirred for 1 hour at room temperature, the reaction solution was concentrated to give a yellow solid 4-(5-(3-amino-6-(4-(isopropylsulfonyl)phenyl)pyrazin-2-yl)isoxazol-3-yl)-N-carbamoylbenzamide dihydrochloride 26 (45 mg).

¹H NMR (400 MHz, DMSO-d₆): δ 12.12 (s, 1H), 8.96 (s, 1H), 8.73 (s, 2H), 8.56 (s, 2H), 8.39-8.37 (m, 2H), 8.34-8.32 (m, 2H), 8.26-8.24 (m, 2H), 7.95-7.93 (m, 3H), 7.23 (s, 1H), 3.50-3.43 (m, 1H), 1.23-1.21 (m, 6H)

MS measured value (ESI⁺) [(M+H)⁺]: 506

### Example 27

### 1-(4-(5-(3-amino)-6-(1,1-dioxo-2,3-dihydrobenzo[b]thiophen-5-yl)pyrazin-2-yl)isoxazol-3-yl)benzyl)guanidine dihydrochloride 27

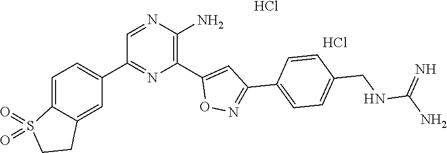 [SMILES: Cl ; N=C(N)NCc1ccc(-c2cc(-c3nc(-c4ccc5c(c4)CCS5(=O)=O)cnc3N)on2)cc1 ; Cl]

### Step One

### 5-bromobenzo[b]thiophene 1,1-dioxide 27a

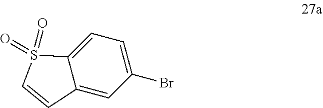 [SMILES: O=S1(=O)C=Cc2cc(Br)ccc21]

To a solution of 5-bromobenzothiophene (1 g, 4.72 mmol) in dichloromethane (20 mL) was added m-chloroperoxybenzoic acid (2.4 g, 14.16 mmol) at room temperature. The reaction mixture was stirred at room temperature for 6 hours. Then, the reaction system was quenched by adding excess saturated aqueous sodium sulfite (40 mL). The organic phase was then separated out, and the aqueous phase was extracted three times with dichloromethane (30 mL). The combined organic phase was dried with anhydrous sodium sulfate and filtered. The filtrate was concentrated under reduced pressure and give a white solid 5-bromobenzo[b]thiophene 1,1-dioxide 27a (0.97 g) through the column chromatography.

### Step Two

### 5-bromo-2,3-dihydrobenzo[b]thiophene 1,1-dioxide 27b

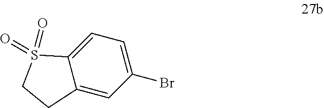 [SMILES: O=S1(=O)CCc2cc(Br)ccc21]

To a solution of 5-bromobenzo[b]thiophene 1,1-dioxide 27a (0.5 g, 2.05 mmol) in ethanol (8 mL) was added sodium borohydride (0.12 g, 3.07 mmol) at room temperature. The reaction mixture was stirred at room temperature (˜20° C.) for 6 hours. The reaction system was quenched by adding saturated aqueous ammonium chloride solution (4 mL). Then, ethyl acetate (40 mL) and water (40 mL) were added. The organic phase was separated out, and the aqueous phase was extracted three times with ethyl acetate (30 mL). The combined organic phase was dried with anhydrous sodium sulfate and was filtered. The filtrate was concentrated under reduced pressure to give a white solid 5-bromo-2,3-dihydrobenzo[b]thiophene 1,1-dioxide 27b (0.50 g) which was a crude product and used directly in the next step without a further purification.

### Step Three

### (1,1-dioxo-2,3-dihydrobenzo[b]thiophen-5-yl)boronic acid 27c

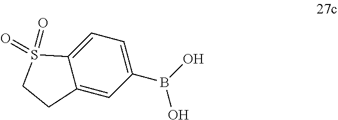 [SMILES: O=S1(=O)CCc2cc(B(O)O)ccc21]

To a mixed solution of 5-bromo-2,3-dihydrobenzo[b]thiophene 1,1-dioxide 27b (500 mg, 2.03 mmol) in N,N-dimethylformamide (12 mL) were sequentially added bis(pinacolato)borate (775 mg, 3.05 mmol), potassium acetate (497 mg, 0.51 mmol) and [1,1′-bis(diphenylphosphino)ferrocene]palladium (II) dichloride (150 mg, 0.20 mmol) at room temperature. After three times of nitrogen replacements, the reaction solution was stirred at 80° C. for three hours. After cooling to room temperature, ethyl acetate (40 mL) and water (40 mL) were added to the reaction solution. The mixed solution was filtered through diatomaceous earth, and the resulting solution was separated. The aqueous phase was extracted three times with ethyl acetate (20 mL), and the combined organic phase was concentrated to give (1,1-dioxo-2,3-dihydrobenzo[b]thiophen-5-yl)boronic acid 27c (800 mg) which was a crude product and used directly in the next step without a further purification.

### Step Four

### tert-butyl (tert-butoxycarbonyl)(3-(3-(4-cyanophenyl)isoxazol-5-yl)-5-(1,1-dioxo-2,3-dihydrobenzothiophen-5-yl)pyrazin-2-yl)carbamate 27d

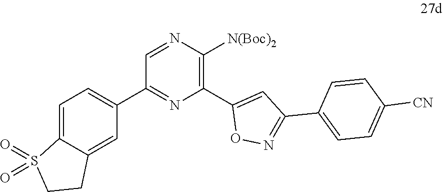

Under nitrogen protection, to a mixed solution of tert-butyl (5-bromo-3-(3-(4-cyanophenyl)isoxazol-5-yl)pyrazin-2-yl)(tert-butoxycarbonyl)carbamate 1b (0.6 g, 1.11 mmol) in 1,4-dioxane (15 mL) and water (1.5 mL) was sequentially added (1,1-dioxo-2,3-dihydrobenzo[b]thiophen-5-yl)boronic acid 27c (0.65 g, 2.22 mmol), potassium carbonate (0.44 g, 3.33 mmol) and [1,1′-bis(diphenylphosphino)ferrocene]palladium(II) chloride (0.16 g, 0.22 mmol) at room temperature (˜20° C.). After three times of nitrogen replacements, the reaction solution was stirred at 95° C. for three hours. After cooling to room temperature, ethyl acetate (40 mL) and water (40 mL) were added to the reaction solution. The mixed solution was filtered through diatomaceous earth and the resulting solution was separated. The aqueous phase was extracted three times with ethyl acetate (20 mL), and the combined organic phase was concentrated and isolated by column chromatography to give tert-butyl (tert-butoxycarbonyl)(3-(3-(4-cyanophenyl)isoxazol-5-yl)-5-(1,1-dioxo-2,3-dihydrobenzothiophen-5-yl) pyrazin-2-yl)carbamate 27d (0.25 g).

### Step Five

### tert-butyl (tert-butoxycarbonyl) (3-(3-(4-(aminomethyl)phenyl)isoxazol-5-yl)-5-(1,1-dioxo-2,3-dihydrobenzothiophen-5-yl)pyrazin-2-yl) carbamate 27e

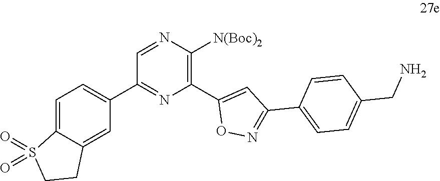

To a solution of tert-butyl (tert-butoxycarbonyl)(3-(3-(4-cyanophenyl)isoxazol-5-yl)-5-(1,1-dioxo-2,3-dihydrobenzothiophen-5-yl) pyrazin-2-yl)carbamate 27d (250 mg, 0.40 mmol) in methanol (5 mL) were added nickel chloride hexahydrate (284 mg, 1.20 mmol) and sodium borohydride (23 mg, 1.2 mmol) sequentially at room temperature. The reaction solution was stirred for 1 hour at room temperature and then quenched with saturated aqueous ammonium chloride solution (2 mL). Ethyl acetate (40 mL) and saturated aqueous sodium bicarbonate solution (40 mL) were then added. The mixed solution was filtered through diatomaceous earth and the filtrate was poured into a separatory funnel. The aqueous phase was extracted three times with ethyl acetate (40 mL) and the combined organic phase was concentrated and isolated by column chromatography to give tert-butyl (tert-butoxycarbonyl) (3-(3-(4-(aminomethyl)phenyl)isoxazol-5-yl)-5-(1,1-dioxo-2,3-dihydrobenzothiophen-5-yl)pyrazin-2-yl) carbamate 27e (62 mg).

### Step Six

### tert-butyl (tert-butoxycarbonyl)(5-(1,1-dioxo-2,3-dihydrobenzo[b]thiophen-5-yl)-3-(3-(4-(guanidinomethyl) phenyl)isoxazol-5-yl)pyrazin-2-yl) carbamate 27f

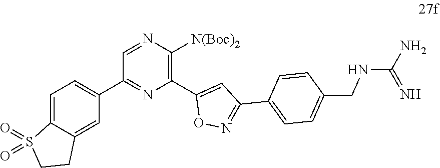

To a solution of tert-butyl (tert-butoxycarbonyl) (3-(3-(4-(aminomethyl)phenyl)isoxazol-5-yl)-5-(1,1-dioxo-2,3-dihydrobenzothiophen-5-yl)pyrazin-2-yl) carbamate 27e (62 mg, 0.10 mmol) in acetonitrile (3 mL) were added 1H-pyrazole-1-carboxamidinium hydrochloride (17 mg. 0.11 mmol) and N,N-diisopropylethylamine (32 μL) sequentially. The reaction solution was stirred at room temperature for 16 hours. Ethyl acetate (20 mL) and water (20 mL) were added to the reaction solution, and the resulting solution was separated. The aqueous phase was extracted three times with ethyl acetate (20 mL), and the combined organic phase was concentrated and isolated by column chromatography to give tert-butyl (tert-butoxycarbonyl)(5-(1,1-dioxo-2,3-dihydrobenzo[b]thiophen-5-yl)-3-(3-(4-(guanidinomethyl) phenyl)isoxazol-5-yl)pyrazin-2-yl) carbamate 27f (25 mg).

### Step Seven

### 1-(4-(5-(3-amino)-6-(1,1-dioxo-2,3-dihydrobenzo[b]thiophen-5-yl)pyrazin-2-yl)isoxazol-3-yl)benzyl)guanidine dihydrochloride 27

To a solution of tert-butyl (tert-butoxycarbonyl)(5-(1,1-dioxo-2,3-dihydrobenzo[b]thiophen-5-yl)-3-(3-(4-(guanidinomethyl) phenyl)isoxazol-5-yl)pyrazin-2-yl) carbamate 27f (25 mg, 0.037 mmol) in ethyl acetate (2 mL) was added a solution of ethyl acetate in HCl (4 M, 2 mL). The reaction solution was stirred at room temperature for 1 h. The reaction solution was concentrated to give a yellow solid 1-(4-(5-(3-amino)-6-(1,1-dioxo-2,3-dihydrobenzo[b]thiophen-5-yl)pyrazin-2-yl)isoxazol-3-yl)benzyl)guanidine dihydrochloride 27 (14 mg).

¹H NMR (400 MHz, DMSO-d₆): δ 8.92 (s, 1H), 8.27-8.25 (m, 2H), 8.19-8.16 (m, 1H), 8.06-8.04 (m, 2H), 7.84-7.82 (m, 1H), 7.79 (s, 1H), 7.51-7.49 (m, 2H), 7.32 (m, 1H), 7.19-7.17 (m, 3H), 7.06 (s, 1H), 4.50-4.48 (m, 2H), 3.67-3.63 (m, 2H), 3.46-3.43 (m, 2H)

MS measured value (ESI⁺) [(M+H)⁺]: 476

### Example 28

### N-(4-(5-(3-amino-6-(4-(isopropylsulfonyl)phenyl)pyrazin-2-yl)isoxazol-3-yl)benzyl)acetamidine dihydrochloride 28

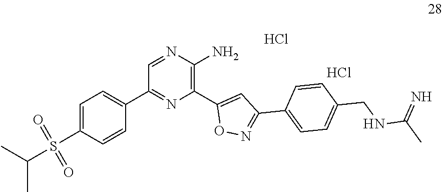 [SMILES: Cl ; CC(=N)NCc1ccc(-c2cc(-c3nc(-c4ccc(S(=O)(=O)C(C)C)cc4)cnc3N)on2)cc1 ; Cl]

### Step One

### (E)-4-(bromomethyl)benzaldehyde oxime 28a

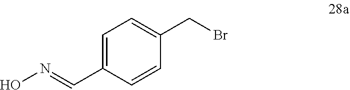 [SMILES: O/N=C/c1ccc(CBr)cc1]

The white solid (E)-4-(bromomethyl)benzaldehyde oxime 28a (500 mg) was prepared by 4-(bromomethyl)benzaldehyde (500 mg, 2.51 mmol) and hydroxylamine hydrochloride (346.7 mg, 5.02 mmol) using a method similar to step one of Example 1.

### Step Two

### tert-butyl (5-bromo-3-(3-(4-bromomethylphenyl)isoxazol-5-yl)pyrazin-2-yl)(tert-butoxycarbonyl)carbamate 28b

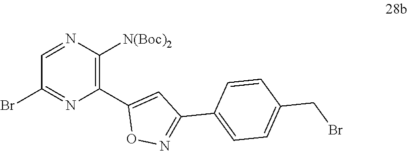

The tert-butyl (5-bromo-3-(3-(4-bromomethylphenyl)isoxazol-5-yl)pyrazin-2-yl)(tert-butoxycarbonyl) carbamate 28b was prepared by tert-butyl (5-bromo-3-ethynyl-pyrazin-2-yl)(tert-butoxycarbonyl)carbamate (0.5 g, 1.26 mmol) and (E)-4-(bromomethyl)benzaldehyde oxime 28a (0.40 g, 1.88 mmol) using a method similar to step two of Example 1.

### Step Three

### tert-butyl (3-(3-(4-acetamidomethylphenyl)isoxazol-5-yl)-5-bromopyrazin-2-yl)(tert-butoxycarbonyl)carbamate 28c

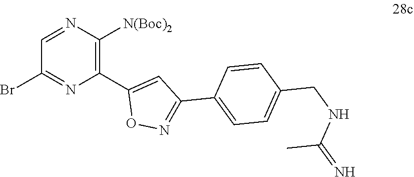

To a solution of tert-butyl (5-bromo-3-(3-(4-bromomethylphenyl)isoxazol-5-yl)pyrazin-2-yl)(tert-butoxycarbonyl) carbamate 28b (3 ml) at room temperature were added acetamidine hydrochloride (46.4 mg, 0.49 mmol), potassium carbonate (113.8 mg, 0.83 mmol) and potassium iodide (54.8 mg, 0.33 mmol). The reaction solution was stirred at room temperature for 3 h. Ethyl acetate (40 mL) and water (40 mL) were added to the reaction solution. The organic phase was washed three times with water (20 mL). The organic phase was separated out and dried by anhydrous sodium sulfate, and filtered, concentrated and isolated by column chromatography to give tert-butyl (3-(3-(4-acetamidomethylphenyl)isoxazol-5-yl)-5-bromopyrazin-2-yl)(tert-butoxycarbonyl)carbamate 28c (60 mg).

### Step Four

### tert-butyl (3-(3-(4-acetamidomethylphenyl)isoxazol-5-yl)-5-(4-(isopropylsulfonyl)phenyl)pyrazin-2-yl)(tert-butoxycarbonyl) carbamate 28d

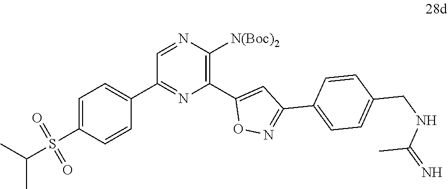

The tert-butyl (3-(3-(4-acetamidomethylphenyl)isoxazol-5-yl)-5-(4-(isopropylsulfonyl)phenyl)pyrazin-2-yl)(tert-butoxycarbonyl) carbamate 28d was prepared by tert-butyl (3-(3-(4-acetamidomethylphenyl)isoxazol-5-yl)-5-bromopyrazin-2-yl)(tert-butoxycarbonyl)carbamate 28c (60 mg, 0.10 mmol) and 4-(isopropylsulfonyl)phenylboronic acid (35.1 mg, 0.15 mmol) using a method similar to step three of Example 1.

### Step Five

### N-(4-(5-(3-amino-6-(4-(isopropylsulfonyl)phenyl)pyrazin-2-yl)isoxazol-3-yl)benzyl)acetamidine dihydrochloride 28

The yellow solid N-(4-(5-(3-amino-6-(4-(isopropylsulfonyl)phenyl)pyrazin-2-yl)isoxazol-3-yl)benzyl)acetamidine dihydrochloride 28 was prepared by tert-butyl (3-(3-(4-acetamidomethylphenyl)isoxazol-5-yl)-5-(4-(isopropylsulfonyl) phenyl)pyrazin-2-yl)(tert-butoxycarbonyl) carbamate 28d using a method similar to step three of Example 1.

¹H NMR (400 MHz, DMSO-d₆): δ 10.18 (br. s, 1H), 9.45 (s, 1H), 9.01 (s, 1H), 8.98 (s, 1H), 8.42-8.40 (m, 2H), 8.09-8.07 (m, 2H), 7.97-7.95 (m, 2H), 7.85 (s, 1H), 7.62-7.60 (m, 2H), 7.48 (brs, 3H), 7.35 (brs, 3H), 7.22 (brs, 2H), 4.63-4.62 (m, 2H), 3.49-3.42 (m, 1H), 2.29 (s, 3H), 1.23-1.21 (m, 2H)

MS measured value (ESI⁺) [(M+H)⁺]: 491

### Example 29

### N-(4-(5-(3-Amino-6-(4-(isopropylsulfonyl)phenyl)pyrazin-2-yl)isoxazol-3-yl)phenyl)acetimidate dihydrochloride 29

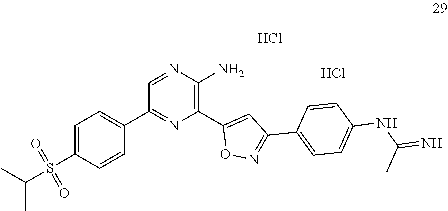 [SMILES: Cl ; CC(=N)Nc1ccc(-c2cc(-c3nc(-c4ccc(S(=O)(=O)C(C)C)cc4)cnc3N)on2)cc1 ; Cl]

### Step One

### (E)-4-iodobenzaldehyde oxime 29a

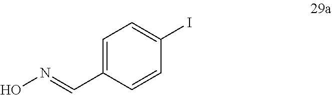

The white solid (E)-4-iodobenzaldehyde oxime 29a was prepare by 4-iodobenzaldehyde (500 mg, 2.16 mmol) and hydroxylamine hydrochloride (223.6 mg, 3.24 mmol) using a method similar to step one of

### Example 1

### Step Two

### tert-butyl (5-bromo-3-(3-(4-iodophenyl)isoxazol-5-yl)pyrazin-2-yl)(tert-butoxycarbonyl) carbamate 29b

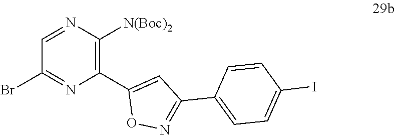

The tert-butyl (5-bromo-3-(3-(4-iodophenyl)isoxazol-5-yl)pyrazin-2-yl)(tert-butoxycarbonyl) carbamate 29b was prepared by tert-butyl (5-bromo-3-ethynyl-pyrazin-2-yl)(tert-butoxycarbonyl) carbamate (500 mg, 1.26 mmol) and (E)-4-iodobenzaldehyde oxime 29a using a method similar to step two of Example 1.

### Step Three

### tert-butyl (tert-butoxycarbonyl)(3-(3-(4-iodophenyl)isoxazol-5-yl)-5-(4-(isopropylsulfonyl)phenyl)pyrazin-2-yl) carbamate 29c

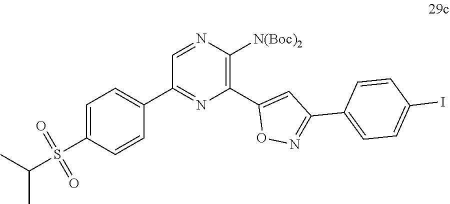

Under nitrogen protection, tert-butyl (5-bromo-3-(3-(4-iodophenyl)isoxazol-5-yl)pyrazin-2-yl)(tert-butoxycarbonyl) carbamate 29b (400.0 mg, 0.62 mmol) and 4-isopropylsulfonylbenzeneboronic acid (169.6 mg, 0.74 mmol) were used to prepare tert-butyl (tert-butoxycarbonyl)(3-(3-(4-iodophenyl)isoxazol-5-yl)-5-(4-(isopropylsulfonyl)phenyl)pyrazin-2-yl) carbamate 29c (90 mg) using a method similar to step three of Example 1.

### Step Four

### tert-butyl (3-(3-(4-acetimidophenyl)isoxazol-5-yl)-5-(4-(isopropylsulfonyl)phenyl)pyrazin-2-yl)(tert-butoxycarbonyl) carbamate 29d

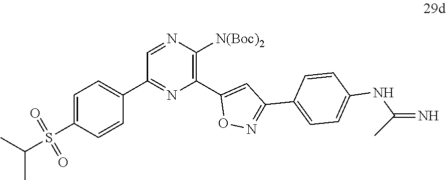

Under nitrogen protection, to a solution of tert-butyl (tert-butoxycarbonyl)(3-(3-(4-iodophenyl)isoxazol-5-yl)-5-(4-(isopropylsulfonyl)phenyl)pyrazin-2-yl) carbamate 29c (50.0 mg, 0.067 mmol) in tert-butanol (2 ml) were sequentially added acetamidine hydrochloride (12.6 mg, 0.13 mmol), tert-butanol sodium (19.3 mmol, 0.20 mmol), 2-(dicyclohexylphosphino)-3,6-dimethoxy-2′-4′-6′-tri-I-propyl-11′-biphenyl (7.2 mg, 0.013 mmol), and chloro(2-dicyclohexylphosphino-3,6-dimethoxy-2′,4′,6′-tri-isopropyl-1,1′-biphenyl)[2-(2-amino ethylphenyl)]palladium(II) (9.7 mg, 0.013 mmol). The reaction solution was stirred at 95° C. for five hours. After cooling to room temperature, ethyl acetate (40 mL) and water (40 mL) were added to the reaction solution. The mixed solution was filtered through diatomaceous earth and the resulting solution was separated. The aqueous phase was extracted three times with ethyl acetate (20 mL), and the combined organic phase was concentrated and isolated by column chromatography to give tert-butyl (3-(3-(4-acetimidophenyl)isoxazol-5-yl)-5-(4-(isopropylsulfonyl)phenyl)pyrazin-2-yl)(tert-butoxycarbonyl) carbamate 29d (8 mg).

### Step Five

### N-(4-(5-(3-Amino-6-(4-(isopropylsulfonyl)phenyl)pyrazin-2-yl)isoxazol-3-yl)phenyl)acetimidate dihydrochloride 29

The N-(4-(5-(3-Amino-6-(4-(isopropylsulfonyl)phenyl)pyrazin-2-yl)isoxazol-3-yl)phenyl)acetimidate dihydrochloride 29 was prepared by tert-butyl (3-(3-(4-acetimidophenyl)isoxazol-5-yl)-5-(4-(isopropylsulfonyl)phenyl)pyrazin-2-yl)(tert-butoxycarbonyl) carbamate 29d using a method similar to step six of Example 1.

¹H NMR (400 MHz, DMSO-d6): δ11.66 (s, 1H), 9.7 (br. s, 1H), 8.99 (s, 1H), 8.78 (s, 1H), 8.42-8.40 (m, 2H), 8.22-8.20 (m, 2H), 7.98-7.96 (m, 2H), 7.90 (s, 1H), 7.57-7.55 (m, 2H), 7.27-7.20 (m, 2H), 3.56-3.53 (m, 1H), 2.42 (s, 3H), 1.23-1.21 (m, 6H)

MS measured value (ESI⁺) [(M+H)⁺]: 477

### Example 30

### 1-(4-(5-(3-amino-6-(4-(isopropylsulfonyl)phenyl)pyrazin-2-yl)isoxazol-3-yl)-3-(trifluoromethyl)benzyl)guanidine dihydrochloride 30

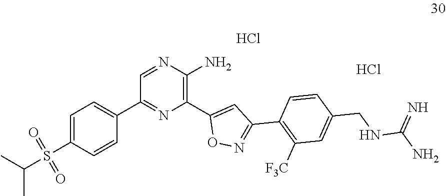 [SMILES: Cl ; CC(C)S(=O)(=O)c1ccc(-c2cnc(N)c(-c3cc(-c4ccc(CNC(=N)N)cc4C(F)(F)F)no3)n2)cc1 ; Cl]

### Step One

### 4-formyl-3-(trifluoromethyl)benzonitrile 30a

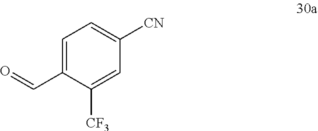 [SMILES: N#Cc1ccc(C=O)c(C(F)(F)F)c1]

Under nitrogen protection, a solution of 4-bromo-2-(trifluoromethyl)benzaldehyde (2 g, 7.96 mmol) in DMF (15 ml) were sequentially added zinc cyanide (1.0 g, 8.8 mmol) and tetrakis(triphenylphosphine)palladium (459.7 mg, 0.40 mmol). The reaction solution was stirred at 90° C. for four hours. After cooling to room temperature, the reaction solution was concentrated, and then added with ethyl acetate (50 mL) and water (50 mL). After separating the resulting solution, the aqueous phase was extracted twice with ethyl acetate (30 mL). The combined organic phase was concentrated and isolated by column chromatography to give 4-formyl-3-(trifluoromethyl)benzonitrile 30a (1.1 g).

### Step Two

### (E)-4-((hydroxyimino)methyl)-3-(trifluoromethyl)benzonitrile 30b

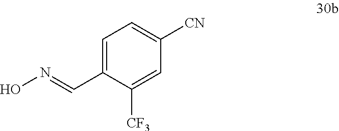 [SMILES: N#Cc1ccc(/C=N/O)c(C(F)(F)F)c1]

The white solid (E)-4-((hydroxyimino)methyl)-3-(trifluoromethyl)benzonitrile 30b (400 mg) was prepared by 4-formyl-3-(trifluoromethyl)benzonitrile 30a (500 mg, 2.5 mmol) and hydroxylamine hydrochloride (223.6 mg, 3.24 mmol) using a method similar to the step one of Example 1.

### Step Three

### tert-butyl (5-bromo-3-(3-(2-trifluoromethyl-4-cyanophenyl) isoxazol-5-yl)pyrazin-2-yl)(tert-butoxycarbonyl) carbamate 30c

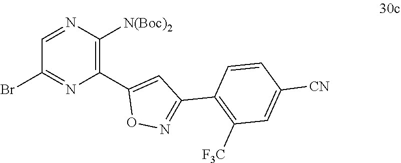

The tert-butyl (5-bromo-3-(3-(2-trifluoromethyl-4-cyanophenyl) isoxazol-5-yl)pyrazin-2-yl)(tert-butoxycarbonyl) carbamate 30c (300 mg) was prepared by (E)-4-((hydroxyimino)methyl)-3-(trifluoromethyl)benzonitrile 30b (400 mg, 1.87 mmol) using a method similar to step two of Example 1.

### Step Four

### tert-butyl (tert-butoxycarbonyl) (3-(3-(4-cyano-2-(trifluoromethyl)phenyl)isoxazol-5-yl)-5-(4-(isopropylsulfonyl)phenyl)pyrazin-2-yl) carbamate 30d

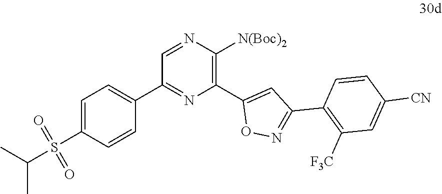

Under nitrogen protection, the tert-butyl (5-bromo-3-(3-(2-trifluoromethyl-4-cyanophenyl) isoxazol-5-yl)pyrazin-2-yl)(tert-butoxycarbonyl) carbamate 30c (300.0 mg, 0.49 mmol) and 4-isopropylsulfonylbenzeneboronic acid (169.6 mg, 0.74 mmol) were used to prepare tert-butyl (tert-butoxycarbonyl) (3-(3-(4-cyano-2-(trifluoromethyl)phenyl)isoxazol-5-yl)-5-(4-(isopropylsulfonyl)phenyl)pyrazin-2-yl) carbamate 30d (100 mg) using a method similar to step three of Example 1.

### Step Five

### tert-butyl (3-(3-(4-aminomethyl-2-(trifluoromethyl)phenyl)isoxazol-5-yl)-5-(4-(isopropylsulfonyl)phenyl)pyrazin-2-yl)(tert-butoxycarbonyl)carbamate 30e

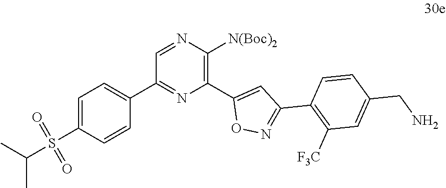

The tert-butyl (3-(3-(4-aminomethyl-2-(trifluoromethyl)phenyl)isoxazol-5-yl)-5-(4-(isopropylsulfonyl)phenyl)pyrazin-2-yl)(tert-butoxycarbonyl)carbamate 30e (10 mg) was prepared by tert-butyl (tert-butoxycarbonyl) (3-(3-(4-cyano-2-(trifluoromethyl)phenyl)isoxazol-5-yl)-5-(4-(isopropylsulfonyl)phenyl)pyrazin-2-yl) carbamate 30d (100 mg, 0.14 mmol) using a method similar to step four of Example 1.

### Step Six

### tert-butyl (tert-butoxycarbonyl)(3-(3-(4-(guanidinomethylidene)-2-(trifluoromethyl)phenyl)isoxazol-5-yl)-5-(4-(isopropylsulfonyl)phenyl)pyrazin-2-yl)carbamate 30f

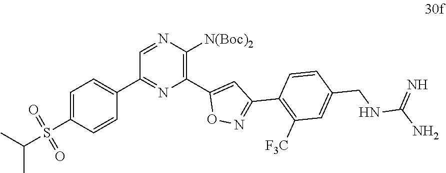

The tert-butyl (tert-butoxycarbonyl)(3-(3-(4-(guanidinomethylidene)-2-(trifluoromethyl)phenyl)isoxazol-5-yl)-5-(4-(isopropylsulfonyl)phenyl)pyrazin-2-yl)carbamate 30f was prepared by tert-butyl (3-(3-(4-aminomethyl-2-(trifluoromethyl)phenyl)isoxazol-5-yl)-5-(4-(isopropylsulfonyl)phenyl)pyrazin-2-yl)(tert-butoxycarbonyl)carbamate 30e (10 mg, 0.014 mmol) and 1H-pyrazole-1-carboxamidine hydrochloride (7.5 mg, 0.051 mmol) using a method similar to step five of Example 1.

### Step Seven

### 1-(4-(5-(3-amino-6-(4-(isopropylsulfonyl)phenyl)pyrazin-2-yl)isoxazol-3-yl)-3-(trifluoromethyl)benzyl)guanidine dihydrochloride 30

The 1-(4-(5-(3-amino-6-(4-(isopropylsulfonyl)phenyl)pyrazin-2-yl)isoxazol-3-yl)-3-(trifluoromethyl)benzyl)guanidine dihydrochloride 30 (2 mg) was prepared by tert-butyl (tert-butoxycarbonyl)(3-(3-(4-(guanidinomethylidene)-2-(trifluoromethyl)phenyl)isoxazol-5-yl)-5-(4-(isopropylsulfonyl)phenyl)pyrazin-2-yl)carbamate 30f (5 mg, 0.006 mmol) using a method similar to step six of Example 1.

¹H NMR (400 MHz, DMSO-d₆): δ 8.96 (s, 1H), 8.36-8.34 (m, 2H), 8.30-8.27 (m, 1H), 8.18-8.16 (m, 1H), 7.98-7.97 (m, 1H), 7.94-7.92 (m, 2H), 7.87-7.85 (m, 1H), 7.79-7.77 (m, 1H), 7.45 (s, 1H), 7.32 (s, 1H), 7.19 (s, 1H), 7.06 (s, 1H), 4.61-4.59 (m, 1H), 3.55-3.53 (m, 1H), 1.19-1.17 (m, 6H)

MS measured value (ESI⁺) [(M+H)⁺]: 560

### Example 31

### 5-(5-(3-amino-6-(4-isopropylsulfonyl)phenyl)pyrazin-2-yl)isoxazol-3-yl)-isoindolyl-1-one 31

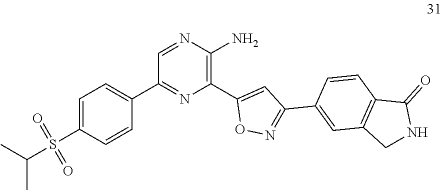 [SMILES: CC(C)S(=O)(=O)c1ccc(-c2cnc(N)c(-c3cc(-c4ccc5c(c4)CNC5=O)no3)n2)cc1]

### Step One

### (E)-1-oxoisoindolyl-5-formyl oxime 31a

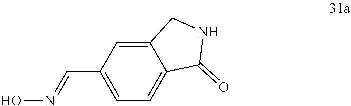 [SMILES: O=C1NCc2cc(/C=N/O)ccc21]

The 1-oxoisoindolyl-5-formaldehyde (0.1 g, 0.62 mmol) and hydroxylamine hydrochloride (0.052 g, 0.74 mmol) are used, through a method similar to step one of Example 1, to prepare the white solid (E)-1-oxoisoindolyl-5-formyl oxime 31a (0.175 g) that was a crude product and used directly in the next step without a further purification.

### Step Two

### tert-butyl (5-bromo-3-(3-(1-oxoisoindol-5-yl)isoxazol-5-yl)pyrazin-2-yl)(tert-butoxycarbonyl)carbamate 31b

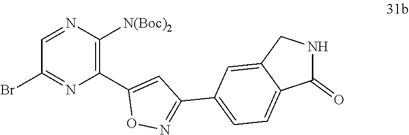

The yellow solid tert-butyl (5-bromo-3-(3-(1-oxoisoindol-5-yl)isoxazol-5-yl)pyrazin-2-yl)(tert-butoxycarbonyl) carbamate 31b (120 mg) was prepared by (E)-1-oxoisoindolyl-5-formyl oxime 31a (175 mg, 0.62 mmol), bis(tert-butyl) (5-bromo-3-ethynyl-2-pyrazinyl)carbamate (160 mg, 0.401 mmol) and bis(trifluoroacetyl)iodobenzene (0.26 g, 0.62 mmol) using a method similar to step two of Example 1.

### Step Three

### tert-butyl (tert-butoxycarbonyl) (5-(4-(isopropylsulfonyl)phenyl)-3-(3-(1-oxoisoindol-5-yl)isoxazol-5-yl)pyrazin-2-yl) carbamate 31c

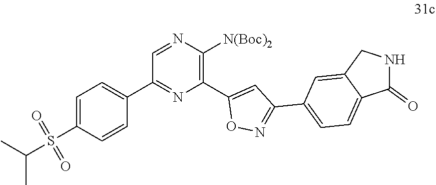

The light yellow solid tert-butyl (tert-butoxycarbonyl) (5-(4-(isopropylsulfonyl)phenyl)-3-(3-(1-oxoisoindol-5-yl)isoxazol-5-yl)pyrazin-2-yl) carbamate 31c (90 mg) was prepared by tert-butyl (5-bromo-3-(3-(1-oxoisoindol-5-yl)isoxazol-5-yl)pyrazin-2-yl)(tert-butoxycarbonyl) carbamate 31b (120 mg, 0.209 mmol), 4-isopropylsulfonylbenzene boronic acid (48 mg, 0.209 mmol), potassium carbonate (58 mg, 0.418 mmol), and [1,1′-bis (diphenylphosphino)ferrocene] palladium dichloride (31 mg, 0.042 mmol) using a method similar to step three of Example 1.

### Step Four

### 5-(5-(3-amino-6-(4-isopropylsulfonyl)phenyl)pyrazin-2-yl)isoxazol-3-yl)-isoindolyl-1-one 31

The yellow solid 5-(5-(3-amino-6-(4-isopropylsulfonyl)phenyl)pyrazin-2-yl)isoxazol-3-yl)-isoindolyl-1-one 31 (24.7 mg) was prepared by tert-butyl (tert-butoxycarbonyl) (5-(4-(isopropylsulfonyl)phenyl)-3-(3-(1-oxoisoindol-5-yl)isoxazol-5-yl)pyrazin-2-yl) carbamate 31c (90 mg, 0.156 mmol) and a mixed solution (4 M, 2 mL) of hydrochloric acid and ethyl acetate using a method similar to step 6 of Example 1.

¹H NMR (400 MHz, DMSO-d₆): δ 8.95 (s, 1H), 8.72 (s, 1H), 8.39-8.37 (m, 2H), 8.24 (s, 1H), 8.15-8.13 (m, 1H), 7.95-7.93 (m, 2H), 7.88-7.84 (m, 2H), 7.20 (s, 2H), 4.50 (s, 2H), 3.49-3.43 (m, 1H), 1.20-1.19 (m, 6H)

MS measured value (ESI⁺) [(M+H)⁺]: 476

### Example 32

### 5-(5-amino-6-(3-(4-((methylamino)methyl)phenyl)isoxazol-5-yl)pyrazin-2-yl)-3-fluoro-2,2-dimethyl-2,3-dihydrobenzo[b]thiophene 1,1-dioxide 32

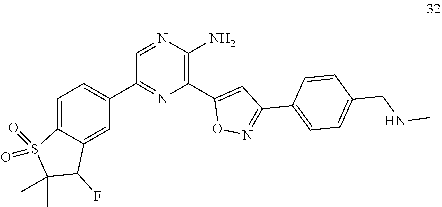 [SMILES: CNCc1ccc(-c2cc(-c3nc(-c4ccc5c(c4)C(F)C(C)(C)S5(=O)=O)cnc3N)on2)cc1]

### Step One

### Methyl-2-((4-bromophenyl)thio)acetate 32a

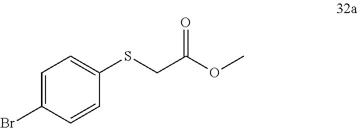

To a solution of 4-bromothiophenol (5.88 g, 31.10 mmol) in tetrahydrofuran (50 mL) were added methyl bromoacetate (7.14 g, 46.65 mmol) and triethylamine (6.29 g, 62.20 mmol) at room temperature. The reaction mixture was heated to 70° C. and stirred for 3 hours. Water (100 mL) and ethyl acetate (100 mL) were added to the reaction system and the organic phase was separated out. The aqueous phase was extracted three times with ethyl acetate (50 mL). The combined organic phases were dried with anhydrous sodium sulfate and filtered. The filtrate was concentrated under reduced pressure and give the white solid methyl-2-((4-bromophenyl)thio)acetate 32a (8.6 g) through column chromatography.

### Step Two

### 2-((4-bromophenyl)thio)acetic acid 32b

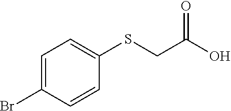 [SMILES: O=C(O)CSc1ccc(Br)cc1]

To a mixed solution of methyl-2-((4-bromophenyl)thio)acetate 32a (8.12 g, 31.10 mmol) in methanol (60 mL) and water (20 mL) was added sodium hydroxide (2.49 g, 62.19 mmol) at room temperature. The reaction mixture was stirred at room temperature (˜20° C.) for 12 hours. Water (300 mL) was added to the reaction system, and then the pH was adjusted to 5˜6 with 2M aqueous hydrochloric acid followed by the addition of ethyl acetate (300 mL). The organic phase was separated out, and the aqueous phase was extracted with ethyl acetate (100 mL) for three times. The combined organic phase was dried with anhydrous sodium sulfate and filtered. The filtrate was concentrated under reduced pressure to give a white solid 2-((4-bromophenyl)thio)acetic acid 32b (6.3 g) that was a crude product and used directly in the next step without a further purification.

### Step Three

### 5-bromobenzo[b]thiophen-3(2H)-one 32c

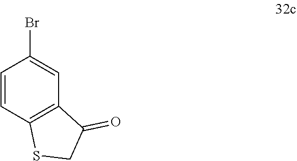 [SMILES: O=C1CSc2ccc(Br)cc21]

The reaction solution of 2-((4-bromophenyl)thio)acetic acid (3.0 g, 12.14 mmol) dissolved in trifluoromethanesulfonic acid (15 mL) was stirred at 80° C. for 0.5 h. After cooling to room temperature, the reaction solution was dropped into ice water followed by the addition of ethyl acetate (100 mL). The organic phase was washed three times with saturated aqueous sodium bicarbonate (30 mL). The resulting organic phase was concentrated and give the yellow solid 5-bromobenzo[b]thiophen-3(2H)-one 32c (1.5 g) through column chromatography.

### Step Four

### 5-bromobenzo[b]thiophen-3(2H)-one-1,1-dioxide 32d

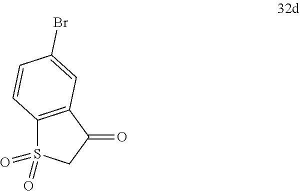 [SMILES: O=C1CS(=O)(=O)c2ccc(Br)cc21]

The yellow solid 5-bromobenzo[b]thiophen-3(2H)-one-1,1-dioxide 32d (1.4 g) was prepared by 5-bromobenzo[b] thiophen-3(2H)-one 32c (1.5 g) and m-chloroperoxybenzoic acid (3.39 g, 19.64 mmol) using a method similar to step one of Example 27.

### Step Five

### 5-bromo-2,2-dimethyl-benzo[b]thiophen-3(2H)-one-1,1-dioxide 32e

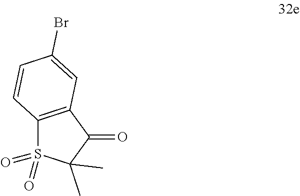

To a solution of 5-bromobenzo[b]thiophen-3(2H)-one-1,1-dioxide 32d (400 mg, 1.53 mmol) in tetrahydrofuran (12 mL) were added 1,5-diazabicyclo[4.3.0]non-5-ene (950 mg, 7.66 mmol) and iodomethane (2.17 g, 15.32 mmol) sequentially at room temperature. After the reaction solution was heated to 70° C. and stirred for 3 hours, ethyl acetate (50 mL) and water (50 mL) were added. The organic phase was separated out and the aqueous phase was extracted three times with ethyl acetate (40 mL). The combined organic phase was concentrated and isolated by column chromatography to give the white solid 5-bromo-2,2-dimethyl-benzo[b]thiophen-3(2H)-one-1,1-dioxide 32e (380 mg).

### Step Six

### 5-bromo-3-hydroxy-2,2-dimethyl-2,3-dihydrobenzo[b]thiophene-1,1-dioxide 32f

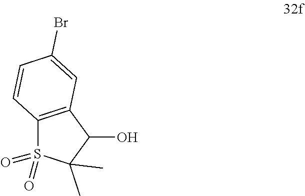

To a solution of 5-bromo-2,2-dimethyl-benzo[b]thiophen-3(2H)-one-1,1-dioxide 32e (210 mg, 0.73 mmol) in methanol (5 mL) was added sodium borohydride (137 mg, 3.63 mmol). The reaction solution was stirred for 1 hour at room temperature and quenched with water (1 mL). Then, ethyl acetate (20 mL) and water (20 mL) were added, and the resulting solution was separated. The aqueous phase was extracted three times with ethyl acetate (20 mL). The combined organic phase was concentrated and isolated by column chromatography to give the white solid 5-bromo-3-hydroxy-2,2-dimethyl-2,3-dihydrobenzo[b]thiophene-1,1-dioxide 32f (150 mg).

### Step Seven

### 5-bromo-3-fluoro-2,2-dimethyl-2,3-dihydrobenzo[b]thiophene-1,1-dioxide 32g

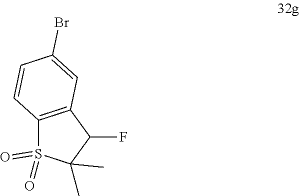

To a solution of 5-bromo-3-hydroxy-2,2-dimethyl-2,3-dihydrobenzo[b]thiophene-1,1-dioxide 32f (150 mg, 0.51 mmol) in dichloromethane (5 mL) was added diethylamino sulfur trifluoride (250 mg, 1.55 mmol) at 0° C. The reaction solution was stirred for 1 hour at room temperature and quenched with water (1 mL). Then, dichloromethane (20 mL) and water (20 mL) were added, and the resulting solution was separated out. The aqueous phase was extracted three times with dichloromethane (20 mL), and the combined organic phase was concentrated and isolated by column chromatography to give the white solid 5-bromo-3-fluoro-2,2-dimethyl-2,3-dihydrobenzo[b]thiophene-1,1-dioxide 32g (120 mg).

### Step Eight

### 3-fluoro-2,2-dimethyl-5-(4,4,5,5-tetramethyl-1,3,2-dioxaborolan-2-yl)-2,3-dihydrobenzo[b]thiophene-1,1-dioxide 32h

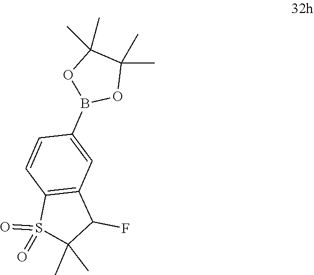 [SMILES: CC1(C)OB(c2ccc3c(c2)C(F)C(C)(C)S3(=O)=O)OC1(C)C]

The 5-bromo-3-fluoro-2,2-dimethyl-2,3-dihydrobenzo[b]thiophene-1,1-dioxide 32g (120 mg, 0.409 mmol), bis(pinacolato)diboron (208 mg, 0.82 mmol), potassium acetate (80 mg, 0.82 mmol), and [1,1′-bis(diphenylphosphino) ferrocene]palladium(II) dichloride (60 mg 0.082 mmol) were used, according to a method similar to step three of Example 27, to prepare 3-fluoro-2,2-dimethyl-5-(4,4,5,5-tetramethyl-1,3,2-dioxaborolan-2-yl)-2,3-dihydrobenzo[b]thiophene-1,1-dioxide 32h (230 mg) that was a crude product and used directly in the next step without a further purification.

### Step Nine

### Methyl tert-butyl (tert-butoxycarbonyl)(3-(3-(3-(4-(((tert-butoxycarbonyl)amino)methyl)phenyl)isoxazol-5-yl)-5-(3-fluoro-2,2-dimethyl-1,1-dioxo-2,3-dihydrobenzo[b]thiophen-5-yl)pyrazin-2-yl)amino acid 32i

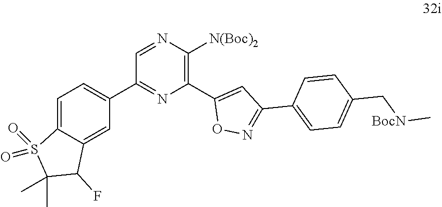

The methyl tert-butyl (tert-butoxycarbonyl)(3-(3-(3-(4-(((tert-butoxycarbonyl)amino)methyl)phenyl)isoxazol-5-yl)-5-(3-fluoro-2,2-dimethyl-1,1-dioxo-2,3-dihydrobenzo[b]thiophen-5-yl)pyrazin-2-yl)amino acid 32i (130 mg) was prepared by 3-fluoro-2,2-dimethyl-5-(4,4,5,5-tetramethyl-1,3,2-dioxaborolan-2-yl)-2,3-dihydrobenzo[b]thiophene-1,1-dioxide 32h (270 mg, 0.409 mmol), 3-fluoro-2,2-dimethyl-5-(4,4,5,5-tetramethyl-1,3,2-dioxaborolan-2-yl)-2,3-dihydrobenzo[b]thiophene-1,1-dioxide (140 mg, 0.408 mmol), potassium carbonate (113 mg, 0.82 mmol), and [1,1′-bis(diphenylphosphino) ferrocene] palladium(II) dichloride (60 mg, 0.082 mmol) using a method similar to step four of Example 27.

### Step Ten

### 5-(5-amino-6-(3-(4-((methylamino)methyl)phenyl)isoxazol-5-yl)pyrazin-2-yl)-3-fluoro-2,2-dimethyl-2,3-dihydrobenzo[b]thiophene 1,1-dioxide 32

The yellow solid 5-(5-amino-6-(3-(4-((methylamino)methyl)phenyl)isoxazol-5-yl)pyrazin-2-yl)-3-fluoro-2,2-dimethyl-2,3-dihydrobenzo[b]thiophene 1,1-dioxide 32 (57.2 mg) was prepared by methyl tert-butyl (tert-butoxycarbonyl)(3-(3-(3-(4-(((tert-butoxycarbonyl)amino)methyl)phenyl)isoxazol-5-yl)-5-(3-fluoro-2,2-dimethyl-1,1-dioxo-2,3-dihydrobenzo[b] thiophen-5-yl)pyrazin-2-yl)amino acid 32i (130 mg, 0.163 mmol) and a mixed solution (4 M, 2 mL) of hydrochloric acid and ethyl acetate using a method similar to step six of Example 1.

### Example 33-1 or Example 33-2

### (R)-5-(5-amino-6-(5-(4-((methylamino)methylene)phenyl)-1,3,4-oxadiazol-2-yl)pyrazin-2-yl)-3-fluoro-2,2-dimethyl-2,3-dihydrobenzo[b]thiophene 1,1-dioxide 33-1

### or

### (S)-5-(5-amino-6-(5-(4-((methylamino)methylene)phenyl)-1,3,4-oxadiazol-2-yl)pyrazin-2-yl)-3-fluoro-2,2-dimethyl-2,3-dihydrobenzo[b]thiophene 1,1-dioxide 33-2

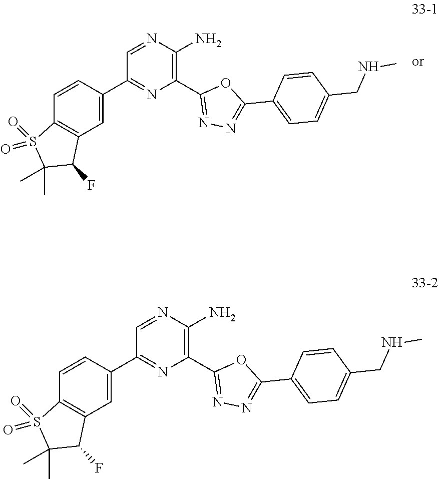 [SMILES: CNCc1ccc(-c2nnc(-c3nc(-c4ccc5c(c4)[C@@H](F)C(C)(C)S5(=O)=O)cnc3N)o2)cc1 ; CNCc1ccc(-c2nnc(-c3nc(-c4ccc5c(c4)[C@H](F)C(C)(C)S5(=O)=O)cnc3N)o2)cc1]

### Step One

### (R)-5-bromo-3-fluoro-2,2-dimethyl-2,3-dihydrobenzo[b]thiophene-1,1-dioxide 33a-1

### or

### (S)-5-bromo-3-fluoro-2,2-dimethyl-2,3-dihydrobenzo[b]thiophene-1,1-dioxide 33a-2

The 5-bromo-3-fluoro-2,2-dimethyl-2,3-dihydrobenzo[b]thiophene-1,1-dioxide 32 g (300 mg, 1.02 mmol) was split by SFC (Apparatus: SHIMADZU LC-30AD, Column: DAICEL AD-H, 4.6 mmI.D.*250 mmL 5 μm, Mobile phase: CO2/MeOH [0.1% NH₃ (7M Solution in MeOH)]=60/40, flow rate: 2.5 mL/min, wavelength: UV 214 nM/254 nM, temperature: 40° C.) to obtain 33a-1 or 33a-2 (144 mg, retention time: 3.812 min).

### Step Two

### (R)-3-fluoro-2,2-dimethyl-5-(4,4,5,5-tetramethyl-1,3,2-dioxaborolan-2-yl)-2,3-dihydrobenzo[b]thiophene-1,1-dioxide 33b-1

### or

### (S)-3-fluoro-2,2-dimethyl-5-(4,4,5,5-tetramethyl-1,3,2-dioxaborolan-2-yl)-2,3-dihydrobenzo[b]thiophene-1,1-dioxide 33b-2

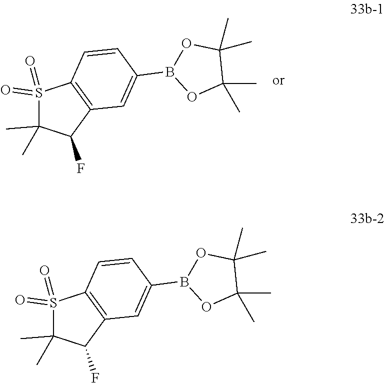

The 33a-1 or 33a-2 (85 mg, 0.289 mmol, retention time: 3.812 min), bis(pinacolato)diboron, potassium acetate (57 mg, 0.58 mmol) and [1,1′-bis(diphenylphosphino)ferrocene]palladium(II) dichloride (42 mg, 0.058 mmol) were used according to the method similar to step three of Example 27 to obtain 33b-1 or 33b-2 (150 mg) that was a crude product and used directly in the next step without a further purification.

### Step Three

### 3-amino-6-bromopyrazinyl-2-acylhydrazine 33c

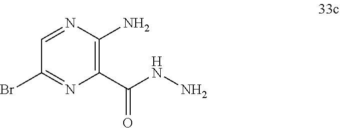 [SMILES: NNC(=O)c1nc(Br)cnc1N]

To a solution of methyl 3-amino-6-bromopyrazinyl-2-carboxylate (3.0 g, 12.93 mmol) in ethanol (15 mL) was added hydrazine hydrate (1.88 mL, 38.79 mmol) at room temperature. The reaction mixture was heated to 80° C. and stirred for 2 hours. The reaction solution was cooled, and concentrated and give the light yellow oil 3-amino-6-bromopyrazinyl-2-acylhydrazine 33c (2.7 g) through the column chromatography.

### Step Four

### tert-butyl (4-(2-(3-amino-6-bromopyrazine-2-carboxylato)hydrazine-1-carboxylato benzyl)(methyl)carbamate 33d

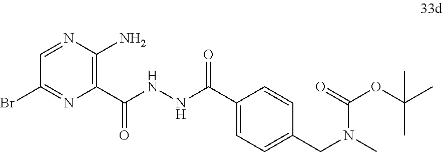 [SMILES: CN(Cc1ccc(C(=O)NNC(=O)c2nc(Br)cnc2N)cc1)C(=O)OC(C)(C)C]

To a solution of 4-(((tert-butoxycarbonyl)(methyl)amino)methyl)benzoic acid (500 mg, 1.88 mmol) in N,N-dimethylformamide (10 mL) was added 3-amino-6-bromopyrazinyl-2-acylhydrazine 33c (525 mg, 2.26 mmol), triethylamine (0.57 mL, 4.15 mmol) and O-(7-azabenzotriazol-1-yl)-N,N,N′,N′-tetramethylurea hexafluorophosphate (726 mg, 2.26 mmol). The reaction mixture was stirred at room temperature for 16 h. Then, water (50 mL) and ethyl acetate (50 mL) were added to the reaction system. The organic phase was separated out, and the aqueous phase was extracted with ethyl acetate (50 mL) three times. The combined organic phase was dried with anhydrous sodium sulfate, filtered, and the filtrate was concentrated under reduced pressure and give the yellow oil tert-butyl (4-(2-(3-amino-6-bromopyrazine-2-carboxylato)hydrazine-1-carboxylato)benzyl)(methyl)carbamate 33d (800 mg) through the column chromatography.

### Step Five

### tert-butyl (4-(5-(3-amino-6-bromopyrazin-2-yl)-1,3,4-oxadiazol-2-yl)benzyl)(methyl) carbamate 33e

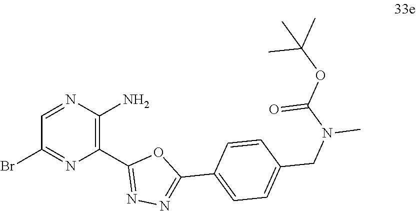 [SMILES: CN(Cc1ccc(-c2nnc(-c3nc(Br)cnc3N)o2)cc1)C(=O)OC(C)(C)C]

To a solution of tert-butyl (4-(2-(3-amino-6-bromopyrazine-2-carboxylato)hydrazine-1-carboxylato)benzyl)(methyl)carbamate 33d (800 mg, 1.67 mmol) in anhydrous acetonitrile (10 mL) was added diisopropylethylamine (0.87 mL, 5.01 mmol) at room temperature. Dibromotriphenylphosphine (915 mg, 2.17 mmol) was slowly added to the mixed reaction solution that was then stirred at room temperature for 16 hours. Water (50 mL) and ethyl acetate (50 mL) were added to the reaction system and the organic phase was separated out. The aqueous phase was extracted three times with ethyl acetate (50 mL), and the combined organic phase was dried with anhydrous sodium sulfate, and filtered. The filtrate was concentrated under reduced pressure and give a white solid tert-butyl (4-(5-(3-amino-6-bromopyrazin-2-yl)-1,3,4-oxadiazol-2-yl)benzyl)(methyl) carbamate 33e (330 mg) through the column chromatography.

### Step Six

### tert-butyl (R)-(4-(5-(3-amino-6-(3-fluoro-2,2-dimethyl-1,1-dioxo-2,3-dihydrobenzo[b]thiophen-5-yl)pyrazin-2-yl)-1,3,4-oxadiazol-2-yl)benzyl)(methyl)carbamate 33f-1

### or

### tert-butyl (S)-(4-(5-(3-amino-6-(3-fluoro-2,2-dimethyl-1,1-dioxo-2,3-dihydrobenzo[b]thiophen-5-yl)pyrazin-2-yl)-1,3,4-oxadiazol-2-yl)benzyl)(methyl)carbamate 33f-2

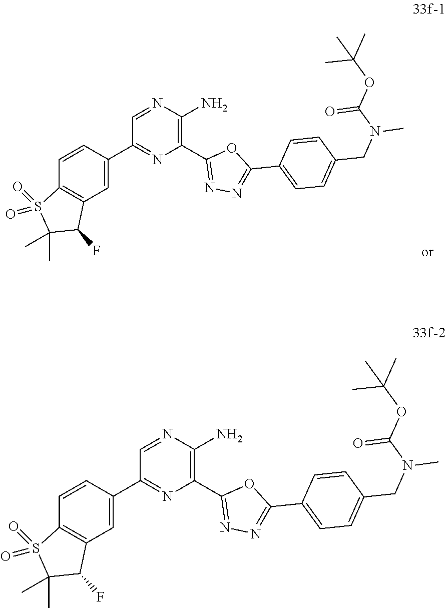 [SMILES: CN(Cc1ccc(-c2nnc(-c3nc(-c4ccc5c(c4)[C@@H](F)C(C)(C)S5(=O)=O)cnc3N)o2)cc1)C(=O)OC(C)(C)C ; CN(Cc1ccc(-c2nnc(-c3nc(-c4ccc5c(c4)[C@H](F)C(C)(C)S5(=O)=O)cnc3N)o2)cc1)C(=O)OC(C)(C)C]

The -butyl (4-(5-(3-amino-6-bromopyrazin-2-yl)-1,3,4-oxadiazol-2-yl)benzyl)(methyl) carbamate 33e (130 mg, 0.282 mmol), 33b-1 or 33b-2 (96 mg, 0.281 mmol), potassium carbonate (78 mg, 0.563 mmol), and [1,1′-bis(diphenylphosphino) ferrocene] palladium(II) dichloride (42 mg, 0.056 mmol) were used according to a method similar to step four of Example 27 to obtain the title product 33f-1 or 33f-2 (100 mg).

### Step Seven

### (R)-5-(5-amino-6-(5-(4-((methylamino)methylene)phenyl)-1,3,4-oxadiazol-2-yl)pyrazin-2-yl)-3-fluoro-2,2-dimethyl-2,3-dihydrobenzo[b]thiophene 1,1-dioxide 33-1

### or

### (S)-5-(5-amino-6-(5-(4-((methylamino)methylene)phenyl)-1,3,4-oxadiazol-2-yl)pyrazin-2-yl)-3-fluoro-2,2-dimethyl-2,3-dihydrobenzo[b]thiophene 1,1-dioxide 33-2

The compound 33f-1 or 33f-2 (100 mg, 0.168 mmol) and a mixed solution (4 M, 2 mL) of hydrochloric acid and ethyl acetate were used to prepare the yellow solid title product 33-1 or 33-2 (51.2 mg) using a method similar to step six of Example 1.

¹H NMR (400 MHz, DMSO-d₆): δ 9.12 (s, 1H), 8.56-8.53 (m, 2H), 8.24-8.21 (m, 2H), 8.07-8.05 (m, 1H), 7.78-7.76 (m, 2H), 6.11 (s, 0.5H), 5.98 (s, 0.5H), 4.16 (s, 2H), 2.54 (s, 3H), 1.51-1.50 (m, 3H), 1.38 (s, 3H)

MS measured value (ESI⁺) [(M+H)⁺]: 495

### Example 34

### 5-(5-amino-6-(5-(4-((methylamino)methyl)phenyl)isoxazol-3-yl)pyrazin-2-yl)-3-fluoro-2,2-dimethyl-2,3-dihydrobenzo[b]thiophene 1,1-dioxide 34

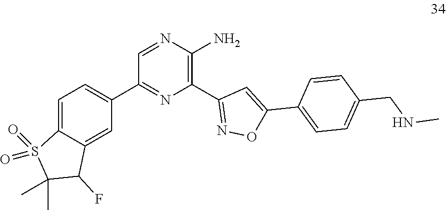 [SMILES: CNCc1ccc(-c2cc(-c3nc(-c4ccc5c(c4)C(F)C(C)(C)S5(=O)=O)cnc3N)no2)cc1]

### Step One

### (E)-3-amino-6-bromopyrazine-2-carbaldehyde oxime 34a

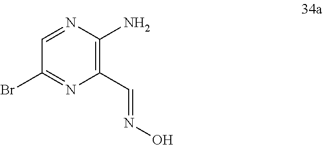 [SMILES: Nc1ncc(Br)nc1/C=N/O]

To a solution of 3-amino-6-bromopyrazine-2-carbaldehyde (200 mg, 1.00 mmol) in tetrahydrofuran (4 mL) was added hydroxylamine hydrochloride (138 mg, 2.00 mmol) at room temperature. The reaction mixture was stirred at room temperature for 4 hours. Water (10 mL) was added to the reaction system and the organic phase was separated out. The aqueous phase was extracted three times with ethyl acetate (10 mL), and the combined organic phase was dried with anhydrous sodium sulfate and filtered. The filtrate was concentrated under reduced pressure to give a white solid (E)-3-amino-6-bromopyrazine-2-carbaldehyde oxime 34a, which was a crude product and used directly in the next step without a further purification.

### Step Two

### 1-(4-ethynlphenyl)-N-methylmethylamine 34b

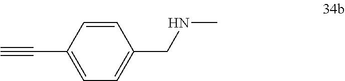

To a solution of 4-ethynylbenzaldehyde (1 g, 7.69 mmol) in methanol (10 mL) were added methylamine hydrochloride (572 mg, 8.46 mmol), sodium cyanoborohydride (966 mg, 15.38 mmol) and acetic acid (50 mg, 0.77 mmol) at room temperature. The reaction mixture was stirred at room temperature for 4 hours. Water (50 mL) and ethyl acetate (50 mL) were added to the reaction system, and the organic phase was separated out. The aqueous phase was extracted three times with ethyl acetate (50 mL), and the combined organic phase was dried with anhydrous sodium sulfate and filtered. The filtrate was concentrated under reduced pressure and column chromatographed to give the colorless oil 1-(4-ethynylphenyl)-N-methylmethylamine 34b (500 mg).

### Step Three

### tert-butyl (4-ethynylbenzyl)(methyl)carbamate 34c

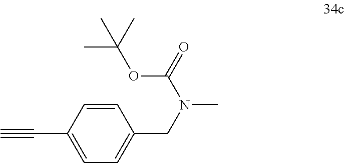 [SMILES: C#Cc1ccc(CN(C)C(=O)OC(C)(C)C)cc1]

To a solution of 1-(4-ethynylphenyl)-N-methylmethylamine 34b (500 mg, 2.04 mmol) in dichloromethane (13 mL) was added triethylamine (413 mg, 4.08 mmol) and di-tert-butyl dicarbonate (668 mg, 3.06 mmol) at room temperature. The reaction mixture was stirred at room temperature for 4 hours. Then, water (50 mL) and dichloromethane (50 mL) were added to the reaction system and the organic phase was separated out. The aqueous phase was extracted with ethyl acetate (50 mL) three times, and the combined organic phase was dried with anhydrous sodium sulfate and filtered. The filtrate was concentrated under reduced pressure and column chromatographed to give tert-butyl (4-ethynylbenzyl)(methyl)carbamate 34c (550 mg).

### Step Four

### tert-butyl (4-(3-(3-amino-6-bromopyrazin-2-yl)isoxazol-5-yl)benzyl)(methyl)carbamate 34d

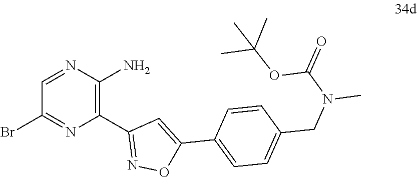 [SMILES: CN(Cc1ccc(-c2cc(-c3nc(Br)cnc3N)no2)cc1)C(=O)OC(C)(C)C]

To a mixed solution of tert-butyl (4-ethynylbenzyl)(methyl)carbamate 34c (0.48 g, 1.20 mmol) in methanol (10 mL) and water (2 mL) were added (E)-3-amino-6-bromopyrazine-2-carboxaldehyde oxime (0.135 g, 0.63 mmol) and bis(trifluoroacetyl)iodobenzene (0.404 g, 0.94 mmol)sequentially. The reaction solution was stirred at room temperature overnight and were added with ethyl acetate (40 mL) and water (40 mL). The organic phase was washed three times with water (20 mL). The organic phase was separated out, dried with anhydrous sodium sulfate, filtered, concentrated and isolated by column chromatography to give tert-butyl (4-(3-(3-amino-6-bromopyrazin-2-yl)isoxazol-5-yl)benzyl)(methyl) carbamate 34d (0.10 g).

### Step Five

### tert-butyl (4-(3-(3-amino-6-(3-fluoro-2,2-dimethyl-1,1-dioxo-2,3-dihydrobenzo[b]thiophen-5-yl)pyrazin-2-yl)isoxazol-5-yl)benzyl)(methyl) carbamate 34e

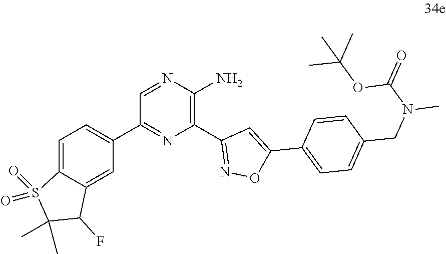 [SMILES: CN(Cc1ccc(-c2cc(-c3nc(-c4ccc5c(c4)C(F)C(C)(C)S5(=O)=O)cnc3N)no2)cc1)C(=O)OC(C)(C)C]

The tert-butyl (4-(3-(3-amino-6-(3-fluoro-2,2-dimethyl-1,1-dioxo-2,3-dihydrobenzo[b]thiophen-5-yl)pyrazin-2-yl)isoxazol-5-yl)benzyl)(methyl) carbamate 34e (80 mg) was prepared by tert-butyl (4-(3-(3-amino-6-bromopyrazin-2-yl)isoxazol-5-yl)benzyl)(methyl) carbamate 34d (100 mg, 0.169 mmol), 3-fluoro-2,2-dimethyl-5-(4,4,5,5-tetramethyl-1,3,2-dioxaborolan-2-yl)-2,3-dihydrobenzo[b]thiophene-1,1-dioxide 32h (57 mg, 0.169 mmol), potassium carbonate (69 mg, 0.507 mmol) and [1,1′-bis(diphenylphosphino)ferrocene]palladium(II) dichloride (12 mg, 0.017 mmol) using a method similar to step four of Example 27.

### Step Six

### 5-(5-amino-6-(5-(4-((methylamino)methyl)phenyl)isoxazol-3-yl)pyrazin-2-yl)-3-fluoro-2,2-dimethyl-2,3-dihydrobenzo[b]thiophene 1,1-dioxide 34

The yellow solid 5-(5-amino-6-(5-(4-((methylamino)methyl)phenyl)isoxazol-3-yl)pyrazin-2-yl)-3-fluoro-2,2-dimethyl-2,3-dihydrobenzo[b]thiophene 1,1-dioxide 34 (31.7 mg) was prepared by tert-butyl (4-(3-(3-amino-6-(3-fluoro-2,2-dimethyl-1,1-dioxo-2,3-dihydrobenzo[b]thiophen-5-yl)pyrazin-2-yl)isoxazol-5-yl)benzyl)(methyl) carbamate 34e (80 mg, 0.134 mmol) and a mixture (4 M, 2 mL) of hydrochloric acid and ethyl acetate using a method similar to step six of Example 1.

¹H NMR (400 MHz, DMSO-d₆): δ 9.12 (s, 1H), 8.63-8.59 (m, 2H), 8.02-8.00 (m, 3H), 7.87 (s, 1H), 7.57-7.55 (m, 2H), 6.04 (s, 0.5H), 5.91 (s, 0.5H), 3.78 (s, 2H), 2.32 (s, 3H), 1.51-1.50 (m, 3H), 1.38 (s, 3H)

MS measured value (ESI⁺) [(M+H)⁺]: 494

### Example 35-1 or 35-2

### (R)-5-(5-amino-6-(3-(4-((methylamino)methyl)phenyl)isoxazol-5-yl)pyrazin-2-yl)-3-fluoro-2,2-dimethyl-2,3-dihydrobenzo[b]thiophene 1,1-dioxide 35-1

### or

### (S)-5-(5-amino-6-(3-(4-((methylamino)methyl)phenyl)isoxazol-5-yl)pyrazin-2-yl)-3-fluoro-2,2-dimethyl-2,3-dihydrobenzo[b]thiophene 1,1-dioxide 35-2

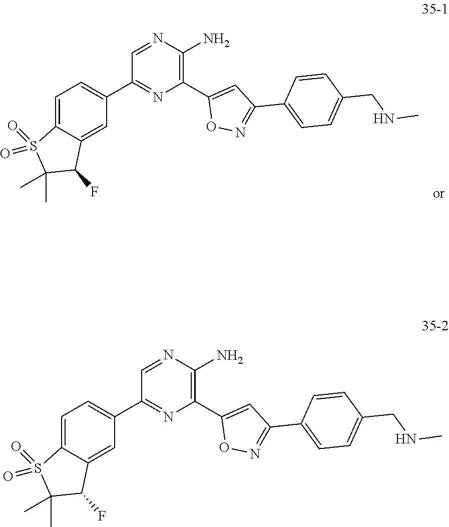 [SMILES: CNCc1ccc(-c2cc(-c3nc(-c4ccc5c(c4)[C@@H](F)C(C)(C)S5(=O)=O)cnc3N)on2)cc1 ; CNCc1ccc(-c2cc(-c3nc(-c4ccc5c(c4)[C@H](F)C(C)(C)S5(=O)=O)cnc3N)on2)cc1]

### Step One

### (5-(bis(tert-butoxycarbonyl)amino)-6-(3-(4-(((tert-butoxycarbonyl)(methyl)amino)methyl)phenyl)isoxazol-5-yl)pyrazin-2-yl)boronic acid 35a

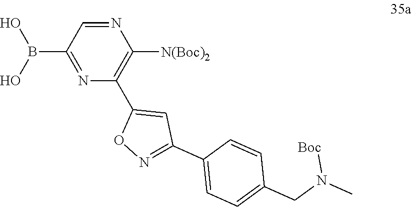

To a solution of tert-butyl (5-bromo-3-(3-(4-(((tert-butoxycarbonyl) (methyl) amino) methyl) phenyl) isoxazole-5-yl) pyrazine-2-yl) (tert-butoxycarbonyl) carbamate 2e (220 mg, 0.33 mmol) in ethylene glycol dimethyl ether solution (2 mL) were added bis(pinacolato)diboron (101 mg, 0.40 mmol), potassium acetate (98 mg, 1.00 mmol) and [1,1′-bis (diphenylphosphino) ferrocene] palladium(II) dichloride (24 mg, 0.03 mmol). The reaction mixture was stirred at 60° C. for 3 hours under nitrogen protection, and then water (10 mL) was added to the reaction system. The organic phase was separated out, and the aqueous phase was extracted with ethyl acetate (10 mL) for three times. The combined organic phase was dried with anhydrous sodium sulfate and filtered. The filtrate was concentrated under reduced pressure to give the black oil (5-(bis(tert-butoxycarbonyl)amino)-6-(3-(4-(((tert-butoxycarbonyl)(methyl)amino)methyl)phenyl)isoxazol-5-yl)pyrazin-2-yl) boronic acid 35a (230 mg) that was a crude product and used directly in the next step without a further purification.

### Step Two

### tert-butyl (R)-(tert-butoxycarbonyl)(3-(3-(3-(4-(((tert-butoxycarbonyl)(methyl)amino)methyl)phenyl)isoxazol-5-yl)-5-(3-fluoro-2,2-dimethyl-1,1-dioxide-2,3-dihydrobenzo[b]thiophen-5-yl)pyrazin-2-yl)carbamate 35b-1

### or

### tert-butyl (S)-(tert-butoxycarbonyl)(3-(3-(3-(4-(((tert-butoxycarbonyl)(methyl)amino)methyl)phenyl)isoxazol-5-yl)-5-(3-fluoro-2,2-dimethyl-1,1-dioxide-2,3-dihydrobenzo[b]thiophen-5-yl)pyrazin-2-yl) carbamate 35b-2

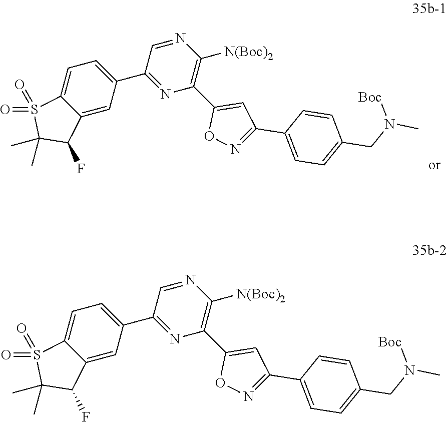

To a solution of (5-(bis(tert-butoxycarbonyl)amino)-6-(3-(4-(((tert-butoxycarbonyl)(methyl)amino)methyl)phenyl) isoxazol-5-yl)pyrazin-2-yl) boronic acid 35a (213.7 mg, 0.34 mmol) in 1,4 dioxane (3 mL) and water (0.6 mL) was added compound 33a-1 or 33a-2 (100 mg, 0.34 mmol), potassium carbonate (94.3 mg, 0.68 mmol) and [1,1′-bis-(diphenylphosphino) ferrocene] palladium(II) dichloride (25 mg, 0.03 mmol). The reaction mixture was stirred under nitrogen protection and 60° C. for 3 hours. Water (20 mL) and ethyl acetate (20 mL) were added to the reaction system, and the organic phase was separated out. The aqueous phase was extracted three times with ethyl acetate (20 mL), and the combined organic phase was dried with anhydrous sodium sulfate and filtered. The filtrate was concentrated under reduced pressure and column chromatographed to give the brown oil substance 35b-1 or 35b-2 (120 mg).

### Step Three

### (R)-5-(5-amino-6-(3-(4-((methylamino)methyl)phenyl)isoxazol-5-yl)pyrazin-2-yl)-3-fluoro-2,2-dimethyl-2,3-dihydrobenzo[b]thiophene 1,1-dioxide 35-1

### or

### (S)-5-(5-amino-6-(3-(4-((methylamino)methyl)phenyl)isoxazol-5-yl)pyrazin-2-yl)-3-fluoro-2,2-dimethyl-2,3-dihydrobenzo[b]thiophene 1,1-dioxide 35-2

The yellow solid title product 35-1 or 35-2 was prepared by compound 35b-1 or 35b-2 (120 mg, 0.15 mmol) and a mixture (4 M, 2 mL) of hydrochloric acid and ethyl acetate using a method similar to step six of Example 1.

¹H NMR (400 MHz, DMSO-d₆): δ 8.99 (s, 1H), 8.54-8.51 (m, 2H), 8.03-7.96 (m, 3H), 7.79 (s, 1H), 7.50 (m, 2H), 7.24 (s, 2H) 6.06 (s, 0.5H), 5.93 (s, 0.5H), 5.75 (s, 1H), 3.73 (s, 2H), 2.30 (s, 3H), 1.50-1.49 (m, 3H), 1.37 (s, 3H)

MS measured value (ESI⁺) [(M+H)⁺]: 494

### Example 36

### 5-(5-amino-6-(5-(4-((1-methylpiperidin-4-yl)amino)methyl)phenyl)-1,3,4-oxadiazol-2-yl)pyrazin-2-yl)-3-fluoro-2,2-dimethyl-2,3-dihydrobenzo[b]thiophene 1,1-dioxide 36

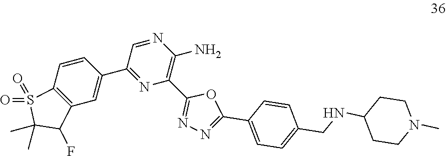 [SMILES: CN1CCC(NCc2ccc(-c3nnc(-c4nc(-c5ccc6c(c5)C(F)C(C)(C)S6(=O)=O)cnc4N)o3)cc2)CC1]

### Step One

### 5-bromo-3-(5-(4-(bromomethyl)phenyl)-1,3,4-oxadiazol-2-yl)pyrazin-2-amine 36a

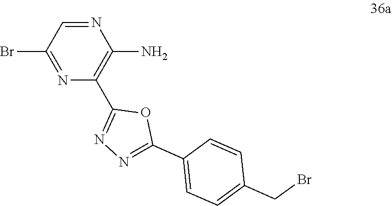 [SMILES: Nc1ncc(Br)nc1-c1nnc(-c2ccc(CBr)cc2)o1]

To a solution of 3-amino-6-bromopyrazine-2-carbohydrazide (1.294 g, 5.58 mmol) in acetonitrile (20 mL) was added 4-(bromomethyl)benzoic acid (1.20 g, 5.58 mmol) and dibromotriphenylphosphorane (10.0 g, 22.321 mmol) at room temperature. The solution was stirred for 1 h under nitrogen protection at 25° C. Then, ethylamine (2.884 g, 22.321 mmol) was added dropwise and the solution was stirred at 30° C. for 12 hours. After the reaction finished, water (100 mL) and ethyl acetate (100 mL) were added and the organic phase was separated out. The aqueous phase was extracted with ethyl acetate (50 mL) three times, and the combined organic phase was dried with anhydrous sodium sulfate and filtered. The filtrate was concentrated under reduced pressure and column chromatographed to give a yellow solid 5-bromo-3-(5-(4-(bromomethyl) phenyl)-1,3,4-oxadiazol-2-yl)pyrazin-2-amine 36a (1.10 g).

### Step Two

### 5-bromo-3-(5-(4-((((1-methylpiperidin-4-yl)amino)methyl)phenyl)-1,3,4-oxadiazol-2-yl)pyrazin-2-amine 36b

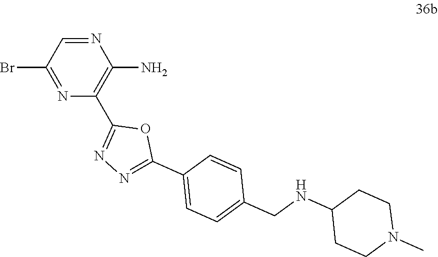 [SMILES: CN1CCC(NCc2ccc(-c3nnc(-c4nc(Br)cnc4N)o3)cc2)CC1]

At room temperature, 5-bromo-3-(5-(4-(bromomethyl) phenyl)-1,3,4-oxadiazol-2-yl)pyrazin-2-amine 36a (500 mg, 1.22 mmol), 1-methylpiperidin-4-amine (167 mg, 1.46 mmol), and sodium carbonate (387 mg, 3.65 mmol) were dissolved in tetrahydrofuran (10 mL) and the mixture was heated to 60° C. and stirred for 3 hours. Then, water (50 mL) and ethyl acetate (50 mL) were added and the organic phase was separated out. The aqueous phase was extracted with ethyl acetate (50 mL) for three times, and the combined organic phase was dried with anhydrous sodium sulfate and filtered. The filtrate was concentrated under reduced pressure and column chromatographed to give a yellow solid 5-bromo-3-(5-(4-((((1-methylpiperidin-4-yl)amino)methyl)phenyl)-1,3,4-oxadiazol-2-yl)pyrazin-2-amine 36b (300 mg).

### Step Three

### 5-(5-amino-6-(5-(4-((1-methylpiperidin-4-yl)amino)methyl)phenyl)-1,3,4-oxadiazol-2-yl)pyrazin-2-yl)-3-fluoro-2,2-dimethyl-2,3-dihydrobenzo[b]thiophene 1,1-dioxide 36

The 5-(5-amino-6-(5-(4-((1-methylpiperidin-4-yl)amino)methyl)phenyl)-1,3,4-oxadiazol-2-yl)pyrazin-2-yl)-3-fluoro-2,2-dimethyl-2,3-dihydrobenzo[b]thiophene 1,1-dioxide 36 (18.68 mg) was prepared by 5-bromo-3-(5-(4-((((1-methylpiperidin-4-yl)amino)methyl)phenyl)-1,3,4-oxadiazol-2-yl)pyrazin-2-amine 36b (120 mg, 0.27 mmol), 3-fluoro-2,2-dimethyl-5-(4,4,5,5-tetramethyl-1,3,2-dioxaborolan-2-yl)-2,3-dihydrobenzo[b]thiophene-1,1-dioxide 32h (96 mg, 0.28 mmol), potassium carbonate (112 mg, 0.81 mmol), and [1,1′-bis(diphenylphosphino)ferrocene]palladium(II) dichloride (20 mg, 0.03 mmol) using a method similar to step four of Example 27.

¹H NMR (400 MHz, DMSO-d₆): δ 9.10 (s, 1H), 8.55-8.52 (m, 2H), 8.10-8.04 (m, 3H), 7.65-7.63 (m, 2H), 6.11 (s, 0.5H), 5.98 (s, 0.5H), 3.84 (s, 2H), 2.70-2.67 (m, 3H), 2.34-2.32 (m, 2H), 2.15 (s, 3H), 1.83-1.87 (m, 4H), 1.50 (m, 3H), 1.38 (s, 3H)

MS measured value (ESI⁺) [(M+H)J]: 578

### Example 37

### 5-(5-amino-6-(5-(4-((methylamino)methyl)phenyl)-1,3,4-thiadiazol-2-yl)pyrazin-2-yl)-3-fluoro-2,2-dimethyl-2,3-dihydrobenzo[b]thiophene 1,1-dioxide 37

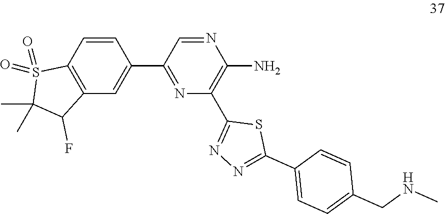

### Step One

### tert-butyl (4-(hydrazinocarbonyl)benzyl)(methyl)carbamate 37a

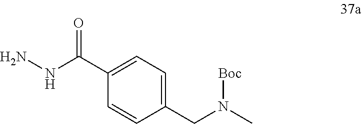 [SMILES: CN(Cc1ccc(C(=O)NN)cc1)C(=O)OC(C)(C)C]

To a solution of methyl 4-(((tert-butoxycarbonyl)(methyl)amino)methyl)benzoate (3.0 g, 10.75 mmol) in ethanol (15 mL) was added hydrazine hydrate (1.57 mL, 32.24 mmol) at room temperature. The reaction mixture was heated to 80° C. and stirred for 2 h. The reaction solution was cooled, concentrated and column chromatographed to give a light yellow oil tert-butyl (4-(hydrazinocarbonyl)benzyl)(methyl)carbamate 37a (2.7 g).

### Step Two

### tert-butyl 2-(4-(((tert-butoxycarbonyl)(methyl)amino)methyl)benzoyl)hydrazine-1-carboxylate 37b

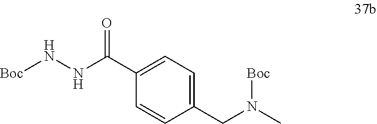 [SMILES: CN(Cc1ccc(C(=O)NNC(=O)OC(C)(C)C)cc1)C(=O)OC(C)(C)C]

At room temperature, tert-butyl (4-(hydrazinocarbonyl)benzyl)(methyl)carbamate 37a (2.5 g, 8.9 mmol) was dissolved in dichloromethane (20 mL). Under nitrogen protection, di-tert-butyl dicarbonate (3.85 g, 17.83 mmol) was added dropwise at room temperature, and then the solution was stirred for 12 hours. After the reaction, water (20 mL) and ethyl acetate (20 mL) were added to the reaction system, and the organic phase was separated out. The aqueous phase was extracted with ethyl acetate (50 mL) three times, and the combined organic phase was dried with anhydrous sodium sulfate and filtered. The filtrate was concentrated under reduced pressure and column chromatographed to give a colorless oil tert-butyl 2-(4-(((tert-butoxycarbonyl)(methyl)amino)methyl)benzoyl)hydrazine-1-carboxylate 37b (2.5 g).

### Step Three

### tert-butyl 2-(4-(((tert-butoxycarbonyl)(methyl)aamino)methyl)phenylthiocarbonyl)hydrazine-1-carboxylate 37c

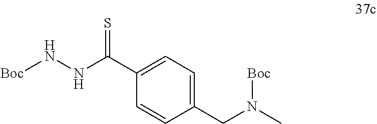 [SMILES: CN(Cc1ccc(C(=S)NNC(=O)OC(C)(C)C)cc1)C(=O)OC(C)(C)C]

The tert-butyl 2-(4-(((tert-butoxycarbonyl)(methyl)amino)methyl)benzoyl)hydrazine-1-carboxylate 37b (2.5 g, 6.25 mmol) and Lawson's reagent (10.1 g, 25.12 mmol) were dissolved in toluene (15 mL) at 0° C. The solution was stirred at 100° C. for 12 h under nitrogen protection. After the reaction, water (10 mL) was added to quench the reaction. Then, water (50 mL) and ethyl acetate (50 mL) were added, and the organic phase was separated out. The aqueous phase was extracted three times with ethyl acetate (50 mL), and the combined organic phase was dried with anhydrous sodium sulfate and filtered. The filtrate was concentrated under reduced pressure and column chromatographed to give the yellow solid tert-butyl 2-(4-(((tert-butoxycarbonyl)(methyl)amino)methyl)phenylthiocarbonyl)hydrazine-1-carboxylate 37c (2 g).

### Step Four

### 4-((methylamino)methyl)benzothiohydrazide 37d

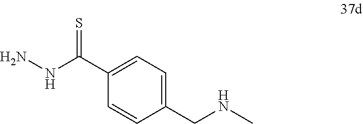

The tert-butyl 2-(4-(((tert-butoxycarbonyl)(methyl)amino)methyl)phenylthiocarbonyl)hydrazine-1-carboxylate 37c (2.0 g, 5.031 mmol) was dissolved in MeOH (10 mL) at room temperature, and then a mixed solution (4 M, 2 mL) of hydrochloric acid and ethyl acetate was added. The reaction solution was stirred under nitrogen protection at 25° C. for 2 hours. After the reaction, the reaction solution was concentrated under reduced pressure to obtain a yellow oil 4-((methylamino)methyl)benzothiohydrazide 37d (650 mg) that was a crude product and used directly in the next step.

### Step Five

### 5-bromo-3-(5-(4-((methylamino)methyl)phenyl)-1,3,4-thiadiazol-2-yl)pyrazin-2-amine 37e

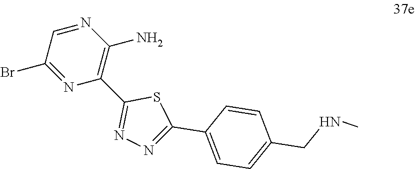 [SMILES: CNCc1ccc(-c2nnc(-c3nc(Br)cnc3N)s2)cc1]

To a solution of 4-((methylamino)methyl)benzothiohydrazide 37d (500 mg, 2.56 mmol) in anhydrous acetonitrile (10 mL) was added diisopropylethylamine (1.985 g, 15.36 mmol) at room temperature. Dibromotriphenylphosphine (4.860 g, 10.24 mmol) was slowly added to the mixed solution which was then stirred at room temperature for 48 hours. Water (50 mL) and ethyl acetate (50 mL) were added to the reaction system, and the organic phase was separated out. The aqueous phase was extracted three times with ethyl acetate (50 mL), and the combined organic phase was dried with anhydrous sodium sulfate and filtered. The filtrate was concentrated under reduced pressure and column chromatographed to give a yellow solid 5-bromo-3-(5-(4-((methylamino)methyl)phenyl)-1,3,4-thiadiazol-2-yl)pyrazin-2-amine 37e (200 mg).

### Step Six

### 5-(5-amino-6-(5-(4-((methylamino)methyl)phenyl)-1,3,4-thiadiazol-2-yl)pyrazin-2-yl)-3-fluoro-2,2-dimethyl-2,3-dihydrobenzo[b]thiophene 1,1-dioxide 37

The mixture of 5-bromo-3-(5-(4-((methylamino)methyl)phenyl)-1,3,4-thiadiazol-2-yl)pyrazin-2-amine 37e (200 mg, 0.53 mmol), 3-fluoro-2,2-dimethyl-5-(4,4,5,5-tetramethyl-1,3,2-dioxaborolan-2-yl)-2,3-dihydrobenzo[b]thiophene-1,1-dioxide 32h (216 mg, 0.64 mmol), sodium carbonate (168 mg, 1.59 mmol) and tetrakis(triphenylphosphine)palladium (61 mg, 0.053 mmol) reacted in microwave at 150° C. for 30 mins. After the reaction, it was spun-dried and purified by column chromatography to give a yellow solid 5-(5-amino-6-(5-(4-((methylamino)methyl)phenyl)-1,3,4-thiadiazol-2-yl)pyrazin-2-yl)-3-fluoro-2,2-dimethyl-2,3-dihydrobenzo[b]thiophene 1,1-dioxide 37 (20.26 mg).

¹H NMR (400 MHz, DMSO-d₆): δ 9.06 (s, 1H), 8.48-8.45 (m, 2H), 8.07-8.05 (m, 5H), 7.56-7.54 (m, 2H), 6.09 (s, 0.5H), 5.96 (s, 0.5H), 3.77 (s, 2H), 2.34 (s, 3H), 1.50-1.49 (m, 3H), 1.38 (s, 3H)

MS measured value (ESI⁺) [(M+H)⁺]: 511

### Example 38-1 or 38-2

### (R)-5-(5-amino-6-(3-(4-((methylamino)methyl)phenyl)isoxazol-5-yl-4-deuterium)pyrazin-2-yl)-3-fluoro-2,2-dimethyl-2,3-dihydrobenzo[b]thiophene 1,1-dioxide 38-1

### or

### (S)-5-(5-amino-6-(3-(4-((methylamino)methyl)phenyl)isoxazol-5-yl-4-deuterium)pyrazin-2-yl)-3-fluoro-2,2-dimethyl-2,3-dihydrobenzo[b]thiophene 1,1-dioxide 38-2

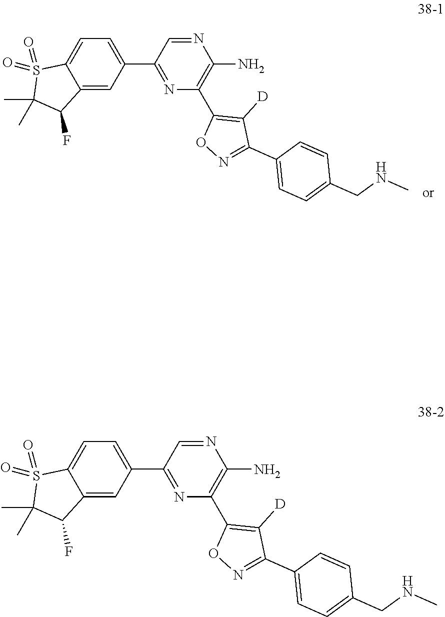

### Step One

### tert-butyl (5-bromo-3-(deuteroethynyl)pyrazin-2-yl)carbamate 38a

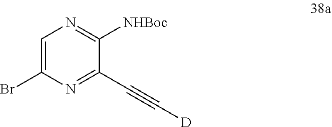 [SMILES: [2H]C#Cc1nc(Br)cnc1NC(=O)OC(C)(C)C]

Tert-butyl (5-bromo-3-ethynyl-2-pyrazinyl)(tert-butoxycarbonyl)carbamate (1 g, 3.3 mmol) was dissolved in deuterated methanol solution (10 mL), and the mixture was heated to 70° C. and stirred under nitrogen protection for 48 hours. After the reaction, the reaction solution was directly spun-dried to give the brown solid tert-butyl (5-bromo-3-(deuteroethynyl)pyrazin-2-yl)carbamate 38a (0.7 g) that was a crude product and used in the next step without a further purification.

### Step Two

### tert-butyl (4-(5-(6-bromo-3-((tert-butoxycarbonyl)amino)pyrazin-2-yl)isoxazol-3-yl-4-deuterium)benzyl)(methyl) carbamate 38b

To a mixed solution of tert-butyl (5-bromo-3-(deuteroethynyl)pyrazin-2-yl)carbamate 38a (500 mg, 1.25 mmol) in methanol (12 mL) and water (2.4 mL) was sequentially added tert-butyl (E)-(4-((hydroxyiminomethyl)methyl)benzyl) (methyl) carbamate (332 mg, 1.25 mmol) and [bis(trifluoroacetoxy)iodo]benzene (646.3 mg, 1.50 mmol). The reaction solution was stirred overnight at room temperature and then was added with ethyl acetate (40 mL) and water (40 mL). The organic phase was washed three times with water (20 mL). The organic phase was separated out, dried with anhydrous sodium sulfate, filtered, concentrated and isolated by column chromatography to give tert-butyl (4-(5-(6-bromo-3-((tert-butoxycarbonyl)amino)pyrazin-2-yl)isoxazol-3-yl-4-deuterium)benzyl)(methyl) carbamate 38b (800 mg).

### Step Three

### tert-butyl (4-(5-(3-((tert-butoxycarbonyl)amino)-6-(4,4,5,5-tetramethyl-1,3,2-dioxobenzaldehyde-2-yl)pyrazin-2-yl)isoxazol-3-yl-4-deuterium) benzyl) meth y) carbamate 38c

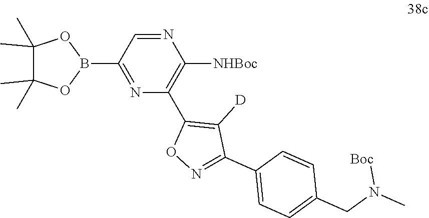

To a solution of tert-butyl (4-(5-(6-bromo-3-((tert-butoxycarbonyl)amino)pyrazin-2-yl)isoxazol-3-yl-4-deuterium) benzyl)(methyl) carbamate 38b (100 mg, 0.18 mmol) in ethylene glycol dimethyl ether solution (2 mL) was added bis(pinacolato)diboron (54 mg, 0.21 mmol), potassium acetate (52 mg, 0.53 mmol) and [1,1′-bis(diphenylphosphino) ferrocene] palladium(II) dichloride (13 mg, 0.02 mmol) at room temperature. The reaction mixture was stirred under nitrogen protection at 60° C. for 3 hrs. Water (10 mL) was added to the reaction system, and the organic phase was separated out. The aqueous phase was extracted with ethyl acetate (10 mL) for three times, and the combined organic phase was dried with anhydrous sodium sulfate and filtered. The filtrate was concentrated under reduced pressure to give the black oil tert-butyl (4-(5-(3-((tert-butoxycarbonyl)amino)-6-(4,4,5,5-tetramethyl-1,3,2-dioxobenzaldehyde-2-yl)pyrazin-2-yl)isoxazol-3-yl-4-deuterium) benzyl)(methyl) carbamate 38c (180 mg) that was a crude product and used directly in the next step without a further purification.

### Step Four

### tert-butyl (R)-(4-(5-(3-((tert-butoxycarbonyl)amino)-6-(3-fluoro-2,2-dimethyl-1,1-dioxo-2,3-dihydrobenzo[b]thiophen-5-yl)pyrazin-2-yl)isoxazol-3-yl-4-deuterium)benzyl(methyl)carbamate 38d-1

### or

### tert-butyl (S)-(4-(5-(3-((tert-butoxycarbonyl)amino)-6-(3-fluoro-2,2-dimethyl-1,1-dioxo-2,3-dihydrobenzo[b]thiophen-5-yl)pyrazin-2-yl)isoxazol-3-yl-4-deuterium)benzyl(methyl)carbamate 38d-2

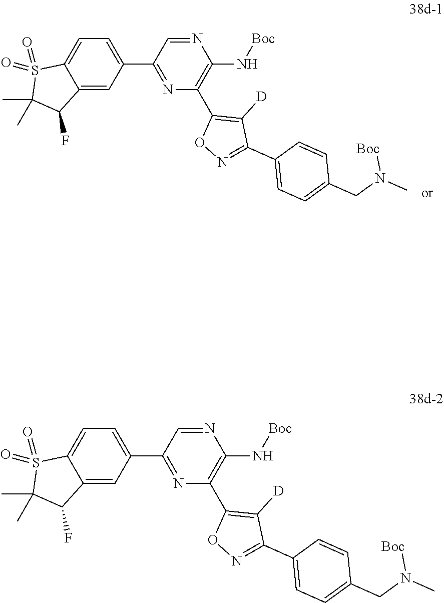

To a solution of tert-butyl (4-(5-(3-((tert-butoxycarbonyl)amino)-6-(4,4,5,5-tetramethyl-1,3,2-dioxobenzaldehyde-2-yl)pyrazin-2-yl)isoxazol-3-yl-4-deuterium) benzyl)(methyl) carbamate 38c (180 mg, 0.34 mmol) in 1,4 dioxane (3 mL) and water (0.6 mL) were added 33a-1 or 33a-2 (105 mg, 0.36 mmol), potassium carbonate (142 mg, 1.02 mmol) and [1,1′-bis(diphenylphosphino)ferrocene] palladium (II) dichloride (25 mg, 0.03 mmol). The reaction mixture was stirred under nitrogen protection at 100° C. for 3 hrs. Water (20 mL) and ethyl acetate (20 mL) were added to the reaction system, and the organic phase was separated out. The aqueous phase was extracted three times with ethyl acetate (20 mL), and the combined organic phase was dried with anhydrous sodium sulfate and filtered. The filtrate was concentrated under reduced pressure and column chromatographed to give the brown oil substance 38d-1 or 38d-2 (120 mg).

### Step Five

### (R)-5-(5-amino-6-(3-(4-((methylamino)methyl)phenyl)isoxazol-5-yl-4-deuterium)pyrazin-2-yl)-3-fluoro-2,2-dimethyl-2,3-dihydrobenzo[b]thiophene 1,1-dioxide 38-1

### or

### (S)-5-(5-amino-6-(3-(4-((methylamino)methyl)phenyl)isoxazol-5-yl-4-deuterium)pyrazin-2-yl)-3-fluoro-2,2-dimethyl-2,3-dihydrobenzo[b]thiophene 1,1-dioxide 38-2

The yellow solid title compound 38-1 or 38-2 (66.12 mg) was prepared by compound 38d-1 or 38d-2 (120 mg, 0.17 mmol) and the mixed solution (4 M, 2 mL) of hydrochloric acid and ethyl acetate using a method similar to step six of Example 1.

¹H NMR (400 MHz, DMSO-d₆): δ 8.99 (s, 1H), 8.53-8.51 (m, 2H), 8.01-7.99 (m, 3H), 7.56-7.54 (m, 2H), 7.24 (s, 2H), 6.06 (s, 0.5H), 5.93 (s, 0.5H), 3.84 (s, 2H), 2.37 (s, 3H), 1.50-1.49 (m, 3H), 1.37 (s, 3H)

MS measured value (ESI⁺) [(M+H)⁺]: 495

### Biological Evaluation

Biological implementation data is described in detail below to further elaborate the technical aspects of the present disclosure.

### Test Example 1: ATR Enzyme Activity Inhibition Assay

The inhibitory ability to ATR kinase for selected compounds was tested by the HTRF (Homogeneous Time-Resolved Fluorescence). In the reaction system, After the substrate p53 is phosphorylated by ATR, the anti-phospho-p53-Eu that serves as an energy donor and specifically binds to the phosphorylation site on p53 is added. Then, the anti-GST-d2 as an energy acceptor that specifically binds to the GST tag carried on p53 is added. Due to the excitation of a certain wavelength of laser (340 nm), the energy donor emits the emission light at a wavelength of 615 nm. Due to the occurrence of energy transfer between the donor and the acceptor, the energy acceptor emits the emission light at a wavelength of 665 nm. A plate reader is used to detect the two emission lights and the ratio of the two signals of 665 nm and 615 nm is calculated. The IC50 of the sample to be tested can be obtained through graphing and calculation.

Prepare various buffer systems required for the experiment:
1. Prepare ATR reaction buffer containing 25 mM HEPES (Gibco, Cat #15630-080), 5 mM DTT (Sigma, Cat #D0632-10G), 10 mM MnCl2 (Sigma, Cat #7773-01-5), 5 mM DTT (Sigma, Cat #D0632-10G), 1 mg/mL of BSA (Sigma, Cat #B2064-50G), 0.01% Brij35 (Sigma, Cat #9002-92-0), 1% Glycerol (Sigma, Cat #G5516-500 ML), and H₂O. The full-length ATR enzyme (eurofins, Cat #14-953M) was diluted to 60 nM using the prepared ATR buffer.
2. Prepare reaction substrate containing 80 nM p53 (eurofins, Cat #14-952M) and 300 nM ATP (Sigma, Cat #R0441).
3. Prepare detection buffer: Dilute each of the anti-phospho-p53-Eu cryptate (Cisbio, Cat #61P08KA) and anti-GST-d2 (Cisbio, Cat #61GSTDLB) to 1 unit using HTRF detection buffer (Cisbio, Cat #62SDBRDF).

Powdered compounds were dissolved in DMSO to from a stock solution at a concentration of 10 mM. The solution of compounds to be tested at a concentration of 1M was performed a 3-fold dilution for 10 concentrations with DMSO. Then, the diluted solution was added to 384-well plate at 10.05 μL per well (containing 0.498% DMSO). 5 μL of ATR was added to each well accordingly. Each well is incubated at 25° C. for 10 min, added with 5 μL of reaction substrate, incubated at 25° C. for 90 min and added with 10 μL of detection solution. The reaction was carried out overnight, and the data at 665/615 nm were read by an Envision 2104 Multilabel Reader. The inhibition curves of the compounds were made by the four-parameter Rogers regression equation of XLfit software, and the inhibition rates of the compounds were calculated. Table 1 below shows the relative inhibition values of the disclosed compounds in present application against ATR enzymes:

TABLE 1
Relative inhibition values of compounds on ATRase

| Example No. | IC₅₀ (nM) |
|---|---|
| Example 1 | 0.97 |
| Example 2 | 2.79 |
| Example 3 | 1.60 |
| Example 4 | 0.83 |
| Example 5 | 2.32 |
| Example 6 | 2.90 |
| Example 7 | 1.04 |
| Example 8 | 3.64 |
| Example 9 | 1.83 |
| Example 10 | 1.75 |
| Example 11 | 0.82 |
| Example 12 | 2.53 |
| Example 13 | 1.68 |
| Example 14 | 1.42 |
| Example 15 | 2.00 |
| Example 16 | 9.74 |
| Example 17 | 0.93 |
| Example 18 | 1.16 |
| Example 19 | 1.26 |
| Example 20 | 1.81 |
| Example 21 | 1.31 |
| Example 22 | 1.21 |
| Example 23 | 0.70 |
| Example 24 | 1.08 |
| Example 25 | 1.98 |
| Example 26 | 2.06 |
| Example 27 | 0.75 |
| Example 28 | 1.29 |
| Example 29 | 3.12 |
| Example 30 | 3.35 |
| Example 31 | 4.00 |
| Example 32 | 2.09 |
| Example 33-1 or 33-2 | 0.83 |
| Example 34 | 0.92 |
| Example 35-1 or 35-2 | 0.41 |
| Example 36 | 0.57 |
| Example 37 | 0.77 |
| Example 38-1 or 38-2 | 0.75 |
| VX-970 (Berzosertib) | 1.94 |

CONCLUSION: Analyzing the above example data, the inhibitory ability of the compounds on ATR is comparable or superior to that of VX-970.

### Test Example 2: In Vivo Pharmacodynamics of Tested Compounds in the NCI-H23 Subcutaneous Graft Tumor Model

### 1. Experimental Methods:

1, cell culture: human lung cancer NCI-H23 cells (ATCC, Item No.: CRL-5800) are performed with an in vitro monolayer culture. The culture conditions are the culture in the RPMI 1640 medium added with 1000 fetal bovine serum, 1% penicillin/streptomycin/amphotericin B, and the incubation at 37° C. in a 500 CO₂ incubator. Trypsin-EDTA was used for conventional digestion and treatment of passaging twice a week. When the cell saturation is 80%-0% and the number meets the requirement, the cells are collected, counted and inoculated.
2, Animals: CB.17/SCID mouse, female, 6-8 weeks old, weighing 18-22 grams. A total of 84 mice are needed, which are provided by Beijing Viton Lihua Laboratory Animal Technology Co., Ltd. or equivalent qualified suppliers.
3. The test compound information is shown in Table 2 below:

TABLE 2
Test compound information

| Test compound | Item No./Purity |
|---|---|
| Cisplatin | TCI/D3371-100 MG |
| VX-970 | 99.68% |
| Example 33-1 or 33-2 | 99.73% |
| Example 38-1 or 38-2 | 99.12% |

4. Tumor inoculation: 0.2 mL (10×10⁶ cells) of NCI-H23 cells (plus stromal gel, volume ratio 1:1) were subcutaneously inoculated into the right back of each mouse. Mice are grouped for drug administration when the average tumor volume reached about 200 mm³. The experimental grouping and dosing regimen are shown in Table 3 below. Animals were weighed before dosing and tumor volumes were measured. Groups were randomized according to tumor volume (randomized area group design).

TABLE 3
Animal experimental grouping and drug administration

| | Mice | Compound | | Dosing | | |
|---|---|---|---|---|---|---|
| | num- | for | Dose | volume | Dosing | Dosing |
| Group | ber | treatment | (mg/kg) | (μL/g)² | route | frequency |
| 1 | 7 | Vehicle | — | 10 | PO | QD*2 weeks |
| 2 | 7 | Cisplatin + | 1.5 + 12.5 | 10 | IP + PO | (QW + |
| | | VX-970 | | | | QD)*2 |
| | | | | | | weeks |
| 3 | 7 | Cisplatin + | 1.5 + 12.5 | 10 | IP + PO | (QW + |
| | | Example | | | | QD)*2 |
| | | 33-1 or 33-2 | | | | weeks |
| 4 | 7 | Cisplatin + | 1.5 + 12.5 | 10 | IP + PO | (QW + |
| | | Example | | | | QD)*2 |
| | | 38-1 or | | | | weeks |
| | | 38-2 | | | | |

### II. Experimental Indicators:

1 [symbol] The experimental index is to examine whether the tumor growth is inhibited, delayed or cured. Tumor diameter was measured three times a week with vernier calipers.
2 [symbol] Tumor volume was calculated as V=0.5a×b², with a and b indicating the long and short diameters of the tumor, respectively.
3 [symbol] Tumor inhibitory efficacy of compounds was evaluated by TGI (%) and Tumor change (%).
4 [symbol] TGI (%) reflects the tumor growth inhibition rate. Calculation of TGI (%): TGI (%)=[1−(average tumor volume at the end of the administration of a treatment group−average tumor volume at the beginning of the administration of this treatment group)/(average tumor volume at the end of the treatment of the solvent control group−average tumor volume at the beginning of the treatment of the solvent control group)]×100%. TGI (%): greater than 100, non-growth of the tumor in the mice after the drug administration, and the existence of inhibition.
5. Calculation of Tumor change (%): Tumor change (%)=(average tumor volume at the end of administration of a treatment group−average tumor volume at the beginning of administration of a treatment group)/average tumor volume at the beginning of administration of a treatment group×100%. Tumor change (%): less than 0, non-growth of the tumor in the mice after the drug administration, and the existence of inhibition.
6. Data analysis: T-test was used for comparison between two groups. p<0.05 was considered to be significantly different.

### III. Experimental Results:

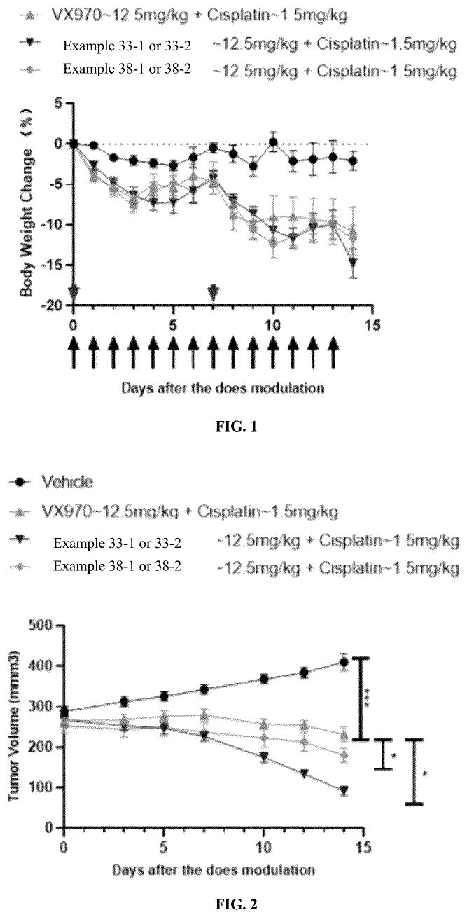

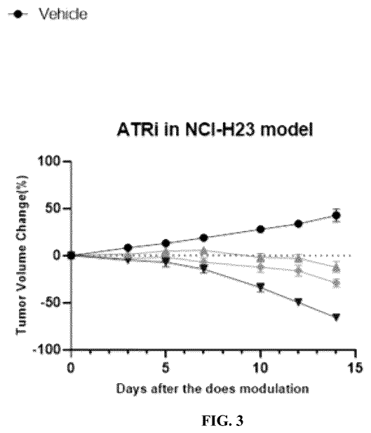

After drug administration, the mice in the Cisplatin+VX-970 group, the Cisplatin+ Example 33-1 or 33-2 group and the Cisplatin+ Example 38-1 or 38-2 group had a decrease in body weight, but there was no difference in body weight loss between the groups, as shown in FIG. 1 (Note: in FIG. 1, the lower black arrows means administration of the ATR kinase inhibitor, and the upper gray arrows means administration of cisplatin). Tumor volume tended to decrease in all groups after drug administration compared to the tumor volume before drug administration. As shown in FIGS. 2 and 3, compared to the Cisplatin+VX-970 group, Cisplatin+ Example 33-1 or 33-2 group and the Cisplatin+ Example 38-1 or 38-2 group show a more obvious decrease in tumor volume with p<0.05 in T-test, suggesting that the combination of Cisplatin with Example 33-1 or 33-2 or with Example 38-1 or 38-2 is more effectively than the combination of Cisplatin with VX-970 in tumor growth inhibition.

At 14 days after administration, the Cisplatin+VX-970 group shows a Tumor change (%) of −12% and a TGI (%) of 128%; the Cisplatin+ Example 33-1 or 33-2 group shows a Tumor change (%) of −66% and a TGI (%) of 245%; the Cisplatin+ Example 38-1 or 38-2 group shows a Tumor change (%) of −29% and a TGI (%) of 159% (Table 4).

TABLE 4
TGI(%) and Tumor change(%)

| | Tumor | Tumor | | |
|---|---|---|---|---|
| | volume | volume | Tumor | |
| | (mm³) | (mm³) | change | |
| Group | day 0 | day 14 | (%) | TGI(%) |
| Vehicle | 289 | 410 | 42 | — |
| VX970~12.5 mg/kg + Cisplatin~1.5 mg/kg | 266 | 231 | −13 | 128 |
| Example 33-1 or 33-2~12.5 mg/kg + | 267 | 92 | −66 | 245 |
| Cisplatin~1.5 mg/kg | | | | |
| Example 38-1 or 38-2~12.5 mg/kg + | 252 | 180 | −29 | 159 |
| Cisplatin~1.5 mg/kg | | | | |

It should be understood that the above embodiments are exemplary and are not intended to encompass all possible embodiments encompassed by the claims. Various modifications and changes can also be made based on the above embodiments without departing from the scope of the present disclosure. Similarly, any combination of the various technical features from the above embodiments may be made to form additional embodiments of the present disclosure which may not be expressly described. Therefore, the above embodiments express only several embodiments of the present disclosure and do not limit the protection scope of the present disclosure.

1. A compound of formula I-a: 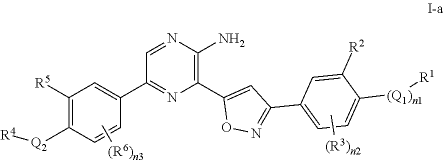
or a pharmaceutically acceptable salt, ester, isomer, solvate, prodrug or isotopically labeled compound thereof,
wherein:
R¹ is selected from hydrogen, deuterium, cyano, halogen, and substituted or unsubstituted hydroxyl, amino, C₁₋₆ alkyl, C₁₋₆ alkoxy, 3- to 7-membered cycloalkyl, 3- to 7-membered heterocycloalkyl, guanidino, ureido, amido, aminosulfonamido, sulfonamido, and HN—C(═NH)—C₁₋₆ alkyl;
R² and R⁵ are each independently selected from hydrogen, deuterium, halogens, C₁₋₆ alkyl, amino and
C₁₋₆ alkoxy;
or R¹ and R² together with the atoms to which they are attached form a 5 to 6-membered heteroaryl or heterocyclic group containing 1 to 3 atoms selected from oxygen, nitrogen and sulphur, said 5 to 6-membered heteroaryl or heterocyclic group having 0 or 1 methylene group on its ring optionally substituted with —C(═O)— or —C(═NR⁷)—;
if present, each R³ is independently selected from hydrogen, deuterium, halogen, C₁₋₆ alkyl and C₁₋₆ alkyl substituted with a substituent selected from cyano, amino, hydroxy, halogen, C₁₋₆ alkyl and C₁₋₆ alkoxy;
R⁴ is selected from hydrogen, deuterium, C₁₋₆ alkyl, 3- to 7-membered cycloalkyl, 3- to 7-membered heterocycloalkyl, C₁₋₆ alkylamino, and (C₁₋₃ alkyl)₂amino;
or R⁴ and R⁵ together with the atoms to which they are attached form a 5- to 7-membered heterocyclic group containing 1 to 3 atoms selected from oxygen, nitrogen and sulphur, said 5- to 7-membered heterocyclic group optionally substituted with a substituent selected from deuterium, hydroxyl, C₁₋₆ alkyl, halogen, C₁₋₆ alkoxy and C₁₋₆ alkylamino; said heterocyclic group having 1 to 3 methylene groups on its ring optionally substituted by —SO₂—, —SO(═NR⁷)—, —SO₂NH—, —CO—, —C(═O)NH—, C₁₋₆ alkylamino, or 3- to 6-membered cycloalkylamino; wherein said C₁₋₆ alkylamino or 3- to 6-membered cycloalkylamino has 0 to 2 methylene groups optionally substituted with oxygen, nitrogen, sulfur, or C₁₋₃ alkylamino;
if present, each R⁶ is independently selected from hydrogen, deuterium, halogen, amino, C₁₋₆ alkoxy, C₁₋₆ alkyl and C₁₋₆ alkyl substituted with cyano, amino, hydroxy, halogen, C₁₋₆ alkyl or C₁₋₆ alkoxy;
Q¹ is selected from C₁₋₆ alkylene, —CO—, amino, —SO—, —SO₂—, —C(═NR⁷)—, and 3- to 7-membered heterocyclic group, wherein said C₁₋₆ alkylene has 0 to 4 methylene groups optionally substituted with amino, —CO—, —SO—, —SO₂ or —C(═NR⁷)—;
Q² is selected from —SO₂—, SO, —SO(═NR⁷)—, —SO₂NR⁷— and —CO—;
each R⁷ is independently selected from hydrogen, deuterium, C₁₋₆ alkyl, cyano, 3- to 7-membered cycloalkyl, 3- to 7-membered heterocyclic group, C₁₋₆ alkoxy, C₁₋₆ alkylamino, and C₁₋₆ alkyl, 3- to 7-membered cycloalkyl and 3- to 7-membered heterocyclic group substituted by a substituent selected from cyano, amino, hydroxyl, halogen, C₁₋₆ alkyl, and C₁₋₆ alkoxy; and
each of n1, n2 and n3 is any integer from 0 to 2.

2. The compound of claim 1, or a pharmaceutically acceptable salt, ester, isomer, solvate, prodrug or isotopically labeled compound thereof, wherein:
n1 is 0 or 1; n2 is 1 or 2; and n3 is preferably 0 or 1.

3. (canceled)

4. (canceled)

5. The compound of claim 1, or a pharmaceutically acceptable salt, ester, isomer, solvate, prodrug or isotopically labeled compound thereof, wherein:
R¹ is selected from hydrogen, deuterium, cyano, halogen, and substituted or unsubstituted hydroxyl, amino, C₁₋₆ alkyl, C₁₋₆ alkoxy, 3- to 7-membered cycloalkyl, 3- to 7-membered heterocyclic group, guanidino, ureido, amide, aminosulfonamido, sulfonamido and HN—C(═NH)—C₁₋₆ alkyl; wherein said substitution is substituted by a substituent selected from C₁₋₆ alkyl or 3- to 7-membered cycloalkyl; and/or
R² and R⁵ are each independently hydrogen or deuterium.

6. (canceled)

7. The compound of claim 1, or a pharmaceutically acceptable salt, ester, isomer, solvate, prodrug or isotopically labeled compound thereof, wherein:
R¹ and R², together with the atoms to which they are attached, form a 5- to 6-membered heteroaryl or heterocyclic group containing 1 to 3 atoms selected from oxygen, nitrogen and sulphur, said 5- to 6-membered heterocyclic group having 0 or 1 methylene group on its ring optionally substituted by —C(═O)— or —C(═NR⁷)—; wherein said 5- to 6-membered heteroaryl or 5- to 6-membered heterocyclic group is any of the following structures:

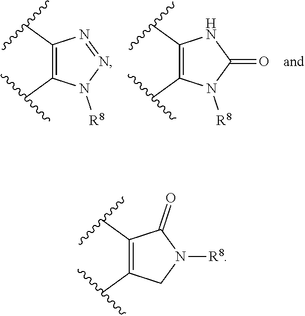

8. The compound of claim 1, or a pharmaceutically acceptable salt, ester, isomer, solvate, prodrug or isotopically labeled compound thereof, wherein:
R³ is halogen or halogen-substituted C₁₋₆ alkyl, preferably F or CF₃; and/or
each R⁶ is independently selected from hydrogen, deuterium and fluorine.

9. The compound of claim 1, or a pharmaceutically acceptable salt, ester, isomer, solvate, prodrug or isotopically labeled compound thereof, wherein:
R⁴ is methyl or any of the following structures:

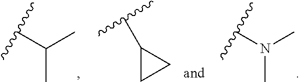

10. The compound of claim 1, or a pharmaceutically acceptable salt, ester, isomer, solvate, prodrug or isotopically labeled compound thereof, wherein:
R⁴ and R⁵ together with the atoms to which they are attached form a 5- to 7-membered heterocyclic group containing 1 to 3 atoms selected from oxygen, nitrogen and sulphur, said 5- to 7-membered heterocyclic group preferably being any one of the following structures: 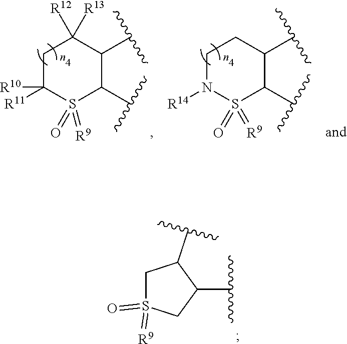
n⁴ is selected from an integer from 0 to 2;
R⁹ is selected from oxygen and NR⁷;
R¹⁰ and R¹¹ are each independently selected from hydrogen, deuterium, C₁₋₆ alkyl, C₁₋₆ alkoxy and C₁₋₆ alkylamino; or R¹⁰ and R¹¹ together with the atoms to which they are attached form a 3 to 7-membered cycloalkyl group or a 3 to 7-membered heterocyclic group containing 1 or 2 atoms selected from oxygen, nitrogen and sulfur;
R¹² and R¹³ are each independently selected from hydrogen, deuterium, halogen, hydroxyl, C₁₋₆ alkyl, C₁₋₆ alkoxy and C₁₋₆ alkylamino; or R¹² and R¹³ form ═O;
R¹⁴ is selected from hydrogen, deuterium, substituted or unsubstituted C₁₋₆ alkyl, C₁₋₆ alkoxy, 3- to 7-membered cycloalkyl and 3- to 7-membered heterocyclic group, wherein said substitution is substituted by a substituent selected from halogen, hydroxyl, amino, C₁₋₆ alkylamino, and (C₁₋₆ alkyl)₂ amino.

11. (canceled)

12. The compound of claim 1, or a pharmaceutically acceptable salt, ester, isomer, solvate, prodrug or isotopically labeled compound thereof, wherein:
Q¹ is selected from C₁₋₆ alkylene, —CO—, amino, —SO—, —SO₂—, —C(═NR⁷)— or is any one of the following structures:

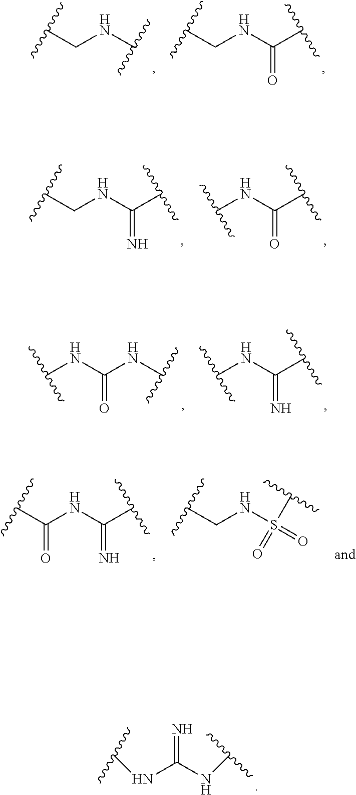

13. The compound of claim 1, or a pharmaceutically acceptable salt, ester, isomer, solvate, prodrug or isotopically labeled compound thereof, wherein:
Q² is selected from —SO₂—, —SO(═NH)—, —SO(═NMe)- and —CO—; and/or
each R⁷ is independently selected from hydrogen, deuterium, methyl and cyano.

14. (canceled)

15. The compound of claim 10, or a pharmaceutically acceptable salt, ester, isomer, solvate, prodrug or isotopically labeled compound thereof, wherein:
n4 is 0 or 1; and/or
R⁹ is selected from oxygen and NH.

16. (canceled)

17. The compound of claim 10, or a pharmaceutically acceptable salt, ester, isomer, solvate, prodrug or isotopically labeled compound thereof, wherein:
R¹⁰ and R¹¹ are each independently selected from hydrogen and methyl.

18. The compound of claim 10, or a pharmaceutically acceptable salt, ester, isomer, solvate, prodrug or isotopically labeled compound thereof, wherein:
R¹⁰ and R¹¹ together with atoms to which they are attached form a 3 to 7-membered cycloalkyl or a 3 to 7-membered heterocyclic group containing 1 or 2 atoms selected from oxygen, nitrogen and sulphur, and said 3 to 7-membered cycloalkyl is cyclopropyl.

19. The compound of claim 10, or a pharmaceutically acceptable salt, ester, isomer, solvate, prodrug or isotopically labeled compound thereof, wherein:
R¹² and R¹³ are each independently selected from hydrogen, deuterium, methyl, fluorine, and hydroxyl.

20. The compound of claim 10, or a pharmaceutically acceptable salt, ester, isomer, solvate, prodrug or isotopically labeled compound thereof, wherein:
R¹⁴ is selected from hydrogen, methyl, ethyl, isopropyl, cyclopropyl, hydroxyethyl and

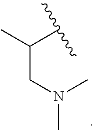 [SMILES: *C(C)CN(C)C]

21. A compound, or a pharmaceutically acceptable salt, ester, isomer, solvate, prodrug or isotopically labeled compound thereof, wherein the compound is selected from:

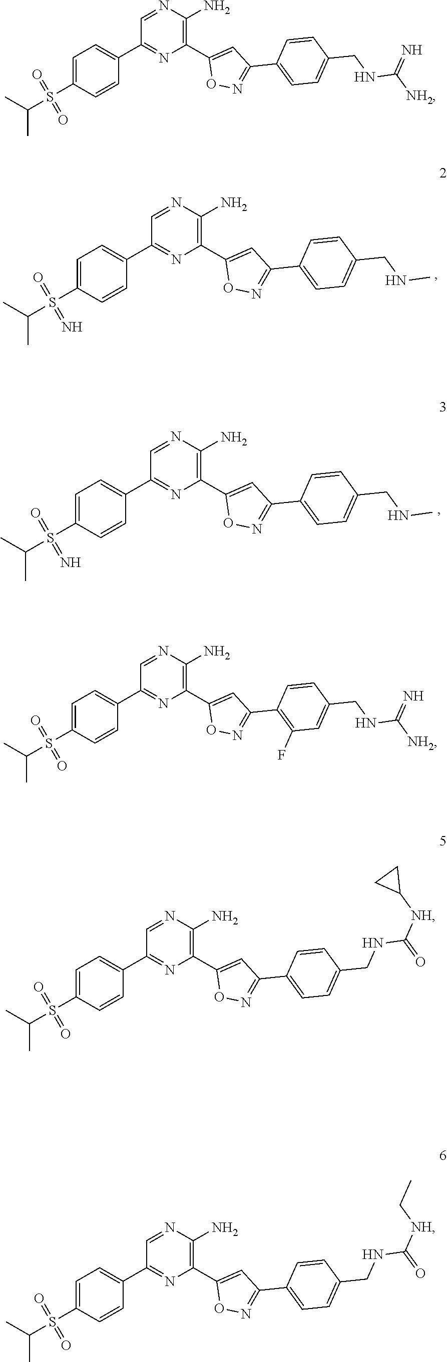

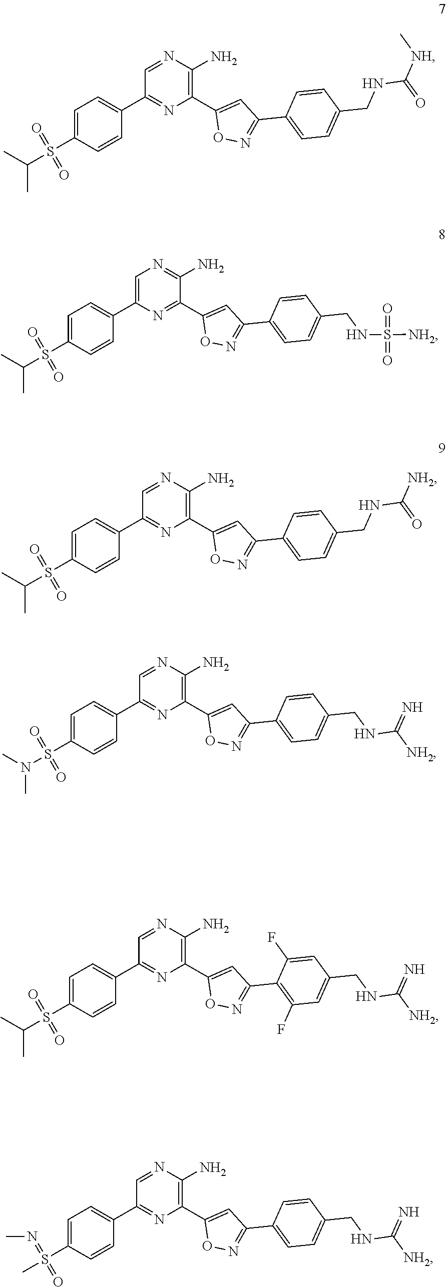 [SMILES: CNC(=O)NCc1ccc(-c2cc(-c3nc(-c4ccc(S(=O)(=O)C(C)C)cc4)cnc3N)on2)cc1 ; CC(C)S(=O)(=O)c1ccc(-c2cnc(N)c(-c3cc(-c4ccc(CNS(N)(=O)=O)cc4)no3)n2)cc1 ; CC(C)S(=O)(=O)c1ccc(-c2cnc(N)c(-c3cc(-c4ccc(CNC(N)=O)cc4)no3)n2)cc1 ; CN(C)S(=O)(=O)c1ccc(-c2cnc(N)c(-c3cc(-c4ccc(CNC(=N)N)cc4)no3)n2)cc1 ; CC(C)S(=O)(=O)c1ccc(-c2cnc(N)c(-c3cc(-c4c(F)cc(CNC(=N)N)cc4F)no3)n2)cc1 ; CN=S(C)(=O)c1ccc(-c2cnc(N)c(-c3cc(-c4ccc(CNC(=N)N)cc4)no3)n2)cc1]

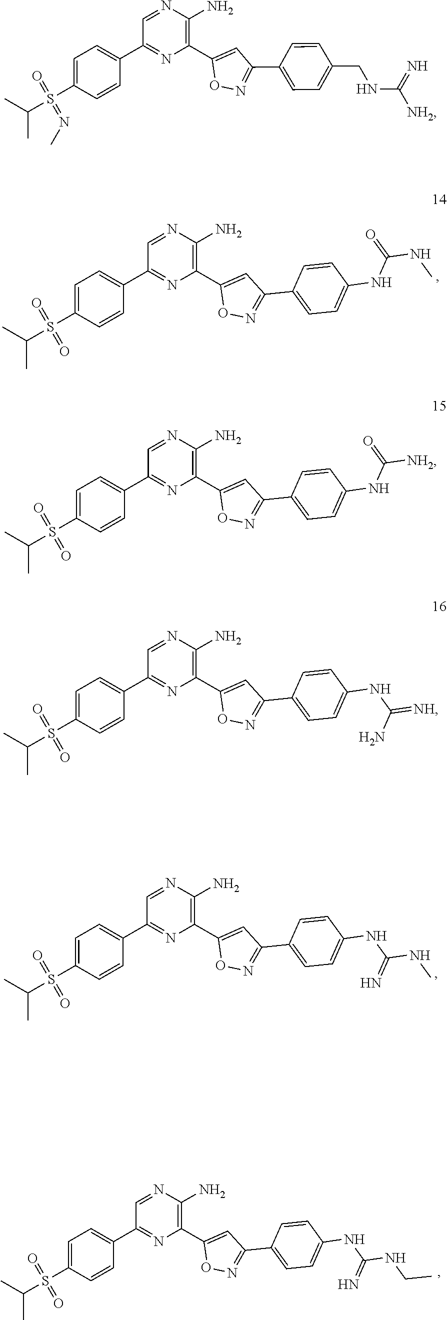

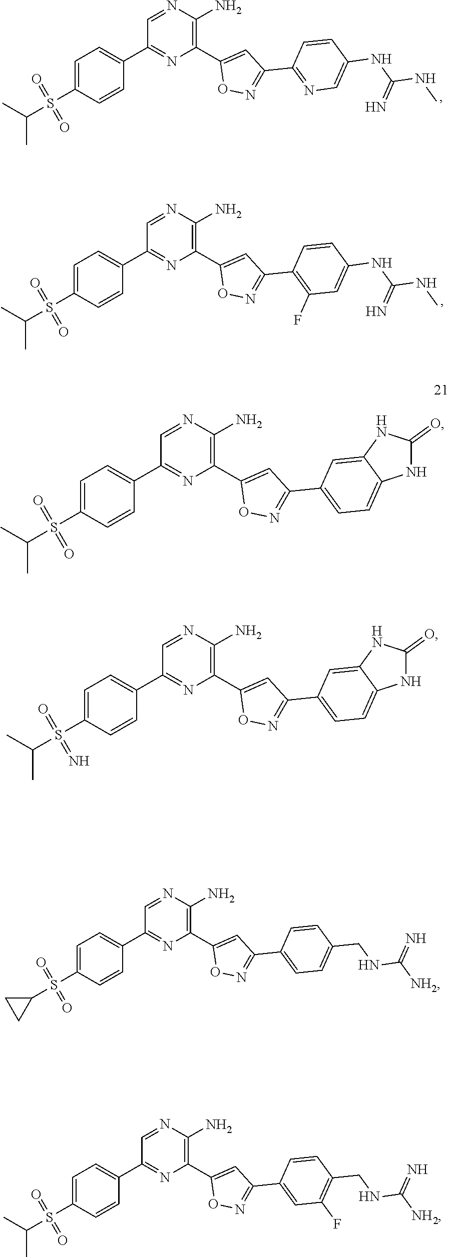

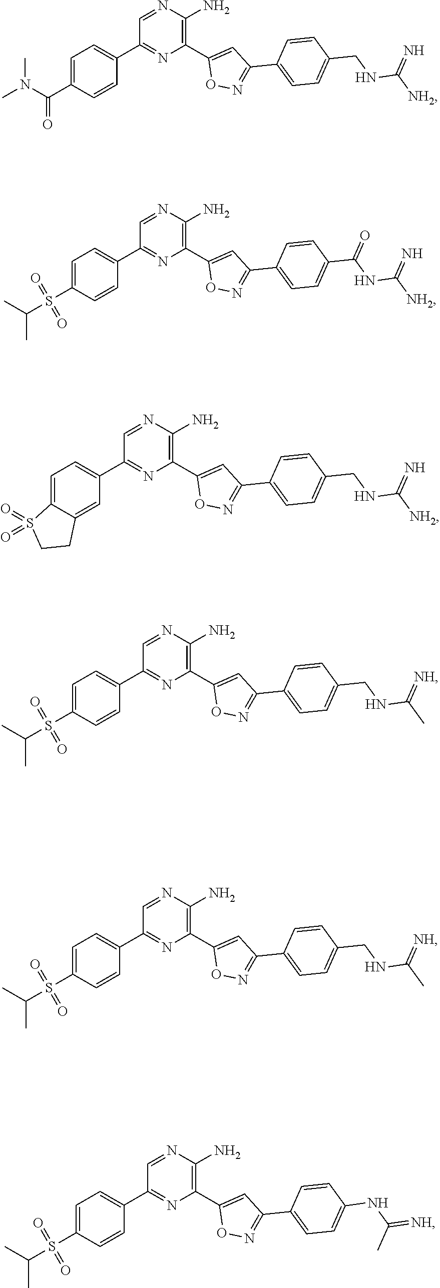

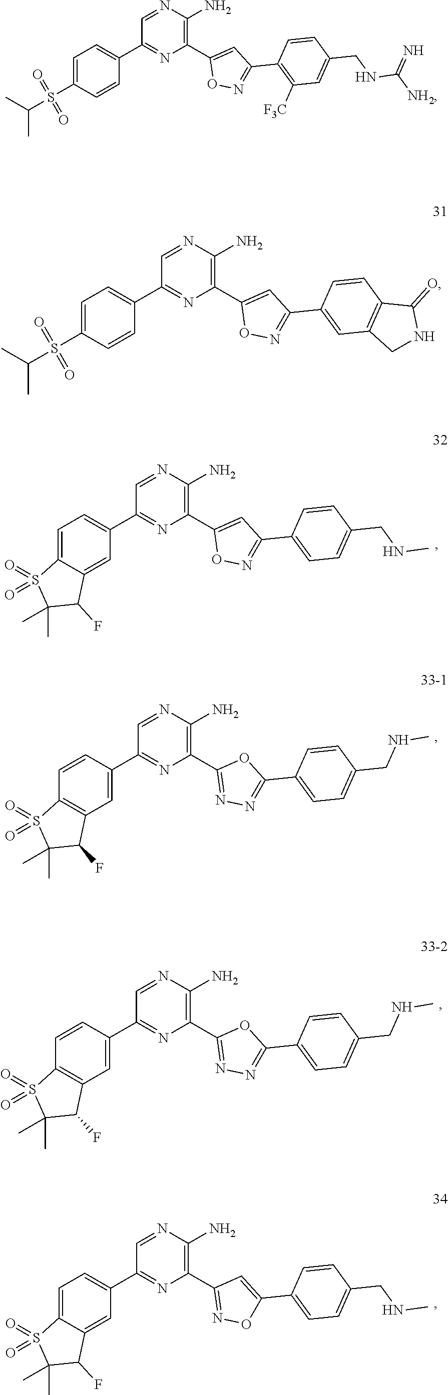 [SMILES: CC(C)S(=O)(=O)c1ccc(-c2cnc(N)c(-c3cc(-c4ccc(CNC(=N)N)cc4C(F)(F)F)no3)n2)cc1 ; CC(C)S(=O)(=O)c1ccc(-c2cnc(N)c(-c3cc(-c4ccc5c(c4)CNC5=O)no3)n2)cc1 ; CNCc1ccc(-c2cc(-c3nc(-c4ccc5c(c4)C(F)C(C)(C)S5(=O)=O)cnc3N)on2)cc1 ; CNCc1ccc(-c2nnc(-c3nc(-c4ccc5c(c4)[C@@H](F)C(C)(C)S5(=O)=O)cnc3N)o2)cc1 ; CNCc1ccc(-c2nnc(-c3nc(-c4ccc5c(c4)[C@H](F)C(C)(C)S5(=O)=O)cnc3N)o2)cc1 ; CNCc1ccc(-c2cc(-c3nc(-c4ccc5c(c4)C(F)C(C)(C)S5(=O)=O)cnc3N)no2)cc1]

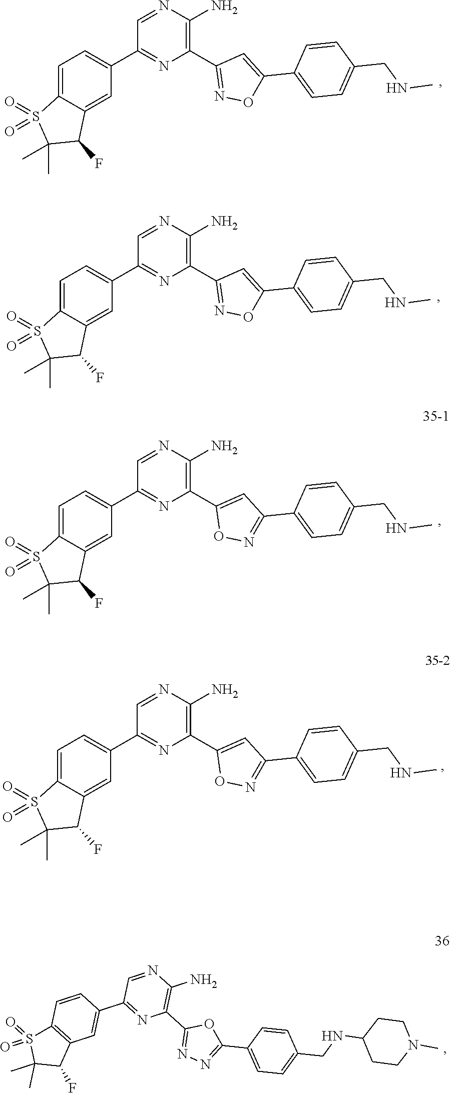 [SMILES: CNCc1ccc(-c2cc(-c3nc(-c4ccc5c(c4)[C@@H](F)C(C)(C)S5(=O)=O)cnc3N)no2)cc1 ; CNCc1ccc(-c2cc(-c3nc(-c4ccc5c(c4)[C@H](F)C(C)(C)S5(=O)=O)cnc3N)no2)cc1 ; CNCc1ccc(-c2cc(-c3nc(-c4ccc5c(c4)[C@@H](F)C(C)(C)S5(=O)=O)cnc3N)on2)cc1 ; CNCc1ccc(-c2cc(-c3nc(-c4ccc5c(c4)[C@H](F)C(C)(C)S5(=O)=O)cnc3N)on2)cc1 ; CN1CCC(NCc2ccc(-c3nnc(-c4nc(-c5ccc6c(c5)[C@H](F)C(C)(C)S6(=O)=O)cnc4N)o3)cc2)CC1]

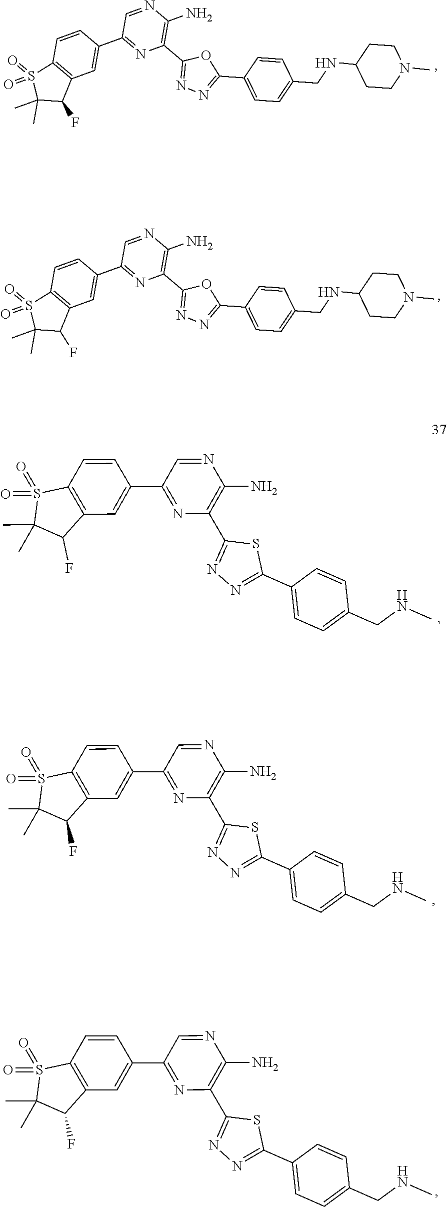

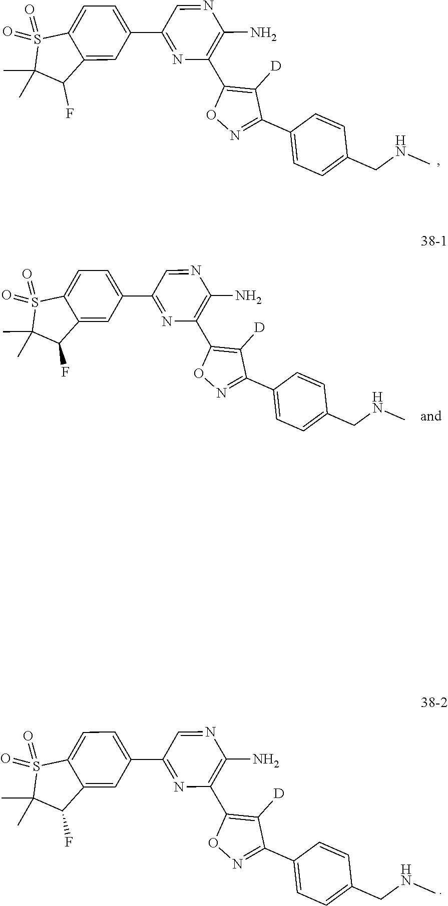

22. A compound, or an ester, isomer, solvate, prodrug or isotopically labeled compound thereof, wherein the compound is selected from:

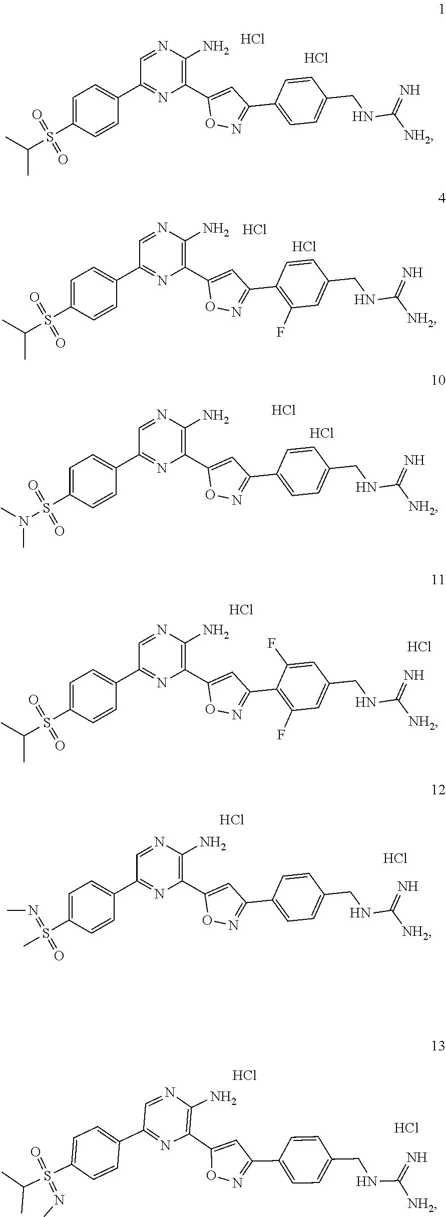

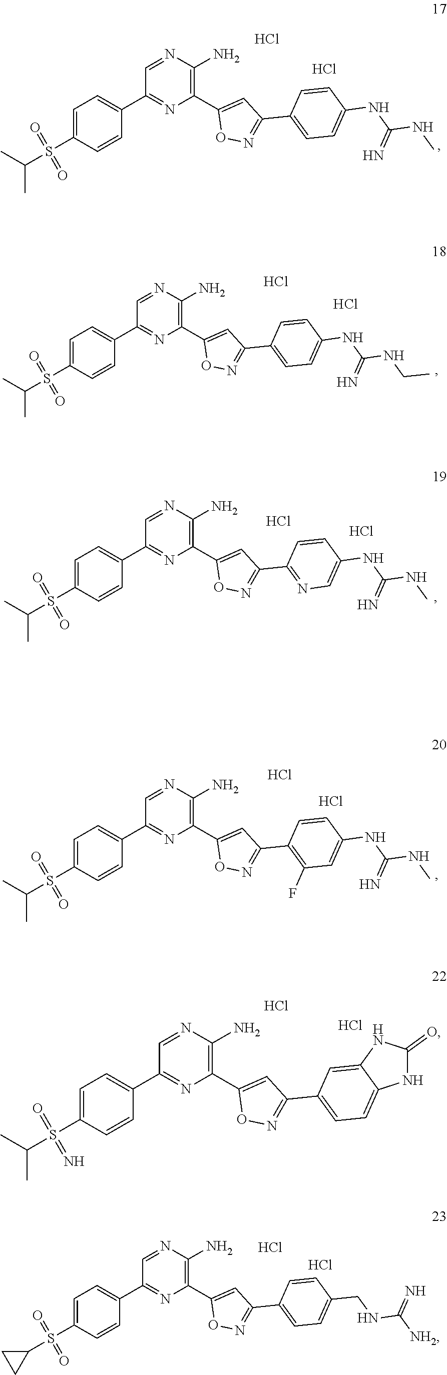

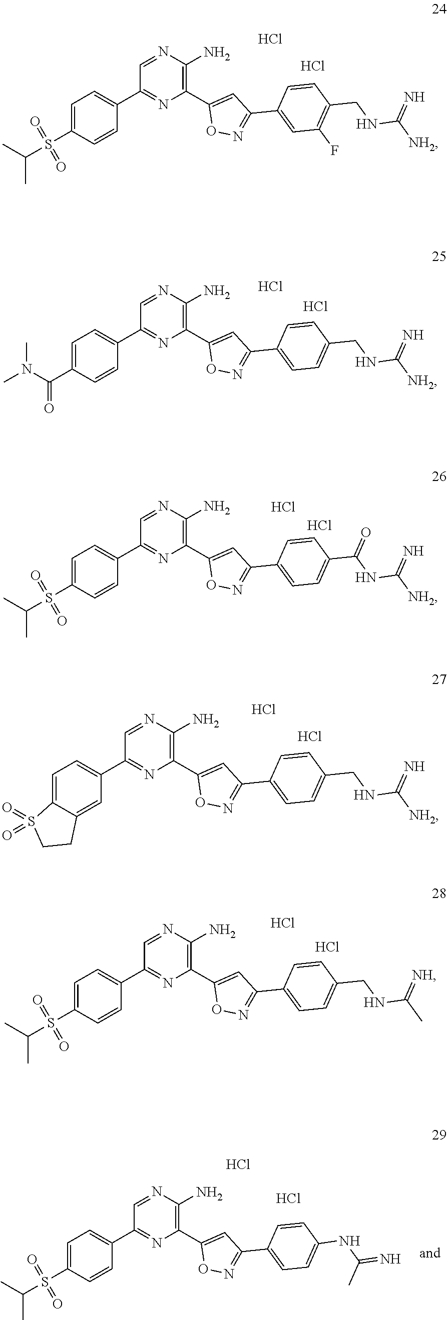

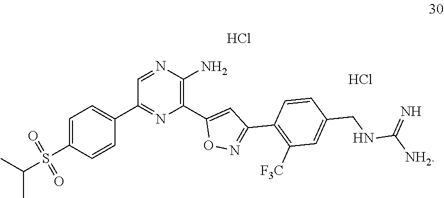 [SMILES: Cl ; CC(C)S(=O)(=O)c1ccc(-c2cnc(N)c(-c3cc(-c4ccc(CNC(=N)N)cc4C(F)(F)F)no3)n2)cc1 ; Cl]

23. The compound of claim 10, or a pharmaceutically acceptable salt, ester, isomer, solvate, prodrug or isotopically labeled compound thereof, wherein said isotope is selected from ²H, ³H, ¹³C, ¹⁴C, ¹⁵N, ¹⁷O ¹⁸O, ³¹P, ³²p ³⁵S, ¹⁸F and ³⁶Cl, preferably said isotope being ²H.

24. A pharmaceutical composition, comprising a therapeutically effective amount of the compound of formula I as claimed in claim 1, or a pharmaceutically acceptable salt, ester, isomer, solvate, prodrug or isotopically labeled compound thereof, and one or more pharmaceutically acceptable carriers, diluents or excipients.

25. (canceled)

26. A method for treating and/or preventing diseases or conditions mediated by an ATR kinase, comprising administering a therapeutically effective amount of a compound of claim 1 to a subject in need thereof.

27. The method of claim 26, wherein the diseases or conditions are diseases, disorders or conditions of excessive or abnormal cell proliferations, preferably, said diseases, disorders or conditions are selected from cancers and myeloproliferative disorders, more preferably, said diseases, disorders or conditions are selected from skin cancer, bladder cancer, ovarian cancer, breast cancer, gastric cancer, prostate cancer, colorectal cancer, lung cancer, bone cancer, brain cancer, oesophageal cancer, tongue cancer, stomach cancer, kidney cancer, cervical cancer, endometrial cancer, testicular cancer, urinary cancer, melanoma, squamous cell carcinoma, glioma, meningioma, Hodgkin's lymphoma, non-Hodgkin's lymphoma, acute lymphoblastic leukemia, chronic lymphatic leukemia, acute myelogenous leukemia, chronic myelogenous leukemia, adult T-cell leukemia lymphoma, liver cancer, bronchial cancer, multiple myeloma, basal cell tumour and sertoliomyeloma tumour; wherein said colorectal cancer is preferably colon cancer and rectal cancer; said kidney cancer is preferably parenchymal renal cancer; said glioma is preferably astrocytoma; said liver cancer is preferably hepatocellular carcinoma; said lung cancer is preferably small cell lung cancer or non-small cell lung cancer; said sarcoma is preferably selected from rhabdomyosarcoma, chondrosarcoma, leiomyosarcoma and fibrosarcoma.
